SEQUENCE LISTING (52 sequences)

SEQ ID NO: 1 - DNA, 26 bp, synthetic construct
  misc_feature 1..26: note = Oligonucleotides
  source 1..26: mol_type = other DNA
caccatgcttaagcagggcaatttct

SEQ ID NO: 2 - DNA, 20 bp, synthetic construct
  misc_feature 1..20: note = Oligonucleotides
  source 1..20: mol_type = other DNA
tcatggttccattggcctct

SEQ ID NO: 3 - DNA, 26 bp, synthetic construct
  misc_feature 1..26: note = Oligonucleotides
  source 1..26: mol_type = other DNA
caccatgggcgtgactaggaatagag

SEQ ID NO: 4 - DNA, 24 bp, synthetic construct
  misc_feature 1..24: note = Oligonucleotides
  source 1..24: mol_type = other DNA
ctaagaggagtctacttcgacctc

SEQ ID NO: 5 - DNA, 25 bp, synthetic construct
  misc_feature 1..25: note = Oligonucleotides
  source 1..25: mol_type = other DNA
caccatgggtactttttcagttctg

SEQ ID NO: 6 - DNA, 22 bp, synthetic construct
  misc_feature 1..22: note = Oligonucleotides
  source 1..22: mol_type = other DNA
ctaccgggcctctaatgacatc

SEQ ID NO: 7 - DNA, 23 bp, synthetic construct
  misc_feature 1..23: note = Oligonucleotides
  source 1..23: mol_type = other DNA
ggggtcaaagctttctacagagg

SEQ ID NO: 8 - DNA, 21 bp, synthetic construct
  misc_feature 1..21: note = Oligonucleotides
  source 1..21: mol_type = other DNA
gccaaccaaaccacagtttgc

SEQ ID NO: 9 - DNA, 24 bp, synthetic construct
  misc_feature 1..24: note = Oligonucleotides
  source 1..24: mol_type = other DNA
gcttttgtgaagaggttgccatgg

SEQ ID NO: 10 - DNA, 20 bp, synthetic construct
  misc_feature 1..20: note = Oligonucleotides
  source 1..20: mol_type = other DNA
gaccccattttgagacaccc

SEQ ID NO: 11 - DNA, 19 bp, synthetic construct
  misc_feature 1..19: note = Oligonucleotides
  source 1..19: mol_type = other DNA
cgtttcaaccggaggaagc

SEQ ID NO: 12 - DNA, 22 bp, synthetic construct
  misc_feature 1..22: note = Oligonucleotides
  source 1..22: mol_type = other DNA
ttacattcagagcccccaaagc

SEQ ID NO: 13 - DNA, 20 bp, synthetic construct
  misc_feature 1..20: note = Oligonucleotides
  source 1..20: mol_type = other DNA
catggattccgaagatcaca

SEQ ID NO: 14 - DNA, 19 bp, synthetic construct
  misc_feature 1..19: note = Oligonucleotides
  source 1..19: mol_type = other DNA
ctggccacaccaatgacaa

SEQ ID NO: 15 - DNA, 22 bp, synthetic construct
  misc_feature 1..22: note = Oligonucleotides
  source 1..22: mol_type = other DNA
aaatcagccctcccaagaaacg

SEQ ID NO: 16 - DNA, 25 bp, synthetic construct
  misc_feature 1..25: note = Oligonucleotides
  source 1..25: mol_type = other DNA
cttcacgacagtctcttctctctgc

SEQ ID NO: 17 - DNA, 20 bp, synthetic construct
  misc_feature 1..20: note = Oligonucleotides
  source 1..20: mol_type = other DNA
aggctcatcgattgtcagca

SEQ ID NO: 18 - DNA, 20 bp, synthetic construct
  misc_feature 1..20: note = Oligonucleotides
  source 1..20: mol_type = other DNA
ctgcactacgattctcggct

SEQ ID NO: 19 - DNA, 20 bp, synthetic construct
  misc_feature 1..20: note = Oligonucleotides
  source 1..20: mol_type = other DNA
gttgtcggtgatggttgtgc

SEQ ID NO: 20 - DNA, 20 bp, synthetic construct
  misc_feature 1..20: note = Oligonucleotides
  source 1..20: mol_type = other DNA
ccggatcttgccggaatctt

SEQ ID NO: 21 - DNA, 20 bp, synthetic construct
  misc_feature 1..20: note = Oligonucleotides
  source 1..20: mol_type = other DNA
aggccatcaaggaggaatct

SEQ ID NO: 22 - DNA, 23 bp, synthetic construct
  misc_feature 1..23: note = Oligonucleotides
  source 1..23: mol_type = other DNA
gaaaatgcttgacctgttgtcac

SEQ ID NO: 23 - protein, 120 aa, synthetic construct
  VARIANT 1..120
  VARIANT 2..4
  VARIANT 11..15
  VARIANT 22..24
  VARIANT 35..44
  VARIANT 46..48
  VARIANT 51..54
  VARIANT 56
  VARIANT 64
  VARIANT 67..79
  VARIANT 81
  VARIANT 90
  VARIANT 93..106
  VARIANT 109
  VARIANT 118..119
CXXXLIVMFYXXXXXLIVMFYXXXDENQLIVMFYXXXXXXXXXXCXXXCTXXXXCXLIVMFYFXFYXXXXXXXXXXXXXCXLIVMFYRKXSTXXXXXXXXXXXXXXSGXSTLIVMFYXXC

SEQ ID NO: 24 - protein, 85 aa, Salix purpurea
CERTQNDGQVPKEDGFSKLERVKVPDFAEWSSSITEQKCRDDCLNNCSCIAYAYYSGIYCMLWRGNLTDIKKFSSGGADLYIRLA

SEQ ID NO: 25 - protein, 82 aa, Salix purpurea
CSDKVGFLKYTGMKLPDTSSSWYDKSISLKECEGLCLKNCSCTAYANLDIRNGGSGCLIWFGDLIDTRRSAGDGQDLYVRMN

SEQ ID NO: 26 - protein, 79 aa, Salix purpurea
DSDYNMDTYEHTFLYGIYPPNEAIIIASLQQCREVCMQDPGCTAATFTNDGTSQCRMKTSPYFSGHQNPSLSSISFVKT

SEQ ID NO: 27 - protein, 83 aa, Salix purpurea
CDHGEGFVKIENYCLPDTSSAAWVDKKKSRADCELECKRNCSCSAFSIIRIPGKGEGCLAWYRELVDIRYGRSESDELYVRVD

SEQ ID NO: 28 - protein, 81 aa, Salix purpurea
CKMNTSMMVMRQTFLYGLYPPQDVDIMLSEKACKEYCSNDTNCIAATSKNDGSGICTIKRTSFITGYGNPSVSATSFLKVC

SEQ ID NO: 29 - protein, 87 aa, Arabidopsis thaliana
CQAKSSMKTQGKDTDIFYRMTDVKTPDLHQFASFLNAEQCYQGCLGNCSCTAFAYISGIGCLVWNGELADTVQFLSSGEFLFIRLAS

SEQ ID NO: 30 - protein, 85 aa, synthetic construct
  VARIANT 1..85
CQNKKSTGKKDGFLKMSILTLPENSKAYQKVSVARCRLYCMKNCYCVAYAYNSSGCFLWEGDLINLKQSEIAAGRAGAEIYIRLA

SEQ ID NO: 31 - protein, 81 aa, synthetic construct
  VARIANT 1..81
CSGDGFQKLSGLKLPETKTSWFNTSMNLEECKKKCIKNCSCTAYSNLDIRNGGSGCLLWFGDLIDIRVIAVNEQDVYIRMA

SEQ ID NO: 32 - protein, 89 aa, Homo sapiens
NTIHEFKKSAKTTLIKIDPALKIKTKKVNTADQCANRCTRNKGLPFTCKAFVFDKARKQCLWFPFNSMSSGVKKEFGHEFDLYENKDYI

SEQ ID NO: 33 - protein, 77 aa, Homo sapiens
QVLRGTELQHLLHAVVPGPWQEDVADAEECAGRCGPLMDCWAFHYNVSSHGCQLLPWTQHSPHSRLWHSGRCDLFQE

SEQ ID NO: 34 - protein, 77 aa, Homo sapiens; identical to SEQ ID NO: 33

SEQ ID NO: 35 - protein, 77 aa, Homo sapiens; identical to SEQ ID NO: 33

SEQ ID NO: 36 - protein, 77 aa, Homo sapiens; identical to SEQ ID NO: 33

SEQ ID NO: 37 - protein, 77 aa, Homo sapiens; identical to SEQ ID NO: 33

SEQ ID NO: 38 - protein, 77 aa, Homo sapiens; identical to SEQ ID NO: 33

SEQ ID NO: 39 - protein, 77 aa, Homo sapiens; identical to SEQ ID NO: 33

SEQ ID NO: 40 - protein, 77 aa, Homo sapiens; identical to SEQ ID NO: 33

SEQ ID NO: 41 - protein, 77 aa, Homo sapiens; identical to SEQ ID NO: 33

SEQ ID NO: 42 - protein, 77 aa, Homo sapiens; identical to SEQ ID NO: 33

SEQ ID NO: 43 - protein, 77 aa, Homo sapiens; identical to SEQ ID NO: 33

SEQ ID NO: 44 - protein, 77 aa, Homo sapiens; identical to SEQ ID NO: 33

SEQ ID NO: 45 - protein, 77 aa, Homo sapiens; identical to SEQ ID NO: 33

SEQ ID NO: 46 - protein, 77 aa, Homo sapiens; identical to SEQ ID NO: 33

SEQ ID NO: 47 - DNA, 4023 bp, Populus trichocarpa
  source 1..4023: mol_type = other DNA
atgtgtgttattacaaagccaagtttctggttctttgtgctgttgttgctattcgtttcccactggaattgcttctccattgaaggtgatacccttttgattggccagtctctctctgcaagccagacactgatatctcaaaatggcatttttgaactcggtttcttcaagccaggcgcttctttaaacatttaccttggaatttggtataagaactccgcagataagatgattgtttgggtggcaaacagggagagccctttaaacaaccctgcttcatcgaagcttgaattatcaccggatggcattcttgtcctactgacaaatttcaccaaaacagtttggtcaacagctcttgcatcttcagtgccgaataacagtacagcacaagcagcacttcttgataatggaaactttgtcattaaagatggctcaaacccatccgctatttactggcagagttttgacaatccaactgatacattgctacctggtggaaagcttggaatcaacaagcacactgggaaagtgcagaagcttatttcctggaaaaacccagaagatcctgcaccaggtatgttctcgattacgatggaccccaatggcagtagtcagatttttatagagtggaacaaggtcacacatgtattggagcagtggggtttggaatggacaaagattttccatggttcctgagatgaatttgaactattatttcaattatagttatatatcgaatgaaaatgaaagctatttcaccttttctgtgtacaatgctgaaatgctctcaagatacgtgattgatgtttcaggacaaatcaaacaattaaattggttagcaggtgttaggaattggtcagaattctgggcccagcccagtgaccaagctggtgtttatggtttatgcggggtttttggagtctttcatggaaactcatcgagctcttgtgaatgcttgaaaggttttgaaccattagtacaaaatgattggtcaagcggttgtgttaggaaatctcctttgcagtgtcaaaataagaaaagtactgggaaaaaagatgggttcctgaagatgtcgattctgacattaccagaaaattcaaaagcatatcaaaaagtgagtgttgcaagatgtagattgtattgcatgaaaaattgttattgcgtggcttatgcctataatagcagtgggtgttttttatgggaaggagatcttataaacttaaaacagtcagagattgctgctgggagggctggagcagaaatttacatcagacttgctgcttctgagcttgaacctcagattggtagtggcagtatccgaacaggtaaggtagaaaggttttatatatatagttataaatgatccaatagatgttgagcaatgatttcttacaaaataacagcatagtctttccagataaaatggtggatgaaatgtttcctgtagagaagtgtggaagacataacctaacttcccatatctttctatcaaaataaaaacatcttaattcgttaaaagtaaatttcttggtttatgatttggttaatgtttgatctatgcttacaagtaggcaaatatcaaatggaaaatacggacaaccttggctgtggctgttccagtaactctgattaccttaggcctcttcatatacttcagctgtctgcgcaagggaaagctcatacacaaaggtacatcctgttcttgctttaatttctgctgtctcctggtaggctttaattaaccctcaagttactggtgactagaaaaaaccctcctttgtaattgattatgtcttaggtatgcagatggtaaggttcattttgatggagatagagggacagaaatgttttgttttgcataaaatattttaccgaaataagtttttattttttatttatttatatatgtttgtttcttgtaaaatatctgacaagaagagtgatcaatataaattattttatagttaaccttcatattagttcatttattaaaaatatttctcatctcttaaaattttataaaatattttatgaaaaataaaacaatcacaatatcttacaacaaatttatccatataaaatgtttaggaggtaaaatattttatgtcaaacaaacaccaaagtttcttatttatgaaaaaaactgacatttagaataccttttaacgtccgaattgtggttatgctaacctgaagttgttaccttttttgcttgatattatcagaatagctggatattgttcaacaaagtgaatgtgttcaatcatttttatttttatatttttcttgaaacagcgaaggaacgtgcaagtcacaatttattgcgttttaatttcgatgccgatcctaactcaactaccaatgaatctagctctgttgacaatcggaagaaaagatggagtaaaaatatagaatttccattattcagttatgagagcgtatcagtggcaactggacagttctcagataagcttggagagggaggattcggacctgtttataaggtaaatttacgttcaaatgagaaatgctgattttcctaagaaaaatgttaaaatagttcgaatgaactttcagaatgacttttggttcatttaacagggcaaattacccacgggactggaaatagcagtgaagaggctttcagaaagatctgggcaggggcttgaggagttcagaaatgagacaactctaatcgccaaactccagcaccggaatcttgtcaggctactgggttcctgtattgaatgggatgagaaaatgctaatctatgagtacatgccaaataaaagcttggatttctttctctatggtcagcatttagttcttttctaatttcattaacttttcaaatgcttcaagaaatttataacagatttgtgtataaaatataaacagatgcaaacagaggacaaatcttagattggggtgcacggattcggataatcgaaggaattgctcaaggccttctgtatctacatagatactcgcggttacgaatcattcacagggatttaaagcctagcaacattctattagacagtgagatgaatccaaaaatatccgatttcgggatggctcgaattttcggaggcaacgaaactcaagcacacaccaacaggatcgttggaacatagtaagtttcttaaattctgttttccaggacatgctacttaacatgatctgcgctgaccttttcaaatgttagtggctatatgtcccctgaatatgctatggagggtctcttctcaataaaatctgatgtgtttagcttcggggtgctggtacttgagattgtcagcggcaagaagaacactagtttctaccacagcgacaccctccatcttcttggacatgtggggagctttcatacaccctcttttctttttattcctccttgaattgtatttgaatactttcctagtctccacactaatggtgcagcatttgcgttgttatacaatagacatggaagttatggaattctaataaagctttggacttgatggatccaatcctgggagatcctccttcaactgctacgctgttgagatacataaacatagggcttctttgtgtccaggaaagtcctgctgatcggcctacaatgtctgatgttatatccatgattgcaaacgaacacgtagctctcccagaaccaaagcaacctgcttttgttgcatgcagaaacatggcagaacaaggaccattgatgagctcttctggggtaccttccgcgaataatgtgacaataacagcgatagatgggagatagttttttcatcaagaggtctaaaggacatgaagctacttcaatatcagagcgaggaagctatgagtatggagcttagatcaagcttatttgtataagttctccgatttgaatcgtacaatagtttgtttcagtttttcctattattctcgttgtattgaaacatgaacaaaatcaaatcaagtattcataaaacacatttaattcagatcttaaatatattaaaaactcattttccttttagtacaaat

SEQ ID NO: 48 - protein, 724 aa, Populus trichocarpa
MCVITKPSFWFFVLLLLFVSHWNCFSIEGDTLLIGQSLSASQTLISQNGIFELGFFKPGASLNIYLGIWYKNSADKMIVWVANRESPLNNPASSKLELSPDGILVLLTNFTKTVWSTALASSVPNNSTAQAALLDNGNFVIKDGSNPSAIYWQSFDNPTDTLLPGGKLGINKHTGKVQKLISWKNPEDPAPGMFSITMDPNGSSQIFIEWNKMNLNYYFNYSYISNENESYFTFSVYNAEMLSRYVIDVSGQIKQLNWLAGVRNWSEFWAQPSDQAGVYGLCGVFGVFHGNSSSSCECLKGFEPLVQNDWSSGCVRKSPLQCQNKKSTGKKDGFLKMSILTLPENSKAYQKVSVARCRLYCMKNCYCVAYAYNSSGCFLWEGDLINLKQSEIAAGRAGAEIYIRLAASELEPQIGSGSIRTGKGKLPTGLEIAVKRLSERSGQGLEEFRNETTLIAKLQHRNLVRLLGSCIEWDEKMLIYEYMPNKSLDFFLYDANRGQILDWGARIRIIEGIAQGLLYLHRYSRLRIIHRDLKPSNILLDSEMNPKISDFGMARIFGGNETQAHTNRIVGTYGYMSPEYAMEGLFSIKSDVFSFGVLVLEIVSGKKNTSFYHSDTLHLLGHTWKLWNSNKALDLMDPILGDPPSTATLLRYINIGLLCVQESPADRPTMSDVISMIANEHVALPEPKQPAFVACRNMAEQGPLMSSSGVPSANNVTITAIDGR

SEQ ID NO: 49 - protein, 728 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 50 - protein, 121 aa, synthetic construct
  VARIANT 1..121
  VARIANT 2..4
  VARIANT 11..15
  VARIANT 22..24
  VARIANT 35..44
  VARIANT 46..48
  VARIANT 51..54
  VARIANT 56
  VARIANT 64
  VARIANT 67..79
  VARIANT 81
  VARIANT 90
  VARIANT 93..107
  VARIANT 110
  VARIANT 119..120
CXXXLIVMFYXXXXXLIVMFYXXXDENQLIVMFYXXXXXXXXXXCXXXCTXXXXCXLIVMFYFXFYXXXXXXXXXXXXXCXLIVMFYRKXSTXXXXXXXXXXXXXXXSGXSTLIVMFYXXC

SEQ ID NO: 51 - protein, 121 aa, synthetic construct
  VARIANT 1..121
  VARIANT 2..4
  VARIANT 11..15
  VARIANT 22..24
  VARIANT 35..44
  VARIANT 46..48
  VARIANT 51..54
  VARIANT 56
  VARIANT 64
  VARIANT 67..80
  VARIANT 82
  VARIANT 91
  VARIANT 94..107
  VARIANT 110
  VARIANT 119..120
CXXXLIVMFYXXXXXLIVMFYXXXDENQLIVMFYXXXXXXXXXXCXXXCTXXXXCXLIVMFYFXFYXXXXXXXXXXXXXXCXLIVMFYRKXSTXXXXXXXXXXXXXXSGXSTLIVMFYXXC

SEQ ID NO: 52 - protein, 122 aa, synthetic construct
  VARIANT 1..122
  VARIANT 2..4
  VARIANT 11..15
  VARIANT 22..24
  VARIANT 35..44
  VARIANT 46..48
  VARIANT 51..54
  VARIANT 56
  VARIANT 64
  VARIANT 67..80
  VARIANT 82
  VARIANT 91
  VARIANT 94..108
  VARIANT 111
  VARIANT 120..121
CXXXLIVMFYXXXXXLIVMFYXXXDENQLIVMFYXXXXXXXXXXCXXXCTXXXXCXLIVMFYFXFYXXXXXXXXXXXXXXCXLIVMFYRKXSTXXXXXXXXXXXXXXXSGXSTLIVMFYXXC

(19) United States
(12) Patent Application Publication
(10) Pub. No.: US 2025/0164462 A1
(43) Pub. Date: May 22, 2025

(54) METHODS FOR IMMUNOREGULATION BY MODULATING PLASMINOGEN-APPLE-NEMATODE (PAN) DOMAIN-CONTAINING PROTEINS

(71) Applicant: UT-Battelle, LLC, Oak Ridge, TN (US)

(72) Inventors: Wellington Muchero; Carly M. Shanks, New York, NY (US); Debjani Pal, Knoxville, TN (US); Kuntal De, Knoxville, TN (US)

(73) Assignee: UT-Battelle, LLC, Oak Ridge, TN (US)

(21) Appl. No.: 18/974,906

(22) Filed: Dec. 10, 2024

Related U.S. Application Data
(62) Division of application No. 17/012,139, filed on Sep. 4, 2020, now Pat. No. 12,203,927.
(60) Provisional application No. 62/896,605, filed on Sep. 6, 2019.

(51) Int. Cl. G01N 33/50; A61P 15/08; A61P 35/00; C12N 9/22; C12N 15/11; C12N 15/113; C12N 15/82; G01N 33/68

(52) U.S. Cl. CPC G01N 33/5041; A61P 15/08; A61P 35/00; C12N 9/22; C12N 15/11; C12N 15/113; C12N 15/8279; G01N 33/6818; C12N 2310/11; C12N 2310/20; G01N 2500/00

(57) ABSTRACT

The present disclosure based on the inventors' recognition that PAN domain containing proteins play important immune regulating functions. Disclosed herein are methods for modulating immune responses in plants and animals, improving in vitro fertilization efficiency, and inhibiting human cell division and cellular migration in cancer cells. Also disclosed herein are genetically modified plants that are resistant to pathogenic infections.

### CROSS REFERENCE TO RELATED APPLICATIONS

The present application is a divisional of U.S. patent application Ser. No. 17/012,139 filed Sep. 4, 2020, which claims benefit of U.S. Provisional Application No. 62/896,605, filed Sep. 6, 2019, the entire contents of which are incorporated herein by reference.

### STATEMENT REGARDING FEDERALLY SPONSORED RESEARCH OR DEVELOPMENT

This disclosure was made with government support under a research project supported by Prime Contract No. DE-AC05-000R22725 awarded by the U.S. Department of Energy. The government has certain rights in this invention.

### INCORPORATION BY REFERENCE OF SEQUENCE LISTING

The Sequence Listing in an XML file, named as 38529A_4200_4_SubstituteSequenceListing.xml of 73,728 bytes, created on Dec. 31, 2024, and submitted to the United States Patent and Trademark Office via Patent Center, is incorporated herein by reference.

### BACKGROUND

Immunoregulation (e.g. immunosuppression) is an essential phenomenon governing fertilization during sexual reproduction, cell growth and proliferation as well as recruitment of beneficial symbionts. However, the same phenomenon predisposes host organisms to uncontrolled cell growth and infection by parasites, pathogens, and viruses. Consequently, understanding the molecular basis of immunoregulation (e.g., immunosuppression or immunostimulation) has broad implications.

The Plasminogen-Apple-Nematode (PAN) domain (IPR003609) was first characterized by Tordai et al., (FEBS Letters, 461(1-2), 63-67, (1999)) in which they noted that the domain was shared by the plasminogen/hepatocyte growth factor protein family, the prekallikrein/coagulation factor XI protein family and nematode proteins. The domain possesses the characteristic 6-cysteine residues in its core that are highly conserved. These cysteine residues coordinate three disulfide bridges to form a hair-pin loop structure.

Plasma kallikrein (EC 3.4.21.34) and coagulation factor XI (EC 3.4.21.27) are two related plasma serine proteases activated by factor XIIA and which share the same domain topology: an N-terminal region that contains four tandem repeats of about 90 amino acids and a C-terminal catalytic domain. The 90 amino-acid repeated domain contains 6 conserved cysteines. Three disulfide bonds link the first and sixth, second and fifth, and third and fourth cysteines. The domain can be drawn in the shape of an apple and has been accordingly called the “apple domain”.

The apple domains of plasma prekallikrein are known to mediate its binding to high molecular weight kininogen, the apple domains of factor XI bind to factor XIIa, platelets, kininogen, factor IX and heparin.

The apple domains display some sequence similarity with the N domain of plasminogen/hepatocyte growth factor (HGF) and to some nematode and protozoan proteins. They all belong to the same domain superfamily that have been called the PAN module. The N domain of hepatocyte growth factor binds to the c-Met receptor and to the heparin molecule. The structure of the PAN module of HGF has been solved. It contains a characteristic hairpin-loop structure stabilised by two disulfide bridges, Cys-1 and 6 are not conserved in HGF PAN modules.

Apart from the cysteines, there are a number of other conserved positions in the apple domain. The PAN domain is thought to mediate protein-protein or carbohydrate-protein interactions and facilitate receptor dimerization.

Immunoregulation has been well recorded in plants. Plasma membrane bound pattern recognition receptors (PRR) are integral part of surveillance system against pathogens. Among the PRR which has a receptor kinase domain are known as receptor kinases. These receptor kinases are further classified into specific sub-types including Lectin-rich receptor kinases (L-type RLKs). L-type RLKs are well known for their role in triggering plant defense responses upon pathogen invasion. In contrast, it has been shown that a different class of receptor kinases, the PAN domain-containing D-mannose lectin binding domain receptor-like protein kinases (G-type RLKs), function to suppress defense response in plants. G-type RLKs were shown to function as negative regulators of defense signaling during pathogenesis by the fungal pathogen Sphaerulina musiva, parasitism of Arabidopsis by nematodes, and conversion of Arabidopsis into a host of the fungal symbiont Laccaria bicolor. Moreover, PAN domain carrying S-locus kinases, reported to mediate self-incompatibility during pollination, fall under the same class of G-LecRLKs. However, the specific role of the PAN domain in all these processes has not been fully described.

In animal systems, PAN domain proteins function in cell growth and proliferation. One example is the Hepatocyte growth factor (HGF) which has a similar domain structure with N terminal PAN domain, multiple kringle domain and a serine protease domain. HGF consists of characteristic heterodimer connected by a disulfide bond. HGF acts as an important paracrine mediator of growth, invasion and angiogenesis in several cancers. All biological impacts of HGF are triggered by stimulating its cell surface receptor. Both high and low affinity receptors have been identified for HGF. The high affinity receptor c-MET proto-oncogene is a receptor tyrosine kinase that is turned on by HGF ligand binding leading to a series of phosphorylation events including STAT3 phosphorylation, eventually turning on the HGF/c-MET signaling cascade. Hyperactivation of HGF/c-MET for instance, has been reported in lung cancer, colorectal cancer, glioblastoma, acute myeloid lymphoma among others with higher mortality rate. Recent evidence suggests that HGF can confer significant resistance to targeted cancer drugs including multiple kinase inhibitors interfering with current anti-cancer effort.

However, HGF/c-MET signaling is a key step for embryogenesis and tissue regeneration in the adult life which suggests a strict regulatory mechanism is important to determine the fate of the cells from a healthy one to tumorigenic. Recently, HGF/MET pathway emerged as a key target for anti-cancer therapy, although the major problem associated with small molecule inhibitors of MET and anti HGF-antibodies is that most of them are unstable and expensive. Although the interaction between MET and HGF were well studied, it was not clear which amino acids in α-chain of HGF serve as the binding region for cMET receptor.

### SUMMARY OF THE DISCLOSURE

In one aspect the disclosure is directed to a method for screening for an immunoregulatory protein comprising assessing the sequence of a candidate protein, and determining that the candidate protein is an immunoregulatory protein when at least one Plasminogen Apple Nematode (PAN) domain is found in the sequence.

In some embodiments, the PAN domain has a consensus sequence as shown in SEQ ID NO: 23.

Another aspect of the disclosure is directed to a method for screening for a therapeutic compound targeting a PAN domain in a protein, comprising contacting the protein comprising the PAN domain with a candidate therapeutic compound, and assessing whether the compound binds to the PAN domain.

In some embodiments, the PAN domain-containing protein is selected from proteins listed in List 1.

Another aspect of the disclosure is directed to a method for screening for a MET signaling inhibitor, comprising expressing a c-MET receptor on the surface of a cell; incubating the cell with Hepatocyte Growth Factor (HGF) in the presence of a candidate inhibitor of PAN domain interactions; and determining that the candidate inhibitor is an inhibitor of c-MET signaling if the interaction with c-MET and HGF is inhibited.

In some embodiments, the candidate inhibitor of PAN domain interactions is a peptide comprising a consensus sequence as shown in SEQ ID NO: 23. In some embodiments, the candidate inhibitor of PAN domain interactions is a small molecule.

Another aspect of the disclosure is directed to a method for enhancing immune response to a pathogen in a plant comprising inactivating a gene encoding a PAN domain-containing protein in the plant. In some embodiments, the inactivating is achieved by introducing a non-silent mutation in the gene at a position encoding a conserved cysteine residue of the PAN domain. In some embodiments, the PAN domain-containing protein is selected from plant G-type receptor-like protein kinases. In some embodiments, the conserved cysteine residue is selected from a residue equivalent to a cysteine at position 1, 32, 36, 40, 42, or 57 of SEQ ID NO: 25.

In some embodiments, the non-silent mutation is a nonconservative mutation. In some embodiments, the nonconservative mutation is a cysteine to alanine mutation.

In some embodiments, the inactivating of the gene encoding a PAN domain-containing protein is achieved by introducing an exogenous nucleic acid inhibitor of the gene to the plant. In some embodiments, the nucleic acid inhibitor is selected from the group consisting of an antisense RNA, a small interfering RNA, an RNAi, a microRNA, an artificial microRNA, and a ribozyme.

In some embodiments, the inactivating of the selected gene is achieved by genome editing, which is achieved by a method selected from the group consisting of CRISPR/Cas system, Cre/Lox system, TALEN system, ZFNs system and homologous recombination.

In some embodiments, the CRISPR-mediated genome editing comprises introducing into the plant a first nucleic acid encoding a Cas9 nuclease, a second nucleic acid comprising a guide RNA (gRNA), wherein said gRNA is specific to the gene encoding a PAN domain containing protein.

In some embodiments, the CRISPR-mediated genome editing comprises introducing into the plant a first nucleic acid encoding a Cas9 nuclease, a second nucleic acid comprising a guide RNA (gRNA), wherein said gRNA is specific to the gene encoding a PAN domain containing protein gene, and a third nucleic acid comprising a template for homologous recombination.

In some embodiments, the pathogen is selected from a bacterium, a virus or a fungus.

Another aspect of the disclosure I directed to a genetically engineered plant, wherein an endogenous gene encoding a PAN domain-containing protein has been inactivated as a result of a genetic modification.

In some embodiments, the inactivating is achieved by introducing a non-silent mutation in the gene at a residue encoding a conserved cysteine residue of the PAN domain.

In some embodiments, the PAN domain-containing protein is selected from G-type receptor-like protein kinases. In some embodiments, the conserved cysteine residue is selected from a residue equivalent to a cysteine at position 1, 32, 36, 40, 42, or 57 of SEQ ID NO: 25.

In some embodiments, the non-silent mutation is a nonconservative mutation. In some embodiments, the nonconservative mutation is a cysteine to alanine mutation.

Another aspect of the disclosure is directed to a method for preventing cell division of a human cell comprising inactivating a gene encoding a PAN domain containing protein in the human cell. In some embodiments, the inactivating is achieved by introducing a non-silent mutation in the gene at a residue encoding a conserved cysteine residue of the PAN domain.

In some embodiments, the PAN domain containing protein gene is Hepatocyte Growth Factor (HGF).

In some embodiments, the conserved cysteine residue is selected from a residue equivalent to a cysteine at position 34, 38, 48, or 60 of SEQ ID NO: 32.

In some embodiments, the non-silent mutation is a nonconservative mutation. In some embodiments, the nonconservative mutation is a cysteine to alanine mutation.

In some embodiments, the inactivating of the gene encoding a PAN domain-containing protein is achieved by introducing an exogenous nucleic acid inhibitor of the gene to the cancer cell.

In some embodiments, the nucleic acid inhibitor is selected from the group consisting of an antisense RNA, a small interfering RNA, an RNAi, a microRNA, an artificial microRNA, and a ribozyme.

In some embodiments, the inactivating of the gene encoding a PAN domain-containing protein is achieved by genome editing, which is achieved by a method selected from the group consisting of CRISPR/Cas system, Cre/Lox system, TALEN system, ZFNs system and homologous recombination.

In some embodiments, the CRISPR-mediated genome editing comprises introducing into the cancer cell a first nucleic acid encoding a Cas9 nuclease, and a second nucleic acid comprising a guide RNA (gRNA), wherein said gRNA is specific to the gene encoding a PAN domain containing protein.

In some embodiments, the CRISPR-mediated genome editing comprises introducing into the cancer cell a first nucleic acid encoding a Cas9 nuclease, a second nucleic acid comprising a guide RNA (gRNA), wherein said gRNA is specific to the gene encoding a PAN domain containing protein, and a third nucleic acid comprising a template for homologous recombination.

Another aspect of the disclosure is directed to a method for improving in vitro fertilization (IVF) efficiency in a non-human subject suffering from infertility comprising determining the sequence of a gene that encodes a PAN domain-containing protein, and obtaining an ovum or a sperm from the subject when a mutation is found in a conserved cysteine of the PAN domain of the PAN domain-containing protein, correcting the mutation in the PAN domain-containing protein in the ovum or the sperm.

In some embodiments, the PAN domain-containing protein gene is a Zona Pellicuda(ZP) protein.

In some embodiments, wherein the conserved cysteine residue is selected from a residue equivalent to a cysteine residue in SEQ ID NO: 23.

In some embodiments, the correcting of the PAN domain is achieved by genome editing, which is achieved by a method selected from the group consisting of CRISPR/Cas system, Cre/Lox system, TALEN system, ZFNs system and homologous recombination.

In some embodiments, the CRISPR-mediated genome editing comprises introducing into the ovum or the sperm a first nucleic acid encoding a Cas9 nuclease, a second nucleic acid comprising a guide RNA (gRNA), wherein said gRNA is specific to the gene encoding a PAN domain-containing protein, and a third nucleic acid comprising a template for homologous recombination that corrects the mutated residue to cysteine.

In some embodiments, the ovum or the sperm is from a domesticated animal.

Another aspect of the disclosure is directed to a method for preventing pregnancy or implantation of a fetus in a non-human subject comprising inactivating a gene encoding a PAN domain-containing protein in an ovum or a sperm.

In some embodiments, the inactivating is achieved by introducing a non-silent mutation in the gene at a residue encoding a conserved cysteine residue of the PAN domain.

In some embodiments, the PAN domain containing protein gene is a Zona Pellicuda protein.

In some embodiments, the conserved cysteine residue is selected from a residue equivalent to a cysteine a cysteine residue in SEQ ID NO: 23.

In some embodiments, the non-silent mutation is a nonconservative mutation. In some embodiments, the nonconservative mutation is a cysteine to alanine mutation.

In some embodiments, the inactivating of the gene encoding a PAN domain-containing protein is achieved by introducing an exogenous nucleic acid inhibitor of the gene to the ovum or the sperm. In some embodiments, the nucleic acid inhibitor is selected from the group consisting of an antisense RNA, a small interfering RNA, an RNAi, a microRNA, an artificial microRNA, and a ribozyme.

In some embodiments, the inactivating of the gene encoding a PAN domain-containing protein is achieved by genome editing, which is achieved by a method selected from the group consisting of CRISPR/Cas system, Cre/Lox system, TALEN system, ZFNs system and homologous recombination.

In some embodiments, the CRISPR-mediated genome editing comprises introducing into the ovum or the sperm a first nucleic acid encoding a Cas9 nuclease, a second nucleic acid comprising a guide RNA (gRNA), wherein said gRNA is specific to the gene encoding a PAN domain-containing protein.

In some embodiments, the CRISPR-mediated genome editing comprises introducing into the ovum or the sperm a first nucleic acid encoding a Cas9 nuclease, a second nucleic acid comprising a guide RNA (gRNA), wherein said gRNA is specific to the selected gene, and a third nucleic acid comprising a template for homologous recombination.

In some embodiments, the ovum or the sperm is from a domesticated animal.

Another aspect of the disclosure is directed to amethod for screening for an oncoprotein protein in a mammalian cell comprising assessing the sequence of a candidate protein, and determining that the candidate protein is an oncoprotein when at least one Plasminogen Apple Nematode (PAN) domain is found in the sequence.

In some embodiments, the PAN domain has a consensus sequence as shown in SEQ ID NO: 23.

Another aspect of the disclosure is directed to a method for inactivating a PAN domain-containing protein comprising introducing a non-silent mutation in the protein at a residue encoding a conserved cysteine residue of the PAN domain.

In some embodiments, the PAN domain-containing protein is selected from proteins listed in List 1.

In some embodiments, the conserved cysteine residue is selected from a residue equivalent to a cysteine residue in SEQ ID NO: 23.

### BRIEF DESCRIPTION OF THE DRAWINGS

The patent or application file contains at least one drawing executed in color.

Copies of this patent or patent application publication with color drawing(s) will be provided by the Office upon request and payment of the necessary fee.

FIG. 1. Amino acid conservation within different G-LecRK PAN domains and naturally occurring variation in non-immunosuppressive Salix orthologs SapurV1A.0037s0270.1 and SapurV1A.0918s0020. The aligned PAN domains are the following: SapurV1A.0387s0100.1 (SEQ ID NO: 24), SapurV1A.1175s0020.1 (SEQ ID NO: 25), SapurV1A.0037s0270.1 (SEQ ID NO: 26), SapurV1A.1316s0010.1 (SEQ ID NO: 27), SapurV1A.0918s0020.1 (SEQ ID NO: 28), AT1G61380 (SEQ ID NO: 29), Potri.005G018000.1 (SEQ ID NO: 30), Potri.T022200.1 (SEQ ID NO: 31).

FIG. 2. Domain architecture of PAN domain proteins in Anopheles gambiae and Diaphorina citri.

FIG. 3. Proportion of protein domains co-occurring with the PAN domain in Fusarium oxysporum species complex.

FIGS. 4A-4B. Proportion of protein domain co-occurring with the PAN domain in oomycetes Aphanomyces astaci and Phytophthora ramorum.

FIGS. 5A-5B. Tandem expansion of G-LecRKs (orange blocks) on chromosome 4 and 9 of Oryza sativa compared to the syntenic region in the Oryzapunctata genome.

FIGS. 6A-6K. Protein domain co-occurring with the PAN domain across organismal categories. (A) Alveolata, (B) Amoebozoa, (C) Archea, (D) Bacteria, (E) Cryptophyta; (F) Euglenozoa, (G) Haptophyceae, (H) Meta Genomics, (I) Opisthokonka, (J) Stramenoplies, (K) Viridplantae.

FIG. 7. Domain composition of PAN domain-containing proteins in viruses.

FIG. 8. Amino acid conservation within the PAN domain in different human proteins. NM_000601.5 represents HGF. Arrows show the four conserved Cysteine (C) and one conserved Asparagine (N). PAN domains used in the alignment are from the following proteins: NM_000601.5 (SEQ ID NO: 32); NP_001258662.1 (SEQ ID NO: 33); XP_016861955.1 (SEQ ID NO: 34); XP_016861956.1 (SEQ ID NO: 35); NP_066278.3 (SEQ ID NO: 36); XP_016861952.1 (SEQ ID NO: 37); XP_011532040.1 (SEQ ID NO: 38); XP_016861957.1 (SEQ ID NO: 39); XP_016861954.1 (SEQ ID NO: 40); XP_016861953.1 (SEQ ID NO: 41); XP_016861949.1 (SEQ ID NO: 42); XP_016861950.1 (SEQ ID NO: 43); XP_016861951.1 (SEQ ID NO: 44); XP_006713229.1 (SEQ ID NO: 45); XP_011532034.1 (SEQ ID NO: 46).

FIG. 9. Conserved cysteine residues in HGF-PAN domain are essential for HGF/c-MET signaling.

FIG. 10. Mutating cysteine residues in HGF results in enhanced protein stability and attenuation of c-MET signaling.

FIG. 11. Schematic representation of the impact of wild type and PAN mutant HGF on c-MET signaling. Wild type HGF can bind with c-MET receptor on the cell membrane and turn on the subsequent signaling cascade whereas the PAN mutant HGF cannot.

FIGS. 12A-12B. (A) Disruption of homodimerization by minimal mutations in the PAN domain of RLK5 protein. (B) Restoration of PAN amino acids to consensus sequence enhances RLK5R homodimerization.

FIGS. 13A-13B. (A) A functional PAN domain enhances receptor proteolysis (RLK7R) whereas minimal mutations in the PAN domain enhance RLK7 stability. (B) Additionally, receptor proteolysis retards MPK3 and 6 expression in RLK7R transgenic lines while the stable RLK7 exhibits induction of both MPK3 and MPK6.

FIGS. 14A-14C. Transcriptional activation of MAP kinases in Arabidopsis transgenic lines overexpressing RLK5 and 7. RLK25 is an L-type RLK that functions as positive defense regulator. (A) MAPKKK13; (B) MAPKKK14; (C) MPK3.

FIGS. 15A-15F. Transcriptional activation of jasmonic acid and ethylene defense marker genes in Arabidopsis transgenic lines overexpressing RLK5 and 7. RLK25 is an L-type RLK that functions as positive defense regulator. (A) JAZ5, (B) LOX4, (C) ERF13, (D) ACS11, (E) ACS7, (F) LOX3.

FIGS. 16A-16B. Transcriptional activation of jasmonic acid and ethylene defense marker genes in Arabidopsis transgenic lines overexpressing RLK5 and 7. RLK25 is an L-type RLK that functions as positive defense regulator. (A) PR1; (B) NPR3.

FIGS. 17A-17C. Transcriptional activation of canonical defense-related WRKY transcriptional factors in Arabidopsis transgenic lines overexpressing RLK5 and 7. RLK25 is an L-type RLK that functions as positive defense regulator. (A) WRKY18; (B) WRK33; (C) WRKY40.

FIG. 18. Proposed model showing RLKs with mutated PAN domains specifically activate ET/JA pathway and attenuates SA and ABA pathway in plant innate immunity. Scheme of activation of JA and ET pathway in RLK PAN overexpressed transgenic pant in Arabidopsis. The green arrow indicates upregulation, and the red arrow indicates the downregulation. These results suggest that RLK PAN transgenic plants shows activated ET/JA pathway and attenuated SA and ABA signaling pathway.

FIG. 19. HGF PAN mutants inactivate MAPK and AKT signaling in HEK293T cells.

FIG. 20. HGF PAN mutants downregulate the activation of cMET signaling cascade in glioblastoma cell line U87.

FIG. 21. Cell proliferation following HGF stimulations in cell HEK 293T cells. Cells show higher absorbance at 24 and 48 hr following WT HGF stimulation compared to 4Cys-4Ala HGF.

FIGS. 22A-22E. Expression of (A) cMET and (B) MMP9 in HEK 293T cells upon HGF stimulation as determined by real time quantitative PCR. Expression of (C) cMET, (D) MMP9 and (E) MMP2 in U87 cells upon HGF stimulation as determined by real time quantitative PCR.

### DETAILED DESCRIPTION

### Definitions

As used herein, the phrase “immunoregulatory protein” or “immune regulatory protein” refers to a protein that regulates a cell's response to a foreign entity. In some embodiments, an immunoregulatory protein suppresses the ability of a cell to respond to the foreign entity (i.e., it is an “immunosuppressor”). The cell can be an animal cell, a plant cell, a fungal cell, or a bacterial cell. In some embodiments, an immunoregulatory protein increases or enables the ability of a cell to respond to the foreign entity (i.e., it is an “immunostimulant”). The ability of a cell to respond to a foreign entity refers to, for example, binding to a foreing entity triggering signaling within the cell that leads to, for example, release of a defensive substance (e.g., a cytokine, an antibacterial or antiviral substance) or rejection of the foreign entity. Overexpression of an immunosuppressor immunoregulatory protein in a cell suppresses/inhibits the cell's immune response to a foreign entity apart and independent of the general immune system of the organism to which the cell belongs. Overexpression of an immunostilmulant immunoregulatory protein in a cell potentiates the cell's immune response to a foreign entity apart and independent of the general immune system of the organism. For example, the inventors have discovered that overexpressing two Salix purpurea G-LecRKs (PAN domain containing proteins) (SapurV1A.0037s0270.1 and SapurV1A.0918s0020) stimulate immune responses in Arabidopsis cells, whereas overexpressin othr G-LecRKs (e.g., SapurV1A.0387s0100.1, SapurV1A.1175s0020.1, and SapurV1A.1316s0010.1) suppress immune responses in Arabidopsis cells.

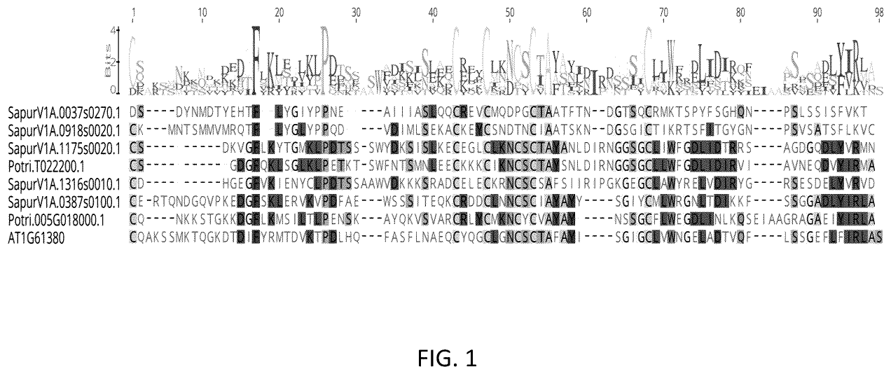

The term “homolog” or “homologous” means a gene related to a second gene by descent from a common ancestral DNA sequence, therefore, the corresponding polynucleotide/polypeptide has a certain degree of homology, i.e., sequence identity (at least 40%, 60%, 65%, at least 66%, 68%, 70%, 75%, 80%, 86%, 88%, 90%, 92%, 95%, 97% or 99% sequence identity). A “homolog” furthermore means that the function is equivalent to the function of the original gene. Homologs of a given gene and corresponding or equivalent positions in the homologous genes or proteins can be determined by sequence alignment programs, e.g., including but not limited to, NCBI BLAST, ClustalW, DIAMOND, CS-BLAST, and MAFFT. For example, an alignment of PAN domains of SapurV1A.0387s0100.1 (SEQ ID NO: 24), SapurV1A.1175s0020.1 (SEQ ID NO: 25), SapurV1A.0037s0270.1 (SEQ ID NO: 26), SapurV1A.1316s0010.1 (SEQ ID NO: 27), SapurV1A.0918s0020.1 (SEQ ID NO: 28), AT1G61380 (SEQ ID NO: 29), Potri.005G018000.1 (SEQ ID NO: 30), and Potri.T022200.1 (SEQ ID NO: 31) is shown in FIG. 1. The alignment shows that while the conserved cysteine residues in SapurV1A.1175s0020.1 are at positions 1, 32, 36, 40, 42, and 57 of SEQ ID NO: 25, corresponding/equivalent conserved cysteine residues in SapurV1A.0387s0100.1 are at positions 1, 39, 43, 47, 49 and 60 of SEQ ID NO: 24.

As used herein, the phrase “nonconservative mutation” refers to a change in a DNA or RNA sequence that results in the replacement of an amino acid with one that is not biochemically similar. In some embodiments, a nonconservative mutation of a cysteine residue is a changing the systeine residue to any amino acid other than serine, selenocysteine, threonine, and methionine (because serine, selenocysteine, threonine, and methionine are biochemically similar to cysteine).

The term “small molecule compound” herein refers to small organic chemical compound, generally having a molecular weight of less than 2000 daltons, 1500 daltons, 1000 daltons, 800 daltons, or 600 daltons.

### Targeted Genome Engineering

Targeted genome engineering (also known as genome editing) has emerged as an alternative to classical plant breeding and transgenic (Genetically Modified Organism—GMO) methods to improve crop plants. Available methods for introducing site-specific double strand DNA breaks include zinc finger nucleases (ZFNs), TAL effector nucleases (TALENs) and CRISPR/Cas system. ZFNs are reviewed in Carroll, D. (Genetics, 188.4 (2011): 773-782), and TALENs are reviewed in Zhang et al. (Plant Physiology, 161.1 (2013): 20-27), which are incorporated herein in their entirety. CRISPR/Cas system is a method based on the bacterial type II CRISPR (clustered regularly interspaced short palindromic repeats)/Cas (CRISPR-associated) immune system. The CRISPR/Cas system allows targeted cleavage of genomic DNA guided by a customizable small noncoding RNA, resulting in gene modifications by both non-homologous end joining (NHEJ) and homology-directed repair (HDR) mechanisms. Belhaj et al. (Plant Methods, 2013, 9:39) summarizes and discusses applications of the CRISPR/Cas technology in plants and is incorporated herein in its entirety.

In a specific embodiment genome editing is achieved by CRISPR (Clustered regularly-interspaced short palindromic repeats)/Cas technology. CRISPR-Cas and similar gene targeting systems are well known in the art with reagents and protocols readily available. Exemplary genome editing protocols are described in Jennifer Doudna, and Prashant Mali, “CRISPR-Cas: A Laboratory Manual” (2016) (CSHL Press, ISBN: 978-1-621821-30-4) and Ran, F. Ann, et al. (Nature Protocols (2013), 8 (11): 2281-2308).

In a specific embodiment, CRISPR-mediated gene repair comprises introducing said plant a first nucleic acid encoding a wild type Cas9 nuclease, a second nucleic acid comprising a guide RNA (gRNA) specific for targeting the mutated genomic region and a third nucleic acid comprising a homologous repair template (aka. Homology Directed Repair—HDR template) of said RLP1, RLP2, L-type lecRLK genes with said deleterious mutation. In this embodiment, the specific guide RNA targets the Cas9 nuclease to the mutated genomic region and the Cas9 nuclease introduces a double strand break in the targeted DNA region. In the presence of the homology template which contains a substantially functional copy of the mutated genomic region (with the deleterious mutation corrected), DNA repair mechanism favors Homology Directed Repair (HDR) and the mutation in the targeted gene is corrected.

### The Plasminogen Apple Nematode (PAN) Domain

The PAN domain (Pfam ID: PF00024; InterPro ID: IPR003609; PROSITE ID: PDOC00376) contains 4 to 6 conserved cysteine residues. The conserved residues are involved in the formation of disulfide bond links between the first and sixth, second and fifth, third and fourth cysteines to form a hairpin-loop structure. Cysteine residues 1 and 6 are not conserved in some PAN domain containing proteins such as the Hepatocyte Growth Factor (HGF). As used herein, a PAN domain has the following consensus sequence: C-x(3)-[LIVMFY]-x(5)-[LIVMFY]-x(3)-[DENQ]-[LIVMFY]-x(10)-C-x(3)-C-T-x(4)-C-x-[LIVMFY]-F-x-[FY]-x(13-14)-C-x-[LIVMFY]-[RK]-x-[ST]-x(14-15)-S-G-x-[ST]-[LIVMFY]-x(2)-C(SEQ ID NO: 23). In some embodiments, the consensus sequence has the sequence as shown in any one of SEQ ID NO: 50-52. The skilled artisan can determine whether a protein comprises a PAN domain by searching the amino acid sequence of the protein using the consensus PAN domain sequence as a query sequence and any sequence alignment software.

The inventors of the instant disclosure have curated >28,000 proteins across eukaryotes, archaea, bacteria and viruses carrying the PAN domain. See List 1. After classifying 2,496 organisms representing 959 genera, into 13 categories (Alveolata, Archea, Amoebazoa, Bacteria, Cryptophyta, Euglenozoa, Haptophyceae, Opisthokonta, Rhizaria, Rhodophyta, Stramenopilles, Viridplantae, and Viruses) the inventors observed that proteins carrying the PAN domain have been implicated in modulation of immune responses across divergent organisms (e.g., from plants to animals, from viruses to bacteria and fungi), suggesting that the domain may have been co-opted to serve the same immunosuppressive function under different biological circumstances.

### Methods for Screening for an Immunoregulatory Protein

The inventors of the instant disclosure have discovered that proteins that contain at least one PAN domain are involved in immunoregulation. In some embodiments, a PAN domain-containing protein functions as an immunosuppressor.

One aspect of the disclosure is directed to a method for screening for an immune regulating protein comprising assessing the sequence of a candidate protein, and determining that the candidate protein is an immune regulating protein when at least one Plasminogen Apple Nematode (PAN) domain is found in the sequence.

It has been found by the present inventors that a PAN domain is present in proteins having an immunoregulatory function. For example, a GO-enrichment analysis by the present inventors using >28,000 PAN domain-containing proteins listed in List 1 revealed 39 GO-terms that were enriched at p<0.05. These included terms like cell recognition (p <1E-30), cell communication (p<1E-30), proteolysis (p<1E-30), pollen-pistil interaction (p<1E-30), reproduction (p<1E-30), response to stimulus (p<1E-30) and response to stress (p<1E-30). These processes are consistent with roles in modulation of immune responses.

In some embodiments, the PAN domain has a consensus sequence as shown in SEQ ID NO: 23.

In some embodiments, the candidate protein is selected from a plant protein, an animal protein, a bacterial protein, a viral protein and a fungal protein. In some embodiments, the animal is a domesticated animal (cow, sheep, cat, dog, goat, etc) or a human.

In some embodiments, at least four conserved cysteine residues in the PAN domain of the immunoregulatory protein. In some embodiments, the at least four conserved cysteine residues comprise cysteines corresponding to the second cysteine to the fifth cysteine of SEQ ID NO: 23. In some embodiments, all six cysteine residues that correspond to the six cysteine residues of SEQ ID NO: 23 are conserved in the protein.

### Methods for Screening for a Therapeutic Compound Targeting a PAN Domain

Another aspect of the disclosure is directed to a method for screening for a therapeutic compound targeting a PAN domain in a protein, comprising contacting the protein comprising the PAN domain with a candidate therapeutic compound, and assessing whether the compound binds to the PAN domain.

In some embodiments, the candidate compound is a small molecule compound.

In some embodiments, the candidate compound binds to the PAN domain with a KD of 10⁻⁷M, 10⁻⁸ M, 10⁻⁹ M, 10⁻¹⁰ M, 10⁻¹¹ M, 10⁻¹² M or less.

In some embodiments, the binding affinity of the candidate compound is measured by a competitive binding assay.

In some embodiments, the PAN-domain containing protein is selected from the proteins listed in List 1.

In some embodiments, the PAN domain in the PAN domain containing protein has a consensus sequence as shown in SEQ ID NO: 23.

In some embodiments, the protein is selected from a plant protein, an animal protein, a bacterial protein, a viral protein and a fungal protein.

In some embodiments, a compound, which is identified as binding to the PAN domain, inhibits the immunoregulatory function of the PAN domain-containing protein. For example, hepatocyte growth factor (HGF), a PAN domain-containing protein, is an immunoregulator that acts through its interaction with the cMET receptor. The inventors of the instant disclosure have found that the PAN domain in HGF is critical for its interaction with cMET receptor and its role in immunoregulation.

### Methods for Screening for a MET Signaling Inhibitor

A different aspect of the disclosure is directed to a method for screening for a MET signaling inhibitor, comprisingexpressing a c-MET receptor on the surface of a cell; incubating the cell with Hepatocyte Growth Factor (HGF) in the presence of a candidate inhibitor of PAN domain interactions; and determining that the candidate inhibitor is an inhibitor of c-MET signaling if the interaction with c-MET and HGF is inhibited.

In some embodiments, the candidate inhibitor of PAN domain interactions is a competitive inhibitor of PAN domain binding. In some embodiments, the candidate inhibitor of PAN domain interactions is an allosteric inhibitor of PAN domain binding.

In some embodiments, the candidate inhibitor of PAN domain interactions is a peptide comprising a consensus sequence as shown in SEQ ID NO: 23.

In some embodiments, the inhibitor candidate is a small molecule compound.

In some embodiments, the inhibitor candidate binds to the PAN domain with a KD of 10⁻⁷ M, 10⁻⁸ M, 10⁻⁹ M, 10⁻¹⁰ M, 10⁻¹¹ M, 10⁻¹² M or less.

In some embodiments, the cell is a mammalian cell.

### Methods for Enhancing Immune Response to Pathogens in Plants

It has been found in accordance with this disclosure that PAN domain-containing proteins in plants have the function of suppressing the plant immune response to foreign entities. While in some instances the foreign entity is a symbiont beneficial to the plant, in other instances the foreign entity is a pathogen.

Another aspect of the disclosure is directed to a method for enhancing immune response or resistance to a pathogen in a plant comprising inactivating a gene encoding a PAN domain-containing protein in the plant.

In some embodiments, the inactivating is achieved by introducing a non-silent mutation in the gene at a residue encoding a conserved cysteine residue of the PAN domain.

In some embodiments, the PAN domain containing protein gene is selected from a plant G-type receptor-like protein kinase gene. In some embodiments, a G-type receptor-like protein kinase gene has a nucleotide sequence that is homologous to SEQ ID NO: 47. In some embodiments, a G-type receptor-like protein kinase gene has a nucleotide sequence that is at least 70%, 73% 75%, 78%, 80%, 83%, 85%, 88%, 90%, 93%, 95% or 99%, or more identical to SEQ ID NO: 47. In some embodiments, a G-type receptor-like protein kinase gene encodes a protein that has an amino acid sequence that is homologous to SEQ ID NO: 48. In some embodiments, a G-type receptor-like protein kinase gene encodes a protein that has an amino acid sequence that is at least 70%, 73% 75%, 78%, 80%, 83%, 85%, 88%, 90%, 93%, 95% or 99%, or more identical to SEQ ID NO: 48.

In some embodiments, the conserved cysteine residue is selected from a residue equivalent to a cysteine at position 1, 32, 36, 40, 42, or 57 of SEQ ID NO: 25.

In some embodiments, the non-silent mutation is a nonconservative mutation.

In some embodiments, the nonconservative mutation is a cysteine to alanine mutation.

In some embodiments, the inactivating of the gene encoding a PAN domain containing protein is achieved by introducing an exogenous nucleic acid inhibitor of the gene to the plant.

In some embodiments, the nucleic acid inhibitor is selected from the group consisting of an antisense RNA, a small interfering RNA, an RNAi, a microRNA, an artificial microRNA, and a ribozyme.

In some embodiments, the inactivating of the selected gene is achieved by genome editing, which is achieved by a method selected from the group consisting of CRISPR/Cas system, Cre/Lox system, TALEN system, ZFNs system and homologous recombination.

In some embodiments, the CRISPR-mediated genome editing comprises introducing into the plant a first nucleic acid encoding a Cas9 nuclease, a second nucleic acid comprising a guide RNA (gRNA), wherein said gRNA is specific to the gene encoding a PAN domain containing protein.

In some embodiments, the CRISPR-mediated genome editing comprises introducing into the plant a first nucleic acid encoding a Cas9 nuclease, a second nucleic acid comprising a guide RNA (gRNA), wherein said gRNA is specific to the gene encoding a PAN domain containing protein, and a third nucleic acid comprising a template for homologous recombination.

In some embodiments, the pathogen is selected from a bacterium, a virus or a fungus.

In some embodiments, the pathogen is a necrotropic fungus selected from the group consisting of Sphaerulina abeliceae, Sphaerulina aceris, Sphaerulina acetabulum, Sphaerulina acori, Sphaerulina aechmeae, Sphaerulina affinis, Sphaerulina albispiculata, Sphaerulina alni, Sphaerulina amelanchier, Sphaerulina amicta, Sphaerulina amphilomatis, Sphaerulina amygdali, Sphaerulina anemones, Sphaerulina annae, Sphaerulina antarctica, Sphaerulina arctica, Sphaerulina arthoniae, Sphaerulina assurgens, Sphaerulina aucubae, Sphaerulina azaleae, Sphaerulina baccarum, Sphaerulina bambusicola, Sphaerulina berberidis, Sphaerulina betulae, Sphaerulina blyttii, Sphaerulina bonariana, Sphaerulina boudieriana, Sphaerulina bryophila, Sphaerulina callista, Sphaerulina camelliae, Sphaerulina camelliae, Sphaerulina carestiae, Sphaerulina caricae, Sphaerulina caricis, Sphaerulina ceanothi, Sphaerulina centellae, Sphaerulina cercidis, Sphaerulina cetraricola, Sphaerulina cetrariicola, Sphaerulina chlorococca, Sphaerulina cibotii, Sphaerulina citri, Sphaerulina codiicola, Sphaerulina coffaeicola, Sphaerulina coffeicola, Sphaerulina concinna, Sphaerulina conflicta, Sphaerulina coriariae, Sphaerulina cornicola, Sphaerulina corniculata, Sphaerulina coronillae-junceae, Sphaerulina corynephora, Sphaerulina cucumeris, Sphaerulina cucurbitae, Sphaerulina datiscae, Sphaerulina diapensiae, Sphaerulina dioscoreae, Sphaerulina divergens, Sphaerulina dolichotera, Sphaerulina dryadis, Sphaerulina dryophila, Sphaerulina dubiella, Sphaerulina empetri, Sphaerulina endococcoidea, Sphaerulina epigaea, Sphaerulina eucalypti, Sphaerulina ferruginosa, Sphaerulina frondicola, Sphaerulinafuegiana, Sphaerulina gei, Sphaerulina gentianae, Sphaerulina gigantea, Sphaerulina giliae, Sphaerulina hainensis, Sphaerulina halophila, Sphaerulina hamadryadum, Sphaerulina hederae, Sphaerulina helicicola, Sphaerulina hyperici, Sphaerulina inaequalis, Sphaerulina inquinans, Sphaerulina intermedia, Sphaerulina intermixta, Sphaerulina ipomoeae, Sphaerulina islandica, Sphaerulina iwatensis, Sphaerulina juglandis, Sphaerulina leightonii, Sphaerulina lepidiotae, Sphaerulina limnanthemi, Sphaerulina lini, Sphaerulina linicola, Sphaerulina ludwigiae, Sphaerulina mappiae, Sphaerulina marattiae, Sphaerulina marginata, Sphaerulina maroccana, Sphaerulina marsileae, Sphaerulina maydis, Sphaerulina menispermi, Sphaerulina microthyrioides, Sphaerulina mimosae-pigrae, Sphaerulina miyakei, Sphaerulina musae, Sphaerulina muscicola, Sphaerulina muscorum, Sphaerulina musicola, Sphaerulina musiva, Sphaerulina myriadea, Sphaerulina myriadea subsp. myriadea, Sphaerulina myrtillina, Sphaerulina naumovii, Sphaerulina nephromiaria, Sphaerulina oleifolia, Sphaerulina orae-maris, Sphaerulina oryzae, Sphaerulina oryzina, Sphaerulina oxalidis, Sphaerulina oxyacanthae, Sphaerulina pallens, Sphaerulina parvipuncta, Sphaerulina patriniae, Sphaerulina paulistana, Sphaerulina peckii, Sphaerulina pedicellata, Sphaerulina pelargonii, Sphaerulina phalaenopsidis, Sphaerulina phellogena, Sphaerulina phoenicis, Sphaerulina phyllostachydis, Sphaerulina pini, Sphaerulina plantaginea, Sphaerulina pleuropogonis, Sphaerulina polygonorum, Sphaerulina polypodii, Sphaerulina polypodii, Sphaerulina polyspora, Sphaerulina populi, Sphaerulina populicola, Sphaerulina porothelia, Sphaerulina potebniae, Sphaerulina potentillae, Sphaerulina poterii, Sphaerulina primulicola, Sphaerulina pruni, Sphaerulina pseudovirgaureae, Sphaerulina pterocarpi, Sphaerulina pulii, Sphaerulina quercicola, Sphaerulina quercifolia, Sphaerulina quitensis, Sphaerulina rehmiana, Sphaerulina rhabdoclinis, Sphaerulina rhodeae, Sphaerulina rhododendri, Sphaerulina rhododendricola, Sphaerulina rubi, Sphaerulina saccardiana, Sphaerulina saccardoana, Sphaerulina sacchari, Sphaerulina salicina, Sphaerulina sambucina, Sphaerulina sasae, Sphaerulina schaereri, Sphaerulina scirpi, Sphaerulina sepincola, Sphaerulina serograpta, Sphaerulina silacincola, Sphaerulina smilacincola, Sphaerulina socia, Sphaerulina spartii, Sphaerulina staphyleae, Sphaerulina staurochili, Sphaerulina steganostroma, Sphaerulina subgen. Pharcidiella, Sphaerulina subgen, Sphaerulina, Sphaerulina subglacialis, Sphaerulina subtropica, Sphaerulina suchumica, Sphaerulina tabacinae, Sphaerulina tanaceti, Sphaerulina tarda, Sphaerulina taxi, Sphaerulina taxicola, Sphaerulina thujopsidis, Sphaerulina tiliaris, Sphaerulina tirolensis, Sphaerulina todeae, Sphaerulina trapae-bispinosae, Sphaerulina trifolii, Sphaerulina tritici, Sphaerulina umbilicata, Sphaerulina valerianae, Sphaerulina viciae, Sphaerulina vincae, Sphaerulina violae, Sphaerulina vismiae, Sphaerulina vulpina, Sphaerulina westendorpii, Sphaerulina worsdellii, Sphaerulina xerophylli, Sphaerulina yerbae, Sphaerulina ziziphi, Sphaerulina zizyphae, and Sphaerulina zizyphi.

### Genetically-Engineered Plants Resistant to Pathogens and Parasites

Another aspect of the disclosure is directed to a genetically engineered plant, wherein an endogenous gene encoding a PAN domain-containing protein in the plant has been inactivated as a result of a genetic modification.

In some embodiments, the inactivation is achieved by introducing a non-silent mutation in the gene at a residue encoding a conserved cysteine residue of the PAN domain.

In some embodiments, the gene encoding a PAN domain-containing protein is selected from a plant G-type receptor-like protein kinase gene. In some embodiments, a G-type receptor-like protein kinase gene has a nucleotide sequence that is homologous to SEQ ID NO: 47. In some embodiments, a G-type receptor-like protein kinase gene has a nucleotide sequence that is at least 70%, 73% 75%, 78%, 80%, 83%, 85%, 88%, 90%, 93%, 95% or 99%, or more identical to SEQ ID NO: 47. In some embodiments, a G-type receptor-like protein kinase gene encodes a protein that has an amino acid sequence that is homologous to SEQ ID NO: 48. In some embodiments, a G-type receptor-like protein kinase gene encodes a protein that has an amino acid sequence that is at least 70%, 73% 75%, 78%, 80%, 83%, 85%, 88%, 90%, 93%, 95% or 99%, or more identical to SEQ ID NO: 48.

In some embodiments, the conserved cysteine residue is selected from a residue equivalent to a cysteine at position 1, 32, 36, 40, 42, or 57 of SEQ ID NO: 25.

In some embodiments, the non-silent mutation is a nonconservative mutation.

In some embodiments, the nonconservative mutation is a cysteine to alanine mutation.

In some embodiments, the pathogen is selected from a bacterium, a virus or a fungus.

In some embodiments, the pathogen is a necrotropic fungus selected from the group consisting of Sphaerulina abeliceae, Sphaerulina aceris, Sphaerulina acetabulum, Sphaerulina acori, Sphaerulina aechmeae, Sphaerulina affinis, Sphaerulina albispiculata, Sphaerulina alni, Sphaerulina amelanchier, Sphaerulina amicta, Sphaerulina amphilomatis, Sphaerulina amygdai, Sphaerulina anemones, Sphaerulina annae, Sphaerulina antarctica, Sphaerulina arctica, Sphaerulina arthoniae, Sphaerulina assurgens, Sphaerulina aucubae, Sphaerulina azaleae, Sphaerulina baccarum, Sphaerulina bambusicola, Sphaerulina berberidis, Sphaerulina betulae, Sphaerulina blyttii, Sphaerulina bonariana, Sphaerulina boudieriana, Sphaerulina bryophila, Sphaerulina callista, Sphaerulina camelliae, Sphaerulina camelliae, Sphaerulina carestiae, Sphaerulina caricae, Sphaerulina caricis, Sphaerulina ceanothi, Sphaerulina centellae, Sphaerulina cercidis, Sphaerulina cetraricola, Sphaerulina cetrariicola, Sphaerulina chlorococca, Sphaerulina cibotii, Sphaerulina citri, Sphaerulina codiicola, Sphaerulina coffaeicola, Sphaerulina coffeicola, Sphaerulina concinna, Sphaerulina conflicta, Sphaerulina coriariae, Sphaerulina cornicola, Sphaerulina corniculata, Sphaerulina coronillae-junceae, Sphaerulina corynephora, Sphaerulina cucumeris, Sphaerulina cucurbitae, Sphaerulina datiscae, Sphaerulina diapensiae, Sphaerulina dioscoreae, Sphaerulina divergens, Sphaerulina dolichotera, Sphaerulina dryadis, Sphaerulina dryophila, Sphaerulina dubiella, Sphaerulina empetri, Sphaerulina endococcoidea, Sphaerulina epigaea, Sphaerulina eucalypti, Sphaerulina ferruginosa, Sphaerulina frondicola, Sphaerulinafuegiana, Sphaerulina gei, Sphaerulina gentianae, Sphaerulina gigantea, Sphaerulina giliae, Sphaerulina hainensis, Sphaerulina halophila, Sphaerulina hamadryadum, Sphaerulina hederae, Sphaerulina helicicola, Sphaerulina hyperici, Sphaerulina inaequalis, Sphaerulina inquinans, Sphaerulina intermedia, Sphaerulina intermixta, Sphaerulina ipomoeae, Sphaerulina islandica, Sphaerulina iwatensis, Sphaerulina juglandis, Sphaerulina leightonii, Sphaerulina lepidiotae, Sphaerulina limnanthemi, Sphaerulina lini, Sphaerulina linicola, Sphaerulina ludwigiae, Sphaerulina mappiae, Sphaerulina marattiae, Sphaerulina marginata, Sphaerulina maroccana, Sphaerulina marsileae, Sphaerulina maydis, Sphaerulina menispermi, Sphaerulina microthyrioides, Sphaerulina mimosae-pigrae, Sphaerulina miyakei, Sphaerulina musae, Sphaerulina muscicola, Sphaerulina muscorum, Sphaerulina musicola, Sphaerulina musiva, Sphaerulina myriadea, Sphaerulina myriadea subsp. myriadea, Sphaerulina myrtillina, Sphaerulina naumovii, Sphaerulina nephromiaria, Sphaerulina oleifolia, Sphaerulina orae-maris, Sphaerulina oryzae, Sphaerulina oryzina, Sphaerulina oxalidis, Sphaerulina oxyacanthae, Sphaerulina pallens, Sphaerulina parvipuncta, Sphaerulina patriniae, Sphaerulina paulistana, Sphaerulina peckii, Sphaerulina pedicellata, Sphaerulina pelargonii, Sphaerulina phalaenopsidis, Sphaerulina phellogena, Sphaerulina phoenicis, Sphaerulina phyllostachydis, Sphaerulina pini, Sphaerulina plantaginea, Sphaerulina pleuropogonis, Sphaerulina polygonorum, Sphaerulina polypodii, Sphaerulina polypodii, Sphaerulina polyspora, Sphaerulina populi, Sphaerulina populicola, Sphaerulina porothelia, Sphaerulina potebniae, Sphaerulina potentillae, Sphaerulina poterii, Sphaerulina primulicola, Sphaerulina pruni, Sphaerulina pseudovirgaureae, Sphaerulina pterocarpi, Sphaerulina pulii, Sphaerulina quercicola, Sphaerulina quercifolia, Sphaerulina quitensis, Sphaerulina rehmiana, Sphaerulina rhabdoclinis, Sphaerulina rhodeae, Sphaerulina rhododendri, Sphaerulina rhododendricola, Sphaerulina rubi, Sphaerulina saccardiana, Sphaerulina saccardoana, Sphaerulina sacchari, Sphaerulina salicina, Sphaerulina sambucina, Sphaerulina sasae, Sphaerulina schaereri, Sphaerulina scirpi, Sphaerulina sepincola, Sphaerulina serograpta, Sphaerulina silacincola, Sphaerulina smilacincola, Sphaerulina socia, Sphaerulina spartii, Sphaerulina staphyleae, Sphaerulina staurochili, Sphaerulina steganostroma, Sphaerulina subgen. Pharcidiella, Sphaerulina subgen, Sphaerulina, Sphaerulina subglacialis, Sphaerulina subtropica, Sphaerulina suchumica, Sphaerulina tabacinae, Sphaerulina tanaceti, Sphaerulina tarda, Sphaerulina taxi, Sphaerulina taxicola, Sphaerulina thujopsidis, Sphaerulina tiliaris, Sphaerulina tirolensis, Sphaerulina todeae, Sphaerulina trapae-bispinosae, Sphaerulina trifolii, Sphaerulina tritici, Sphaerulina umbilicata, Sphaerulina valerianae, Sphaerulina viciae, Sphaerulina vincae, Sphaerulina violae, Sphaerulina vismiae, Sphaerulina vulpina, Sphaerulina westendorpii, Sphaerulina worsdellii, Sphaerulina xerophylli, Sphaerulina yerbae, Sphaerulina ziziphi, Sphaerulina zizyphae, and Sphaerulina zizyphi.

### Method for Preventing Cell Division of a Human Cell

It has been found in accordance with this disclosure that PAN domain-containing proteins are involved in regulating the rate of cell division in mammalian cells, and cellular migration in cancer cells. Thus, another aspect of the disclosure is directed to a method for preventing cell division of a human cell and cellular migration in cancer cells comprising inactivating a gene encoding a PAN domain containing protein in the human cell.

In some embodiments, the inactivation is achieved by introducing a non-silent mutation in the gene at a residue encoding a conserved cysteine residue of the PAN domain.

In some embodiments, the PAN domain-containing protein is Hepatocyte Growth Factor (HGF), or a homolog thereof. In some embodiments, the HGF binds to cMET receptor and has an amino acid sequence that is at least 70%, 73% 75%, 78%, 80%, 83%, 85%, 88%, 90%, 93%, 95% or 99%, or more identical to SEQ ID NO: 49.

In some embodiments, the conserved cysteine residue is selected from a residue homologous to a cysteine at position 34, 38, 48, or 60 of SEQ ID NO: 32.

In some embodiments, the non-silent mutation is a nonconservative mutation. In some embodiments, the nonconservative mutation is a cysteine to alanine mutation.

In some embodiments, the inactivating of the selected gene is achieved by introducing an exogenous nucleic acid inhibitor of the gene encoding a PAN domain-containing protein to the cancer cell.

In some embodiments, the nucleic acid inhibitor is selected from the group consisting of an antisense RNA, a small interfering RNA, an RNAi, a microRNA, an artificial microRNA, and a ribozyme.

In some embodiments, the inactivating of the gene encoding a PAN domain containing protein is achieved by genome editing, which is achieved by a method selected from the group consisting of CRISPR/Cas system, Cre/Lox system, TALEN system, ZFNs system and homologous recombination.

In some embodiments, the CRISPR-mediated genome editing comprises introducing into the cancer cell a first nucleic acid encoding a Cas9 nuclease, and a second nucleic acid comprising a guide RNA (gRNA), wherein said gRNA is specific to the gene.

In some embodiments, the CRISPR-mediated genome editing comprises introducing into the cancer cell a first nucleic acid encoding a Cas9 nuclease, a second nucleic acid comprising a guide RNA (gRNA), wherein said gRNA is specific to the gene encoding a PAN domain containing protein, and a third nucleic acid comprising a template for homologous recombination.

In some embodiments, the cancer cell is selected from breast cancer, colon cancer, lung cancer, skin cancer, brain cancer, blood cancer, cervical cancer, liver cancer, prostate carcinoma, pancreas carcinoma, gastric carcinoma, ovarian carcinoma, renal cell carcinoma, mesothelioma, and melanoma.

### Methods for Improving In Vitro Fertilization (IVF) Efficiency

It has been found in accordance with this disclosure that sperms and oocytes contain PAN domain-containing proteins that play crucial roles in the fertilization process. Without being bound by a particular theory, PAN domain containing proteins are believed to suppress the immune response by the oocyte, or other cells in the uterus, against the sperm (a foreign entity) and the embryo (partially foreign entity). Some cases of infertility can result from mutations in a PAN domain of a PAN domain containing protein expressed in the reproductive system.

Therefore, another aspect of the disclosure is directed to a method for improving in vitro fertilization (IVF) efficiency in a non-human subject suffering from infertility comprising determining the sequence of a gene that encodes a PAN domain-containing protein, and when a mutation is found in a conserved cysteine of the PAN domain of the PAN domain-containing protein obtaining an ovum or a sperm from the subject, correcting the mutation in the PAN domain-containing protein in the ovum or the sperm.

In some embodiments, the PAN domain containing protein gene is a Zona Pellicuda(ZP) protein.

In some embodiments, the conserved cysteine residue is selected from a residue homologous to a cysteine residue in SEQ ID NO: 23.

In some embodiments, the correcting of the PAN domain is achieved by genome editing by a method selected from the group consisting of CRISPR/Cas system, Cre/Lox system, TALEN system, ZFNs system and homologous recombination.

In some embodiments, the CRISPR-mediated genome editing comprises introducing into the ovum or the sperm a first nucleic acid encoding a Cas9 nuclease, a second nucleic acid comprising a guide RNA (gRNA), wherein said gRNA is specific to the gene encoding a PAN domain containing protein, and a third nucleic acid comprising a template for homologous recombination that corrects the mutated residue to cysteine.

In some embodiments, wherein the ovum or the sperm are from a domesticated animal. In some embodiments, the domesticated animal is selected from the group consisting of horse, camel, donkey, cat, goat, dog, pig, cow, and sheep.

### Methods for Preventing Pregnancy (Immunocontraception)

Another aspect of the disclosure is directed to a method for preventing pregnancy or implantation of a fetus in a non-human subject comprising inactivating a gene encoding a PAN domain-containing protein in an ovum or a sperm.

In some embodiments, the inactivating is achieved by introducing a non-silent mutation in the gene at a residue encoding a conserved cysteine residue of the PAN domain.

In some embodiments, the PAN domain containing protein gene is a Zona Pellicuda protein.

In some embodiments, the conserved cysteine residue is selected from a residue homologous to a cysteine a cysteine residue in SEQ ID NO: 23.

In some embodiments, the non-silent mutation is a nonconservative mutation.

In some embodiments, the nonconservative mutation is a cysteine to alanine mutation.

In some embodiments, the inactivating of the selected gene is achieved by introducing an exogenous nucleic acid inhibitor of the selected gene to the ovum or the sperm.

In some embodiments, the nucleic acid inhibitor is selected from the group consisting of an antisense RNA, a small interfering RNA, an RNAi, a microRNA, an artificial microRNA, and a ribozyme.

In some embodiments, the inactivating of the selected gene is achieved by genome editing, which is achieved by a method selected from the group consisting of CRISPR/Cas system, Cre/Lox system, TALEN system, ZFNs system and homologous recombination.

In some embodiments, the CRISPR-mediated genome editing comprises introducing into the ovum or the sperm a first nucleic acid encoding a Cas9 nuclease, a second nucleic acid comprising a guide RNA (gRNA), wherein said gRNA is specific to the selected gene.

In some embodiments, the CRISPR-mediated genome editing comprises introducing into the ovum or the sperm a first nucleic acid encoding a Cas9 nuclease, a second nucleic acid comprising a guide RNA (gRNA), wherein said gRNA is specific to the selected gene, and a third nucleic acid comprising a template for homologous recombination.

In some embodiments, the ovum or the sperm are from a domesticated animal. In some embodiments, the domesticated animal is selected from the group consisting of horse, camel, donkey, cat, goat, dog, pig, cow, and sheep.

### Methods for Discovering Oncoproteins

As discussed above, PAN domain-containing proteins have been found to play a role in regulating the rate of cell division. Therefore, another aspect of the disclosure is directed to a method for screening for a novel oncoprotein protein in a mammalian cell comprising assessing the sequence of a candidate protein, and determining that the candidate protein is an oncoprotein when at least one PAN domain is found in the sequence.

In some embodiments, the Plasminogen Apple Nematode (PAN) domain has a consensus sequence as shown in SEQ ID NO: 23.

In some embodiments, the mammalian cell is an animal cell. In some embodiments, the mammalian cell is a human cell.

### Method for Inactivating a PAN Domain Containing Protein

PAN domains are involved in protein-protein interactions (as in HGF and cMET). In addition, it has also been found in accordance with this disclosure that introducing a mutation in at least one conserved cysteine of a PAN domain prevents the protein-protein interactions of the PAN domain, thereby inactivating the PAN domain-containing protein and inhibiting its function.

Another aspect of the disclosure is directed to a method for inactivating a PAN domain-containing protein comprising introducing a non-silent mutation in the protein at a conserved cysteine residue of the PAN domain.

In some embodiments, the PAN domain-containing protein gene is selected from the proteins listed in List 1.

In some embodiments, the conserved cysteine residue is selected from a residue equivalent to one of the six cysteine residues in SEQ ID NO: 23.

Unless defined otherwise, all technical and scientific terms used herein have the same meaning as commonly understood by one skilled in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

### EXAMPLES

### Example 1: Materials and Methods

### Cloning, Sequencing and Overexpression of G-RLKs

The specific examples listed below are only illustrative and by no means limiting.

The coding sequences of SapurV1A.0037s0270.1, SapurV1A.0918s0020 and SapurV1A.1175s0020 were amplified by PCR from cDNA derived from Salix purpurea leaves using HiFi polymerase (Clontech). Sequences were determined using Phytozyme using the Salix purpurea v.1.0 genome. The pENTR/D-TOPO entry vector was used for the insertion of the PCR products using the pENTR Directional Topo Cloning Kit (Thermo Fisher Scientific). LR Clonase II Enzyme Mix (Thermo Fisher Scientific) was used to transfer the coding sequences into the pGWB402-omega destination vector and transformed into One Shot TOP10 Chemically Competent cells. Vectors were transformed into the Agrobacterium strain GV3101 and transformed into Col-0 Arabidopsis thaliana using the floral dip method. T1 seeds were selected with Kanamycin and grown in long day growth chambers at 16/8 hours light/dark at 22° C. Three week old leaf tissue was collected for RNA extractions using the Spectrum Plant Total RNA Kit (Sigma) and cDNA synthesized using the Superscript III Reverse Transcriptase with oligo-d(T) primers (Thermo Fisher Scientific). qRT-PCR was performed using the PowerUP SYBR Green Master Mix (Thermo Fisher Scientific) and relative expression for each gene was determined with the 2ΔΔCt method with GAPDH as an internal control.

TABLE 1
Primer sequences

| | | SEQ |
|---|---|---|
| | | ID |
| Primer name | Primer sequence | NO |
| 1_0037s0270csF | CACCATGCTTAAGCAGGGCAATTTCT | 1 |
| 2_0037s0270csR | TCATGGTTCCATTGGCCTCT | 2 |
| 5_0918s0020csF | CACCATGGGCGTGACTAGGAATAGAG | 3 |
| 6_0918s0020csR | CTAAGAGGAGTCTACTTCGACCTC | 4 |
| 77_RLK53_GWF | CACCATGGGTACTTTTTCAGTTCTG | 5 |
| 78_RLK53_GWRs | CTACCGGGCCTCTAATGACATC | 6 |
| 23_0037s0270rtF | GGGGTCAAAGCTTTCTACAGAGG | 7 |
| 24_0037s0270rtR | GCCAACCAAACCACAGTTTGC | 8 |
| 27_0918s0020rtF | GCTTTTGTGAAGAGGTTGCCATGG | 9 |
| 28_0918s0020rtR | GACCCCATTTTGAGACACCC | 10 |
| 89_RLK53_RTF | CGTTTCAACCGGAGGAAGC | 11 |
| 90_RLK53_RTR | TTACATTCAGAGCCCCCAAAGC | 12 |
| 177_Arab_WRKY70_RTF | CATGGATTCCGAAGATCACA | 13 |
| 178_Arab_WRKY70_RTR | CTGGCCACACCAATGACAA | 14 |
| 179_Arab_WRKY40_RTF | AAATCAGCCCTCCCAAGAAACG | 15 |
| 180_Arab_WRKY40_RTR | CTTCACGACAGTCTCTTCTCTCTGC | 16 |
| 181_Arab_WRKY33_RTF | AGGCTCATCGATTGTCAGCA | 17 |
| 182_Arab_WRKY33_RTR | CTGCACTACGATTCTCGGCT | 18 |
| 183_Arab_WRKY8_RTF | GTTGTCGGTGATGGTTGTGC | 19 |
| 184_Arab_WRKY8_RTR | CCGGATCTTGCCGGAATCTT | 20 |
| 185_Arab_GAPDH_RTF | AGGCCATCAAGGAGGAATCT | 21 |
| 186_Arab_GAPDH_RTR | GAAAATGCTTGACCTGTTGTCAC | 22 |

TABLE 2
Matching primers to genes and use

| Gene Name | Primer name | Primer use |
|---|---|---|
| SapurV1A.0037s0270 | 1_0037s0270csF | Cloning CDS |
| | 2_0037s0270csR | Cloning CDS |
| SapurV1A.0918s0020 | 5_0918s0020csF | Cloning CDS |
| | 6_0918s0020csR | Cloning CDS |
| SapurV1A.1175s0020 | 77_RLK53_GWF | Cloning CDS |
| | 78_RLK53_GWRs | Cloning CDS |
| SapurV1A.0037s0270 | 23_0037s0270rtF | RT-qPCR |
| | 24_0037s0270rtR | RT-qPCR |
| SapurV1A.0918s0020 | 27_0918s0020rtF | RT-qPCR |
| | 28_0918s0020rtR | RT-qPCR |
| SapurV1A.1175s0020 | 89_RLK53_RTF | RT-qPCR |
| | 90_RLK53_RTR | RT-qPCR |
| WRKY40 (AT1G80840) | 179_Arab_WRKY40_RTF | RT-qPCR |
| | 180_Arab_WRKY40_RTR | RT-qPCR |
| WRKY33 (AT2G38470) | 181_Arab_WRKY33_RTF | RT-qPCR |
| | 182_Arab_WRKY33_RTR | RT-qPCR |
| GAPDH (AT3G26650) | 185_Arab_GAPDH_RTF | RT-qPCR |
| | 186_Arab_GAPDH_RTR | RT-qPCR |

Synteny Analysis Between Oryza sativa and O. Punctata

The CDS sequences and corresponding annotation files for O. sativa (IGRSP 1.0) and O. punctata (Oryza_punctata_vi.2) were downloaded from Ensembl Plants. The CDS files and sequences were then filtered to only retain protein coding genes. The protein coding genes were then annotated with Interproscan 5 (Jones et al., 2014, Bioinformatics, 30.9 (2014): 1236-1240). Synteny analysis was then performed via MCScanX and genes containing a PAN domain were searched against resulting synteny blocks. Regions that showed evidence of PAN domain expansion were extracted and visualized with the JCVI utility libraries.

### Alignments of G-RLK Sequences

Interproscan 5 was used to identify the PAN domain regions of the 8 G-RLKs in FIG. 1. These regions were then extracted and aligned with MAFFT linsi (Katoh et al., Molecular biology and evolution, 30.4 (2013): 772-780) and then visualized with Geneious R11.

Domain Prediction in Anopheles gambiae and Diaphorina Citri

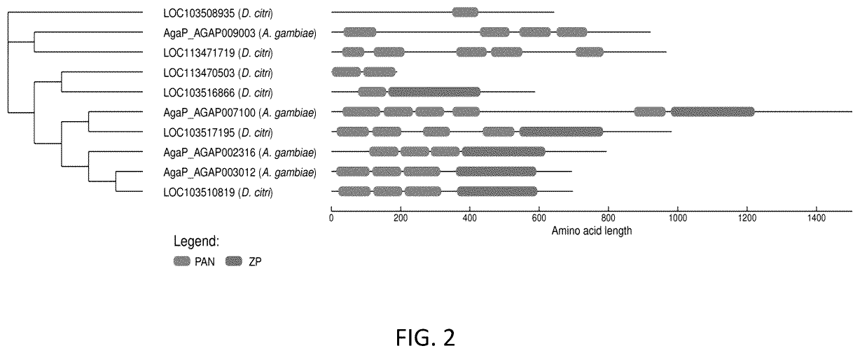

Interproscan 5 was used to identify domains for the 10 proteins in FIG. 2. The proteins were then aligned with MAFFT linsi and a Neighbor-Joining tree was generated with Geneious. The domain structure was then visualized with GSDS 2.0 (Hu et al., Bioinformatics, 31.8 (2014): 1296-1297).

### Example 2

A protein database survey revealed that the PAN domain is found in >28,000 proteins derived from 2,496 organisms representing 959 genera. These included organisms with a high propensity to recruit symbionts, vector parasites and exhibit parasitic or pathogenic behavior. Additionally, the core conserved cysteine residues of the domain are structurally similar to cysteine-rich peptides that have been implicated in modulation of immune responses. Given the cumulative observations, the inventors propose that the PAN domain is a unifying feature in immunoregulation (e.g., immunosuppression) across divergent organism and propose the existence of an ‘immunoregulome’ comprised of PAN domain proteins functioning to mediate host-cell invasion.

In revealing a novel role for self-immunosuppression in plants during host-cell invasion by microbes, the PAN domain proteins, D-mannose lectin kinases (G-LecRKs), were shown to function as negative regulators of defense signaling during pathogenesis by the fungal pathogen Sphaerulina musiva, parasitism of Arabidopsis by nematodes, and conversion of Arabidopsis into a host of the fungal symbiont Laccaria bicolor. Moreover, PAN domain carrying S-locus kinases, reported to mediate self-incompatibility during pollination, fall under the same class of G-LecRKs.

In Salix purpurea, the inventors discovered two G-LecRKs (SapurV1A.0037s0270.1 (RLK5) and SapurV1A.0918s0020 (RLK7)) that, upon overexpression in Arabidopsis, triggered defense responses instead of functioning as immunosuppressors. Domain annotation using the PFAM database did not reveal presence of a PAN domain in both proteins, however, upon further analyses, the inventors observed that these two G-LecRKs carried a vestigial PAN domain. Specifically, the inventors compared the PAN domains of the two Salix G-LecRKs with ones demonstrated to be immunosuppressive, AT1G61380, Potri.T022200.1 and Potri.005G018000.1 and noted that both had mutations on the conserved asparagine (N) and cysteine (C) amino acids at positions 50 and 51 while SapurV1A.0037s0270 was also missing the cysteine residue at position 1 (FIG. 1). With this observation, the inventors hypothesized that the PAN domain was essential for immunosuppression.

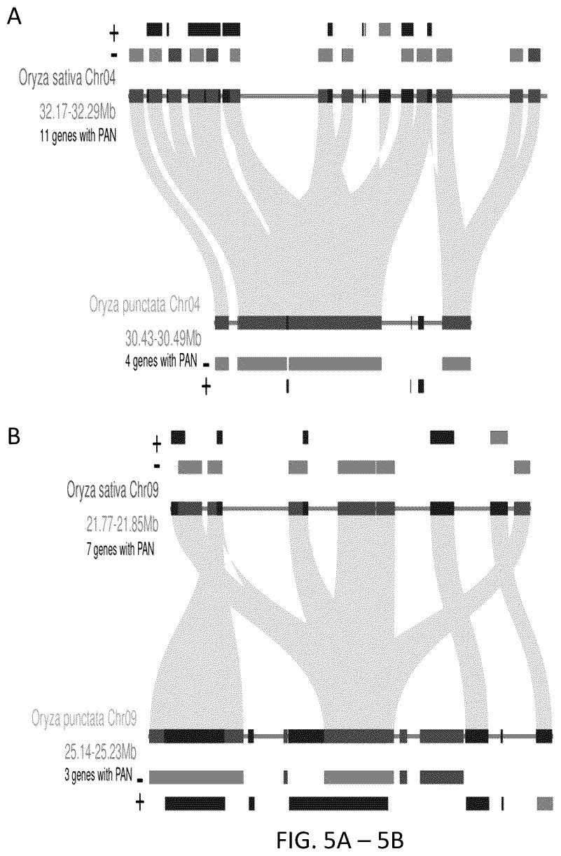

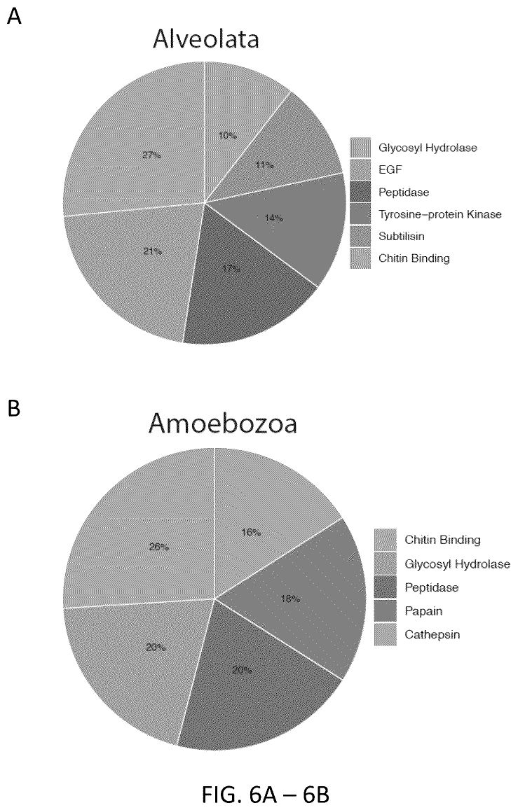

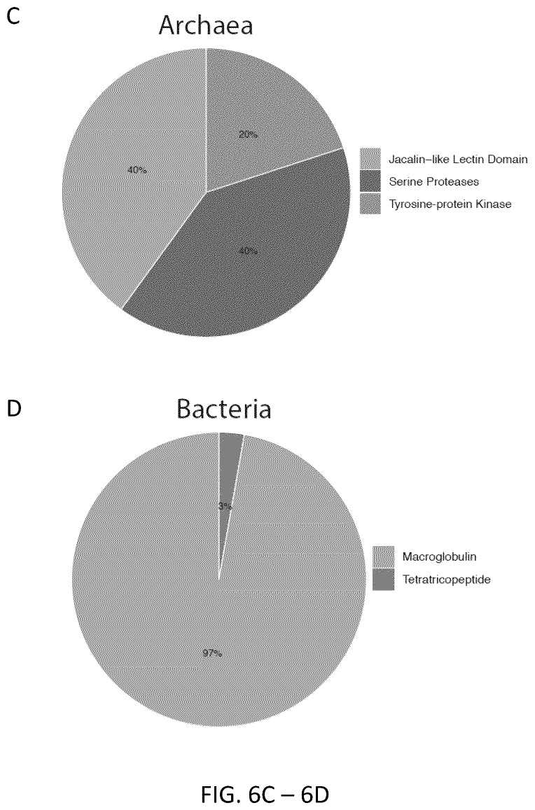

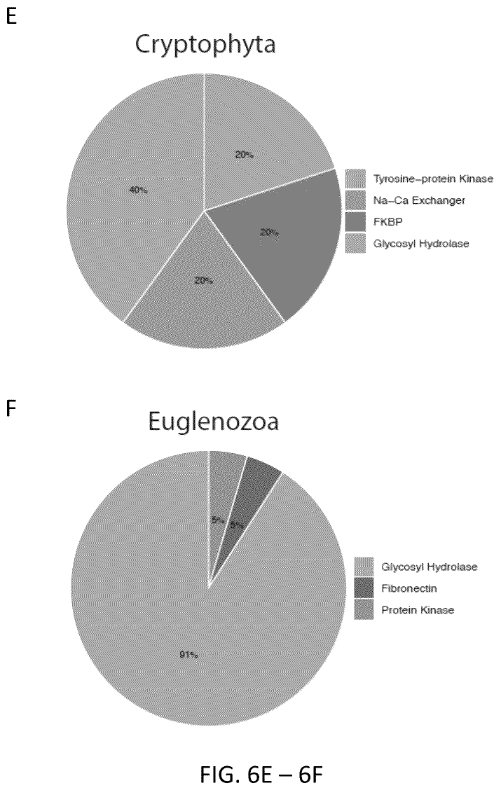

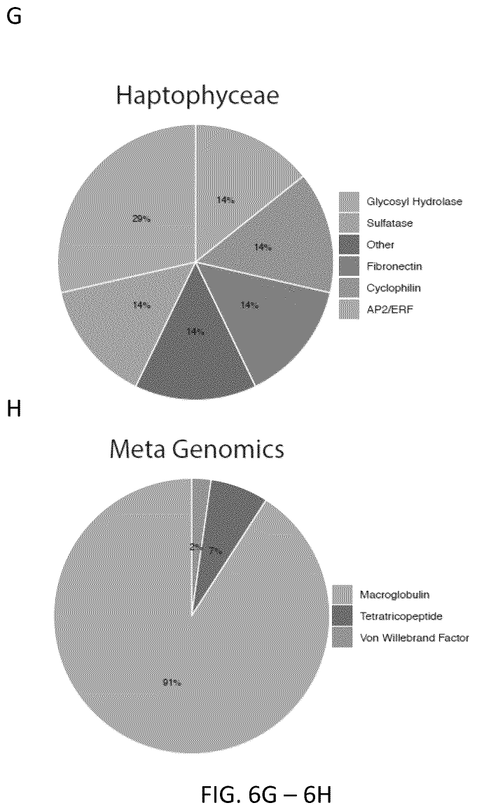

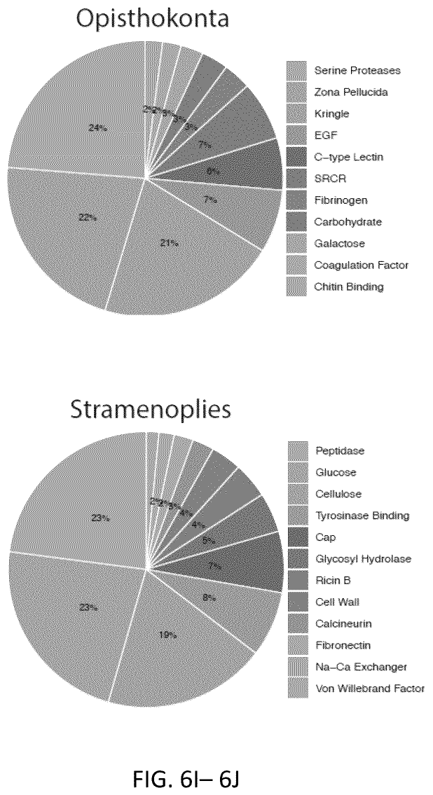

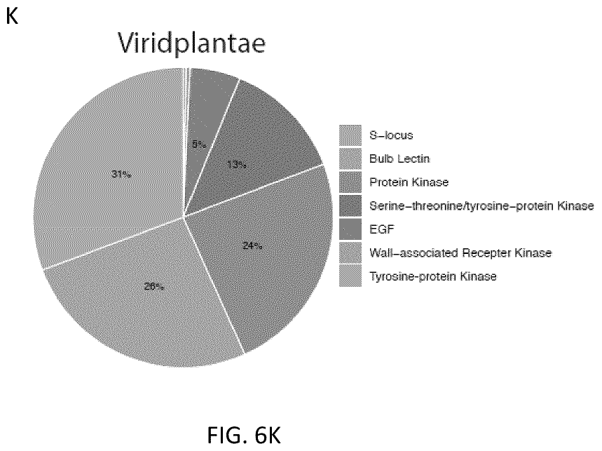

To test the hypothesis that the PAN domain is necessary for immunosuppression, the inventors sought to evaluate the context in which it is found in other proteins. We scanned protein databases, InterPro and UniProt (https://G-LecRK.uniprot.org), and found >28,000 proteins across eukaryotes, archaea, bacteria and viruses carrying the PAN domain. After classifying 2,496 organisms representing 959 genera, into 13 categories (Alveolata, Archea, Amoebazoa, Bacteria, Cryptophyta, Euglenozoa, Haptophyceae, Opisthokonta, Rhizaria, Rhodophyta, Stramenopilles, Viridplantae, and Viruses), the inventors observed that the domain had high fidelity to co-occurring domains and was found in specific protein families within each category. For example, in bacteria, the PAN domain was almost exclusively found in association with the alpha 2-macroglobulin (A2M) or related domains whereas in euglenozoa, it was found almost exclusively in association with the glycosyl hydrolase protein domain (FIGS. 6A-6K).

To assess the validity of association with immune modulation, the inventors performed a GO-enrichment analysis using the >28,000 protein. This analysis revealed 39 GO-terms that were enriched at p<0.05. These included terms like cell recognition (p<1E-30), cell communication (p<1E-30), proteolysis (p<1E-30), pollen-pistil interaction (p<1E-30), reproduction (p<1E-30), response to stimulus (p<1E-30) and response to stress (p<1E-30). These processes are consistent with roles in modulation of immune responses.

### Example 3: Role of PAN Domain Proteins in Gamete Fertilization

Since male gametes are essentially foreign invading bodies, immunosuppression is essential during gamete fertilization, failure of which leads to incompatibility and sterility. As the first interface at which male gametes are perceived, the Zona Pellucida (ZP) occurs outside the female gamete as a layer of glycoproteins that has been reported to release species-specific chemoattractants to attract sperms to eggs. Within this glycoprotein matrix, PAN domain-carrying ZP proteins, also regarded as sperm receptors, are found as transmembrane proteins. ZP proteins have been widely implicated in modulating fertility across a wide variety of eukaryotes including mammalian, avian, amphibian, fish, ascidian and molluscan systems. Bertschinger et al. (Bothalia-African Biodiversity & Conservation, 48(2), 1-8 (2018)) reported that ZP proteins have been successfully used in immunocontraceptive approaches to manage populations of close to 90 animal species.

In plants, the PAN domain-harboring S-locus, serine-threonine, kinases have been shown to mediate pollen recognition and incompatibility in plants. PAN domain mediates S-locus receptor dimerization in the absence of a ligand and suggested that dimerization primes the receptor for interaction with its ligand to enhance the binding affinity. The S-locus receptor kinase (SRK) protein, located in the epidermal cells of the stigma, binds to its cognate pollen coat localized S-locus cysteine-rich (SCR) protein to initiate a signaling cascade to reject self-pollination. However, a non-self SCR protein will not be able to bind the SRK receptor and cross-pollination will occur unimpeded. This example shows that there is specificity between SRK-SCR interactions that is likely coordinated by the PAN domain as both the SCR and SRK proteins contain cysteine-rich regions capable of forming disulfide bonds.

### Example 4: PAN Domain Proteins Implicated in Cell Growth and Proliferation

In animal systems, the PAN domain is found in plasminogen, hepatocyte growth factor (HGF), coagulation factor XI (FXI) and plasma kallikrein (PK), all protein classes implicated in cell growth, proliferation and regulation of immune system responses. Plasminogen and HGF have a similar domain structure with a N-terminal PAN domain, multiple Kringle domains and a serine protease domain. HGF in complex with the MET receptor activates a signaling cascade that initiates cell mobility, tissue regeneration, and angiogenesis. HGF/MET has been shown to have both an immunosuppressive and an immuno-positive role with dysregulation of HGF/MET signaling in tumors leading to invasion and metastasis.

### Example 5: Cell Growth and Proliferation Machinery as a Target for Host-Cell Invasion

FXI and PK are members of the contact system and consist of 4 N-terminal PAN domains followed by a serine protease domain. The contact system is a group of proteins that are activated by coagulation factor FXII. They bind to surfaces to cause blood coagulation and initiate inflammation responses. The contact system is activated by surface proteins of specific pathogens including, Streptococcus pyogenes, Salmonella, and Escherichia coli. Previous research by Krem and Di Cera (Trends in biochemical sciences, 27(2), 67-74 (2002)), has suggested that the blood coagulation system in vertebrates is an evolutionary by-product of the innate immune system. This is supported by the role of the contact system in initiating a signaling cascade that traps microorganisms in a blood clot to stop their invasion. For example, during Streptococcus pyogenes infection, FXII will bind to the surface of the bacteria and activate FXI and PK to trap the bacteria in a fibrin clot and induce a proinflammatory response to clear the infection. Activation of the contact system may also be beneficial to the microbe because the release of bradykinin, a product of contact activation system, will increase vascular permeability and provide a nutrient-rich environment for the bacteria to thrive.

Pathogens have evolved mechanisms to evade this host defense response by targeting another PAN domain-containing protein, plasminogen. In response to the formation of the fibrin clot, S. pyogenes secretes streptokinase, a protein that converts plasminogen to its active form of plasmin. Plasmin will degrade the fibrin clot, which allows the bacteria to escape and the infection to spread. S. pyogenes is one of multiple organisms that target plasminogen to evade the host defense responses. Additionally, parasitic organisms like plasmodium use surface enolase to bind the kringle domain of plasminogen and release plasmin to degrade the extracellular matrix of surrounding tissues and spread the infection.

### Example 6: PAN Domain Proteins Involved in Host-Cell Invasion by Parasites

Apicomplexan parasites including, Plasmodium spp., Toxoplasma gondii, Eimeria spp., and Cryptosporidium spp. secrete micronemal proteins that contain the PAN domain. Apical membrane antigen 1 (AMA1), is a micronemal protein with orthologs in Plasmodium, the causal agent of malaria in humans., Toxoplasma spp., obligate parasites with among the broadest host range in animals, and Eimeria spp., which infects poultry and cattle. Numerous studies have demonstrated that AMA1 is essential for erythrocyte invasion where it has been implicated as a key member of the moving junction (MJ) complex. The MJ complex forms at the contact point between the parasite and the host cell where inward invagination of the erythrocyte membrane occurs. Interaction between AMA1 and rhoptry neck protein 2 were shown to be essential for MJ formation. AMA1 has been widely targeted for development of vaccines against multiple parasites and anti-AMA1 antibodies were shown to have efficacy in reducing invasion of the host cell by these parasites. In T. gondii, Gong et al. (2012) reported that the PAN domain-containing protein (P104), which carries multiple PAN domains was necessary for optimal invasion efficiency. Similarly, genomes of parasitic worms including Trichinella spp., Ancylostoma spp., Pristionchus spp., which infect humans, carry numerous PAN domain proteins including antiplatelet proteins utilized by Leeches (Haementeria officinalis) to inhibit blood coagulation.

### Example 7: PAN Domain Protein Among Insect Vectors of Pathogen and Parasites

Insect vectors of pathogens and parasites pose major disease epidemiological challenges. As such, management of their population using chemicals and, more recently, gene-drives to diminish their populations has been a major focus in efforts to contain transmission of diseases. Given their global economic importance, the inventors compared PAN domain proteins from Diaphorina citri, a psyllid vectoring the bacterium Candidatus liberibacter which causes citrus greening and Anopheles gambiae, a vector of the malaria parasite, plasmodium. Consistent with the inventors' hypothesis and despite major differences in their biology, PAN domains in both organism were found in association with the same ZP domain implicated in immunosuppression during gamete fertilization above (FIG. 2). The inventors propose that these proteins could be targeted by parasites during infection to suppress immune responses, thus enabling persistence of the parasite inside the insect body. This proposition offers the opportunity to target these proteins to lower vector capacity of insects and thus reduce disease transmission.

### Example 8: Prevalence of PAN Domain Proteins in Infectious Viruses and Pathogens

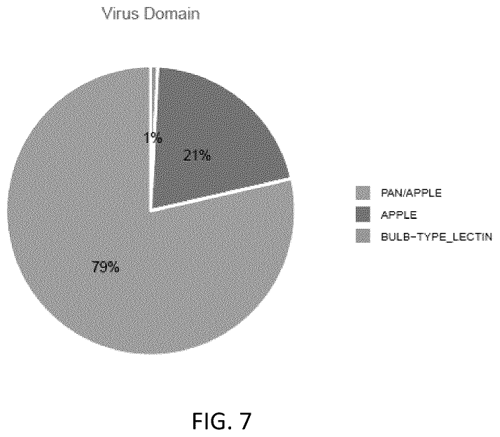

In viruses, long recognized as minimal infectious units, the PAN domain is found in envelope proteins that mostly contain no other recognizable domains except its close relative, the APPLE domain (FIG. 7). This might suggest that, the PAN domain by itself is sufficient for immunosuppression. However, we also found that in Mimiviruses, which include, the African swine virus, Emiliania huxleyi virus and Niemeyer virus, the PAN domain is found in association with HGF, TMhelix, transmembrane, non-cytoplasmic and a cytoplasmic domain and signal peptide domains. Mimiviruses, known as giant virus, are recognized for their significantly larger genomes that encode proteins with functions not typically found in common viruses. Consistent with other complex parasites described above, it appears that Mimiviruses require additional manipulation of the host post-immunosuppression to persist in their host cells.

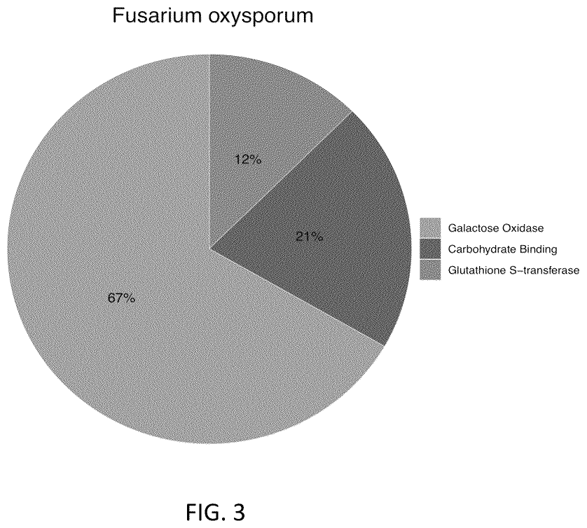

Among fungi, proteins from Ascomycetes represent 97% of PAN domain proteins reported found in our survey. This class of fungi is well recognized for pathogenesis in animals and plants. For example, Fusarium spp. are generally considered harmless, but a subset of these fungi are potent pathogens causing severe diseases in both plants and humans. It is noteworthy that based on our survey, five pathogenic Fusarium species complexes carried disproportionately higher numbers of proteins with the PAN domain compared to other netriaceaes (List 1). In order of descending-predicted number of PAN domain proteins, these were F. oxysporum, F. fujikuroi, F. sambucinum, F. solani, and Fusarium incarnatum-equiseti. Galactose oxidase, carbohydrate binding and glutathione S-transferase contributed the largest proportion of co-occurring domains (FIG. 3).

Among heterokonts, proteins of oomycete origin represented 96% of all PAN domain proteins identified in our survey. Aphanomyces astaci, the causal agent for crayfish plague and Phytophthora spp., one of the most damaging plant pathogens and the causal agent of the sudden oak death epidemic, had disproportionately higher numbers of predicted PAN domain proteins (List 1). Interestingly, the glucose-methanol-choline oxidoreductase (GMC) domain co-occurs with the PAN domain in both organisms. This domain has been implicated in asexual development in Aspergillus nidulans, chemical defenses in leaf beetles and cell extension and expansion during metamorphosis in crustaceans. The involvement of these PAN domain proteins in reproduction and cell proliferation is reminiscent of other eukaryotic systems described above. Moreover, GMC family proteins are recognized for their role in sequestration of the plant host-defense metabolite salicin and hence adaptation of beetle larvae to specific host plants. It is noteworthy that infection of oaks by P. ramorum is often followed by heavy infestation by beetles, although the exact cause has not been identified. Here, the inventors propose that the sequestration of oak defense-related secondary metabolite by the PAN harboring GMC family proteins in P. ramorum could mediate the observed attraction of beetles to infected trees. Similarly, functions performed by GMC proteins in A. astaci could mimic metamorphosis-mediated immunosuppression to facilitate infection leading to crayfish plague.

### Example 9: PAN Domain Penetrance Among Bacterial Symbionts

Assessment of domain presents in the bacteria kingdom revealed that the PAN domain is found predominantly among alpha proteobacteria, well recognized for their host-cell invasion and symbiosis behavior. Of the 1,783 bacterial proteins carrying the domain, 1,587 (89%) were found in Rhizobium sp. and Rhodobacter sp. (List 1). These bacteria are specialized in nodulation of land plants and symbiosis with marine danoflagellates, respectively. The PAN domain was almost exclusively found in anti-peptidase proteins and co-occurred with the A2M domain or related macroglobulin domains (FIG. 2). A2M is recognized as a broad-spectrum protease inhibitor that has been widely implicated in immune modulation and is associated with a wide array of diseases. Aside from its anti-protease activity, A2M has binding affinity to a wide variety of molecules. In both Rhizobium sp. and Rhodobacter sp., heme sequestration is an essential process for the survival of bacteria inside host cells. The exact mechanism underlying this process has remained largely elusive with limited progress in identifying proteins related in to heme sequestration. Here, the inventors note that A2M has been previously reported to function as a Hx:heme receptor, known as the Low-density lipoprotein receptor-related protein-1 (LRP1). LRP1 was shown to play a central role in heme scavenging after intracerebral hemorrhaging in mice. With this observation, the inventors propose that the PAN domain may have been co-opted to act as an immunosuppressant while the A2M domains functions to sequester heme and create the necessary environment for bacterial survival in plant host cells.

### Example 10: Domain Expansion in Organisms Specialized in Symbiont Recruitment

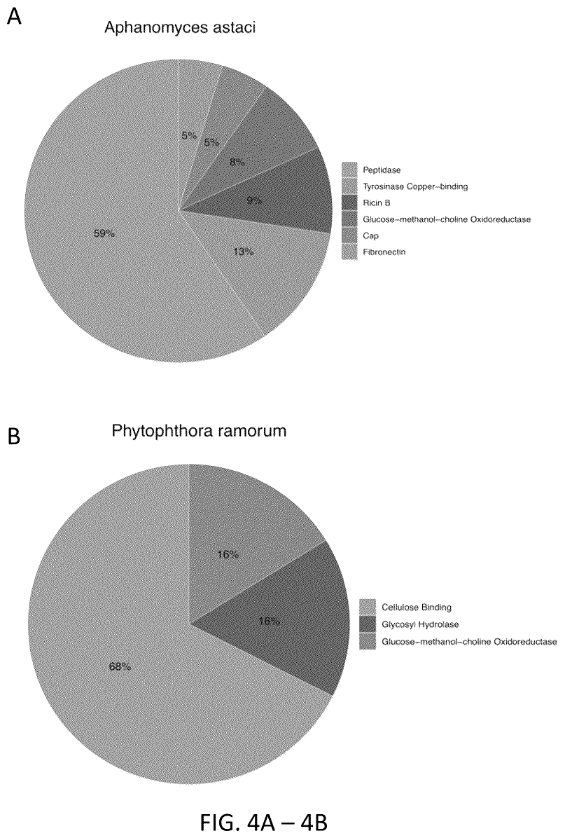

Numerous organisms such as corals, planktonic crustaceans and plants depend on symbionts to perform essential functions. In these instances, immunosuppression is a key step in conferring compatibility between hosts and symbionts. For example, Daphnia magna is a planktonic crustacean that is considered a model system to study host-parasite interaction since it has highly diverse internal microbiome. Evaluation of PAN domain genes in its genome revealed occurrence of duplicated gene in the protein families such as Brain chitinase, Chorion peroxidase, Class A scavenger receptor (cA-SR) with a serine protease domain, Coagulation factor XI, No mechanoreceptor potential (nomp) A-like, Peroxinectin (PX)-like, putative Transmembrane protease serine, ZP as well as multiple uncharacterized protein. The participation of Chitinase, Coagulation factor XI, proteases and ZP protein in immunosuppression-related proteins was discussed above and cA-SRs have been studied for their function in innate immunity. Akin to D. magna, plants also recruit symbionts to perform essential functions including nutrient mobilization. The inventors note that G-LecRKs implicated in immunosuppression during recruitment of fungal symbiont above occur as a large protein family with hundreds of members and exhibit frequent gene-expansion across plant genomes (FIG. 5). The inventors hypothesize that both these organisms coopted immunosuppressive proteins to facilitate their symbiotic lifestyle by allowing rapid expansion of these genes within their genomes.

### Example 11: PAN Domain Similarity to Cysteine-Rich Antimicrobial Peptides

Cysteine-rich peptides (CRPs) are produced by multicellular organisms and are known for their use as antimicrobials. While these peptides are not considered PAN domain-containing peptides, they have functional and structural similarities to the PAN domain in that they consist of conserved cysteine residues that form disulfide bonds and are involved in immune modulation. The CRP protein, TAP, was first isolated from bovine tracheal mucosa and were noted to have antimicrobial properties. Defensins are a class of CRPs common to both plants and animals and are involved in host innate immunity. CRPs are secreted by plants and have been shown to be involved in plant defense, symbiotic relationships with bacteria, development, reproduction and pollen-tube guidance. The nodule-specific cysteine-rich (NCR) peptides are a subclass of CRPs in plants that are secreted by the host plant and regulate symbiosis between legumes and rhizobia during nodule development and nitrogen fixation. The structure of NCRs resembles plant defensins that are involved in regulating plant innate immunity, but they have a different function in that they regulate symbiosis with rhizobia during nitrogen fixation. Given their structural similarity, it is plausible that CRPs function as ligands targeting PAN domain proteins to modulate immunosuppression.

### Example 12: Functional Role of the HGF PAN Domain in Human Cell Lines

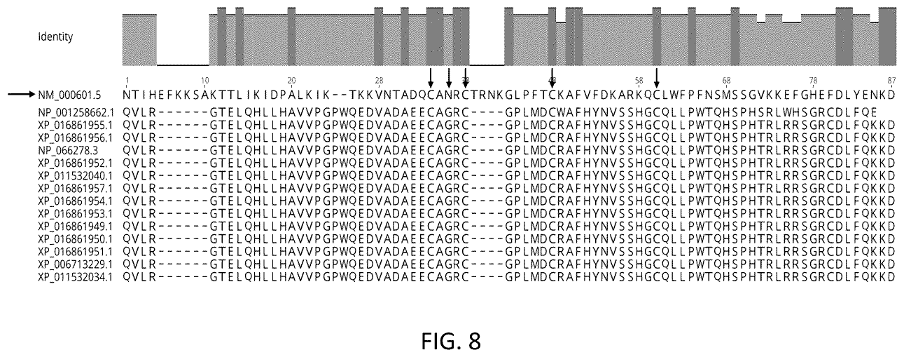

c-MET is a receptor tyrosine kinase which is present on the surface of various epithelial cells. c-MET gets activated by binding with its ligand, HGF. HGF is an important paracrine mediator of growth, invasion and angiogenesis in several cancers. All biological impacts of HGF are triggered by stimulating its cell surface receptor, c-MET with related activation of downstream effector pathway. Upon binding with its ligand, HGF ligand bound c-MET receptor gets internalized via endosomal internalization and undergo lysosomal degradation. HGF possesses a canonical 4 conserved cysteine residues in its PAN domain whose function remains to be determined (FIG. 8).

### Example 13: Mutating Cysteine Residues in PAN Domain Results in Enhanced HGF Stability

To determine the function of the cysteine residues on the PAN domain, wild type HGF was subjected to mutagenesis and four conserved cysteine (C) residues on PAN domain were mutated to alanine (A) and one conserved asparagine (N) was mutated to aspartic acid (D). HEK293T cells were transfected with Flag tagged wild type and Flag tagged PAN mutants HGF which are HGF 4Cys-4Ala and HGF N-D. Lysates were resolved on a SDS polyacrylamide gel, transferred to PVDF membrane and probed sequentially with antibodies for Flag tagged HGF and endogenous Actin.

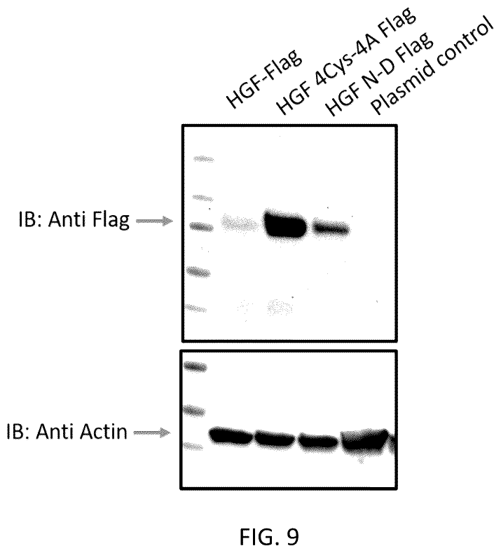

Lane 2 of FIG. 9 shows that HGF with four cysteine mutations in PAN domain (HGF 4Cys-4A) had higher stability compared to wild type HGF (Lane 1) when probed against HGF-Flag (FIG. 9). Actin was shown as a loading control. This data suggests that cysteine residues on the PAN domain of HGF mediate proteolysis of the HGF protein.

### Example 14: Activation of MET Signaling is Attenuated by the Cysteine Residues Mutations in HGF PAN Domain

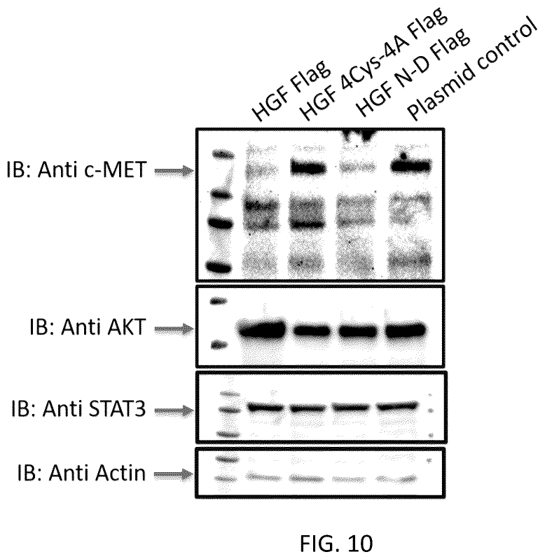

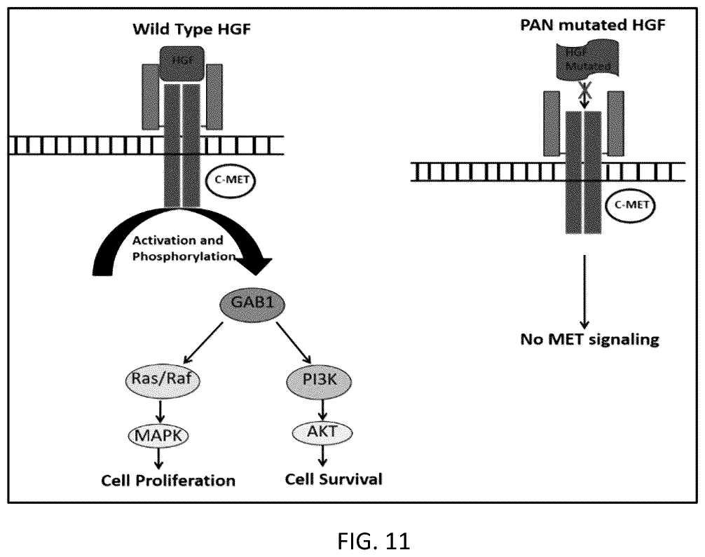

Once activated, the HGF/c-MET undergoes a rapid internalization from cell membrane to cytoplasm for degradation/recycling depending on the cellular demand. To address whether the PAN mutant HGF displays more stability as a result of MET binding capability, the inventors studied the endogenous c-MET expression in HEK 293T cells overexpressed with wild type HGF and PAN mutants HGF. FIG. 10 topmost panel shows 4Cys-4Ala HGF was unable to activate HGF-c-MET signaling pathway as evident by stable MET expression. The enhanced stability of the MET receptor indicates that the receptor is retained on the cell surface.

Both wild type HGF and N-D HGF could bind c-MET and activates the cascade results in MET degradation. Signaling downstream from MET receptor is mediated through the recruitment of other adaptor proteins including STAT3 and AKT which are stably expressed in cells even after signaling cascade activation. In FIG. 10, both middle and lower panel showed the presence of both STAT3 and AKT in cells under all circumstances demonstrating the specificity of the HGF PAN for c-MET. This data for the first time, demonstrates a direct link between Cysteine rich PAN domain with HGF function and c-MET signaling.

HEK 293T cells were transfected with Flag tagged wild type HGF and Flag tagged PAN mutants HGF which are HGF 4Cys-4Ala and HGF N-D. Lysates were resolved on SDS-polyacrylamide gel, transferred to PVDF membrane and probed sequentially with antibodies for c-MET, AKT, STAT3 and Actin. FIG. 10, lane 2 in top panel shows significantly higher c-MET stability compared to lane 1 with overexpressed wild type HGF. Lane 4 with overexpressed empty plasmid showed same c-MET stability as lane 2. This confirms HGF 4Cys-4Ala cannot turn on the c-MET signaling and thereby c-MET is not getting degraded. Immunoblot against AKT and STAT3 showed the degradation is c-MET specific as both proteins were stable under all conditions. Actin was used as a loading control.

c-MET is a heterodimer consisting of an extracellular α-chain and a transmembrane β-chain connected through a disulfide bridge. Activation of the MET receptor through HGF binding promotes the autophosphorylation on the tyrosine residues on the intracellular domain of c-MET. This opens a docking site for the recruitment of other signaling protein ultimately establish a downstream signaling cascade and biological response. HGF-cMET signaling cascade triggers the activation of phosphatidylinositol 3-kinase (PI3K) and AKT pathway well defined in cell migration. Besides that, the mitogen activated protein kinase (MAPK) and the extracellular kinases (ERK1/2) are well known downstream mediator of this MET signaling cascade. Abnormal activation of c-MET-HGF cascade has been reported to promote the progress of multiple cancers including glioblastoma, liver, lung, breast, colon to name a few. In all those cancers, activated PI3K/AKT, JAK/STAT and Ras/MAPK pathways have been reported to be elevated. All these have negative impacts on the cancer prognosis.

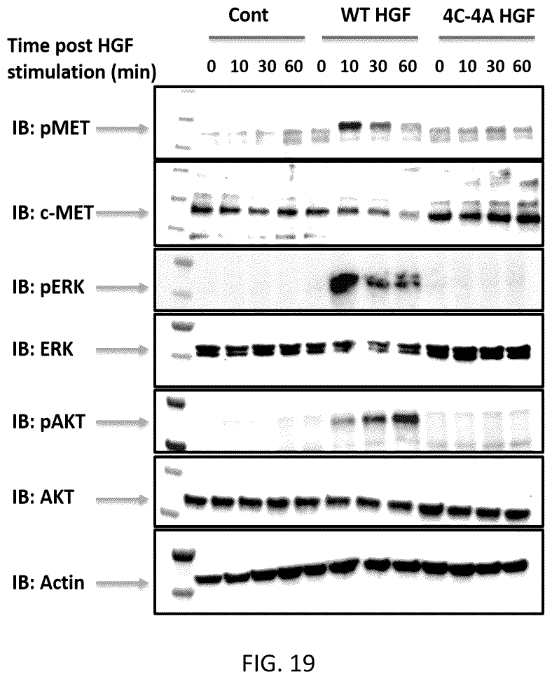

To evaluate the effect of PAN mutations on HGF at a biochemical level, the inventors performed western blotting on the protein lysates derived from HEK293T cells stimulated with wild type HGF and 4Cys-4Ala HGF PAN mutant proteins for the indicated time (FIG. 19). FIG. 19 topmost panel shows, 4Cys-4Ala HGF PAN mutant was unable to activate c-MET as evident by the lack of phosphorylation compared to the wild type (WT) HGF.

However, activation of MET receptor with WT HGF results with WT HGF results the downstream activation of MET signaling cascade as pAKT and pERK levels increased (FIG. 19, panel third and fifth) over time. Mutations on Cysteine residues on PAN were able to completely block the AKT and MAPK pathways.

HEK 293T cells were grown on 6 cm plates and stimulated with both WT HGF and 4Cys-4Ala HGF proteins for the indicated amount of time. Lysates were resolved on SDS-polyacrylamide gel, transferred to PVDF membrane and probed sequentially with antibodies for pMET,c-MET, pAKT, AKT, pERK, ERK and Actin.

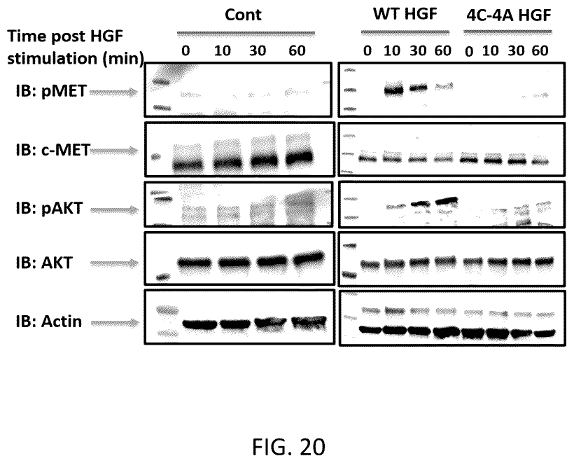

The proto-oncogenic receptor tyrosine kinase c-MET and HGF reported to be deregulated and promote malignancy in cancer and brain tumors. The inventors investigated the impact of PAN mutations on cMET activation in glioblastoma cell lines U87. Human glioblastoma cell line was used and western blotting on cell lysate were performed following the WT and 4Cys-4Ala HGF stimulations for the indicated amount of time (FIG. 20). The results show that PAN mutations on HGF had similar negative impact on cMET signaling cascade activation in glioblastoma cell line.

Analysis of HGF PAN Mutations on cMET Activity in Cells. Mutations in HGF PAN Domain Alters the Localization of cMET in Cells.

It has been well described before that upon HGF stimulation, cMET gets activated by tyrosine autophosphorylation and is rapidly internalized and translocate to the nucleus. Following the stimulation, c-MET is rapidly internalized to early endosomes and translocate to the nucleus either to initiate calcium signals and or to upregulate PAX5, a transcription factor, in the nuclear compartment. Here we demonstrate that 4Cys-4Ala PAN mutant HGF failed to activate the cMET translocation to nucleus as compared to the WT HGF stimulation. HeLa cells were grown on 24 well plates before stimulated with both WT and 4Cys-4Ala PAN mutant HGF proteins for 30 minutes where indicated. Cells were then fixed, permeabilized and observed against cMET using fluorescence microscope. The fluorescence images confirmed that cMET redistributes to the region of nucleus from cell membrane following WT HGF stimulation but not by the 4Cys-4Ala PAN mutant HGF.

### PAN Domain Positively Impacts on Cell Proliferation.

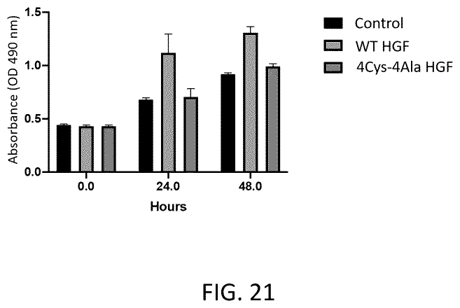

To investigate the role of HGF on cell cycle, HGF WT and 4Cys-4Ala PAN mutant HGF were used as an exogenous factor to determine its impact on cell proliferation. HEK 293T cells were grown in 96 well plates and stimulated with 100 ng/mL WT HGF and 4Cys-4Ala PAN mutant HGF for 0, 24 and 48 hr. MTT, (3-(4,5 dimethylthiazol-2-yl)-2,5-diphenyltetrazolium bromide) reagents were added and absorbance were measured according to manufacturer instructions. There was a significant drop in the absorbance of the cells treated with PAN mutant HGF compared to WT HGF stimulated one (FIG. 21). The absorbance is related to cell proliferation. This observation confirmed a direct evidence of attenuation of cell proliferation over HGF PAN mutations. This also confirmed that targeting four cysteine residues on the PAN domain of HGF can attenuate the cell proliferation which is otherwise elevated in different cancers.

Investigation of Transcriptional Profiles Upon the Stimulation of Cells with PAN Mutant HGF.

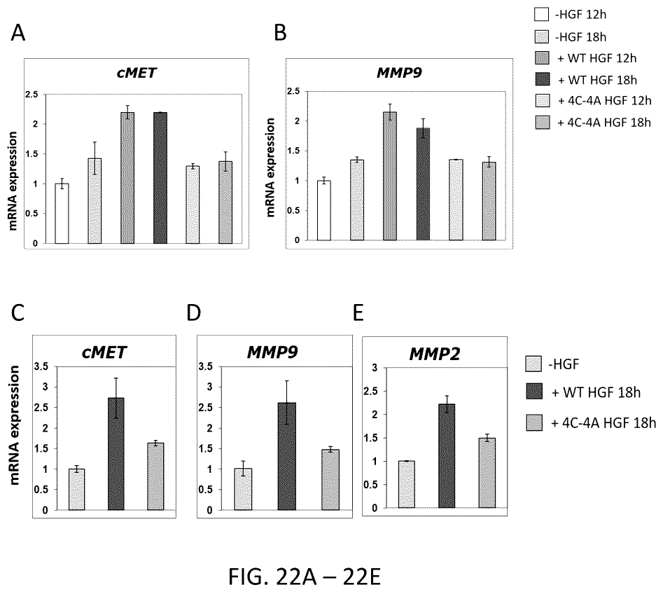

HGF has been demonstrated to stimulate cancer proliferation, migration and metastasis by increasing the migration and expression of matrix metalloproteinases (MMPs) in human. To further address the impacts of PAN mutations on HGF towards cell, the inventors quantified relative mRNA level of cMET, MMP9, and 3-actin by real time-PCR according to the protocol following stimulation with WT HGF and PAN mutant HGF. FIGS. 22A-22B show that interaction between HGF-cMET is important for the upregulation of cMET and MMP9 in HEK 293T cells.

Glioblastoma is the most severe brain tumor type with the highest malignancy and lowest prognosis. MMPs can enhance the invasive ability of tumor cells by degrading extracellular matrix proteins (such as collagen, fibronectin and proteoglycan) as well as growth factor binding protein, growth factor precursor, receptor tyrosine kinase, cell adhesion molecule and other proteases. Among the MMPs, the elevated expression level of MMP2 is meticulously related to the progression of glioblastoma malignancy. The inventors further explored the relative mRNA level of MMP9 and MMP2 along with c-MET in glioma cells following HGF stimulation (FIGS. 22C-22E). U87 cells were stimulated with WT HGF and 4Cys-4Ala PAN mutant HGF for 18 hr. Cells were collected and RNA expression level were determined. FIGS. 22D-22E show the relative abundance of MMP9 and MMP2 were low upon PAN mutant HGF treatment as compared to WT HGF stimulation.

### Example 15: RLK PAN Domain Mediates G-lecRLK Dimerization

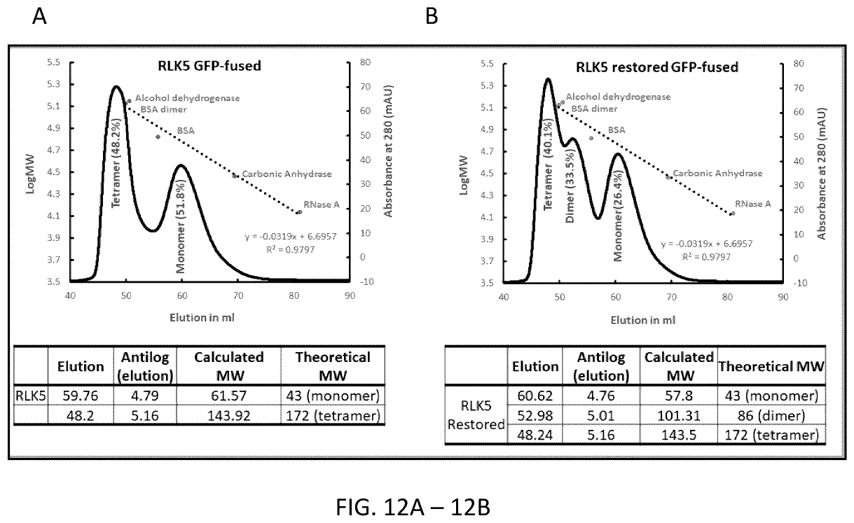

The PAN domain possesses the characteristic 6-cysteine residues in its core that are highly conserved. These cysteine residues coordinate three disulfide bridges to form a hairpin structure. The inventors hypothesized that cysteine rich PAN domain of G-lecRLKs form disulfide bonds involved in dimerization and subsequent receptor proteolysis to prevent downstream signaling cascades. To address this hypothesis, a GFP tagged PAN domain construct of RLK5 was made. In addition, mutated amino acids in the RLK5 vestigial domain were restored to consensus PAN domain amino acids based on the alignment in FIG. 1, this construct was designated RLK5-restored (RLK5R). Both RLK5 and RLK5R constructs were transfected and overexpressed in HEK293 cells. Proteins were purified on GFP column and loaded into size exclusion column (SEC) to analyze the oligomeric state. Consistent with the inventors' hypothesis, this biochemical assay showed that RLK5R could form dimer in SEC whereas RLK5 could not (FIGS. 12A-12B).

### Example 16: RLK PAN Domain Enhances Receptor Degradation Via MAP Kinase Pathway

Pattern recognition receptors recognize invasive microbe-associated signals and confer host immunity by triggering the initial layer of plant defense to prevent pathogen proliferation. Previous studies have shown the importance of MAP kinase (MAPK) in plant innate immunity. This MAPK cascade consists of the MAP kinase kinase kinase, YODA, MAP kinase kinases MKK4 and MKK5, and MAP kinases MPK3 and MPK6. MAP kinases undergo phosphorylation mediated activation. In Arabidopsis, prolonged activation of MPK3 and MPK6, two kinases critical for pathogen resistance, results in the inhibition of photosynthesis and accumulation of reactive oxygen species (ROS) in chloroplasts.

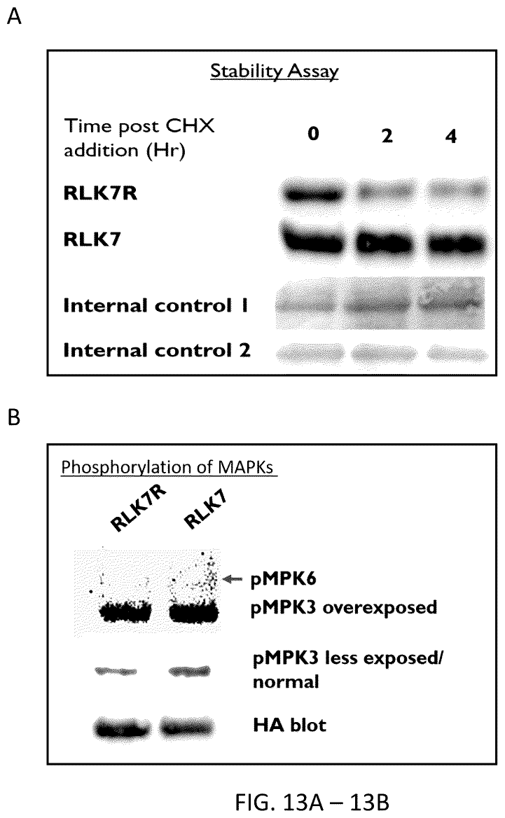

The inventors wanted to test whether PAN domain containing G-lecRLKs undergo dimerization which prevents the phosphorylation mediated activation of downstream signaling cascade. To inventors first performed Cycloheximide (CHX) stability assay. Both RLK7 (PAN mutated variant from Salix described above) and RLK7R (PAN domain restored to consensus amino acid sequence) were overexpressed in Arabidopsis protoplast followed by CHX treatment at indicated time points, to inhibit the protein turn over. Lysates were resolved with SDS-PAGE gel and documented with chemiluminescence BIO-RAD gel doc system. Resulting data indicated a higher half-life or extended stability for RLK7 over RLK7R following 0-4 hr time points (FIG. 13A). Consistent with expected downstream phosphorylated of MAP kinases, the inventors observed an overall increase in the MPK3 and MPK6 phosphorylation (FIG. 13B).

Since the apparent protein instability of RLK7R resulted in its inability to trigger phosphorylation of MPK3 and 6 as well as subsequent defense response, the inventors hypothesized that RLK5 and RLK7 with mutated PAN domains lost their immunosuppressive roles and thus should trigger defense signaling.

### Example 17: Central Player of Innate Immune Cascade, MAPKs are Activated in RLK5 and 7 Overexpressed Transgenic Lines

Mitogen activated protein kinases (MAPKs) are well conserved protein kinases in eukaryotes where MAPK kinase kinase (MAPKKK) activates MAPK kinase (MAPKK) which ultimately activates MAPKs. Upon the detection of any external stimulus or environmental changes at the cell surface, MAPKs gets upregulated and participates in the classical signal transduction of immune responsive genes. After overexpression of RLK5 and RLK7 in Arabidopsis, the inventors were interested to evaluate whether any signature MAPKKK and MAPK were activated. Upstream regulators of this pathway like MAPKKK13 and MAPKKK14 were highly upregulated in transgenic lines (FIGS. 14A-14B). Additionally, the inventors were interested in studying the regulation of downstream MAPKs. MAPK3 is one of the most well-known signature gene of this pathway and showed a significant upregulation in transgenic lines suggesting RLKs with mutated PAN domains could induce immune signaling via MAPK pathway in plants (FIG. 14C).

### Example 18: RLK PAN Activates Proteins Involved in JA/ET Pathway

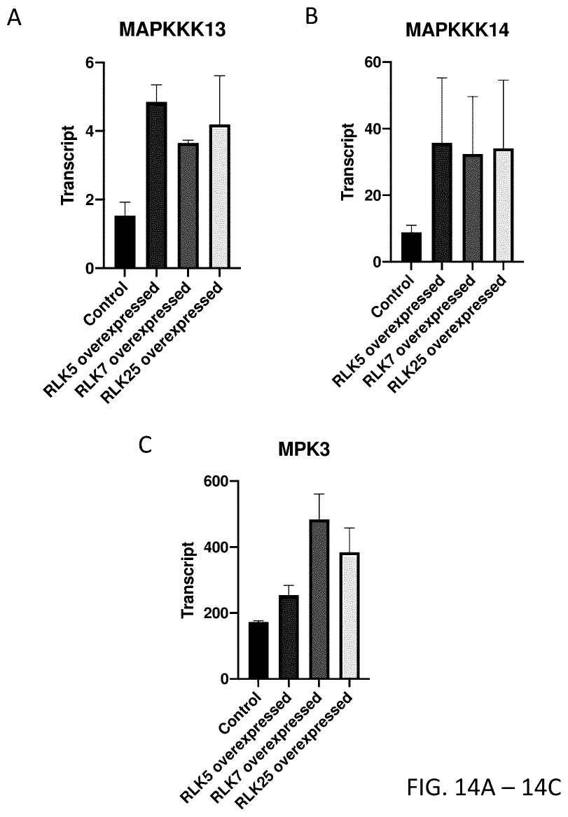

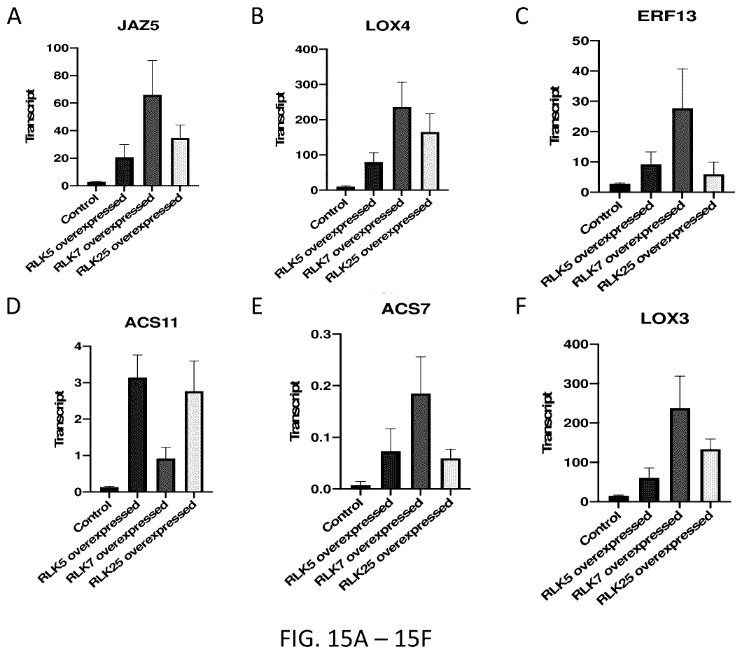

The expression level of the genes related to Jasmonic acid (JA) synthesis were also examined. As shown in FIG. 15B, the inventors were interested to observe the signature genes of the JA and ethylene signal transduction (ET) pathway. LOX3, LOX4 and JAZ5 are the signature genes of JA pathway and are upregulated in transgenic lines compared to wild type. Other signature genes involved in ET and JA pathways are ERF13, ACS11 and ACS7. These mediators are also upregulated in our transgenic lines. This data suggests that constitutive overexpression of RLK5 and RLK7 in Arabidopsis activates JA and ET pathway (FIGS. 15A-15F).

### Example 19: RLK PAN Attenuates Salicylic Acid Pathway of Plant Innate Immunity

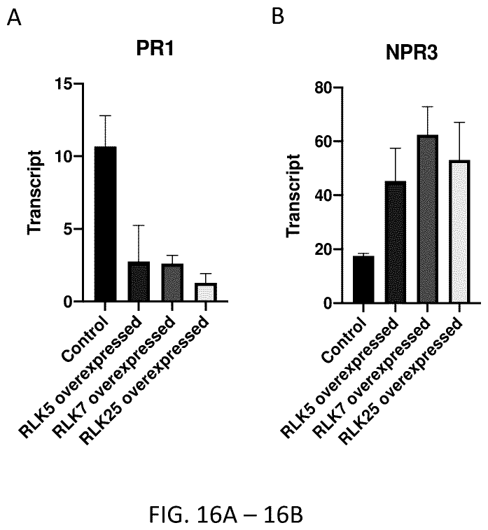

Salicylic acid (SA) is an important signal molecule in plant defense response. One essential player in the SA defense response pathway is non-expresser of pathogen related genes 1 (NPR1). NPR1 is a positive regulator of SA pathway. It is well known that Arabidopsis npr1 mutant shows increased disease susceptibility and downregulation of genes specifically pathogen related (PR1) gene. PR1 genes were downregulated in transgenic Arabidopsis constitutively expressing RLK5 and RLK7 (FIG. 16A). In contrast, NPR3 was significantly overexpressed in transgenic lines compared to wild type (FIG. 16B). From these results, the inventors conclude that constitutive expression of RLK5 and RLK7 contributed to the regulation of SA related genes and appeared to suppress the activation of SA signaling pathway.

### Example 20: Classical WRKY Genes are Hyperactivated in RLK PAN Transgenic Plants

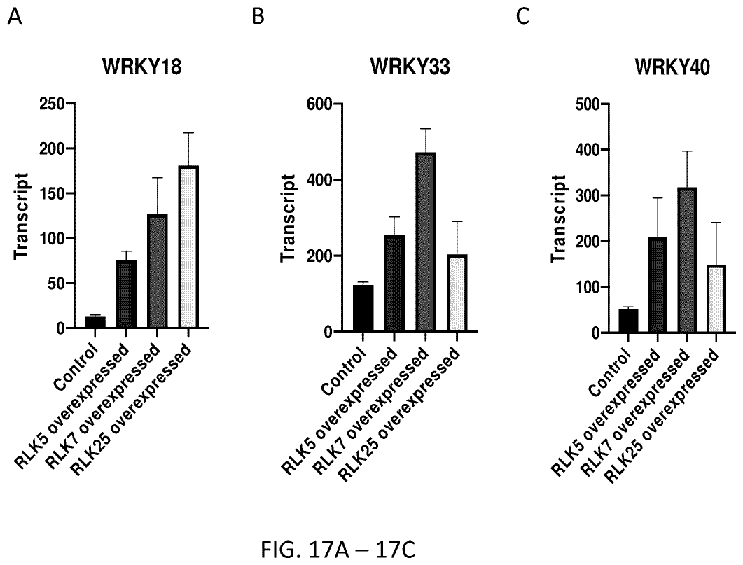

WRKYs are transcription factors involved in various plant processes but notably in fighting against diverse biotic and abiotic stresses. WRKY18, WRKY33 and WRKY40 are the prominent WRKY genes involved in plant immunity and the inventors determined significant upregulation of these transcription factors in transgenic lines as shown in FIGS. 17A-17C.

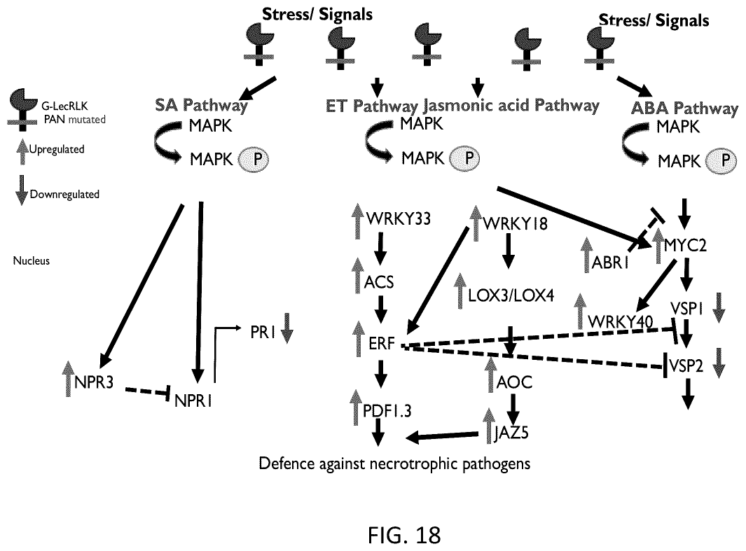

A proposed model for function of the PAN domain in plant G-lecRLKs is found in FIG. 18.

### List 1: List of UniProt IDs for PAN Domain Containing Proteins Across Species.

R4G2C1, R4G7D0, G8EBH2, G8EBN0, G8EDK5, A0A3T1CXD4, A0A3T1CXP1, A0A1E1EXB1, A0A1E1ETK2, A0A1E1EUC2, L8GVM1, L8GRF2, L8GX08, L8GY48, L8H5C9, L8GYA1, J3E810, J2YWZ6, J3IYU5, A0A2L2DLV0, A0A2L2DIZ7, A0A0G2Y717, E3VZX8, E3VYF9, F8V6R0, A0A0G2Y378, A0A0G2Y8U4, A0A2L2DJ09, Q5UPR5, Q5UNW8, Q5UQG0, A0A2L2DM38, A0A0G2Y3S5, A0A0G2YB83, A0A0G2Y7I9, L7RC65, L7RC22, L7RCU4, A0A091MSS1, A0A091MQE1, A0A3Q1FYN5, A0A3Q1FW77, A0A3Q1EDF5, A0A3Q1G8B6, A0A3Q1F1Z0, A0A3Q1I407, A0A316YDH8, A8ZR36, A0A0A7CPG5, A0A0A7CP60, A0A0A7CN07, A0A0A7CNR8, A0A0A7CN57, A0A0A7CP35, A0A0A7CMN8, A0A0A7CP65, A0A0A7CP71, A0A0A7CN98, A0A0A7CP31, A0A1V9ZRA5, A0A1V9ZDE5, A0A1V9YLY7, A0A1V9Z1A6, A0A1V9ZKZ6, A0AIV9Z451, A0A0A7CNG8, A0AIV9YJ62, A0A0A7CMR5, A0A0A7CPD7, A0A1V9Z629, A0A0A7CPH5, A0AIV9YJK3, A0AIV9YSE1, A0AIV9YDS4, A0AIV9YHW9, A0A0A7CMJ7, A0A0A7CN31, A0AIV9ZMC6, A0A0A7CNG9, A0AIV9YH26, A0AIV9YUS3, A0AIV9ZRD0, A0A0A7CNY9, A0A0A7CM59, A0AIV9ZKR0, A0AIV9ZUM1, A0AIV9YUE0, A0AIV9YRF9, A0AIV9ZB07, A0A0A7CN88, A0AIV9YXW4, A0A1V9Z9X3, A0A0A7CLM9, A0A1V9Z933, A0A1V9YWJ8, A0A2E4CB18, A0A2E4CBG1, A0A2E4CB13, A0A2E4CBJ3, A0A2V9ZGI7, A0A150V788, A0A150VFP3, A0A3A1WDE8, A0A444V1E0, A0A444V1E2, F4X7R3, F4X293, F4X6U6, F4X815, F4WMQ5, F4X291, A0A058ZM17, A0A037ZPD6, A0A2R6Q4L0, A0A2R6RAZ9, A0A2R6Q6W9, A0A2R6QV86, A0A2R6QWW6, A0A2R6PXQ8, A0A2R6R4I5, A0A2R6PR71, A0A2R6RB30, A0A2R6P2X1, A0A2R6R7U2, A0A2R6QA16, A0A2R6PTF8, A0A2R6Q077, A0A2R6QTI6, A0A2R6R4H6, A0A2R6QBV2, A0A2R6PQK3, A0A2R6QCM9, A0A2R6RF49, A0A2R6QDU5, A0A2R6QLP2, A0A2R6PTF3, A0A2R6PGN0, A0A2R6RRI6, A0A2R6RGA0, A0A2R6PWC1, A0A2R6PXW3, A0A2R6Q3G8, A0A2R6Q7Z3, A0A2R6QBX6, A0A2R6QLN4, A0A2R6PTE6, A0A2R6QWB9, A0A2R6PJ76, A0A2R6RP34, A0A2R6RB22, A0A2R6QXS9, A0A2R6PEE6, A0A2R6NZR5, A0A2R6RB09, A0A2R6PQU7, A0A2R6QLP8, A0A2R6QC53, A0A2R6PUZ3, A0A2R6PTE5, A0A2R6QBV5, A0A2R6RB02, A0A2R6QCD9, A0A2R6QBW7, A0A2R6R4H9, A0A2R6QWW7, A0A2R6PK86, A0A2R6NKZ3, A0A2R6PRE4, A0A2R6PY69, A0A2R6RB03, A0A2R6QDU9, A0A2R6P887, A0A2R6QXB5, A0A2R6QA21, A0A2R6PRM8, A0A2R6QMJ8, X1WJB0, J9K2R9, J9LS79, J9KAI5, J9KPT6, J9JPB7, J9KOU0, J9JMX3, B3G4G6, B3G448, B3G4F0, A0A1S4G3R5, Q1HRG8, A0A1S4FK22, Q16Y69, A0A1S4FG50, Q16VE9, Q17JC8, Q171P7, Q17FY1, Q16VE8, A0A182H4K1, A0A182H5Q1, A0A182G6M4, A0A182G9D3, A0A182G9D4, A0A452XH92, A0A452ZGN2, A0A453BYP1, A0A453LJQ2, A0A452ZGI7, A0A453HQH7, A0A453AAT9, A0A453DBA5, A0A453BBE0, A0A453BYQ0, A0A453LK80, A0A453ADR6, A0A453Q2G0, A0A452Z039, A0A452ZI33, A0A453D3G9, A0A453DYZ0, A0A453JHI0, A0A453Q2D1, A0A453LKE2, A0A453F7F0, A0A453GM45, A0A452Y396, A0A453BYT2, A0A453MEH1, A0A453EGG7, A0A453D372, A0A453SRN1, A0A453FPM7, A0A453BYQ9, A0A453ADP5, A0A453JI06, A0A453DD86, A0A453LKD6, A0A453DYQ0, A0A453ADU5, A0A452XJR0, A0A453JGU6, A0A453NKE5, A0A453LKC6, A0A453D3I3, A0A453JIH7, A0A453LKE3, A0A453DBY7, A0A453SVX0, A0A453FPN7, A0A453DDB2, A0A453DB66, A0A453FPT3, A0A453DAW9, A0A452XJW5, A0A453DYR4, A0A453LKH1, A0A453DBP7, A0A453NKT3, A0A453ADR2, A0A453D3G3, A0A453AAM1, A0A453D3A4, A0A453PQK2, A0A453Q1Y2, A0A453LKA1, A0A453MGE8, A0A453F7I6, A0A453FPR4, A0A453D383, A0A452Z010, A0A453QTP8, A0A453FPN3, A0A453JHT4, A0A453FPT0, A0A453FPK6, A0A453EGB1, A0A453QZE9, A0A453BYN4, A0A453KBQ0, A0A452Z4D8, A0A452ZEX2, A0A453SHI0, A0A453D306, A0A453FPP1, A0A453F7D3, A0A453LN14, A0A453LK85, A0A453LK71, A0A453H3Z1, A0A453DYX5, A0A453FPQ5, A0A453DBC1, A0A452ZGJ2, A0A452ZGK8, A0A452ZEZ6, A0A453LK88, A0A452YZZ8, A0A453D3A8, A0A453LKC7, A0A453GLW6, A0A453LK95, A0A453LK89, A0A453D358, A0A453QZ76, A0A453GMC5, A0A453BYQ7, A0A453T0W6, A0A453DD79, A0A453BBQ0, A0A453Q244, A0A453DBN0, A0A453GM60, A0A453D325, A0A453Q771, A0A453BCU3, A0A452XK72, A0A453DBI2, A0A453QZN5, A0A453HAH1, A0A453FPI6, A0A452Z002, A0A453LKB1, A0A452XK48, A0A453BBD4, A0A453Q2E7, A0A453QZF2, A0A453DBB2, A0A453JEW1, A0A453SU32, A0A453FPN4, A0A453DBG7, A0A453D337, A0A453LKF9, A0A453F7G5, A0A453SVW0, A0A453BC35, A0A453D3B0, A0A453DA54, A0A453TAB1, A0A453D3G4, A0A453JHJ1, A0A453SRL9, A0A453ADQ2, A0A453D349, A0A453PFF2, A0A453DA76, A0A452XH56, A0A453DC09, A0A453QTY7, A0A453DB70, A0A453DBM7, A0A453FPK2, A0A453DBH5, A0A453Q2F9, A0A453ADR3, A0A453DDD0, A0A452Z027, A0A453FPS3, A0A453D6J6, A0A453J9S0, A0A453Q2M7, A0A453LJR3, A0A453D329, A0A453GM00, A0A453BYP5, A0A453BBD9, A0A453BYM4, A0A453Q291, A0A453QU71, A0A453H3Z4, A0A453SGZ7, A0A453ADQ4, A0A453LKC4, A0A453LKG3, A0A453DBI3, A0A453DC06, A0A453SRP6, A0A453SRK1, A0A453ADR9, A0A453QZE3, A0A453D3H5, A0A452Z019, A0A452Y3L8, A0A453DB32, A0A453D353, A0A453H9U8, A0A453DBK3, A0A453DBC7, A0A453NLT6, A0A453ILUO, A0A452ZQ21, A0A453DBV6, A0A452Z009, A0A453DDA0, A0A453G5Z3, A0A452ZGN5, A0A453D6L7, A0A453BYQ2, A0A453D2Z9, A0A453DBL2, A0A453DBD7, A0A453FPV1, A0A453D352, A0A453Q591, A0A452ZI59, A0A453Q2L2, A0A453D333, A0A453TA51, A0A453DDL7, A0A453G617, A0A453D2X4, A0A452XD19, A0A453Q1X4, A0A453MGQ3, A0A453HRF8, A0A453EGH4, A0A453HQJ5, A0A453LN67, A0A453DB22, A0A453BDB4, A0A453LK93, A0A453DDM1, A0A453D369, A0A453DBK6, A0A453SRN7, A0A452ZGM5, A0A453D6Q2, A0A453LKB4, A0A453BYR3, A0A453D3C0, A0A452XD37, A0A453DDM6, A0A453ADV9, A0A453FPM3, A0A453TAE6, A0A453BC47, A0A452XK40, A0A453F7D6, A0A453NKD5, A0A453DC51, A0A453DBE4, A0A453T114, A0A453BYS4, A0A453H3Z7, A0A453ATP0, A0A453G6T0, A0A452XZ51, A0A453DBW3, A0A452XD28, A0A453LJR8, A0A453BB79, A0A453D382, A0A453LK83, A0A452ZI63, A0A452XZD5, A0A452Z4E2, A0A453T139, A0A453DB02, A0A453FPR7, A0A453PFD9, A0A453BBJ6, A0A453LK79, A0A453BBV1, A0A453DBL5, A0A453FPR9, A0A453D361, A0A452XD46, A0A453QZU7, A0A453ATS1, A0A453FPJ1, A0A452XB26, A0A453QTW5, A0A453BYP4, A0A453D323, A0A453ADR1, A0A452XJS2, A0A453FPS0, A0A453FPQ0, A0A453JB99, A0A453DBF2, A0A453KC08, A0A452XJG0, A0A453DBT2, A0A453IFX4, A0A452XK35, A0A453D3B3, A0A453LJU7, A0A453JH21, A0A453S9X2, A0A452XJT3, A0A453DBV3, A0A452Z033, A0A452XH89, A0A453SAP2, A0A453BBW1, A0A453DC15, A0A453DBY3, A0A453DDL1, A0A453D3A1, A0A453D396, A0A453H3Z3, A0A453EGJ2, A0A453DBW8, A0A453LKB3, A0A453EGJ7, A0A453G694, A0A453Q4I2, A0A453CF81, A0A453FPL3, A0A453GM65, A0A453GMJ9, A0A452Y3H0, A0A453D3A3, A0A453DDC2, A0A452ZNP1, A0A452ZGK3, A0A453DBU3, A0A453GQY1, A0A453IM88, A0A453D363, A0A453SRL5, A0A453Q2A6, A0A453JHH1, A0A453BBU6, A0A453JFZ5, A0A453H3Z2, A0A453DBR3, A0A453DBP2, A0A453LK56, A0A453TAD7, A0A453JH11, A0A453FPS4, A0A452ZGP0, A0A453QZV2, A0A453Q1W1, A0A452XH97, A0A453D2W3, A0A453SVQ7, A0A453DBR6, A0A453EGJ0, A0A453MGF3, A0A453D3B5, A0A453BC42, A0A453Q2K8, A0A453K9H8, A0A453LJN7, A0A453DBK2, A0A453BBR3, A0A453AAL0, A0A453SRP1, A0A453FPQ4, A0A453DBE5, A0A453QU60, A0A453SRQ7, A0A453QUA8, A0A453SUC2, A0A453A3H4, A0A453BYQ1, A0A452XD39, A0A453TAB6, A0A453FPV0, A0A452ZGM7, A0A452ZI86, A0A453SAJ3, A0A453DBS2, A0A453D394, A0A453DB45, A0A452ZGN4, A0A452Z015, A0A452ZNQ3, A0A453BBE7, A0A453LK78, A0A453F7K0, A0A453LKB7, A0A453D375, A0A453DB42, A0A453FPS1, A0A453D385, A0A453LKJ9, A0A453SRH1, A0A453SVF0, A0A453BBF4, A0A453QU28, A0A453SGI2, A0A453DBL7, A0A453AAP4, A0A453KCJ5, A0A453D310, A0A453DC24, A0A453D3E6, A0A453H3Z9, A0A453TA82, A0A453FPU6, A0A453SGY8, A0A453LK90, A0A452ZQ32, A0A453EGC1, A0A453D390, A0A452XB18, A0A453LKE6, A0A452ZRM6, A0A453LKA2, A0A453EGK8, A0A452Z021, A0A453DDC6, A0A453HR14, A0A453DB15, A0A453GLY9, A0A453LJT7, A0A453FPS5, A0A453BYR6, A0A453JIE9, A0A453FPU5, A0A453T0S7, A0A452Z4F2, A0A453Q2M4, A0A453ILU7, A0A452ZGI2, A0A453SFC7, A0A453LJW8, A0A453Q1Y8, A0A452ZGM9, A0A452XB15, A0A453MGP2, A0A452ZQ12, A0A453DC10, A0A453LKG5, A0A453BBC4, A0A453D2Z4, A0A453Q471, A0A453H401, A0A453SRN8, A0A453DYZ4, A0A453DBH0, A0A453SVS6, A0A453LKB9, A0A453ADT9, A0A453SRL4, A0A452XJY3, A0A453EGB6, A0A453DBX4, A0A453D3D9, A0A453QZF5, A0A452ZEW0, A0A453DBG6, A0A453QZE7, A0A453LJY8, A0A453ADP8, A0A453SRL2, A0A453DDC8, A0A453DC41, A0A453D315, A0A453DBP0, A0A453LKE7, A0A453D3A5, A0A453JGZ8, A0A452XHA9, A0A453DB67, A0A452ZGL3, A0A453Q276, A0A453QZ80, A0A453MGQ6, A0A453FPJ7, A0A453FPR0, A0A453DBN2, A0A453FPS6, A0A452XH94, A0A453BBV6, A0A453LN06, A0A453DBE9, A0A453ADV4, A0A453DDC4, A0A453EGH0, A0A453D3F8, A0A453IG18, A0A453ADQ8, A0A453DBM5, A0A453F7C5, A0A453LKD9, A0A453DBX3, A0A453D3H9, A0A453D302, A0A453BBC9, A0A452XD12, A0A453DBK1, A0A453SKA3, A0A453HRC9, A0A453DBG1, A0A453DBE1, A0A453D359, A0A453HAB3, A0A453DDN3, A0A453D6N7, A0A452XK26, A0A453T0X8, A0A452ZGM1, A0A453EGH5, A0A453BYM8, A0A453D395, A0A453LKC3, A0A453MGP0, A0A453LK53, A0A452XJQ5, A0A453D3A0, A0A453D3C4, A0A452XK20, A0A452XHA4, A0A453JG11, A0A453DBP9, A0A453DBW4, A0A453JHF1, A0A452XHB4, A0A453SRP8, A0A453DBL1, A0A453D309, A0A452YZZ3, A0A453DBN5, A0A453HAG7, A0A453DBJ1, A0A453DDM2, A0A452XB20, A0A453D2X9, A0A453SRM5, A0A453Q4I8, A0A453 DIV2, A0A453BDH9, A0A452XB13, A0A453DDB4, A0A453GMC0, A0A453DBP5, A0A453SRE5, A0A452ZGQ0, A0A453LKG2, A0A453BDI1, A0A452ZNP3, A0A453FPR3, A0A453JIE4, A0A453BD61, A0A453D398, A0A453FPL9, A0A452ZEW7, M8C3G1, N1QZA4, M8BLT1, M8CDU3, M8BQF5, R7W3S1, M8CA09, M8C8E1, R7W6C2, M8BRL3, M8BNQ4, M8BSU9, M8B659, M8B657, M8B6T4, R7W524, R7W6Q0, M8ARV2, N1R0P4, N1QWT2, M8C3Q0, M8BS71, M8C8A2, M8CGA2, M8D7P7, M8BGE8, M8AXL9, N1QQ18, R7W4G4, N1R0J5, M8CMY7, M8BFG4, M8CQW7, R7W4J2, M8BZH7, R7W0V2, M8BFQ4, N1R4A5, M8BVW7, R7W466, N1QUE8, N1QPR6, R7W2M1, M8AQG2, M8D5R8, M8B4Z6, R7WBZ0, N1R267, M8BEL9, M8AXV6, M8BHI2, R7W702, N1QZP8, M8BWJ0, M8AR11, R7W5U0, M8BKQ6, M8C7I9, R7W886, N1QTT0, R7W6D7, M8AR15, M8BFJ7, M8B3Y2, M8BW41, M8BV04, M8BSB0, M8C2A6, N1QZSO, M8B4G8, M8BIP4, N1QVU3, M8CU22, R7WB01, M8C5G3, M8BSH4, M8BH12, M8CC39, N1QZA9, R7W2H2, M8AWU4, R7W5J6, M8BVP9, M8B6H7, M8CQN8, N1QPF3, N1QVJ3, M8CV03, M8C2T7, M8BJJ3, M8BBM6, M8C398, M8C374, M8BRL2, R7W3B4, M8CMA1, M8BMX3, R7WBP2, M8BAQ4, M8BCF9, M8AI36, M8BZV5, N1QRV4, M8AI44, M8CDI5, M8BRQ9, M8BS62, N1QTT3, M8CXN2, R7WAL8, R7W389, M8CS97, R7W2A0, M8BMF1, M8CLL1, M8BBG0, N1R416, M8C0P3, N1QSH2, M8C8P5, M8BC75, R7W9K6, M8BZ85, M8AQX6, M8BRA9, M8BV83, N1QUU5, R7W017, M8AWM2, M8C1A2, M8CIT9, M8BR67, N1R3B9, M8BVQ9, M8APF0, R7W5F9, N1QR69, N1QWL3, R7W6I7, A0AIG5P0D6, K8P0P5, A0A2M8ZRI2, K8P4L2, A0A090MQ82, A0A380WCA0, A0A0M3DZ05, D6V6Y8, A0AIM2ZI81, A0A1Q3W3V1, A0AIH0JMN5, W3RJC5, A0A0Q6ZHE5, A0A2D6Z1S1, P0C9W5, A0A0C5AWG9, A0A1W4XI26, A0A1W4WER7, A0A1W4WCW5, A0A1W4WMX9, K2PXK1, A0A0D8KTY2, A0A135P8H9, A0A1S7TYJ4, A0A1S7R665, A0A1S7QSC7, Q7CT39, A0A1S7R1K3, A0A2W5HEG2, A0A3G2D9W1, A0A3G2CW11, A0A1S7N2M1, A0A1S7N2Q8, A0A1S7S8C0, A0AIS7MTQ7, A0A1S7N332, A0A1S7R6H0, A0A1S7SOT3, B9J7G8, W1KEF2, W1LDB8, A0A061MPX5, A0A2T7V3G2, A0A2T7VDF4, A0A178H301, A0A071HXB3, A0A071HYF2, A0A2S4E9Q9, A0A081CTJ4, A0A1B9TQL4, A0A1S7UAP0, A0A1S9E9P5, A0A1S9E6I0, F0LCJ2, F5J606, A0A3S2U7W8, A0AIR3TJ16, A0AIR3TLG8, A0A427QHU1, A0A0X6ZW52, A0A0X6ZP94, A0A328F968, A0A328ESI4, A0AIS7RVT6, A0A1B3P384, A0A1E3YAW1, A0A0L6K5Z5, A0A349XP92, A0A357X7H8, H0H495, H0HI51, F7UCM7, A0A1S7QYX0, A0A1S7RCW1, A0A1S7T9V4, M8B9N1, A0A1S7RU05, B9JSY8, A0A202F108, A0AIS2DVC4, A7XED0, A0A1S2E625, A0A368NW97, A0A2E0IUI6, A0A2E2RQX8, A0A2E0IF36, A0A2E2RKN7, G1M174, G1LEP0, G1MBX3, G1LET2, G1M194, D2HM20, D2HEU5, G1MDT1, D2H7K2, A0A0A7LYW8, A0A024GUK6, A0A024GTY2, A0A024FUV0, A0A024FUS4, F0WGZ2, F0W170, F0WDJ8, F0WDJ9, F0WGX7, A0A1B9PIN4, A0A151M2C0, A0A151NPI2, A0A151PBF9, A0A151NPX2, A0A151M2C2, A0A151MTI6, A0A151PAG7, A0A1U7S3Q2, A0A1U7R1W7, A0A3Q0GI02, A0A1U8DHUO, D3RPG7, A0AIH3HNT0, A0A0L0S2D3, A0A0L0RXQ9, A0A0U2S302, A0A0U2JFZ1, A0A0U2RH63, A0A1V1PR56, A0A1Q7BNX2, A0A1Q3K2D4, A0A1L9Q6D6, A0A2N3CAF3, A0A2N3CN55, A0A2N3BHU6, A0A2N3BWC8, A0A3M1A8N1, A0A3M1CCK9, A0A3B9YVH5, A0A3B9YUR4, A0A3C0MDP2, A0A177E5Y0, A0A177DMN5, A0A177DB16, A0A177DW37, A0A177D586, A0A177DA37, A0A399GD40, A0A399HQ58, A0A399G9V7, A0A2M9PBP4, A0A0Q3U1E2, A0A0Q3UR44, A0A0Q3MLN8, A0A0Q3MGK6, A0A1ElX339, A0A1ElXND8, W1PSU2, W1NHI6, W1NS07, W1PUY6, U5D4B7, W1PK20, W1NP94, W1PWB1, W1PiF1, W1PJ97, U5CZI6, W1PNMO, W1PYM5, U5D3T0, W1NII4, W1NTK9, W1PI96, W1NKX3, W1PU85, W1PHD9, W1P824, W1P1U3, W1PS15, W1P261, A0A380WPF7, A0A380WLF7, A0A142M5P6, A0A316H1V4, A0A2SOXJL2, A0A0Q6M906, A0A3Q0QWF0, A0A3Q0RP91, A0A3Q0QW88, A0A3Q0QZA4, A0A3Q0QXII, A0A3Q0QYJ9, A0A3Q0QX28, A0A3Q0RIQ3, A0A3Q0QX05, A0A3Q0RNB8, A0A3Q0QX18, A0A3Q0RNE4, A0A3Q0T1F7, A0AIX7VF86, A0AIX7VJI8, A0A3Q1CUE1, A0A3Q1AZ85, A0A3Q1BNV0, A0A3Q1ASI9, A0A3Q1CEN8, A0A3P8SBR5, A0A3P8U3P7, A0A3P8RV74, A0A3P8SZJ0, A0A3P8T1W7, A0AIW6EWC0, A0AIW6EWB2, A0A2N5IM96, A0AIB7WGH8, A0A3Q1JM01, A0A3Q1JGW1, A0A3Q1HBQ8, A0A3Q1JNC7, A0A3Q1HBU2, A0A3Q1K4Z1, A0A3Q1HBM8, A0A3Q1JWC6, A0A3Q1H376, A0A3Q1JWD0, A0A3Q1H8P6, A0A1F3C361, A0A199VKR8, A0A199W1P5, A0A199VMM6, A0A199V5G5, A0A199V0P4, A0A199VXD4, A0A199UUR3, A0A199UNR1, A0A199UFB6, A0A199V3G9, A0A199UF66, A0A199VMS2, A0A199W3M8, A0A199VXV5, A0A199UW40, A0A199VG00, A0A199VR05, A0A199UW09, A0A199VZH8, A0A199VCY3, A0A199UMA3, A0A199VZ99, A0A199VI10, A0A199UUQ7, A0A199UV66, A0A199UQ24, U3IRP5, U3I6X0, U3II31, R0K1Z7, R0JPB7, R0M4Q0, R0LQI0, A0A368GRP9, A0A368GP95, A0A368GMN0, A0A368H349, A0A368GJB9, A0A368GWF9, A0A368HB09, A0A368G5Y2, A0A368GRQ0, A0A368GVT4, A0A368GWX4, A0A368H7D6, A0A368H3B6, A0A368FPC8, A0A368GP49, A0A368GXY9, A0A368EXZ3, A0A368GJW8, A0A368FA73, A0A368GWI0, A0A368GX96, A0A368GTK6, A0A368H705, A0A368GFH7, A0A368H7B8, A0A368HDM2, A0A368GFI5, A0A368HEE4, A0A368GI28, A0A368GRE3, A0A368FCY8, A0A368FCZ3, A0A368G2Q2, A0A016VKR6, A0A0D6M3P1, A0A016WI38, A0A016V0U2, A0A016S814, A0A016RWJ5, A0A0D6LC48, A0A016WHK2, A0A016SGK6, A0A016T8L1, A0A016SHF1, A0A016WSY7, A0A016S7S9, A0A016S6Y5, A0A0D6LU43, A0A016TUW3, A0A016T0N8, A0A016W3A0, A0A0D6LRJ3, A0A016WIX9, A0A016WRA4, A0A016RV21, A0A016VJZ2, A0A016STN6, A0A0D6LUD3, A0A016SU80, A0A0D6M9L2, A0A016SMB0, A0A016VCU4, A0A016SS42, A0A016W8B6, A0A016V243, A0A016SU41, A0A016V216, A0A016SRZ7, A0A016V3K2, A0A016W7N7, A0A016UI89, A0A016WS77, A0A016UHN2, A0A016TCL6, A0A016T8A6, A0A016WA14, A0A0D6MAD7, A0A016VDI1, A0A016T206, A0A016V2S3, A0A016WI79, A0A016W8D8, A0A0D6M851, A0A016V2R6, A0A016W998, A0A016SR62, A0A016V233, A0A016WTI9, A0A016UJN5, A0A016V3J7, A0A016TTS6, A0A0D6LG45, A0A016VDY5, A0A016WNP3, A0A016STB4, A0A016VIC1, A0A016V2R1, A0A016SS83, A0A016WSZ0, A0A016V1H8, A0A0D6M4L0, A0A016TCJ1, A0A016VFG3, A0A016VEL5, A0A016WGP1, A0A016UG75, A0A0D6LIH3, A0A016TCI8, A0A016VL99, A0A016SU43, A0A016SHA2, A0A016TVN9, A0A016WI62, A0A016T0X6, A0A016WQP5, A0A016SU38, A0A016VJ86, A0A016SL82, A0A016UGP0, A0A016VA51, A0A016WJ45, A0A016WEZ6, A0A016T7T4, A0A016TXR8, A0A0D6LZI8, A0A0D6LVB3, A0A016WN18, A0A016WV69, A0A016RVX3, A0A0D6LXD0, A0A016WHK9, A0A016UHG4, A0A016V2P8, A0A0D6M3G6, A0A016WUK9, A0A0D6M3L0, A0A0D6M584, A0A016RVW0, A0A0C2DVX2, A0A0C2FMV1, A0A0C2G7Y5, A0A0C2CRJ1, A0A0C2GDQ6, A0A0C2H4Q3, A0A0C2GLS5, A0A0C2G2S8, A0A0C2DFM8, A0A0C2DQD8, A0A0C2H0D7, A0A0C2CKU6, A0A0C2C4H3, A0A0C2HCQ8, A0A0C2BR75, A0A0C2FCW8, A0A0C2FEG0, A0A0C2D4J6, A0A0C2F8I9, A0A0C2FGE8, A0A0C2FP16, A0A0C2GFH0, A0A0C2G7Z8, A0A0C2H638, A0A0C2FGY3, A0A0C2CYI0, A0A0C2FI47, A0A0C2F9X1, A0A0C2GVY3, A0A0C2DNE1, A0A0KODQKO, A0A0KOCXV8, A0A0KODEV9, A0A158PCD7, A0A0KOD0D1, A0A0KOD3I0, A0A0KOCUG2, A0A0KODH19, A0A0KOD5J4, A0A0KOD1B4, A0A0KODJR6, A0A0KOD008, A0A158PB05, A0A158P5X6, A0A158P8Q5, A0A158PAM2, A0A0KODL22, A0A3P7JT81, A0A158PIZ8, A0A3P7HR75, A0A158PEZ2, A0A3P7J3A0, A0A158PKV1, A0A0R3PDF2, A0A158PGM2, A0A158PE13, A0A158PFJ6, A0A0R3PME5, A0A0R3PZF2, A0A0R3PL23, A0A0R3PMU4, A0A0R3PYM1, A0A158PJ68, A0A158PLM5, A0A0R3PB90, A0A0R3Q188, A0A158PGG5, A0A0R3Q0R2, A0A0E9X3V9, A0A0E9RJH8, A0A3P6NGQ5, A0A0M3JX10, A0A0M3K2F0, A0A3P6SFK2, A0A3P6RLJ8, A0A0M3JD59, A0A0M3KAT5, A0A0M3KD76, A0A3P6T6E9, A0A3P6RTW8, A0A0M3K8I9, A0A0M3K2Z5, A0A0M3J4P1, A0A0M3J2X5, A0A0M3JVW0, A0A0M3J5I5, A0A0M3KA02, A0A0M3KCUO, A0A0M3JKG2, A0A0M3K200, A0A0M3JKG6, A0A0M3K1Z4, A0A0M3KIH9, A0A0M3K2C6, A0A0M3JY76, A0A158PN22, A0A0M3K2T5, A0A0M3J493, G1KNL2, G1K9G8, GlKBV2, GlKIB0, R4GD12, A0A182F6L9, A0A182F5Z9, A0A182F6L8, A0A182FRE6, A0A182F6M0, A0A182F5Z8, A0A182FDG2, A0A182HU77, B2G0D9, B2G0D5, A0A182HTF9, B2G0D4, A0A182HTG0, B2G0D3, A0A182I7L3, A0A2C9GRZ8, A0A2C9GRP2, A0A182I7L4, A0A182HTF8, A0A182IT52, A0A182ITQ2, A0A182JLI3, A0A182ISL2, A0A182J7H9, A0A2M3Z8D8, A0A182JNP2, A0A182KB58, A0A182JQG9, A0A182KEB4, A0A182KAYO, A0A182JNP1, A0A182LFU3, A0A182LFM7, A0A182KQB0, A0A182LMG3, A0A182LFM6, A0A182M3D4, A0A182MI18, A0A182MAX7, A0A182M333, A0A182M9X2, A0A182LRC3, W5J6L5, W5J5H0, W5JK04, W5JQZ0, A0A182NGA9, A0A2Y9D1G5, A0A182NBM5, A0A182NBM6, A0A2Y9D1N7, A0A182N642, A0A182NGA8, A0A182NGA7, A0A182P472, A0A182PEK4, A0A182NZH6, A0A182NZH5, A0A182PEK5, A0A182PEK6, A0A182P5T7, A0A182QEI7, A0A182QKG0, A0A182QSKO, A0A182QWD4, A0A182QWKO, A0A182Q5P2, A0A182RNR5, A0A182RHD6, A0A182RF54, A0A182R769, A0A182RHD7, A0A182R770, A0A182R771, Q7PTF3, B2G0E1, Q7QBU4, Q7QD96, Q7PWD7, A0AIS4H0L9, A0AIS4GZU1, B2G0F5, B2G0E5, A0A182T9U0, A0A182SV91, A0A182SSP0, A0A182SAF1, A0A182T826, A0A182T015, A0A182TPY7, A0A182TF95, A0A182UCD3, A0A182TQP9, A0A182TPR8, A0A182UC50, A0A182VE60, A0A182VK07, A0A2C9H5R2, A0A182V7G1, A0A182WGE9, A0A182WLF8, A0A182W062, A0A182W2N8, A0A182WGF0, A0A182W2N6, A0A182W2N7, A0A182WWW6, A0A182WXP9, A0A182XM10, A0A182XM08, A0A182WXQ0, A0A182XM09, A0A182WRJ9, A0A084VR87, A0A084VZ46, A0A084W3V7, A0A084W3V8, A0A182XVF9, A0A182Y0J9, A0A182Y0K0, A0A182Y4A5, A0A182YEI6, A0A182XVG0, A0A222E9G5, A0A239JZP1, A0A239G1U7, A0A222E1N3, A0A222E9D9, A0AID1YV81, A0AID1YNY0, A0A1D1Z1M7, A0AID1ZCW2, A0AID1XWH9, A0AID1XFS1, A0AID1ZCH5, A0AID1YQR8, A0A1D1Y9Q8, A0A1D1Y7P0, A0AID1YD96, A0AID1YYX4, A0AID1YCC3, A0AID1Z0E3, A0A1D1Z3R3, A0AID1YIR8, A0AID1YXD0, A0AID1ZLQ1, A0A1D1Y6E9, A0AID1XRS7, A0AID1XD27, A0AID1ZCJ4, A0AID1XNX6, A0AID1ZGKO, A0AID1YPM1, A0AID1XWC5, A0AID1YK26, A0AID1XXZ3, A0AID1YPA4, A0A1D1Y8U2, A0AID1XS70, A0AID1ZHQ4, A0AID1ZBF3, A0AID1YFK6, A0A1D1Y7K5, A0A1D1YYY6, A0A1D1YYC2, A0A1D1Z4A8, A0A1D1XX47, A0A1D1YX08, A0AID1YFZ2, A0AID1ZJN6, A0AID1XYK2, A0AID1ZM53, A0AID1ZFV4, A0A1D1YAP6, A0A1D1Y229, A0A1D1XT90, A0A094NGJ6, A0A094L822, A0A094K4U5, A0A2K5DLN0, A0A2K5CCL7, A0A2K5DLM5, A0A2K5DWJ7, A0A2K5DLU9, A0A2K5D8Z0, A0A2K5D8Y5, A0A2K5D923, A0A091N7G4, A0A397C4L9, A0A3R8DBH7, A0A3R6Z9I5, A0A3R6ZA35, A0A397EGH5, A0A418CK86, W4H175, W4FC62, A0A3R7YB83, A0A397C6I0, W4H5C6, W4G226, A0A3R6Y2Z4, A0A397D222, A0A3L6VV49, A0A3L6UWB2, A0A397DZ46, A0A3R7Y206, W4G2B7, A0A397EC98, A0A396ZRW0, A0A397CJH5, A0A397D947, W4F959, A0A3R7B8C5, W4G4H3, A0A3L6V4T6, A0A3R6XZD2, A0A397EMC9, A0A3L6VXF2, A0A3L6VVV3, A0A397FE69, A0A397ER22, A0A3R6ZU09, W4GPE9, A0A3R6Y1Q0, A0A397DAZ4, A0A397FMV1, A0A418C2V2, W4G2D3, A0A425D7X0, A0A3R7BUC6, W4G048, A0A418DV78, A0A418FR37, W4H736, A0A397DY85, A0A3R6Z315, A0A418EI16, A0A397BNB4, A0A396ZQC4, A0A3R7BMT9, A0A397EYS8, A0A396ZVZ8, A0A397ELC4, A0A418FSX8, A0A418BVT6, A0A3R7ABK1, A0A3R7DKS6, W4H234, A0A397B129, A0A425CKN8, A0A418DLV8, A0A418E0M0, W4G2I9, W4FM40, A0A3L6VNC7, A0A418EMF9, W4FJ95, A0A3L6UZY8, A0A418BS39, A0A3R6Y6L2, W4FBD4, A0A397BC55, A0A3R6Z9Q1, W4FAU3, A0A396ZUR5, A0A3R7B511, A0A397CHM2, W4FIU7, A0A397AJZ3, W4FDQ7, A0A3L6V775, A0A397BDL6, A0A3R7AC59, A0A397A4C7, A0A3R8CZN9, W4FZU6, W4G6A2, W4FEN0, A0A3L6VU69, A0A3L6VUC0, A0A3R8D3V5, A0A397DNW1, A0A397E6J1, A0A3R7AVF3, A0A397CYV4, A0A3R6WWC2, A0A397EBU1, A0A397C7D3, W4GYA7, A0A3L6VPA3, A0A3R7CXJ7, A0A397DAQ9, A0A397B2Z6, A0A397C063, A0A397EVE7, A0A397E7V6, A0A397E2F2, W4GZF9, A0A397EAD8, A0A397AMN7, A0A397FFG6, A0A3R7WJU8, A0A397BRS4, A0A397CFN8, A0A397ATR5, A0A397BCR8, A0A3L6VBL8, A0A397DIQ3, A0A397CN17, A0A3R6WP37, A0A397D709, A0A397FYU7, A0A3R7C4F4, W4GZ73, W4H0H9, A0A397G0U1, A0A3L6VNX6, A0A396ZPP3, W4FES1, A0A418D021, A0A418FQK5, A0A397CWE3, A0A3R6XQ00, A0A418F500, A0A3R7E8Z5, A0A3R6XAD8, A0A3R7EK41, A0A3R7AZA1, A0A397EH51, W4GXF5, A0A3R6Y8H5, A0A3R6ZZZ6, W4GZE8, A0A397DGM4, A0A3R7BA06, A0A397FI29, A0A3R7F475, W4FYV5, A0A397FE42, W4GBI6, A0A397BA52, A0A3L6VUC9, A0A3R6WPT4, A0A397BC03, A0A397D6D7, A0A397BR53, A0A397FF82, W4FKP1, A0A396ZTJ8, A0A397ASG3, A0A418FQ73, W4FWW2, A0A418CSY7, A0A418FU02, A0A397CX05, A0A418CAJ3, A0A3R6XZB8, A0A397EM03, W4FWJ0, A0A425DPY9, A0A418DX93, A0A397DI86, A0A3R7AM78, A0A418CFB1, A0A3R6ZM03, A0A397D336, A0A397D2R9, A0A397C5U1, A0A3R6ZVR7, A0A3R7WGA7, A0A397C1U3, A0A418BP49, A0A397EL04, A0A418DXN6, A0A397B3A6, A0A3R6Y8I4, A0A3R7WIS6, A0A418E5K5, W4FMA4, A0A425DM30, A0A3R6XLJ9, A0A3R7ZIF7, A0A3L6UPJ1, W4FYU2, A0A418FGP0, A0A3R7YVR4, A0A3R6XCY1, A0A397A0Q9, A0A397DYY2, A0A397A3C6, A0A425CZ29, A0A418EHM7, A0A418CJF8, A0A425DJP4, A0A397DYB5, A0A397B9P2, A0A425D1E4, W4G0V0, A0A418CJF6, A0A397B5E3, A0A3L6VJM5, A0A3R7Y395, A0A418CUV0, A0A418BSV9, W4GEU8, W4GHR1, A0A425CPR1, A0A3R7APT9, A0A418FS09, A0A397ES50, A0A425CS30, A0A418E063, A0A397EAV1, A0A3R7WQH8, A0A3L6VZA1, A0A418BJ04, A0A3R6WYY8, A0A397C1F4, A0A397FHZ3, A0A3R6WVI8, A0A397CVV7, A0A3L6VF87, A0A397C940, A0A397F613, A0A3R7EHY7, A0A397DXJ3, A0A418DCN0, A0A397ER77, A0A418CWS7, A0A3R7AHK3, A0A3R6WXR4, W4H7L0, A0A418BZY3, A0A397D456, W4FRR2, A0A397EV56, W4FGT5, A0A3L6VXT1, A0A397BP25, W4FGX0, W4H101, A0A397FDH7, A0A425DDM5, A0A3R7A4K4, A0A397CJR0, A0A3L6VMY5, A0A418E4P5, A0A418G3N5, A0A397FAR0, A0A3R6XZM3, A0A3R7A7P4, A0A425CZ94, A0A418D8J3, W4GL89, A0A418FA61, A0A397BAP7, A0A396ZQ03, A0A396ZZ96, W4FL12, A0A397ASM2, A0A418C327, A0A397EKX9, A0A3R7AKW5, A0A3R6XRX3, A0A418FLE6, W4FEW7, A0A418E1G2, A0A418DBB7, A0A418CCH5, A0A3R7Z8J7, A0A418FGH1, A0A418FYZ6, A0A397EMD9, A0A3R6XJW2, A0A3R7Z6P2, W4H6A6, A0A418E9V7, W4GDP9, A0A3R7B8T5, A0A397E2H2, A0A418BIJ8, A0A418E419, A0A397EUF6, A0A397D0N4, A0A396ZWI2, A0A425CZR5, A0A397BFX1, A0A3R6WLH3, A0A397F2W6, A0A418FPV6, A0A397CC55, A0A397EVK5, A0A397CFM8, A0A425DEZ5, A0A397CGA6, A0A3L6VYT2, W4FBP1, A0A3R6XF63, A0A3L6VSI2, A0A3L6V4Z0, W4FLQ6, A0A418BUX3, A0A397D6T6, A0A396ZRZ2, A0A418E7S8, A0A396ZT37, A0A397ANE1, A0A3L6VA85, A0A397A932, A0A3L6VJ13, A0A3R6W2X3, A0A3L6V2L6, A0A3R6ZFR3, A0A397AiS1, W4FPQ1, A0A3L6VS68, A0A397BWJ0, A0A397A9C7, A0A3R6YID9, A0A3R7BR36, A0A3L6UUI8, A0A3L6V0T7, A0A3R7AMU4, A0A397C9E7, W4G1X8, A0A3R6X795, A0A397E6K8, A0A3L6UW97, A0A397AFG4, W4FCE8, A0A3R6VM88, A0A3L6VTJ7, A0A3L6V0S4, A0A3L6VQP2, W4F8J2, A0A397BNT6, A0A397BQ30, A0A397AP79, A0A3L6V0R4, A0A397BQC2, W4FI44, A0A397B2U7, A0A397CA05, W4FB83, A0A397ESKO, A0A3R6W590, A0A397BBH9, W4GB84, W4FRB0, A0A397ESI1, A0A397AHE1, A0A397CD13, A0A397ALA4, A0A3R7XTQ9, A0A397E2L9, A0A397AY23, A0A397BNE7, A0A397A1D7, W4G074, W4GF49, A0A3R7WNJ7, A0A3L6V0A9, W4FTM9, A0A397C9P7, W4FC56, A0A397D5B2, A0A3L6UVS9, A0A3R8DIH2, W4G054, A0A397AF63, A0A3R7AA07, A0A3L6VW95, W4G9H4, A0A397DQE0, A0A397CE78, A0A397BWP1, A0A3R7AS62, A0A397BZC2, A0A397F5K6, A0A397CQE7, W4FUT9, A0A397BX76, A0A397EWY4, A0A3L6UTC6, W4FCI5, A0A3R7XUT3, A0A397AF69, A0A397FDL6, A0A3R6W9N8, A0A3R7FC57, A0A3L6V861, A0A397F9B1, A0A397DBN1, W4GGA2, A0A397AHP6, A0A397DW29, A0A397A7S0, A0A3L6VJZ3, A0A397ED42, A0A3R6WAMO, W4GH53, A0A397CR86, A0A397BPQ1, W4GZ49, A0A397EYN3, A0A397DRN3, A0A397DXZ3, A0A397A3K7, W4FCU5, A0A397B480, A0A397C1F3, A0A397EXX4, W4H088, A0A418D470, A0A3R7A612, A0A397ES75, W4GXA1, A0A397AYL9, A0A3R6WDP1, A0A397EYB4, A0A418CY42, A0A397G009, A0A418FZC5, A0A3L6V1Y4, A0A3L6UQC2, W4GFA8, A0A3R6XZQ9, A0A3R6Y2R4, A0A024UHK3, A0A024T9L5, A0A3R7CTQ5, A0A024TVG3, A0A024UH93, A0A3R7A1Q5, A0A418AHC0, A0A024TRP2, A0A024TEL4, A0A418AME6, A0A3R6Y3Y5, A0A3R6WGF2, A0A3R6V2T3, A0A024TPQ1, A0A024UDS5, A0A418AM46, A0A024U7C9, A0A3R6V2B0, A0A024UQH4, A0A024TK95, A0A024UD11, A0A024UDS2, A0A418AND4, A0A024UAK4, A0A418ARH0, A0A024UD26, A0A418AHK8, A0A418AMH7, A0A418AJY7, A0A3R6VR99, A0A024TS56, A0A024TUE0, A0A024TWF3, A0A3R6VF93, A0A3R7A275, A0A024UR54, A0A3R6WDL0, A0A024T8G1, A0A024TWY4, A0A024TCZ9, A0A024UMA7, A0A024UH78, A0A024TCZ7, A0A024TSD7, A0A024TBE3, A0A3R6Z6F7, A0A024TFF2, A0A024UC37, A0A024UOX8, A0A418BIG9, A0A3R6VQUO, A0A418ASS1, A0A024UP64, A0A3R6WNC5, A0A3R7A440, A0A418AQE9, A0A024UQE9, A0A024UBX1, A0A024UNS3, A0A024TDF0, A0A024U4R8, A0A3R6ZIP0, A0A024U8U9, A0A418AI42, A0A024URA0, A0A024T9U4, A0A418B226, A0A3R6VRZ5, A0A3R7CVB0, A0A3R6VPV8, A0A2A3EFQ3, A0A2A3EQR0, A0A2A3EB22, A0A2A3E6U1, A0A2A3EPT1, A0A2A3EC69, V9IEE8, A0A087ZXY2, A0A088AFH9, A0A088AST6, A0A088AC97, A0A088ANX3, A0A087ZV55, A0A088AG76, A0A2I0ASJ4, A0A2H9ZXD3, A0A2I0B3M2, A0A2I0B5S4, A0A2I0B7B3, A0A2H9ZQW2, A0A2I0ADE7, A0A2I0AA30, A0A2I0B169, A0A2I0ABX1, A0A2I0AA27, A0A2I0B4N6, A0A2I0AQ30, A0A2I0AAT8, A0A2I0B7A3, A0A2I0B7B6, A0A2I0BFH8, A0A2I0A8W9, A0A2I0ACE4, A0A2I0A6S9, A0A2I0AA33, A0A2H9ZQW3, A0A2I0B912, A0A2H9ZYQ7, A0A2H9ZXB8, A0A2I0B2V6, A0A2H9ZX95, A0A2I0A9R7, A0A087QH18, A0A087QYC5, A0A087QH19, A0A087RE68, A0AII3QH19, A0AII3RZC0, A0A0H1AF09, A0A2G5DNG3, A0A2G5EQ76, A0A2G5EQJ1, A0A2G5DCP1, A0A2G5C542, A0A2G5E5T2, A0A2G5EYF5, A0A2G5CKR8, A0A2G5D4L1, A0A2G5C571, A0A2G5CRQ3, A0A2G5EDS8, A0A2G5E7L7, A0A2G5E6D6, A0A2G5CRL5, A0A2G5C563, A0A2G5D4L6, A0A2G5E7H8, A0A2G5C847, A0A2G5ESB6, A0A2G5D4N4, A0A2G5CZF9, A0A2G5E7K7, A0A2G5DFA2, A0A2G5DIL6, A0A2G5ET28, A0A2G5D4L8, A0A2G5CH03, A0A2G5CZE7, A0A2G5DF97, A0A2G5CZE9, A0A2G5CRT5, A0A2G5EGS4, A0A2G5D562, A0A2G5EYR8, A0A2G5C581, A0A2G5DEI8, A0A2G5EYJ4, A0A2G5EYQ4, A0A2G5EYD9, A0A2G5D4M2, A0A2G5EYH4, A0A2G5EQJ0, A0A2G5D4M8, A0A2G5CRN7, A0A2G5ES32, A0A2G5CRM7, A0A2G5E7Q3, A0A2G5DFX8, A0A2G5CMW1, A0A2G5CRX1, A0A2G5CRI1, A0A2G5DG44, A0A2G5EYI8, A0A2G5EYI1, A0A2G5EYH5, A0A2G5EYG4, A0A2G5CRL0, A0A2G5D4K8, A0A2G5CGV5, A0A2G5DCI3, A0A2G5DCM8, A0A2G5E5V6, A0A2G5EYI0, A0A2G5D8X1, A0A2G5D4K5, A0A2G5CRR1, A0A2G5CRM4, A0A2G5E7J1, A0A2G5D5G9, A0A2G5E7K3, A0A2G5DE67, A0A2G5EYE0, A0A2G5DCF9, A0AIG6XXE2, V5J679, V5J697, V5J681, V5J6E2, A0A3S9XJJ1, A0A451ES05, A0A451ES06, A0A3S5XGT4, V5J6V5, A0A3S5XGT3, A0A451ES07, V5J691, V5J6A0, V5J6E6, V5J6V1, V5J6X1, A0A3S5XGT1, A0A3Q9JP76, A0A3S5XGT0, V5J698, V5J688, V5J689, A0A3Q9JP70, A0A3Q9JP72, V5J682, V5J699, V5J6E4, A0A3Q9JP73, V5J692, V5J6E9, V5J6F1, V5J6W1, V5J690, V5J683, A0A451ESO4, A0A3Q9JP74, A0A451ES08, V5J6W5, A0A3S5XGT2, A0A3Q9JP77, A0A451ESO3, V5J680, I7EWH2, I7FWN1, D6NU58, I7FWN4, B2Y4N4, B1P3G7, D0EXX6, I4IY52, D0EXW2, B1P3E5, D0EXX4, B1P3K8, D0EXYl, A7XE46, B1P3H0, A0A0C5JYN3, D0EXW0, D0EXW6, A7XE66, B1P3I4, A7XE70, A0A068CIR0, A7XE48, A0A068CIP9, A0A0C5K603, A0A0C5K8H5, A0A0C5K8B3, D0EXY8, D0EXX7, A0A068CQG1, Q19MC1, B1P3I7, A0A068CM44, A0A0C5KK99, D0EXY2, B1P3G3, A0A0C5KK72, A7XE76, B1P3I5, Q19MC0, B5TD10, B1P3I6, A0A068CM61, D0EXX0, A7XE72, B1P3G6, A0A0C5K5V8, D0EXY3, A7XE85, D0EXW8, A7XE56, B5TD17, B5TD13, B5TD14, A7XE74, A7XE52, A7XE63, A7XE50, D0EXY7, A7XE68, B1P3K0, A0A068CM37, D0EXYO, D0EXW5, B1P3K4, A7XE58, B1P3H3, A7XE83, A0A0C5K8F4, A7XE60, A7XE64, A0A0C5K5X4, D0EXV9, D0EXY6, A0A0C5K8D6, B5TD16, D0EXX1, A7XE81, A7XE79, D0EXW1, A7XE54, B5TD11, B5TD12, I4IY53, B5TD15, B1P3J6, A0A068CQH3, A0A068CM51, I4IY51, B1P3E8, B1P3F7, D0EXW7, B1P3G8, I7FNZ1, I7FXW5, I7EWH7, I7FWN7, I7FNZ6, I7FXW2, I7EWG7, I7F535, D7KVV7, D7KW17, D7KW16, D7KVK3, D7MEU5, D7KMD6, D7KZN2, D7MOB8, D7MEH3, D7KSZ5, D7KVV6, D7LFY7, D7KV44, D7KW05, D7M4K4, D7KSZ4, D7KW07, D7KVI7, D7KMD5, D7KMC8, D7KW12, D7KW14, D7MWF8, D7MJM6, D7KW03, D7KEK1, D7KVJ8, D7LTX6, D7MUF3, D7KW18, D7KYK2, D7L500, D7KMD4, D7KW15, D7MJN8, D7MEU2, D7KW04, D7MWF7, D7LHZ3, D7M9K1, D7KMD7, D7MEU3, D7MIU9, D7M2R0, D7LIA6, D7KMD1, D7KU49, D7MEH4, D7KME3, D7LIX6, D7LTL2, D7KMC5, D7M5Q3, D7MJG2, V5J6F9, V5J6F7, V5J693, V5J6B0, A0A060D3V0, V5J695, V5J6Y3, V5J6X7, V5J6F3, V5J686, V5J6A1, V5J6F5, V5J6A8, V5J6Z0, V5J6Z6, V5J6A3, V5J696, V5J684, V5J6A6, V5J694, D0EXX3, D0EXZ4, G4WHA0, B5TD25, L7UVM3, Q84XJ6, G4WH68, A0A068CIS1, A0A451ERZ8, A0A451ES00, Q9ATK3, Q19MB7, A0A068CL41, Q19MB2, Q9ATK0, Q9ATK4, G4WH69, Q84XI9, Q84XK2, C3VIF, Q27JI1, A0A068CQL8, Q9ATK2, A0A3Q9JP26, R9S9V2, Q19MB5, B5TD23, A0A068CIT0, A0A068CIT5, Q19MB6, A0A3Q9JP81, Q84XK1, A0A068CLV9, B5TD21, Q9ATJ7, Q19MB4, Q84XK3, G4WH91, Q19MB0, Q84XJ0, D0EXX2, Q9AVE1, G4WH89, Q19MB8, Q84XJ2, Q84XJ5, G4WH79, Q84XJ4, A0AIJ0GTD5, Q9ATJ5, B5TD20, G4WH80, A0A451ERZ9, A0A451ES01, G4WH99, C7FE07, A0A068CLU4, A0A3S9XJG2, A0A068CM84, A0A451ESO2, A0A068CL61, Q84XJ9, A0A3Q9JP24, Q84XJ1, A0A3Q9JP42, B5TD24, B5TD18, A0A068CQJ3, B5TD19, A0A3S5XGS9, B5TD27, Q19MB3, G4WH92, A0A3S5XGS8, A8IKA8, Q9ATJ4, G4WHA1, Q9ATJ6, Q9ATK1, Q84XI8, B5TD26, Q84XI4, Q9ATJ9, Q84XI7, Q84XJ3, D0EXV6, Q84XI6, G4WH81, Q84XJ7, B5TD22, Q9ATJ8, C7FE06, R9QT99, Q84XJ8, Q84XI5, D0EXV5, Q9AVE0, Q27JF2, Q27JG5, Q9LPZ9, P0DH86, Q9LW83, Q9S972, 064782, Q39086, 081905, Q39203, 081833, Q39202, Q9ZR08, 064477, Q9LZR8, Q9SXB3, Q9SXB4, 064784, Q9LPZ3, 064778, 064776, 064771, Q9ZVA1, Q9ZVA4, Q9ZVA5, 081906, P0DH87, Q9SXB8, Q9SXB5, 064783, 064774, Q9SY95, 064780, 064770, 064781, Q9SY89, Q9SYA0, 064793, 064777, Q9ZT07, P93756, Q9T058, 081832, A0A1P8AN24, A0A1P8B6N6, A0A1P8AM95, Q9SCT7, A0A1P8B6M9, A0AIP8ASO4, A0AIP8AVJ2, Q9LHM1, A0A178VV00, A0AIP8B912, A0A178V611, A0A1P8B838, K9LQT0, A0A178WKT1, A0A178W8X6, A0A1P8AM91, A0A178WAM2, A0A178W4F8, A0A178WMJ8, A0A178WCF7, A0A384KCW1, A0A1P8AN12, K9LQP6, A0A178V4X9, A0A178WEA5, A0A178V3N4, A0A178V2J5, A0A1P8B6N5, A0A178WN39, A0A178UWV5, A0A178WFA7, A0A1P8AU51, F4I7F8, A0A1P8AS39, A0A1P8AVQ7, Q9FVW1, A0A1P8B837, A0A1P8B840, A0A384KEE5, A0A1P8AN14, A0A178UYZ8, A0A1I9LPL1, A0A1P8B600, L7USG8, A0A178V6V9, A0A1P8ATF5, A0A178VFP7, A0A178WA58, A0A1P8AWX0, A0A178WDF4, A0A178WC58, A0A178WA81, A0A178WI76, A0A178VYR4, A0A1P8ARZ8, A0A1P8AMD4, A0A1P8B859, A0A1I9LPL5, A0A1P8AMY7, A0A178WJN4, A0A178WJI0, A0AIP8ATC6, A0AIP8ARD8, A0AIP8AU37, A0A178WFP0, A0A178V0J5, A0A178VNJ9, A0A1P8ASG9, A0A1P8ASF1, A0A178WB58, A0A178W2U0, A0A1P8AMT2, A0A178W9H1, A0A1P8AM94, Q41272, A0A178WDZ2, F4I8U6, A0AIP8AVL2, A0AIP8AWT8, A0AIP8ASM5, A0A178UVF9, A0A1P8AN35, A0A178USE7, A0A1P8B5P5, A0A1I9LPL2, A0A1P8AMA7, A0A1P8B844, A0A1P8AM92, A0A1P8AQZ9, A0A1P8AMR1, A0A178WDY1, A0AIP8AMR8, A0AIP8AN06, A0AIP8AQN4, A7Y5W8, A0AIP8AMA0, A0A178W877, A0A178WD43, A0AIP8AN03, A0AIP8AMA3, A0A178UCA7, A0A178WK46, A0A1I9LPL3, A0AIP8B6M8, A0AIP8AVI8, A0A178WL67, A0A1I9LPL6, A0A178WAR7, A0A1P8B6N1, A0A1P8AW73, A0A087GL67, A0A087HF14, A0A087G4N3, A0A087HEZ2, A0A087HEY3, A0A087H0M1, A0A087GH82, A0A087GH84, A0A087G682, A0A087H3L7, A0A087H3L8, A0A087GFR5, A0A087GFR4, A0A087H8W0, A0A087HEZ7, A0A087HJG0, A0A087GDA1, A0A087HEZ3, A0A087GH83, A0A087HEZ6, A0A087GX11, A0A087H0M6, A0A087GPZ0, A0A087H0M5, A0A087GSX0, A0A087HCS1, A0A087GDA2, A0A445BB21, A0A444YSR3, A0A444XXG8, A0A444Y4Z7, A0A445C105, A0A444WXX8, A0A445DZ26, A0A445C101, A0A444XXT2, A0A445BY59, A0A445BY66, A0A444XD73, A0A445CY83, A0A445BYC6, A0A445BY70, A0A445BYB8, A0A445DYY7, A0A445ECY2, A0A445BYJ2, A0A445CQB5, A0A445BS77, A0A445CI63, A0A445BYE2, A0A445CU59, A0A445C0ZI, A0A444Y4V5, A0A445AGS4, A0A444Y4X6, A0A445BYN4, A0A445AFB2, A0A445DZ08, A0A445BP40, A0A445BB53, A0A444Y4Z4, A0A445BB59, A0A445BB22, A0A445C0Z7, A0A445ECU2, A0A444Y554, A0A445BY98, A0A444WNK3, A0A445BB41, A0A444Y534, A0A445DYV4, A0A444YP61, A0A445BX15, A0A444XXD3, A0A445BYB1, A0A444Y584, A0A445DZ14, A0A445BYB2, A0A445A4A8, A0A444WXZ0, A0A445BYA3, A0A444WNL4, A0A445A406, A0A445BX05, A0A445ABG7, A0A444WY41, A0A444Y4V2, A0A444YPC9, A0A445DRJ0, A0A445CHB6, A0A444WY06, A0A444Y6V2, A0A444Z5W0, A0A445DZ35, A0A444XXE0, A0A445DZ07, A0A444WXX4, A0A444Y4Y6, A0A445BB20, A0A444WXYI, A0A445BYA9, A0A445BY84, A0A445C0Z5, A0A445BYW2, A0A445ED07, A0A444WY99, A0A445BB37, A0A444Y4W1, A0A444Y4Y7, A0A445DYT5, A0A445BY88, A0A444Y526, A0A444Y2G7, A0A445DRJ8, A0A445DRJ4, A0A444Y562, A0A445DZ00, A0A444Y519, A0A445BP74, A0A444XBKO, A0A444Y3V7, A0A444Y6P6, A0A445BY81, A0A444WXZ1, A0A445BYT7, A0A445BY74, A0A445BB43, A0A444WXZ6, A0A445CI88, A0A445BYA0, A0A445ECS9, A0A445BB25, A0A444Y532, A0A445ECJ8, A0A445BYD0, A0A445CI74, A0A445BB51, A0A444YRD4, A0A444Y514, A0A444Y537, A0A444XDC3, A0A445A439, A0A445EIW0, A0A444Y4Y9, A0A444Y6R2, A0A444WY60, A0A445A5E9, A0A445BYB4, A0A444Y6M3, A0A444Y4X4, A0A445C0Z4, A0A445BYX5, A0A445BYX9, A0A445BB31, A0A445BY77, A0A445BYA5, A0A445CI77, A0A445E8F6, A0A445AII5, A0A445BYX2, A0A445BYE0, A0A445CBB0, A0A444Y566, A0A445DRJ3, A0A444XD58, A0A445DRQ2, A0A444WY33, A0A445E825, A0A445BS20, A0A445C104, A0A445BYZ1, A0A445BB33, A0A445BB67, A0A444WUD9, A0A445BP39, A0A445DG52, A0A445A429, A0A445BB24, A0A444Y4T8, A0A445BYM5, A0A444Y4Y5, A0A445BYV9, A0A444WY17, A0A445BY96, A0A445DRK7, A0A444Y529, A0A445BB45, A0A444Y6M2, A0A444Y6J2, A0A444Y6K9, A0A445BP47, A0A444WY50, A0A445AZM8, A0A445BB36, A0A445C8A4, A0A444WY27, A0A445A408, A0A444XXR6, A0A444Y4W8, A0A444WY44, A0A444Y6V9, A0A444Y594, A0A445BYW3, A0A445BB13, A0A445DRJ9, A0A444WXY2, A0A445A416, A0A445BZ11, A0A445A4D2, A0A290GKF7, A0A445BKE9, A0A444Y540, A0A445BB44, A0A445A4A2, A0A445AIK2, A0A445BY95, A0A445CSW5, A0A445A418, A0A445BB46, A0A444XXR8, A0A444Y4Q5, A0A445BB60, A0A444Z8T4, A0A445DJU8, A0A445BB42, A0A444Y6I9, A0A444WY31, A0A445BZ05, A0A445CIM3, A0A444Y4Y0, A0A445BYI2, A0A444Y542, A0A444Y535, A0A444WXZ9, A0A445E7X5, A0A445E0F1, A0A445BB48, A0A444Y4W6, A0A444Y4Y8, A0A445BB50, A0A445AII6, A0A445BY65, A0A445E0N0, A0A445BY56, A0A445CBC1, A0A444Y553, A0A445A499, A0A445DYX4, A0A444XXS1, A0A445E8D0, A0A444WY11, A0A445BX01, A0A0D6R0F5, A0A0D6QTT4, A0A0D6QT12, A0A0D6QTN1, A0A0D6R4V4, A0A0D6QT17, A0A0D6QRX4, A0A0D6QRZ3, A0A0D6QTH7, A0A0D6QR37, A0A0D6QRW0, A0A0D6QSZ0, A0A0D6QR91, A0A0D6QR45, A0AIQ3HVR3, A0A2P6M7B1, A0A091BFC1, A0A0B6XVM2, A0A444SIQ6, A0A444TRU3, A0A444SZK2, A0A444SL52, A0A444SR30, A0A444SMM6, A0A2U1MNQ4, A0A2U1MVH2, A0A2U1QG88, A0A2U1KI18, A0A2U1QD59, A0A2U1KPH1, A0A2U1KAR9, A0A2U1Q3R8, A0A2U1PGN2, A0A2U1MXM3, A0A2U1Q692, A0A2U1MYT5, A0A2U1QB37, A0A2U1PC96, A0A2U1MCE3, A0A2U1P9R0, A0A2U1LBZ4, A0A2U1PTC0, A0A2U1NVX8, A0A2U1MRL9, A0A2U1MF47, A0A2U1MSA6, A0A2U1MA90, A0A2U1L2N0, A0A2U1K9W3, A0A2U1Q5W4, A0A2U1NCZ8, A0A2U1PHB5, A0A2U1P0Y9, A0A2U1MKO5, A0A2U1LB20, A0A2U1LCW0, A0A2U1P3C5, A0A2U1M6L0, A0A2U1QB05, A0A2U1MFU2, A0A2U1MRH5, A0A2U1L7Y8, A0A2U1Q3Y3, A0A2U1KHM7, A0A2U1L9K1, A0A2U1PVA8, A0A2U1KTL0, A0A2U1PT94, A0A2U1MKG2, A0A2U1MIH0, A0A2U1QHC0, A0A2U1QKQ6, A0A2U1PGR0, A0A2U1MK14, A0A2U1KKI3, A0A2U1L628, A0A2U1LK69, A0A2U1LX06, A0A2U1M3I4, A0A2U1LEW8, A0A2U1KKC2, A0A2U1MXT3, A0A2U1PTA5, A0A2U1KHY4, A0A2U1MUK8, A0A2U1L5S7, A0A2U1N8E3, A0A2U1KIY8, A0A2U1NEM6, A0A2U1P0M6, A0A2U1PDI3, A0A2U1MSU5, A0A2U1LJ61, A0A2U1PQZ4, A0A2U1PD36, A0A2U1LEY7, A0A2U1MJY2, A0A2U1KDS1, A0A2U1KXK7, A0A2U1KWP0, A0A2U1LG99, A0A2U1NQJ3, A0A2U1P450, A0A2U1N3I9, A0A2U1LNH3, A0A2U1PZ37, A0A2U1MKO1, A0A2U1P169, A0A2U1L3H4, A0A2U1NUG1, A0A2U1LPS2, A0A2U1PT69, A0A2U1LZ92, A0A2U1LEV4, A0A2U1KQB6, A0A2U1MFI2, A0A2U1MWD6, A0A2U1QDH1, A0A2U1PD98, A0A2U1KFT4, A0A2U1PG62, A0A2U1M3F8, A0A2U1KBU3, A0A2U1MMKO, A0A2U1KFP7, A0A2U1NQJ1, A0A2U1P8A2, A0A2U1N8W6, A0A2U1MCQ6, A0A2U1QB20, A0A2U1MSB3, A0A2U1MKZ0, A0A2U1MY98, A0A2U1PDB5, A0A2U1MGQ2, A0A2U1KLB8, A0A2U1LNA4, A0A2U1PIG5, A0A2U1PG29, G1XPN8, G1XSL9, G1XGI6, G1XG00, G1XSA2, D4ARU1, E4UUF0, A0A0A9HRX8, A0A0A9TEG4, A0A0A9DPX1, A0A0A8YWY2, A0A0A9F543, A0A0A9E2Y2, A0A0A9E7P7, A0A0A9QLR5, A0A0A9DFU5, A0A0A8YEX4, A0A0A9NPY6, A0A0A9GL28, A0A0A9DM30, A0A0A9S6Y8, A0A0A9DF09, A0A0A9BMR2, A0A0A9LN34, A0A0A9TYC3, A0A0A9QZ24, A0A0A9DW73, A0A0A9H3W6, A0A0M3IPJ6, A0A0M3HP86, A0A0M3IT96, A0A0M3IBY8, A0A0M3HND4, A0A0M3HU83, A0A0M3HEY4, A0A0M3IH35, A0A0M3ILR8, A0A0M3IVC1, A0A0M3IRC3, A0A0M3IAG3, A0A0M3IPG4, A0A0M3HG49, A0A0M3IRJ0, A0A0M3HQ47, A0A0M3I750, A0A0M3ITR0, F1L0P3, F1L0G7, F1LC48, F1KWX7, F1KVZ1, F1KRY6, F1L7L8, A0A2R8B9F5, A0A3N4IAA5, A0A3N4IAW4, A0A167YNV3, A0A1R3L6N1, A0A1R3L7T1, A0A2G7FEN3, A0A2G7G4M6, A0AIF7ZRV8, A0A1F8A4B8, A0AIF7ZUM9, A0A0U5FUR8, A0A0U5GHF6, A0A0U5FUB2, A0A0U5CCI5, A0A2I1DHJ1, A0A2I1D6S1, A0A2I2FIZ7, A0A2I2EZ59, A1CCE8, A1CBV4, A0AIE3BSI6, A0A319DW04, A0A317WGQ7, B8NFC9, B8NFD0, B8NDN6, A0A2P2HFU4, A0A2P2HLC7, A0A2P2H4K6, A0A2P2HM09, A0A2P2HFT0, A0A2P2H9K4, A0A2P2HPX0, A0A3M7JRD9, A0A3M7JTE8, A0A3M7K264, A0A364MKB4, A0A364M579, A0A0J5Q2X8, A0A0J5PMA7, A0A1L9VP26, A0A1L9VC48, A0A1L9VP33, A0AIL9VNY3, A0AIL9VRE9, A0A317WAR2, A0A395HFU3, A0A395GPB8, G7XTE0, A0A319AAV7, A0A319A7A0, A0A0S7DE04, A0A0S7E252, A0A0S7EBW1, A0A1M3TFU6, A0A1M3T8Q8, A0A1M3TTZ5, A0A146F4B8, A0A3D8T616, A0A3D8S6I6, A0A3D8T3A9, A0A3D8QMJ3, A0A318YC13, A0A318YlN8, A0A318YAK2, G3XMA4, A2QYV6, A2QTC1, A0A370BU00, A0A370BW53, A0A3F3R4A3, A0A117E3X4, A0A0L1J937, A0A2I1C107, A0A2I1BTK7, A0A2I1CJW4, A0A2I1CCR0, A0A2I1BTA2, A0A2T5M095, A0A2T5LRQ5, A0A0F8UID4, I8U2J0, I8A614, I8U453, Q2U7K1, Q2UP96, Q2U8A9, Q2U2F0, A0AIS9DBB6, A0AIS9DIX8, A0AIS9DJT8, A0A0F0IBG5, A0A0F0ICT4, A0A370PJK5, A0A370PHC2, A0A0F8X4Q4, A0A017SC51, A0A017SDA8, A0A318ZXC8, A0A318Z9C0, A0A318Z979, A0A3A2ZMA4, A0A319EXR3, A0A319EAA1, A0A319ECG3, A0A317VSX8, A0A2I2GSR7, A0A2J5HZ31, A0A2J5HG69, A0A2J5IA66, Q0CRW2, A0A397GTA6, A0AIL9NER7, A0AIL9NHC1, A0A397I0S7, A0A3R7FTI7, A0A421CZV9, A0A397FZA9, A0A397H5H6, A0A397I608, A0A0K8LIL4, A0A319CFJ0, A0A319C2C5, A0A319CGR1, A0A319B527, A0A2V5HBT7, A0A1L9R4F3, A0AIL9RJD3, A0AIL9RI70, A0A3P8R5U0, A0A3P8QWI7, A0A3P8NAK1, A0A3P8NAL8, A0A3P8NKJ4, A0A3P8PUS6, A0A3P8NAX7, A0A3R5SPP4, W5LAJ7, A0A3B1IZ91, W5JZP1, W5LF06, A0A3B1JB59, W5LF07, W5KKK6, A0A158NQ95, A0A158NRJ0, A0A158NUD3, A0A158NCF5, A0A158NFW1, A0A158P387, A0A195BUK4, A0A195B440, A0A151I1P9, A0A195BWB4, A0A195BVH1, A0A195B228, A0A195BU26, A0A195BWF1, A0AIX0T3J5, A0A1M6C5Z3, A0A0B1Q6J5, A0A0B1Q3S9, A0AIH0C5Q6, A0AII2INE0, A0AII2J8H4, A0A0S2EVY8, A0A0Q6CLW5, A0A0Q6D7V7, A0A0Q6CNY5, A0A0Q6DNE7, A0A0Q6E7R1, A0A175RVT2, A0A175RB81, A0A175RYG2, A0A074VR01, A0A074W6P0, A0A074VFW8, A0A074VY00, A0A074VWR6, A0A074VMM6, A0A074VQQ6, A0A074W863, A0A074VGQ4, A0A074WSI2, A0A074WT58, A0A074W896, A0A074WDM8, A0A074X5F4, A0A074WD87, A0A074WP99, A0A074WYP5, A0A074X7D6, A0A074X9M1, A0A074XKI4, A0A074XMC5, A0A074XW73, A0A074XUY6, A0A074XV26, A0A1A7MGL9, A0A1A7MIQ4, A0A1A7MHG3, A0A1A7MCT8, A0A1A7MLB2, A0A1A7MP46, A0A1A7MAV7, A0A1A7MR78, A0A1A7MEM4, A0A1A7MN21, A0A1A7MAJ0, A0A1A7MFI3, A0A1A7MS81, A0A1A7MDG6, A0A074Z9D4, A0A074XZQ2, A0A074Y4C9, A0A074YXY8, A0A074Y4Q8, A0A074YRD0, A0A074YLW2, A0A074YDL6, A0A074YCF2, A0A074YUS8, A0A074Y8S5, A0A074YQX7, A0A074YG47, A0A074XZ34, A0A074Z895, F0YIS4, A0A2I4AVX2, A0A2I4CU49, A0A2I4CCC7, A0A2I4D285, A0A2I4D8H5, A0A2I4CKP4, A0A2I4AVX1, A0A2I4BJ83, A0A2I4AVX5, A0A2I4CU47, A0AID2AC95, A0AID2ADW2, A0A1D2A527, A0A087SNA2, A0AID2AA88, A0A3M7KY55, A0A3M7L506, A0A087S9Y1, A0A3M7L688, A0A1D2A2T0, A0A1D1ZN49, A0A1D2AGQ5, A0A087SA78, A0A202DKR6, A0A034V924, A0A034V101, A0A034VV04, A0A034VBU4, A0A034VP05, A0A034V269, A0A034VU87, A0A034VB68, A0A034WDUO, A0A034VQT0, A0A034VAC8, A0A034VA94, A0A0K8WFY5, A0A0K8V187, A0A0K8TYA5, A0A0K8U2Z1, A0A0K8W143, A0A0K8W107, A0A0K8WAG5, A0A0K8UYR3, A0A0K8UC22, A0A0K8W0F9, A0A0K8URT7, A0A0K8W7I2, A0A0K8U7H8, A0A0K8VH83, A0A0K8VDD1, A0A383ZG27, A0A383ZV45, A0A383Z1M8, A0A383ZVD0, A0A384AFX7, A0A384AFQ0, A0A087VQ86, A0A087VHF0, A0A2E9UHUO, A0A2P4SWX5, A0A2P4SJS3, A0A2K9V7N6, A0A2K9V7Z4, A0A2K9V7Z1, A0AIR0FC62, A0AIU9MF28, A0AIY1WQS1, K8EB66, K8FDI0, K8F4B2, K8EQC3, K8EJ82, K8EAB0, K8F6I2, E5ESL6, F4NSE3, F4NSY5, A0A177WA49, A0A177WAP2, A0AIS8VW60, A0AIS8VHV0, M2LEM6, M2MRY7, M2LJL5, M2N072, A0A1G6CN88, A0A1G6CP43, A0A2E7GCJ6, A0A2E7G7G0, A0A2E8HW08, J5JQG2, J4WGF4, J4KLZ6, A0A0A2VAIl, A0A0A2VQE8, A0A2N6NCG5, A0A2N6P0V4, A0A2N6NH65, A0A2S7Y029, A0A2S7XWT7, A0A167DY06, A0A162JT95, B2IFP4, A0A2A9MK42, A0A2A9MEK6, A0A2A9MHF7, A0A2A9M2B2, A0A2A9MP64, A0A2A9M947, A0A2A9MH57, A0A2A9MHN4, A0A2A9M1K1, A0A2A9MK31, A0A2A9MLG1, A0A2A9M8Y8, A0A2A9MGA0, A0A2A9M957, A0A2A9M7H0, A0A2A9LZF4, A0A2A9MGC4, A0A2A9MDT6, A0A2A9MEJ6, A0A2A9MEY2, A0A2A9MBU9, A0A2A9MDP9, A0A0J8B6J9, A0A0J8B9T3, A0A0J8DZK2, A0A0J8B8G6, A0A0J8B6H3, A0A0J8B5L4, A0A0J8B9Q2, A0A0J8E0I7, A0A2C9M6E5, A0A2C9M2K3, A0A2C9L085, A0A2C9LUG0, A0A2C9KVYO, A0A2C9JZZ3, A0A2C9LGM4, A0A2C9MAU4, A0A2C9KU12, A0A2C9L1D2, A0A2C9M004, A0A2C9LZ70, A0A2C9L2E0, A0A2C9LWZ3, A0A2C9LD29, A0A2C9KJS2, A0A2C9LP23, A0A2C9KNW3, A0A2C9LTI5, A0A2C9M9Z8, A0A2C9KM74, A0A2C9KKC9, A0A2C9JPS7, A0A2C9M6Q8, A0A2C9KBM1, A0A2C9JPW5, A0A2C9LE46, A0A2C9LTU9, A0A2C9JNC8, A0A2C9LNH2, A0A2C9LXF9, A0A182YU34, A0A2C9JRC5, A0A2C9JBX8, A0A2C9LL38, A0A2C9KHG2, A0A2C9M7I1, A0A2C9KDN6, A0A2C9LWA8, A0A2C9K7K3, A0A2C9KKA8, A0A2C9L3Z3, A0A2C9LBA6, A0A2C9KJ30, A0A2C9M3Q1, A0A2C9M449, A0A2C9KT20, A0A2C9KXA4, W6Z007, W6ZCF9, W6Z4B8, W6ZJUO, W6ZED0, W6ZMD3, W6ZA29, W6Z9A5, W6Z1B7, W7DVV8, W7EBL2, W7ER18, W7EF46, W7EPL2, W7ESE4, W7EV06, W7DVB6, W7E897, W6YTV6, W6Y989, W6YAZ9, W6XJ34, W6Y7X4, W6XU68, W6Y6Y8, W6Y442, W6EIN7, W6EMA3, W6EMA1, W6EIL6, W6EK17, W6EMB1, W6EVT2, W6EVS5, W6EVU1, W6EIM1, W6EIS1, W6EMA7, W6EIT3, W6EVS9, W6EK05, W6EMA5, W6EK24, W6EIM6, W6EK09, W6EK13, W6EIP2, W6EIS9, W6EVT6, W6EIS6, W6EIU2, W6EMA9, A0A352FPY6, A0A2D6H9Y1, A0AIJ9RE20, A0A2P8XY43, A0A2P8YQ78, A0A2P8YMN5, A0A2P8YR42, A0A2P8YJK3, A0A2P8YZ76, A0A2P8YR97, A0A2P8Z5Y9, A0A2H4UUU3, A0A2H4UU38, A0A0S4J9J2, A0A0S4J936, A0A0S4JQX2, H9IXB3, H9JA26, H9JJP0, H9JJP1, H9JE81, H9JE80, L8IUV1, L81A26, L8IEV1, L81Y98, L81C35, P06868, F1MZD6, A0A1L3G6L5, Q6PZ62, Q6Q017, Q2KJB4, E1B726, E1BDW7, A0A3Q1LSS6, A0A3Q1LSX3, A0A3Q1LT55, F1MUT4, Q5NTB3, Q2KJ63, Q24K22, Q76BS1, A7E350, A0A370L6R0, R1GKS9, R1G4N0, A0A384J9B7, A0A384JR75, M7UKG0, M7U138, G2YNK3, G2XPN8, A0A3M7RDQ8, A0A3M7T8X2, A0A3M7RNS1, A0A3M7SMW6, A0A3M7RME1, A0A3M7RDYO, A0A3M7P8R6, A0A3M7PKUO, A0A3M7RGA3, A0A3M7PJ96, A0A3M7SM38, A0A3M7SFE0, A0A3M7RAU7, A0A3M7S8F6, A0A3M7RN83, A0A3M7T9E3, A0A3M7SAU8, A0A3M7RQA8, A0A3M7PUQ9, A0A3M7P3V5, A0A3M7P7Y9, A0A3M7QIW1, A0A3M7REF6, A0A3M7SUT2, A0A3M7S822, A0A3M7QVJ8, A0A3M7P6F2, IlHU53, A0A2K2CXA4, A0A2K2CWA4, A0A0Q3GP11, I1J295, A0A2K2CIR5, I1J2C4, A0A2K2CGC5, I1INT0, I1J2S3, A0A0Q3EAI5, I1GMG6, A0A2K2CIQ8, A0A2K2CIR1, A0A0Q3NIJ9, A0A2K2CIS7, I1IA55, A0A2K2CST1, A0A0Q3EAD2, A0A0Q3JAA8, A0A2K2CGA7, I1J292, A0A2K2CIR0, I1GYM6, A0A0Q3FVF0, 111349, A0A0Q3ED56, A0A2K2CIS4, A0A0Q3QSF6, I1HJD9, A0A0Q3EYJ6, A0A0Q3IF41, A0A2K2D2Y3, I1HHX6, A0A0Q3GIB6, A0A2K2DI89, I1IXN9, A0A2K2D7E6, A0A0Q3GM35, I1INTI, I1IUS5, I1HW64, A0A0Q3R1L3, I1HSB4, I1HSB6, I1GTM1, A0A0Q3HD16, I1IEU4, A0A2K2CZG2, A0A0Q3HM63, I1HU55, A0A2K2DFH8, A0A2K2CGL7, A0A2K2CIU7, A0A0Q3JCV1, A0A0Q3JHU3, A0A2K2CGB5, I1IU59, A0A2K2CIT3, A0A2K2DFD8, A0A2K2DFF0, I1GTK9, A0A0Q3NYQ7, A0A2K2CIR9, A0A0Q3RAK5, I1GL44, IlHAYl, I1IXN8, A0A0Q3HI45, A0A0Q3INA5, A0A2K2CIS0, A0A0Q3RM98, A0A0Q3KXH5, I1IFY2, I1HSA3, A0A0Q3H363, I1GTM2, A0A2K2CIS1, A0A0Q3LFD5, A0A2K2CIT7, I1HW63, A0A0Q3QIL7, A0A0Q3JZ94, A0A0Q3QBI3, A0A2K2DFD4, I1HDE8, A0A0Q3IWS7, A0A2K2CIP9, A0A2K2DFE9, A0A2K2CIT2, A0A0Q3I905, A0A0Q3EAH6, A0A0Q3EAI4, I1GXK1, A0A0Q3IBUO, A0A0Q3GCR1, IlJ2A0, IIHEJ3, A0A0Q3HSSO, A0A0Q3NJG4, A0A2K2CIR6, I1HDE9, A0A0Q3EAE1, A0A0Q3HLT2, A0A2K2CIQ9, A0A2K2DFC3, A0A2K2DFC5, A0A0Q3LK38, A0A2K2DI88, A0A2Z4FIU7, A0A257KNM4, F7QHU6, A0A3SOG3E0, A0A363TXD2, A0A3SOH313, A0A431M7G0, A0A431QX01, A0A3SOEKZ0, A0A1I7FU56, A0A1H1Y2P1, A0A1X3FPA0, A0A1X3GN34, A0AIX3GRS6, Q89V82, A0A2A6N5F9, A0A0E4BN53, A0A0D1MCK2, A0A1E3EC36, A0A1M5KHK6, A0A1N6F532, A0A1M7TM76, A0A1M5HCZ9, A0A1H4ZSB0, A0A410VES2, A0A1B1UPJ0, A0A023XWN2, G7DMB7, A0A1L3FRA1, A0A0A3XV48, A0A1Y2JI88, A0A0R3LBX3, A0A1M7AMK6, A0AIM6WVP9, A0A0R3M8G1, A0A2H9V6F8, A0A151FH31, K5CWF7, A0A0R3E5M9, A0A176YZA1, M4Z0Y5, M4ZBW7, A0A2U8P6V6, A0A0R3DMJ3, A0AIB9ZC66, A0A0R3MRW5, A0AIC3WWF3, A5EA89, A4YQL0, A4Z2E2, H0SMB8, H0SCU6, H0SBZ7, A0A2U8PT28, A0A2U8PN93, A0A2U8PT94, A0A2U8Q528, A0A2S6M8B5, A0A1Q5R4X0, A0A150UDS9, A0A176Z2D2, A0A109JGJ9, A0AIV5EYR3, A0A2A6NCZ9, A0A410W142, A0A109QD69, A0AII3S3F5, A0AII3J718, U1H1Q2, A0A150ZHL9, A0A0S6UQ35, A0A160UQP7, A0AII3V8E7, A0AII5BXB3, A0A2M8RAB9, A0A0Q6A3Y8, A0A1B9Z3Q7, A0A0D7NXE7, A0A0D7N7J5, A0A0D7Q5V0, A0A430MFG0, A0A2T2QQE6, A0A2T2PZ36, A0A2T2PSY2, A0A2T2Q0F5, A0A2T2PT25, A0A2T2PSY6, A0A2T2Q4P1, A0AIQ5SAH3, A0A1Q5R9S2, A0A1I4H0I3, A0A1H8BXF8, A0A1Y6KJQ2, A0A1Y6KP04, A0A2U3PS34, A0A1G7GWB1, A0A1G8B4K1, A0AII4URV1, A0A3N6PAA8, I0FZQ2, A0A2K8Y6Q4, H0STR9, H0TBX2, H0TGS9, A0A2M6UE20, A0AIR1QEF7, A0A2A2VLQ2, I2QLW7, H5YCF8, A0A2A6PYD0, J2W3F2, J3I044, A0A160UAV3, A0A0R3KWP9, A0A0R3CA11, A0A385GLI2, A0A385GLJ0, C3YDL6, C3Z071, C3YAZ6, C3ZYT2, C3ZFD3, C3ZHY1, C3YYM3, C3ZAW6, C3XQ58, C3XQ51, C3Y7N4, C3YP82, C3ZLM9, C3Z0V1, C3XPW8, C3Y305, C3ZYG9, C3ZMW2, C3ZMB3, C3Y3P1, C3ZGD7, C3Y566, C3Z0R2, C3ZJ84, C3YU46, C3XY24, C3YC74, C3ZFT9, C3ZHS3, C3Y7E8, C3XW29, C3YDL9, C3ZT44, C3XUJ8, C3YP09, C3ZDYO, C3YS46, C3YDMO, C3ZZJ9, C3YCI7, C3YU47, C3YNF1, C3ZHW8, C3ZG87, C3XY53, C3YWE1, C3XW28, C3YMU3, C3ZK10, Q8LP65, Q84KY8, A0A397YWD8, Q84KX9, A0A3P5Z2T0, Q1XIH3, A8QWH1, A0A398A0U2, A0A1L7NT06, 023856, A0A397YDG2, B5U9A7, A0A397YVY9, A0A397YF45, 023848, A0A3P6C3Y6, A0A3P5ZPK2, A0A3P5XWYO, A0A397YV83, A0A397YFB7, A0A3P5Z9J5, A0A3P6CY69, A0A3P6D969, A0A3P5YJ21, A0A397YBC1, Q2MHJ1, A0A3P6CB57, Q948X2, A0A397Z250, A0A397YV95, A0A397XSU5, A8QZG2, B5U9A8, Q948X1, A0A397YA29, A0A397YPP3, Q9AYP7, 023850, 023855, Q6SVA0, A0A3P6A0C0, Q7DN95, A0A3P6ASY7, A0A398A9P6, A0A3P5ZV33, A0A3P6BWB7, A0A3P5YSC4, A0A397ZSN3, Q2EMZ8, Q6SVA3, A0A3P6A3C2, A8QZC5, A0A397XRI1, A0A3P5Z8N8, Q39279, A0A3P5YE62, A0A3P5XWD4, Q84KX7, A0A398AQ76, 023859, Q84KX6, Q39276, A0A3P5YCU6, A0A398AQ52, Q39280, Q18PE5, Q84KZ0, A0A3P6C0F7, 023851, A0A398A6C5, 023745, Q84KY9, A0A3P6CGF5, 023847, A0A398ARS1, A4UWM4, A0A3P6C098, A0A398AF65, A0A397YVY3, 023860, A0A3P5ZGB7, 023849, Q04034, 023861, Q84KY2, A0A397YCG0, Q84KY4, A8QWH4, A0A3P5Z9S3, A0A398AMH2, Q84KY1, A0A3P6A681, A0A397Y539, Q2MHJ2, E21V49, A0A3P5Z8U6, Q84KY6, A0A1L7NT17, A0A397Y192, A0A397ZWZ8, A0A1L7NT09, 023852, 023854, A0A397KYV2, A0A398AKR7, Q39277, A0A3P5ZXM2, A0A397XTY3, A0A3P5ZIB6, Q84KY3, Q84KY0, Q41222, A0A3P6AIE3, A8QZG7, A0A3P6BVP4, Q8W407, Q84KY7, G3EXH7, A0A397YV80, Q9AYP5, Q948W9, A0A397Y1U1, A0A397XVY9, Q948X0, A0A398A3C5, A0A397ZLT6, A0A3P5ZRW3, A8QZC9, A0A398ASE3, A0A397KY95, A8QZF8, A0A398AT17, Q9AYP2, A0A3P6ACK6, A0A3P5YRC2, A0A397YRH6, A0A397KZ86, Q8S9B0, B5U9A6, A0A3P6BCU6, Q6SV99, A0A397YN43, A0A397YFK7, A0A397YPJ3, Q6SV98, A0A1L7NT04, E2IV46, A0A397Z248, Q271Z4, A0AIL7NT22, A0A397YJM2, A0AIL7NT10, A8QZG5, A0A398A3A1, A0A3P6C7S0, A0A397L944, Q84N09, Q1XIH1, A0A397XUJ7, Q1XHT4, 080430, A0A397XT43, A0A397YZX3, A8QWG5, 080344, A0A397ZXN6, A0A397YGY4, A0A3P5ZWE1, A0A397XUI4, A0A397Y2G3, E2IV47, A0A398AKX8, A0A3P6C0A8, 023857, A0A3P6C053, A0A397Z461, A0A397ZIG8, A0A1L7NT25, Q9ZNW9, A0AIL7NT13, A0A397Y4Y8, Q9SXK6, A0A3P5YMA1, A0A398A4W5, 023853, Q9AYP6, A0A397YQ80, A0A3P5ZPZ5, Q84KX8, A0A3P6CIH1, 023858, A0A3P5YA39, A0A3P5ZWS7, A0A397XQX1, A0A397L1V4, A0A3P6DPZ9, A0A398A9U1, A0A397ZGZ8, A0A397YV89, A0A3P5YQL4, A0A3P5Y7E1, Q1XIH2, A0A397YNB1, A0A3P6BQI0, A8QZD1, A0A3P6A4U7, A0A397YA55, Q84KY5, Q9SAZ7, A0A397XZ92, Q1XHT5, 080343, Q39278, A0A3P6B4Q3, A0A3P6C2F5, A0A397YX37, Q6SVA2, 048512, Q948X3, Q9AYP8, A0A3P5YM72, A0A3P5YDK4, A0A3P5ZQK6, A0AIL7NT14, A0A3P5ZUK5, E2IV48, Q9SXK7, Q18PE6, A0A397ZIB8, A0A397Z263, Q2PML8, Q39282, Q84N10, Q9ZNW6, A0A397ZR25, Q04168, A0A3N6S4L1, A0A3N6QXX1, A0A3N6QKJ3, A0A3N6TM80, A0A3N6QYA9, A0A3N6QM77, A0A3N6R7H2, A0A3N6QNP4, A0A3N6PZG5, A0A3N6QQN7, A0A3N6UQ88, A0A3N6RPS9, A0A3N6T5B6, A0A3N6S057, A0A3N6Q5N7, A0A3N6S1R2, A0A3N6U997, A0A3N6T699, A0A3N6SZL5, A0A3N6RNN4, A0A3N6UJ64, A0A3N6QYV1, A0A3N6UHA9, A0A3N6RZC7, A0A3N6S1W1, A0A3N6QNP5, A0A3N6RFD8, A0A3N6SMY5, A0A3N6R014, A0A3N6R511, A0A3N6QTL5, A0A3N6QXV3, Q8LP64, Q9SBK7, Q8LP62, Q01963, Q9M4E9, Q7FPE1, A0A078FY82, A0A078JHP5, Q43393, A0A078IH30, A0A078JPB3, A0A078GD60, A0A078G3D0, A0A078FY93, A0A078JQF9, A0A078HBI2, Q08700, A0A078I0E8, A0A078HA89, Q6SV95, A0A078HDD4, Q6SV97, A0A078HAC3, A0A078HEK1, A0A078IMG1, A0A078HR13, A0A078IFP5, A0A078HVX7, A0A078IAX0, A0A078HN20, A0A078F3B5, Q6SV92, A0A078GIA3, Q6SV96, Q39356, A0A078GM80, A0A078JYB1, A0A078H803, A0A078JCU5, A0A078HR81, A0A078J7X9, A0A078IDY2, A0A078GN24, A0A078HD30, A0A078H8L5, A0A078G7I9, A0A078HAD2, A0A078F337, A0A078I9I0, A0A078ILK5, A0A078H3H5, A0A078JMJ4, A0A078HEMO, A4UWL9, A0A078GS40, A0A078JKE7, A0A078FML4, A0A078FHJ4, A0A078FPN5, Q39363, A0A078J557, A0A078II36, A0A078IIQ7, A0A078HDC2, A0A078JFN3, A0A078FRP9, A0A078FIUI, A6P327, A0A078JBM2, A0A078GVV6, A0A078IDX4, Q39354, A0A078FI59, A0A078HT86, A0A078G2V4, A0A078JE37, A0A078FQ97, A0A078I9I3, A0A078FEU1, A0A078FKQ4, A0A078JIW9, A0A078G2W5, A0A078GU68, A0A078J1N7, A0A078G8J1, A0A078JWL9, A0A078J032, A0A078IYH8, A0A078JVW6, A0A078G3S4, A0A078FWL9, A0A078GJI3, A0A078IFM7, A0A078FQ82, A6P326, A0A078G3D4, A0A078FPH2, A0A078HAQ2, A0A078GYC5, A0A078HCX8, A0A078INX3, Q6SV94, A0A078JB85, A0A078HPD1, A0A078FIU7, A0A078GDP5, A0A078JQK2, A0A078EZE7, Q7DMS5, A0A078G200, A0A078IKUO, A0A078H3H1, A6P328, A0A078FC62, A0A078FE33, A4UWL8, A4UWL4, A4UWM3, A4UWL3, Q39357, A4UWL5, A4UWL6, Q04808, Q8LP61, P22552, P22553, P22551, A0A0D3C9A8, A0A0D3BLV5, A0A0D2ZVD2, A0A0D3AAQ0, A0A0D3E522, A0A0D3DXZ3, A0A0D3A6W5, A0A0D3AAP8, A0A0D3CYG2, A0A0D3E1N6, A0A0D3E528, A0A0D3DA71, A0A0D3DP33, A0A0D2ZU01, A0A0D3AAR4, A0A0D3CJQ9, A0A0D2ZVF6, A0A0D3BVD8, A0A0D3DXZ2, A0A0D3BVD9, A0A0D3CM95, A0A0D3CY96, A0A0D3BFE7, A0A0D3E524, A0A0D3C5Q0, A0A0D3BXW1, A0A0D3BCW1, A0A0D3C227, A0A0D3E3G0, A0A0D3C9A3, A0A0D3DGD4, A0A0D3C9A0, A0A0D3DDG1, A0A0D3BW89, A0A0D3DE12, A0A0D3E3F9, A0A0D3AD84, A0A0D3CY99, A0A0D3DC95, A0A0D3AAR5, A0A0D3DMB4, A0A0D3AAP9, A0A0D3BYM9, A0A0D3EAU1, A0A0D3CY95, A0A0D3D1P0, A0A0D3C9A9, A0A0D2ZVS8, A0A0D3DVG2, A0A0D3BT69, A0A0D3A4U3, A0A0D3DXV5, A0A0D3B6D1, A0A0D2ZWS7, A0A0D3C538, A0A0D3C9C2, A0A0D3DXZ4, A0A0D3BVD6, A0A0D3C9A2, A0A0D3C5D3, A0A0D3DZL7, A0A0D3A4U2, A0A0D3ASG5, A0A0D3ASA4, A0A0D3DP20, A0A0D3D3Q8, A0A0D3CVY3, A0A0D3BAW1, A0A0D3AYW3, A0A0D3E523, A0A0D3BVG5, A0A0D3A6W4, A0A0D3DP40, A0A0D3E5Z4, A0A0D3EI38, A0A0D3DDG2, A0A0D3CEW1, A0A0D3B529, A0A0D3C9B0, A0A0D3C999, A0A0D3DP24, A0A0D3A2M9, A8VP90, Q09092, P17840, P17841, P07761, Q9SXH4, Q93VX9, 023743, Q93VR1, Q93VY7, Q93V96, A0A3P6DR72, O04383, Q9MB87, O23832, Q84KU6, Q8S9B2, A0A3P6EAU3, Q84KV5, Q1ET35, A0A3P6AZG2, A0A3P6DSC9, Q9SXH9, Q84KV2, Q39392, Q84KV1, Q93YJ8, A0A3P6E9L0, Q84KX3, A0A3P6CXY8, A0A3P6DPD4, A0A3P6BST4, A0A3P6CG45, A0A3P6CJK8, Q9ZQU7, B5U9A4, Q9T0M1, A0A3P6G216, Q84KW1, Q84KW3, A0A3P6DQ69, A0A3P6DM94, Q93YK2, A0A3P6H645, A0A3P6DJE8, O23835, A0A3P6EPZ7, O23744, Q9ZQU6, A0A3P6GT52, Q7DLP6, Q84KX5, Q93YJ7, Q84KW2, A0A3P6GDI9, Q39390, A0A3P6CMU3, 023844, A0A3P6FD20, A0A3P6EHB3, A0A3P6DJA8, A0A3P6EN76, A0A3P6G8V9, A0A3P6AM61, Q84KX2, Q93YK0, O23836, O23838, Q84KV7, Q84KW7, A0A3P6DCU3, A0A3P6GW39, A0A3P6FM07, A0A3P6CYJ8, A8QZJ0, O23837, Q84KW0, A0A3P6ECQ4, A0A3P6CA48, Q1ET30, A0A3P6BMB0, Q9MB88, Q84KX4, O23842, O04384, Q9SXH6, Q93VN5, O23841, A0A3P6FB97, A8QZI2, A0A3P6GBD6, A0A3P6G6E7, Q8W408, A0A3P6BHJ6, A0A3P6F1R2, A0A3P6FS98, A0A3P6DWV5, Q1XIG9, O23740, Q9SXH7, O23839, Q84KW4, Q9SXH5, T1Q059, A8QZI1, Q39393, Q84KV0, Q84KV3, O23840, A0A3P6F1Q3, A0A3P6GBB9, Q84KU5, 023863, A0A3P6EDW2, Q84KV4, O23895, Q84KU7, Q43410, A0A3P6GFP0, Q84KV9, A0A3P6CQM2, A0A3P6FF23, A0A3P6EV23, Q1ET32, A0A3P6DR77, A0A3P6AP88, Q84KU9, A0A3P6F267, P93068, A0A3P6F441, Q9SAN1, Q9SXH8, Q9T0M0, Q84KW5, Q84KW8, Q39389, Q84KU8, Q84KV6, 023834, Q8W409, 023833, Q39391, A0A3P6C6P4, A0A3P6E4S9, O23843, A0A3P6FEZ1, Q84KW6, A0A3P6G9V7, 023846, Q03349, T1Q7V5, Q84KW9, A0A3P6GQQ4, A0A3P6EI65, Q93YK1, A0A3P6EAA2, Q43411, A8QZH0, Q84KV8, A0A3P6CK21, O23862, Q9XFW5, Q1ET31, Q41177, A0A3P6FAU8, A0A3P6FN43, A0A3P6FGK6, A8QZH1, 023845, Q84KX1, Q93YJ9, Q84KX0, Q84KU4, A0A3P6B1L3, A0A3P6BNH0, A0A3P6GIA4, Q8LP60, M4DRB6, M4F1X0, M4CIZ8, M4DTL2, M4FBV1, M4EZL4, M4CBQ0, M4D8N1, M4ESL3, M4EE76, M4D938, M4CHF2, M4ERK3, M4F4F3, M4DTM0, M4EE77, M4ECM7, M4DE63, M4FBV0, M4ERK7, M4DK16, M4DTL3, M4F8C2, M4DTM3, M4DK08, M4DTL1, M4D939, M4CIU6, M4EC54, M4ESI7, M4FHG6, M4DAL3, M4DTL9, M4CIZ5, M4DKI6, M4ERK8, M4FC19, M4C7T3, M4D4D3, M4CU98, M4EE78, M4E2N1, M4DTM1, M4DK77, M4DTKO, M4FBW3, M4D3D6, M4DK14, M4CZ69, M4CCH3, M4EEI3, M4FCE8, M4ESI6, M4EXD7, M4CIU7, M4EC53, M4CFP8, M4EAX3, M4EDR6, M4CB53, M4EE74, M4FIP3, M4FHL1, M4DK11, M4CIU5, M4ESI5, M4EE73, M4DTM4, M4DAL2, M4EUB0, Q8LP66, Q8LP59, Q8LP58, A0A2T5VC63, A0A346R2I9, A0A346R164, A0A3P1UMN2, A0A0H5SIF6, A0A0H5S642, A0A0KOJ6N5, A0A0J9XW65, A0A0KOJ6N4, A0A0H5S3W5, A0A0KOJDL5, A0A0H5S9G2, A0A0H5SiD5, A0A0H5SAY8, A0A0H5SR05, A0A1I9G1H5, A0A158PX07, A0A158PZU7, A0A158PY61, A0A0J9Y7A3, A0A1I9G322, A0A158PYI4, A0AII9G2Y4, A0A0KOJDU5, A0A0I9R304, A0A0KOJ681, A0A0KOJ644, A0A158PX08, A0A0KOJDU6, A0AII9GCN9, A0A158PY62, A0A0KOJU89, A0A0KOID7, A0A0J9XZT0, A0A0J9Y9P8, A0A0KOIQ14, A0A0KOISEI, A0A0KOJJL0, A0A0J9YOR4, A0A0J9YCN5, A0A0N4TNB0, A0A158PRG8, A0A0N4TMV2, A0A0N4TSSO, A0A158PSR5, A0A158PQG8, A0A0N4SZC0, A0A0N4T3W4, A0A0N4TFB9, A0A0N4TYT6, A0A0N4T2A3, A0A0N4TLH9, A0A0N4SYT2, A0A0N4TPM2, A0A0N4TR02, A0A0N4SWY6, A0A0N4TNG8, A0A0N4TVC8, A0A0N4SZ35, A0A3P7TPN6, A0A158PT29, A0A0R3Q302, A0A3P7SG12, A0A0R3QBN5, A0A0R3QQ91, A0A0R3QX15, A0A0R3R813, A0A0R3QCX7, A0A0R3R1C6, A0A0R3R9R2, A0A0R3R309, A0A0R3QNJ6, A0A0R3QRA9, A0A0R3Q4B1, A0A3P7T0Q5, A0A091GKM8, A0A091I300, A0A091HAC8, A0A091HG60, A0AII7S3Z5, A0AII7RNK5, A0A1I7RQH8, A0A1I7SVU8, A0A1I7RV17, A0AII7S9K1, A0A1I7SLP2, A0A1I7RUO, A0A1I7SAF2, A0AII7RLM5, A0A1I7SQH5, A0A1I7S7L5, A0A1I7SX51, A0A1I7RIX3, A0AII7S4P8, A0A1I7SLSO, A0AII7SB83, A0A1I7SDI4, A0A1I7SER8, A0AII7SST9, A0AII7RHA2, A0AII7RN79, A0AII7RHH1, A0A2V1BVR2, A0A2V1BNQ3, A0A2V1B6F2, A0A2V1BD27, A0A2V1BP60, A0A2V1C858, A0A2V1BJ06, A0A2V1B5B9, A0A2V1BWQ8, A0A2V1CUT5, A0A2V1CF77, A0A2V1BSL5, B6VC06, G0NPZ3, G0MVC6, G0N8P6, G0M8F1, G0PGC1, G0M6R7, G0NE96, G0NN92, G0N7Q3, G0MCH5, G0MRH4, G0MHL2, G0PDT9, G0MVD5, G0M8F2, G0NLF9, G0P5N7, G0NU28, G0NII5, G0PEQ2, G0NE92, G0MHP5, G0NGT2, G0P5E1, B0KOA0, A8XM04, A8XU34, A8X9M4, E3CU51, A8WN02, A8XZL8, A8WT52, A8Y2W2, A8X5P5, A8XZR6, A8WPA1, A8XDB5, A8XK91, A8YOT3, A8X0Q4, A8XQU5, A8WVZ7, A8WRC5, A8X0Q5, A8WXE5, A8Y4J3, Q27394, Q10125, Q09271, Q10952, Q17800, H9G2Z5, H2KZ73, B6EU70, A0A2C9C2X5, A0A2C9C344, Q7YX17, Q22631, ClP662, D0VWP5, Q21782, Q22214, A0A2C9C2R3, O61834, Q9XUE5, Q22815, Q94248, O17347, Q23327, O62201, H8W3Y1, Q18298, G5EBR9, G5ECB3, G5EDB1, Q20167, Q23328, Q22902, O45277, G5ECB4, Q8T3B7, K7GW62, K7HND9, K7GW65, A0A2H2ICP9, A0A2H2I0G9, K7GW61, K7GW66, K7H567, A0A2H2HYS4, K7H566, K7H1H4, A0A2H2I0E1, A0A2H2J0L9, K7GW60, H2WB38, A0A2H2IE65, A0A2H2IWD6, A0A2H2IAG5, A0A2H2IAKO, A0A2H2I6U0, A0A2H2HUZ7, K7GTV4, K7HVV9, A0A2H2I7Z8, K7H652, K7H407, A0A2H2JF03, A0A2H2HU97, A0A2H2IJJ4, K7H1H3, K7H408, K7H6J2, K7HAQ8, A0A2H2I481, K7GTV5, K7H6I4, K7HAQ7, K7HND8, A0A2H2I4H8, K7H6J3, K7HVW0, K7H6I3, A0A261ACA4, A0A261AH64, A0A261CPYO, A0A261CSP6, A0A261AU21, A0A261CNX8, A0A261CR66, A0A261BZ73, A0A261CE90, A0A260ZQN6, A0A261CNC8, A0A261CA21, A0A261CK25, A0A261CGY1, A0A261CP70, A0A261CFW0, A0A261BUT5, A0A261B5J5, A0A261ANV5, A0A260ZQ86, A0A261APP7, A0A261BID9, A0A260ZF97, A0A260ZPE3, A0A2G5T5T2, A0A2G5SYL6, A0A2G5U9C7, A0A2G5U9K5, A0A2G5ULQ0, A0A2G5SZG6, A0A2G5SPU6, A0A2G5UM21, A0A2G5U9C6, A0A2G5V3A7, A0A2G5U4H9, A0A2G5UOG8, A0A2G5T662, A0A2G5SPSO, A0A2G5SIZ1, A0A2G5V2K3, A0A2G5SQ73, A0A2G5U9E1, A0A2G5TVG4, A0A2G5U4R2, A0A2G5VRD3, A0A2G5U9B1, A0A2G5UKX7, A0A2G5TNF2, A0A2G5TY68, A0A2G5UG40, A0A2G5U9E5, A0A2G5U784, A0A2G5V2K1, A0A2G5UH11, A0A2G5SPN4, A0A2G5STL2, A0A2G5UV42, A0A2G5SYP7, A0A2G5THD7, A0A2G5TVG8, A0A2G5SPC7, A0A2G5TNG8, A0A2G5VF01, A0A2G5U9U1, A0A2G5U799, A0A2G5STT6, A0A2G5U4J0, A0A2G5VR02, A0A2G5VEG5, A0A2G5UKSO, A0A2G5STB6, A0A2G5SNI9, A0A2G5U534, A0A2G5U9C2, A0A2G5TI38, A0A2G5U9A0, A0A2G5TNA2, A0A2G5U4J4, A0A2G5TYA1, A0A2G5U787, E3N801, A0A2P4WK92, A0A2P4W3J2, E3M888, E3NWP9, E3M6Q4, E3M5X6, A0A2P4W7N0, A0A261BAQ3, A0A2P4WCC6, A0A261AJI9, A0A260ZJI1, A0A2P4V2Y6, E3M244, A0A2P4WFX4, A0A261AK10, E3NPV1, E3NQB6, E3M5X4, A0A261ATB5, A0A261AI47, A0A2P4VKF5, A0A260Z8D5, A0A261ALS6, A0A2P4WL44, E3M2F3, A0A261B0J8, E3LE81, E3MSS4, A0A2P4V2Z2, A0A2P4W7H8, A0A261BE63, A0A2P4VDA9, A0A2P4VDA1, A0A261AIL2, A0A2P4WLP1, A0A2P4W0U2, A0A2P4VIC1, E3N395, A0A2P4WBE7, E3MUD1, A0A261BE25, E3MQF4, A0A261A2K8, E3LY63, A0A2P4V2Y4, A0A2P4W7D8, E3NF96, E3MSS5, E3NAC3, A0A2P4WMV8, E3NS03, A0A2P4WTN2, A0A260ZUR0, A0A261B7H7, A0A261ASY1, E3LFD4, A0A261BCB7, E3LE82, E3MA09, A0A2P4VZW1, E3MK54, E3NE41, A0A261A2U8, A0A261AY11, E3ND68, A0A261A7H9, A0A2P4WIH5, A0A261AUN6, A0A260Z4Y7, A0A261ADY1, E3M221, E3LFC5, E3LZN5, A0AII7TRC8, A0AII7UFP5, A0AII7TBP9, A0AII7TB17, A0A1I7U139, A0A1I7UEG5, A0A1I7U2M5, A0A1I7U140, A0A1I7U2M6, A0A1I7T3H6, A0A1I7UDC8, A0A1I7V193, A0A1I7T400, A0A1I7U9T6, A0A1I7UTC6, A0A1I7TB16, A0A1I7V489, A0A1I7U190, A0A1I7TL14, A0A1I7U7E4, A0A1I7ULB1, A0A1I7U7E3, A0A1I7V194, A0A1I7T081, A0A1I7T3Z9, A0A1I7U7E6, A0AII7U9T5, A0A1I7UTC7, A0A1I7UEG4, A0AII7TBP8, A0A151SMC5, A0A151SPY9, A0A151UBP4, A0A151SVK7, A0A151S8Z9, A0A151UG46, A0A151TPQ2, A0A151S200, A0A151TU08, A0A151RB11, A0A151RFX6, A0A151TV23, A0A151SVI8, A0A151QZL4, A0A151R1S2, A0A151RF37, A0A151RBF8, A0A151R092, A0A151SY98, A0A151T3Q5, A0A151RFX8, A0A151S5X, A0A151RKY9, A0A151SMF7, A0A151SVJ6, A0A151S231, A0A151SMD4, A0A151S219, A0A151S5A6, A0A151RBKO, A0A151RQ08, A0A151S2F9, A0A151QYZ7, A0A151RPY9, A0A151TBW7, A0A151SJN1, A0A151UDQ9, A0A151RDZ2, A0A151SH91, A0A151S126, A0A151RPW2, A0A151SMF2, A0A151S5R9, A0A151RKS3, A0A151T1R0, A0A151R0Z5, A0A151RPX4, A0A151RGD5, A0A151RFV9, A0A151RP79, A0A151RFY6, A0A151SJP5, A0A151RPZ1, A0A151S982, A0A151RXG6, A0A151RNK8, A0A151SM99, A0A151QZP1, A0A151RP46, A0A151RQ07, A0A151RGA5, A0A151UH94, A0A151T1Q6, A0A151SVE9, A0A151RPX7, A0A151S658, A0A151RL70, A0A151RE53, A0A151RL12, A0A151RQF6, A0A151TTP7, A0A151RBC2, A0A151RKT0, A0A151SWX4, A0A226MM08, A0A226MNE8, A0A226MN47, F6WH76, F6S5L7, F7EDL5, F6ZZG6, F7I8A8, F6VX63, F7AA32, F6X343, F7GKJ7, V9K997, V9KGH2, V9KIE5, A0A3Q7P3N0, A0A3Q7QI46, A0A3Q7NAT6, A0A3Q7RMW6, A0A3Q7Q6J8, A0A3Q7NFH9, A0A3Q7Q880, A0A3Q7QI91, A0A3Q7PEL9, A0A091HYP2, A0A091HU29, A0AIC9EFM3, T0MC86, S9YNR1, E2AYSO, E2AW54, E2AIC2, E1ZYYO, E2A6H3, E2AEN9, A0A2G6E0Y9, A0A0G0Y802, A0A0T5ZUG2, A0A1F5VTJ5, A0A1F6GNK9, A0A1F6IW58, A0AIF6IW51, A0A2E6AVH4, A0A2E1QA46, A0A2E8VEV6, A0A081BMC8, A0A3E1D9A6, A0A2H0KSE9, A0A0J9E870, A0A2K8U7S7, A0A0S6WC51, Q867B7, F1PNV5, F1Q421, J9P7N9, U6C2S1, B5LX43, F1PPK8, E2QX19, X1ZK48, X2A7L8, R7T8C6, R7V393, R7U1A9, R7TZY2, R7V723, R7UC69, R7V5X1, R7TYC5, R7UCC3, R7TUV0, R7U099, R7UD86, R7VAV0, R7U4R0, R7V1P5, R7U5C7, R7VIS4, R7UZN9, R7UPR5, R7TN37, R7TTF7, N1PB13, R7T7C0, R7TW97, R7U2J7, R7VIH6, R7TQC9, R7VEV6, R7UHQ9, R7TNK1, R7U9W1, R7UFI6, R7TTM3, R7TL37, R7VDK3, N1PB94, R7UZW7, R7UR22, R7TMW2, R7TC17, R7U7K0, R7T380, R7TB71, R7TTG1, R7TV63, R7TDJ4, R7UDB7, R7TUJ8, R7TA27, R7T436, R7USY4, R7TG23, R7THU4, R7V2I5, R7TW72, R7U6P1, R7VIM6, R7U4L4, R7UEY2, R7TFT8, R7VJV9, R7TX09, R7UIB6, R7U426, R7VEG9, R7TMY6, R7ULX6, R7U7Q9, R7UCW6, R7UUK4, R7U544, R7UUF8, R7TA51, R7UFR7, R7T4S3, R7VFQ5, R7UDP1, R7UH53, R7TBV9, R7VFH4, R7V8L1, R7UGV2, R7VGL6, R7TAP8, R7TYU2, R7V4I7, R7TIG7, R7V0P1, R7T7U1, R7V6R4, R7U6D2, R7V5V9, R7TI76, R7VF14, R7UVT0, R7VG70, A0A452E0U1, A0A452E5W4, A0A452EN11, A0A452E0T2, A0A452E0U8, A0A452FN28, A0A452E7A0, A0A452E0H7, W9YD76, W9YRK9, Q15B70, Q15B72, Q15B73, B9VWJ3, C3USC5, C3USB9, C3USC3, B9VWJ2, A0A330PLR6, A0A330PLQ4, Q159E8, D6PR15, C3US88, Q159E6, D6PR17, Q159E2, B9VWJ4, D6PR14, C3US92, D6PR16, Q159E1, Q15B71, C3US83, A0A330PI91, D6PR65, C3USB8, A5A5D8, A0A330PL92, Q15B68, C3USC0, C3US90, C3USC9, C3USD0, C3US91, C3USC1, A0A330PL82, Q159E5, D6PR13, C3USC8, Q15B69, Q159E7, B9VWJ5, A0A330PKY6, C3USC6, Q159E4, C3USC4, C3US86, C3USC2, C3US84, Q159E3, C3US87, C3US82, D6PR63, D6PR62, C3US81, C3USC7, R0I6W8, R0GTT4, R0H9B0, R0GNE2, R0FI89, R0HTN8, R0GUQ7, R0HUYI, R0H0H3, R0I9M8, G4WHB0, R0IR13, R0GLW7, R0GG20, R0HTH2, R0HWX1, R0HH21, R0F138, R0G349, R0G9X, R0H4Q7, G4WHA9, R0H3X2, R0IQYO, R0HDW3, R0GEJ4, R0I6F3, Q159E9, R0HEMO, R0GB39, R0EV33, R0IB39, R0GFC4, R0GDL4, R0H6A7, R0G917, R0GC32, R0FRL5, R0GGH9, R0GPB0, R0IKR4, R0I6F0, G3LPX2, C3US76, C3US93, G3LPQ9, Q159F0, A0A2G2Z3C2, A0A1U8HJK3, A0A1U8FLD1, A0A1U8G176, A0A1U8FMT3, A0A1U8G0B3, A0A2G2YXS1, A0A2G2Z497, A0A2G2ZR65, A0A2G3A8T5, A0AIU8E006, A0AIU8GC95, A0AIU8FKZ6, A0A1U8EHH7, A0A1U8FWF1, A0A2G2YVW6, A0A1U8HID5, A0A1U8HB94, A0A2G2XXB9, A0A1U8FUE7, A0A1U8FH35, A0A2G2YHC9, A0A2G3A4Y4, A0AIU8DSV0, A0A2G2ZR66, A0A2G3ACA4, A0AIU8GJN2, A0A2G2Z443, A0AIU8GC84, A0A2G2Z3R4, A0AIU8G424, A0A2G3A4P0, A0A2G2XZF1, A0AIU8HJC3, A0A2G2Z3M0, A0AIU8E541, A0A2G2Y426, A0A2G2XXC9, A0A2G2Z5E8, A0A2G2YRI6, A0AIU8FFY8, A0A2G3A8R0, A0A2G3A8S2, A0A2G2Z7W9, A0AIU8F3X5, A0AIU8FMU1, A0A2G2Z4E1, A0A2G2ZGY9, A0A1U8EBB4, A0A1U8GJN8, A0A1U8F5I0, A0A2G2ZR70, A0A1U8FLJ4, A0A2G2Z477, A0AIU8FUF3, A0AIU8H8D5, A0A2G2ZGX9, A0AIU8HCZ6, A0AIU8FGU3, A0A2G3AHC7, A0A2G2ZRA6, A0A2G2YBW0, A0AIU8HJK7, A0A2G2ZR53, A0A2G2ZR72, A0A1U8HHR9, A0A1U8EQX3, A0A1U8G097, A0A2G2ZGX6, A0AIU8HFC1, A0A2G2Z069, A0AIU8HJC5, A0AIU8HF58, A0A2G3AHK5, A0AIU8GC83, A0A2G2Z4E6, A0A2G2Z592, A0AIU8FWF6, A0A1U8FKF0, A0A1U8HBY4, A0A1U8GC91, A0A1U8FI52, A0A2G2ZR54, A0AIU8GLG7, A0A2G2Z4F9, A0A2G2XZG6, A0A2G2Z588, A0AIU8GKKO, A0AIU8FMT6, A0AIU8E509, A0A2G3A8W8, A0AIU8GL39, A0AIU8H760, A0A1U8HFP2, A0A2G2Z4D1, A0A1U8HC90, A0A1U8FCB4, A0A2G2Z5A7, A0A2G2XTN5, A0A2G3A3F6, A0A2G2XJB3, A0AIU8HJL6, A0A2G3A8Y5, A0A1U8FU12, A0A2G2ZNM4, A0A2G2YUR9, A0A1U8FUG6, A0A1U8FLF2, A0A2G2Y4I0, A0A1U8F5U4, A0A2G2Y412, A0A1U8FUE2, A0A1U8HHS4, A0A1U8FMU9, A0A2G2ZR57, A0A1U8HD42, A0A1U8FL04, A0A1U8HD34, A0A2G2ZR87, A0A2G2ZRA9, A0AIU8HHK2, A0A2G2Z3A1, A0A2G2Z488, A0A2G2ZZ34, A0A2G2YUF5, A0AIU8HD39, A0AIU8H0K4, A0AIU8GC89, A0A2G3A8R4, A0AIU8FMF8, A0AIU8GCX8, A0A2G2YQD6, A0AIU8EA38, A0A2G2Z3H8, A0A1U8FQA6, A0A2G2ZR51, A0A2G2Z5Q1, A0AIU8HD36, A0A1U8FUF9, A0A2G3A8R3, A0A1U8FLD6, A0A2G2ZR86, A0A1U8FLE0, A0A2G3A8S0, A0AIU8GCY4, A0A2G2ZR97, A0A2G3A8U5, A0A2G2Z5B6, A0A2G2XZG5, A0A2G3A8V8, A0AIU8FGL4, A0AIU8HJB8, A0A2G2ZZZ3, A0A2G3A8A0, A0A2G2YVF7, A0A2G2ZR77, A0A2G2Z5J5, A0A2G2ZR62, A0A2G2Z449, A0A2G2Z595, A0AIU8GJM6, A0A2G2Z4K3, A0AIU8FLD5, A0A1U8FFC8, A0A1U8FEC5, A0A1U8GMK6, A0A2G3AFV5, A0A2G2Z5L0, A0A2G2Z584, A0A2G2ZY67, A0AIU8G841, A0A2G2Z100, A0A2G3A8V4, A0A2G2YUT1, A0A1U8GGN2, A0A2G3A8X5, A0A1U8GK83, A0A1U8E139, A0AIU8HLA8, A0AIU8EB93, A0A2G2Z3E9, A0A2G2WEE9, A0A2G2WVY4, A0A2G2WW96, A0A2G2VY63, A0A2G2VQY9, A0A2G2VAD8, A0A2G2WPR1, A0A2G2WDF9, A0A2G2WEK5, A0A2G2VR35, A0A2G2VQY2, A0A2G2WEJ2, A0A2G2WG78, A0A2G2WZR9, A0A2G2X0B4, A0A2G2WGJ3, A0A2G2VBZ9, A0A2G2XNZ0, A0A2G2VX13, A0A2G2WZV8, A0A2G2WG79, A0A2G2XMD7, A0A2G2VAJ1, A0A2G2XEE7, A0A2G2WEJ3, A0A2G2WDD7, A0A2G2X0L9, A0A2G2VAF3, A0A2G2WEF4, A0A2G2WG85, A0A2G2VBR2, A0A2G2WZY5, A0A2G2XMN2, A0A2G2WG76, A0A2G2VAE7, A0A2G2XBY4, A0A2G2WRP5, A0A2G2VAE4, A0A2G2WEK2, A0A2G2XEX2, A0A2G2WZR1, A0A2G2X0F7, A0A2G2WED8, A0A2G2X1N6, A0A2G2VAK1, A0A2G2WPT7, A0A2G2XMC0, A0A2G2WG69, A0A2G2WG90, A0A2G2WG92, A0A2G2VRB8, A0A2G2WED9, A0A2G2WX08, A0A2G2XCD8, A0A2G2XLJ2, A0A2G2XMA3, A0A2G2XHT8, A0A2G2X346, A0A2G2X0C9, A0A2G2VAE9, A0A2G2WGA3, A0A2G2VAD6, A0A2G2WEI6, A0A2G2VAD0, A0A2G2W6E0, A0A2G2WD99, A0A2G2WEF8, A0A2G2WZR7, A0A2G2VAL5, A0A2G2X3S1, A0A2G2VCQ1, A0A2G2WDF4, A0A2G2VAC5, A0A2G2VKF1, A0A2G2VRK8, A0A2G2X0B3, A0A2G2WW24, A0A2G2WG68, A0A2G2VAF6, A0A2G2UYX7, A0A2G2WVX3, A0A2G2X5R0, A0A2G2VPU4, A0A2G2WDS9, A0A2G2WDD3, A0A2G2XFD6, A0A2G2VAF2, A0A2G2X0H2, A0A2G2WGA2, A0A2G3D6E9, A0A2G3C1F1, A0A2G3C1E0, A0A2G3C1G5, A0A2G3C2Z3, A0A2G3CPI0, A0A2G3DCY1, A0A2G3C316, A0A2G3D2F1, A0A2G3C2Z9, A0A2G3C1E1, A0A2G3D6B8, A0A2G3CPH7, A0A2G3BYB2, A0A2G3CJH8, A0A2G3C300, A0A2G3D9N7, A0A2G3CKE6, A0A2G3C305, A0A2G3C0I4, A0A2G3BJQ2, A0A2G3D6E1, A0A2G3C1I7, A0A2G3C0J7, A0A2G3D6F2, A0A2G3C0I0, A0A2G3C307, A0A2G3CK88, A0A2G3D6G2, A0A2G3D6D1, A0A2G3C0J0, A0A2G3C304, A0A2G3B5S2, A0A2G3CF44, A0A2G3CKB8, A0A2G3C314, A0A2G3CPI6, A0A2G3CPF9, A0A2G3D2F8, A0A2G3BYR9, A0A2G3BIS4, A0A2G3CPH6, A0A2G3BNQ8, A0A2G3D5W0, A0A2G3DEU0, A0A2G3D034, A0A2G3C0M5, A0A2G3D6E2, A0A2G3CPH2, A0A2G3C1F2, A0A2G3D4Y8, A0A2G3CUB9, A0A2G3BNC4, A0A2G3D6F6, A0A2G3C2X9, A0A2G3BKG5, A0A2G3D6D5, A0A2G3C180, A0A2G3C355, A0A2G3D6D6, A0A2G3C0G2, A0A2G3CPJ8, A0A2G3CPG3, A0A2G3C353, A0A2G3CPG9, A0A2G3AYH3, A0A2G3DCW4, A0A2G3C265, A0A2G3CZ75, A0A2G3C313, A0A2G3CPH9, A0A2G3BAY7, A0A2G3D2T3, A0A2G3BSY6, A0A2G3ATR3, A0A2G3CPI5, A0A2G3BXI5, A0A2G3D6G1, A0A2G3CL76, A0A2G3DD18, A0A2G3D5A9, A0A2G3D6C0, A0A2G3CF81, A0A2G3CPV5, A0A2G3BN91, A0A2G3D5Y6, M9RTK5, A0A0U2QU59, A0A091LRX5, A0A091LPV4, A0A147B803, A0A250YCG5, A0A0K2A1W0, A0AIY2HTZ2, A0AIY2HS76, A0A091LCZ8, A0A091LCI0, A0A091LH38, A0A091LE22, A0A1V0SBF1, A0A1B1AEB3, H0UU59, H0V4A7, H0VJ51, H0VJK2, H0VMN8, A0A1S6L629, A0A2K5R7R9, A0A2K5S2Q5, A0A2K5S2P6, A0A2K5RX96, A0A2K5S9C9, A0A2K5S2F6, A0A2K5S9D7, A0A2K5S2N2, A0A2R4M5G7, A0AII3X2Y8, A0AIQ3CUV5, A0AIQ3BH96, A0AIQ3CMR9, A0AIQ3DDN1, A0A1Q3C1X7, A0AIQ3DAG8, A0A1Q3C304, A0AIQ3BHR2, A0AIQ3DDT5, A0A1Q3D9K6, A0AIQ3ATB9, A0AIQ3CIF3, A0A1Q3B9L6, A0AIQ3BTA5, A0A0P1BME7, A0A316VR68, A0A316VS87, A0A316VQI0, W8C6U2, W8C174, W8ClF8, W8AT16, W8BF27, W8B238, W8BBW0, W8BCC6, A0A2K5MGN8, A0A2K5M9B1, A0A2K5N9N6, A0A2K5NA06, A0A2K5MGR7, A0A2K5N874, A0A2K5N9U0, A0A2K5LD51, A0A2K5N9Z6, A0A2S6CCY8, A0A2S6BQS9, A0A2S6CDU3, A0A2S6BW05, A0A2S6BSE0, A0A2S6CLP9, A0A2S6C3L0, A0A2S6BTN4, A0A2S6C4G5, A0A2S6C470, A0A2S6BY54, A0A2S6C2K4, A0A2G5HUI6, A0A2G5IDK6, A0A2G5IA94, A0A2G5HDH1, A0A2G5HEC5, A0A2G5HCY6, A0A2G5HUJ2, A0A2G5I2T2, A0A2G5HEX4, A0A2G5HP23, A0A2G5HTF1, A0A2G5I1N9, A0A2G5HCN3, A0A2G5HGT9, A0A2G5IDY8, A0A2G5HL32, A0A2G5HYX7, A0A2G5I599, A0A2I0RLP9, A0A2I0RUR2, A0A2I0S1B2, A0A2I0RXR6, A0A2I0RX04, A0A2I0RUR7, A0A2I0RNL9, A0A2I0RI80, A0A2I0RRK8, A0A2T4JY52, A0A2W7S7A2, A0A212C8S4, A0A212C7P2, A0A212CKS8, A0A212C307, A0A212C2K8, A0A212C346, G0S7E3, A0A3M7NZG2, A0A3M7NLC0, A0A388K5P6, A0A388K2B3, A0A388LIL5, A0A0A0A1C3, A0A0A0ACH9, A0A0A0A4L1, A0A0A0AEL2, Q11FM9, Q11LR2, A0A2W6Y8B9, M7B6M9, M7B4A9, A0A437B6K6, A0A437BD02, A0A3S2P060, A0A3S2NRB4, A0A3S2TGF1, A0A401S8F4, A0A401SUD6, A0A401T7K4, A0A401S098, A0A250WPW7, A0A250WQA5, A0A250WYW3, A0A2K3DSA5, A0A2K3E6A5, A8HZX7, A0A2K3DB77, A8HQ30, A8IBD2, A0A2K3DBP7, A0A2K3D8X6, A0A2K3E6A3, A0A2K3DB83, A0A2K3CTH7, A0A2K3CSI6, A8IFR1, A8IMR7, A8HQN7, A0A2K3E6U0, A0A2K3DBQ5, A0A2K3DBU7, A8J8N4, A0A2K3D2L0, A0A2K3DS95, A0A2K3DBP8, A0A2K3DMT1, A8J1H4, A0A2K3D8R5, A0A2K3DWJ6, A0A2K3DCU6, A8IKN5, A0A2K3E6C5, A0A2K3D9V3, A8HX99, A0A2K3D9V6, A8JBG8, A8IKN3, A0A091L5H5, A0A091L0G7, A0A091KL34, A0A3L8SH23, A0A3L8S1X4, A0A3L8SV11, A0A3L8S925, A0A2P6TKL9, A0A2P6TF11, A0A2P6U331, A0A2P6TIU0, A0A2P6TGU7, A0A2P6TIQ9, A0A2P6TGF4, A0A2P6TFU2, A0A2P6TLH4, A0A2P6TJF6, A0A2P6U011, A0A2P6TXL2, A0A2P6U051, A0A2P6TCKO, A0A2P6TW22, A0A2P6TZN7, A0A2P6TCJ6, A0A2P6TUR6, A0A2P6THR0, A0A2P6TIQ5, A0A2P6TMR9, A0A2P6TR31, A0A2P6TH81, A0A2P6TP21, A0A2P6TVR3, A0A2P6TES6, A0A2P6TXZ7, A0A2P6TMQ7, A0A2P6TKW1, A0A2P6TQS8, A0A2P6TIZ9, A0A2P6TF15, A0A2P6U012, A0A2P6TMT2, A0A2P6U316, E1ZPG0, E1ZRL2, E1Z6K2, E1ZHG6, E1ZJC2, E1ZUC6, E1ZHR7, E1ZF04, E1ZHR6, E1Z7H7, E1Z291, E1Z6Q1, E1ZJG5, E1ZM59, E1ZG06, E1ZAC6, E1Z965, E1ZBZ7, E1Z7U0, A0A0D9RIP2, A0A0D9S2M3, A0A0D9RHM4, A0A0D9S2M6, A0A0D9RQW6, A0A2E1ZYY8, A0A017T0L6, A0A017T6P0, A0A0G4GGC1, A0A0G4G2T2, A0A0G4HXM5, A0A0G4HH89, A0A0G4GSE8, A0A0G4IBR5, A0A0G4H481, A0A0G4I991, A0A0G4FQB4, A0A0G4GMQ5, A0A0G4GZC4, A0A0G4HGC9, A0A0G4FVUO, A0A0G4F227, A0A0G4FYS2, A0A0G4FGP6, A0A0G4FEZ3, A0A0G4I700, A0A0G4GGB9, A0A0G4F023, A0A0G4FZM7, A0A0G4HZX5, A0A0G4HKE3, A0A0G4HYV2, A0A0N9QJ81, A0A0N9R3K7, A0A0MOJLK2, A0A0MOJFA1, A0A0MOJ9M7, A0A0MOJ502, A0A0MOJW38, A0A0MOJSY5, A0A0MOJH75, A0A1S2Z201, A0A1S2Y6R3, A0AIS2YVX3, A0A1S2Y702, A0A1S2Z2P8, A0AIS2XCS8, A0AIS2XT54, A0AIS2XFS7, A0AIS2YU61, A0AIS3E0S5, A0AIS2XB68, A0A3Q7YCB7, A0AIS2YU62, A0AIS2XDH3, A0A1S3E9I2, A0AIS3EJ26, A0AIS3ED37, A0AIS2XP43, A0A3Q7XHP2, A0AIS2XCS7, A0A1S2X9K5, A0AIS2XP07, A0A3Q7YC42, A0A3Q7Y1H8, A0AIS3EDN3, A0AIS2XBI0, A0A3Q7YAUO, A0A3Q7Y285, A0A3Q7Y6L8, A0A1S2Z7A8, A0A3Q7XQV7, A0A3Q7YG81, A0A3Q7YOF7, A0A1S2Z2N4, A0A3Q7XQV2, A0A1S2Y575, A0A1S2Z2N5, A0A3Q7WWB2, A0A1S2Y5U4, A0A1S3E9A9, A0A1S2X9L3, A0A3Q7XBV1, A0AIS2XRR7, A0A1S3E9B2, A0A3Q7XAI7, A0A3Q7YAZ7, A0A3Q7Y008, A0A3Q7YFJ7, A0A1S2Z200, A0A1S3E6X4, A0AIS2YU56, A0A1S2Y443, A0A1S3E803, Q84V84, A0A443PRY7, A0A443NX35, A0A3S3NIB5, A0A443P5K6, A0A443PBN9, A0A443PBP3, A0A443P0S9, A0A443PS04, A0A443PW89, A0A443PS03, A0A3S3MEI8, A0A443PN61, A0A3S3N3K9, A0A443NLN9, A0A3S3MJQ9, A0A3S3NA45, A0A443P5V0, A0A443P2S3, A0A3S3PD76, A0A3S3MXI8, A0A443PCA0, A0A443P2G4, A0A443PBN2, A0A443PS75, A0A3S3MXL1, A0A443PBP0, A0A3S3MR36, A0A3S3NVL5, A0A443PBQ1, A0A3S3P012, A0A3S3NGN2, A0A443PBP1, A0A3S3QL46, A0A443PBQ8, A0A3S3MJ71, A0A443PBP5, A0A3S3PSG0, A0A443PBN3, A0A443PDZ2, A0A3S3N939, A0A3S3PEW5, A0A3S4PKF1, A0A3S3NWW4, A0A3S3MXN2, A0A3S3QIN5, A0A3S3QJN6, A0A3S3NA33, A0A3S3NPA9, A0A443NX39, A0A443N5D8, A0A3S3N3I2, A0A443PBQ2, A0A3S3MLU2, A0A3S3MTZ8, A0A3S3MVZ3, A0A443PBN5, A0A443PS14, A0A3S3NH01, A0A3S3MY34, A0A3S3QYD7, A0A3S3NIC6, A0A443PMN8, A0A3S3N4I9, A0A443PBM7, A0A443P2M2, A0A3S4PQK3, A0A3S3QQQ3, A0A3S3QP22, A0A443PLP3, A0A3S3PD65, A0A443PBN0, A0A443NF22, A0A443PLH6, A0A443PS49, A0A3S3NDMO, A0A3S3PD86, A0A443PJM2, A0A3S3NJS6, A0A443PLJ7, A0A443PBQ0, A0A3S3MXK7, A0A443P0T7, Q69HN0, A0AIW5BT82, A0A3Q0K068, A0A3Q0JVM7, A0AIW2WKK1, A0A3Q0JM87, A0AIW5BJJ9, A0A3Q0K9Z4, F6PY33, F6UI41, A0A1W5B4S5, F6RQT3, A0AIW5BDQ8, A0A3Q0JQ58, H2YF24, H2ZPV7, IIATJ2, D0D4L1, A0A355QKE1, A0A357RS17, A0AIH3MOF3, V4UM33, V4S782, V4WFC0, V4US31, V4UM62, V4W3X3, V4UM47, V4UGX9, V4TXJ0, V4SX72, V4T0F1, V4UGU5, V4TXM4, V4UGV0, V4WFE7, V4UGU3, V4UG60, V4U8W0, V4TPH0, V4TXH2, V4ULH8, V4UM07, V4UAV8, V4WFD4, V4UM16, V4US56, V4TXI0, V4WFB7, V4TXL6, V4US24, V4UGR6, V4RYV2, V4SQ65, V4UM23, V4UR15, V4WFE2, V4TQP7, V4UOT7, V4US39, V4SZW8, V4UGT3, V4W877, V4SUW1, V4UGW8, V4WFB2, V4S160, V4WFC6, V4STW6, V4T683, V4UM36, V4UEI5, V4UM71, V4USA4, V4T894, V4SP31, V4TXG2, V4W5E2, V4SHZ9, V4UOT3, V4SDM6, V4UGS3, V4SNM6, V4TXJ3, V4TVE6, V4RYU8, V4W3W8, V4US66, V4W3G2, V4UCD6, V4WFN6, V4TLL6, V4US48, V4U017, V4US05, V4TXJ7, V4UM67, V4US34, V4US51, V4US13, V4ULH4, V4TXKO, V4UM52, V4SQ60, V4US61, V4U986, V4U5T3, V4WFD7, V4W873, V4TXMO, V4SV65, V4UM41, V4U452, V4STW9, V4STF2, V4STL9, V4UGV5, V4U9H6, V4W3W6, V4U5T6, V4SMY3, V4SSX8, V4UGT8, V4S7S5, V4T687, V4UGS8, V4ULH0, V4UM27, B2CDF6, A0A067GG52, A0A067G6T4, A0A067GFX5, A0A067CZ86, A0A067GG63, A0A067GFU2, A0A067G437, A0A067G408, A0A067GAK8, A0A067GG46, A0A067ERZ0, A0A067G3Z5, A0A067G447, A0A067DNX9, A0A067ESK3, A0A067GFW5, A0A067EC69, A0A067DC17, A0A067FDW2, A0A067GFW9, A0A067DFP7, A0A067DJF5, A0A067G402, A0A067D6A0, A0A067DEZ1, A0A067D709, A0A067E075, A0A067ESUO, A0A067ERZ4, A0A067GG54, A0A067G3X5, A0A067GFU7, A0A067F300, A0A067G6V7, A0A067G416, A0A067F0B3, A0A067G7T6, A0A067GG67, A0A067FCY6, A0A067GFY6, A0A067G429, A0A067EZ26, A0A067G407, A0A067G444, A0A067DHM7, A0A067GG72, A0A067G7U1, A0A067DGG9, A0A067G3W6, A0A067F108, A0A067GG42, A0A067EXK6, A0A067DI35, A0A067G3X9, A0A067F5Z7, A0A067G419, A0A067DP49, A0A067D963, A0A067ESK4, A0A067GAK4, A0A067G6T1, A0A067G3Y0, A0A067DXE4, A0A067GFZ4, A0A067GG78, A0A067G404, A0A067DEU1, A0A067F642, A0A067DP44, A0A067EP43, A0A067ECN7, A0A067EFL5, A0A067G3Y8, A0A067ERY8, A0A067G3Y3, A0A067GFX8, A0A067EMR0, A0A067GG48, A0A067G7Q9, A0A067E705, A0A067G451, A0A067G6R7, A0A067DMY8, A0A067ECA5, A0A067EVI9, A0A067DT16, A0A067DFK5, A0A067DBK4, A0A067G3Z3, A0A067DLD4, A0A067EI62, A0A067EFX3, A0A067ENV7, A0A067EJB2, A0A067GG38, A0A067G3V1, A0A067GG58, A0A067DJW6, A0A067DRX6, A0A067GFV3, A0A067G6U4, A0A067G6W5, A0A067G6X4, A0A067DFT8, A0A067EP41, A0A067EIF8, A0A067G6Z0, A0A067FHX6, A0A067DIS5, A0A067DDY5, A0A067EMP5, A0A067G6S1, A0A067GG04, A0A067GFZ1, A0A067DJF0, A0A067F0Y8, A0A067G412, A0A067G6V2, A0A067G6U9, A0A067G3Z1, A0A067G420, A0A067G3V7, A0A067GK13, A0A067G7V2, A0A067GFY1, A0A067GK25, A0A2H5PKY8, A0A2H5PKZ4, A0A2H5Q9C5, A0A2H5PSJ2, A0A2H5PSH0, A0A2H5QJF9, A0A2H5PKR2, A0A2H5PFQ6, A0A2H5PSI2, A0A2H5PSG3, A0A2H5PSI8, A0A2H5PSM9, A0A2H5PSK6, A0A2H5PKW4, A0A2H5PKV3, A0A2H5PKZ1, A0A2H5PSG8, A0A2H5PSJ9, A0A2H5PSH4, A0A2H5PFR7, A0A2H5PL00, A0A2H5PLJ0, A0A2H5PKR5, A0A2H5P0E0, A0A2H5PYT1, A0A2H5Q8A5, A0A2H5PSK1, A0A2H5PLKO, A0A2H5QSC3, A0A2H5N494, A0A2H5NKK7, A0A2H5PKY5, A0A2H5MW58, A0A2H5PSJ1, A0A2H5QY31, A0A2H5PKT9, A0A2H5N543, A0A2H5PFR2, A0A2H5QX37, A0A2H5NYH0, A0A2H5PKT8, A0A2H5PKS3, A0A2H5PSI7, A0A2H5PKV8, A0A2H5PKW8, A0A2H5QGS6, A0A2H5PLH7, A0A2H5PKW9, A0A2H5N106, A0A2H5PSG4, A0A2H5PBR2, A0A2H5PIJ5, A0A2H5QGL4, A0A2H5PSL1, A0A2H5PSF5, A0A2H5QJI9, A0A2H5PWC1, A0A2H5PLG3, A0A2H5PSL8, A0A2H5PKUO, A0A2H5PKT6, A0A2H5QX47, A0A2H5PXG7, A0A2H5QUK2, A0A2H5PTF6, A0A2H5PCI5, A0A2H5PHZ3, A0A2H5QGN3, A0A2H5PKS2, A0A2H5PHW4, A0A2H5PSI9, A0A2H5QTN3, A0A2H5QDF2, A0A2H5PKW6, A0A2H5PKX5, A0A2H5P8A0, A0A2H5PKW0, A0A2H5PKV5, A0A2H5PKY2, A0A2H5PFQ1, A0A2H5Q8X1, A0A2H5PIKO, A0A2H5PSK7, A0A2H5PSH7, A0A2H5PTD6, A0A2H5PHY6, A0A2H5MVV6, A0A2H5PKT3, A0A2H5N2Y6, A0A2H5PKW2, A0A2H5PLM9, A0A2H5PKW5, A0A2H5PLK9, A0A2H5MW15, A0A2H5PKV2, A0A2H5MXC0, A0A2H5N221, A0A2H5QJF2, A0A2H5N2E3, A0A2H5PTC7, A0A2H5PSI1, A0A2H5PSH6, A0A2H5PSF9, A0A2H5PIL0, A0A2H5PSJ5, A0A2H5QDF6, A0A2H5PYF9, A0A2H5PFR4, A0A2H5PSM2, A0A2H5PLL9, A0A2H5QGR8, A0A2H5QSP4, A0A2H5PTB7, A0A2H5MXP2, A0A2H5QDF7, A0A2H5PW39, A0A2H5PKU4, A0A2H5PKV9, A0A2H5PKT1, A0A2H5PL05, A0A2H5MWP9, A0A2H5PKS5, A0A2H5PKY1, A0A2H5PKZ0, A0A2H5PKX3, A0A2H5PLN9, A0A2H5PSN3, A0A2H5PKY6, A0A2H5PSI0, A0A2H5PKU2, A0A2H5MY89, A0A2H5PKZ5, A0A2H5N248, A0A2H5MVR2, A0A2H5PC29, A0A2H5PSH8, A0A2H5PSJ6, A0A2H5PKZ9, A0A2H5PKV0, A0A2H5PKU3, A0A2H5QX61, A0A2H5PKU8, A0A2H5PXK1, A0A2H5PKS9, A0A2H5PSK2, A0A2H5PXH5, A0A2H5PKZ7, A0A0D2EPW4, A0A0D2H5D0, A0A0D2D5M4, A0A0D1ZW52, A0A0D2D9C0, W9VHA5, A0A1B6E728, A0AIB6DP44, A0AIB6CQM5, A0AIB6CRN7, A0A1B6D4A1, A0AIB6CPV4, A0AIB6CH46, M1VXP1, A0AIY1ZIY5, A0AIY2A3K8, A0AIYIY9H3, A0AIY1ZK83, A0AIY1Z9L6, A0A419PQI0, A0A3R7EPZ0, A0A419QAI4, A0A419PSE2, G7YA90, G7YBT5, A0A3R7DBB2, A0A3R7CQZ5, A0A419PZ32, G7YEU8, G7YWK9, G7Y624, A0AIJ1IGEI, A0AIJ1HN04, A0AIJ1IXY3, A0AIJ1IRI6, A0A1J1I6X9, A0AIJ1HMP6, J3K384, J3K388, A0A0J8USD8, A0A0J8SOH2, A0A0J6Y7D1, A0A0J6Y7C8, A0A0J8QTA0, C5P676, C5P674, E9DHQ1, E9DIX1, E9DIM5, A0A0J6I586, A0A0J6I580, I0YJN3, I0YQC5, I0YZJ2, I0YQC4, I0Z909, I0YXG6, N4WSB7, N4XA49, N4X4B7, N4XWV7, N4XAK1, N4XNR5, N4WIS8, N4XE50, N4WKJ0, M2UDR7, M2TCH5, M2UPP8, M2TUB1, M2UZK2, M2UC59, M2TM44, M2SS15, M2TNS5, M2SEH1, M2TCD4, M2REQ9, M2T729, M2QVUO, M2SRC8, M2SXY7, M2SRR6, A0A068V1N7, A0A068TTW9, A0A068U5I0, A0A068UBR8, A0A068UMF4, A0A068VIN5, A0A068VAH2, A0A068VH78, A0A068UOZ7, A0A068VD61, A0A068UDX9, A0A068UF61, A0A068UEE7, A0A068TT72, A0A068TUQ8, A0A068U920, A0A068UBA7, A0A068UTC4, A0A068TTY9, A0A068V6K4, A0A068UCT7, A0A068VAV0, A0A068UAW2, A0A068TTU4, A0A068TUK9, A0A068UB95, A0A068U7P3, A0A068UAV7, A0A068U9M3, A0A068TR02, A0A068TUN3, A0A068UC35, A0A068VIA2, A0A068V9X1, A0A068TWF3, A0A068V7U6, A0A068U9A9, A0A068V658, A0A068VDF8, A0A068TUN8, A0A068URUO, A0A068TWI8, A0A068TPT8, A0A068UCP1, A0A068UB32, A0A068UU33, A0A068VDL2, A0A068UUD5, A0A068V600, A0A068U9B6, A0A068U735, A0A068UDJ3, A0A068VL89, A0A068V636, A0A068TTS6, A0A068VAU6, A0A068UAW8, A0A068TY94, A0A068V341, A0A068UDX1, A0A068U7P7, A0A068TRE6, A0A068U9C3, A0A068U9L9, A0A068UCP7, A0A068V5D0, A0A068U6Z2, A0A068UAL8, A0A068TUH5, A0A068UVZ3, A0AII0RVR9, A0A285PJ03, A0AII4ZNL6, A0A285M669, A0A2N5XUY7, A0A3D8Q658, A0A3D8S2L7, A0A3D8Q9J9, A0A3D8Q983, A0A3D8Q9G4, A0A3D8R5V5, A0A3D8STW8, A0A3D8Q6N2, A0A3D8Q4Q0, A0A3D8QNJ8, A0A226PF24, A0A226PDM2, A0A226PAM3, A0A226NT22, A0A091L082, A0A091KA09, A0A091K6S3, A0AIQ8RWV1, A0AIQ8RVP1, A0A1Q8S2N5, A0A1Q8S5V0, A0A0I0QTI5, A0A0I0SLX6, A0A0I0RTRI, L2FSS4, L2GE62, L2G3T0, L2FT65, L2FRD2, L2GC03, L2FYL0, T0KBX5, T0KBL3, T0LNX5, T0K3L5, T0M6Z0, T0K282, T0KJUI, T0KX95, T0KV89, E3QKF4, E3Q9X3, E3QHU7, E3R0R1, H1VPP1, H1VE61, A0AIB7XYH5, H1VEI3, A0AIB7XYR0, A0AiB7Yl21, H1W3P5, A0A162PMT3, A0A166LHG7, A0AiS1W2W6, A0AiS1VWS2, A0A162NJN8, A0A162PCP5, A0A135SZV4, A0A135UT38, A0A135T629, A0A135UX69, N4UZE4, N4UZ75, N4VFT2, N4W403, N4V0X7, A0AiG4B117, A0AIG4BBR1, A0AIG4ATIi, A0A135V7W1, A0A135S139, A0A135URM5, A0A135V7C4, A0A135RXN4, A0A135RR28, A0A135T1G0, A0A135T432, A0A135RPI7, A0A066XMA0, A0A066XY08, A0A166RN27, A0A166S4H7, A0A2K5JR89, A0A2K5JX41, A0A2K5JX37, A0A2K5JX55, A0A2K5JK04, A0A2K5HKG1, A0A2K5I747, A0A2K5I7F4, A0A2I0MN33, A0A2I0MN31, A0A2I0MV52, A0A2I0MV53, A0A2I0MH63, A0A2I0MN36, A0A2I0MN41, A0A3R4B871, A0A407GJQ8, A0A408KDV5, A0A409SM37, A0A3R2WYC1, A0A3R4Z561, A0A3R1NI64, A0A405Z9C4, A0A3R4NNR5, A0A2T2ZVH0, A0A2T2ZTN0, A0A2T2ZW89, A0A2T2ZSF1, A0AIJ7IVY8, A0A420YIM1, A0A420Y084, R7Z4G2, R7YJ26, R7YYV7, R7Z2B6, R7YKC6, R7YYF5, A0AIT4MT25, H8MJT7, A0AIR3IQG2, A0AIR3JAU6, A0AIR3JH31, A0AIR3ICX9, A0AIR3KDQ4, A0AiR3DUI, A0A1R3I223, A0AIR3I0S0, A0AIR3JCQ7, A0AIR3IHP6, A0AiR3I8B4, A0A1R3HWR3, A0AiR3I8E3, A0AiR3JH09, A0AiR3IIR2, A0AiR3I8B1, A0AIR3GUC6, A0AiR3G8B2, A0AIR3IQM1, A0AIR3GC79, A0AIR3GXE2, A0AIR3JH02, A0AiR3K5M1, A0AIR3JYE5, A0AIR3IQK9, A0AIR3IES9, A0A1R3I214, A0AIR3IQG7, A0AIR3IQE6, A0A1R3I248, A0AiR3K9G1, A0AIR3H0C3, A0AIR3GS32, A0AiR3J3W4, A0A1R3H636, A0AiR3I8F2, A0AIR3FYR8, A0AIR3KLM1, A0AiR3K8S9, A0AIR3KU50, A0AiR3J5L5, A0AIR3KBY3, A0AIR3JYF5, A0AIR3GIW4, A0AIR3JDE5, A0AIR3KM12, A0AIR3HMG0, A0AiR3I4C7, A0AIR3KA03, A0A1R3H436, A0AIR3HD45, A0AIR3KF17, A0AIR3ICZ6, A0AIR3JDF2, A0AIR3FZ78, A0AiR3K8S8, A0AiR3J7N3, A0A1R3H3T9, A0AIR3GAX3, A0AIR3IDM3, A0AIR3JCH0, A0A1R3H3T1, A0AIR3JZ07, A0AIR3KC06, A0AIR3HV73, A0AIR3JYS9, A0AIR3KRY3, A0A1R3H3Q5, A0AIR3KBU9, A0A1R3H4T5, A0AIR3KBT7, A0AIR3JYI7, A0AIR3KBW6, A0AIR3HEM2, A0AIR3KBS9, A0AIR3KBZ0, A0A1R3I4A2, A0A1R3H934, A0A1R3K8R8, A0AIR3KBZ3, A0AIR3JYI0, A0AIR3JYB7, A0A1R3H3N1, A0A1R3H913, A0AIR3JDG2, A0AIR3KBV2, A0A1R3H8W0, A0AIR3KU57, A0A1R3I4C0, A0A1R3H3R9, A0AIR3JID2, A0AIR3KBV6, A0A1R3H3T2, A0AIR3GSE3, A0AIR3JYJ4, A0AIR3KBX8, A0AIR3KU24, A0AIR3HPV5, A0AIR3KGT6, A0AIR3GJ03, A0A1R3H3P6, A0A1R3K9H9, A0A1R3HSJ9, A0A1R3KYZ9, A0A162K7A9, A0A168IRL7, A0A179IKV7, A0A179IAQ3, A0A167WMS9, G3J8R3, G3J375, G3JSZ4, G3JUF8, A0A2H4SK15, A0A2H4S7F4, A0A2C6AEQ6, A0A2C5ZXP2, A0A091ETI1, A0A091EJ05, A0A091EWYO, A0A091F8A0, A0A091E9Y9, A0A2T2N6K3, A0A2T2NUO7, A0A2T2NYB4, A0A2T2N5T0, A0A2T2NUYO, A0A2T2N6K9, Q8LP57, K1QBT9, K1QP59, K1Q5J6, K1RL61, K1PV69, K1Q202, K1RCS2, K1R451, K1QEX9, K1RFL1, K1QFL8, K1R782, K1PMX4, K1QE58, K1QC63, K1Q2X3, KQ0K1, K1RU37, K1QRX2, K1Q0F5, K1PMX3, K1QH76, K1R7M2, K1PKQ8, K1QEU5, KlQEV1, K1Q3J0, K1PJ39, KiR0X1, K1R8Y0, K1QI12, K1PUC3, K1PZY8, K1QNX1, K1PEE6, K1PQ16, K1RKN1, KiP151, K1PCZ9, K1P8Q7, K1RDUO, K1R0V6, K1RNMO, K1QGI9, K1R168, K1QEL7, KiRBUl, K1QRJ4, K1R5R6, K1QPK8, K1QPU6, K1QNB3, K1RFB4, K1P8F6, K1QVB6, K1QI33, K1QDS6, K1QVX2, K1QKJ9, K1QYN7, K1R234, K1Q9S5, K1PXX6, K1QRJ6, K1RGH6, K1PM36, K1R3S9, K1Q4S4, K1R2E6, K1Q9I9, K1Q9I2, K1QQI2, K1P0S6, KlRPI0, K1QP97, K1Q132, K1PMA2, K1P7M9, K1PQG5, K1QIV6, K1PN45, K1R926, K1Q7G5, K1QUA0, KiPUEl, K1PZF9, K1QZS4, K1QYB3, K1QYH7, K1PQV5, A0A1R4H3L7, A0AIH6XYT5, A0A3L7I648, G3HGQ6, A0A3L7IJG9, A0A3L7IG64, A0A061IMW5, A0A3L7HP47, A0A3L7IIX0, A0A061IRF7, G3HPQ5, A0AIJ4MK57, A0A1J4MVW0, A0A1J4MU07, A0A1J4MTL5, A0A0S4TCD4, A0A0S4TBC4, A0A0S4TAQ1, A0A0S4TDE6, A0A2P4YWR1, A0A2P4Z358, A0A2P4Z5F0, A0A2P4YWR9, B6AEN1, B6AJC3, B6A964, Q5CSA4, Q5CTT8, Q5CSA0, Q5CV27, Q86PQ7, A0A1J4MKT5, A0A1J4MIN1, A0A1J4MFD1, A0A2J7PDR3, A0A2J7QPV9, A0A2J7Q2V5, A0A2J7R433, A0A2J7R431, A0A2J7PHG6, A0A2J7QD69, A0A2J7R420, A0A2J7PDS5, A0A2J7QD68, A0A2J7QD73, A0A2J7R430, A0A2J7RS16, A0A2J7R424, A0A2J7PWJ5, A0A2J7R441, A0A2J7R451, A0A2J7R454, W8FRI5, A0A091FW85, A0A091GEV6, A0A091GKK8, A0A091GI80, A0AIS4DST8, A0A1S3B409, A0A1S3B3R3, A0AIS4DSQ5, A0AIS4DSX1, A0A1S3B440, A0A1S3B585, A0A1S3B4W7, A0AIS3CSP7, A0AIS3AZX8, A0AIS3AZY6, A0A1S3BZ12, A0A1S4DSR7, A0A1S4DSP8, A0A1S4E4E3, A0A1S3B4H2, A0A1S3B453, A0A1S3B3R8, A0A1S3B5C7, A0AIS4DTJ6, A0AIS4DSS7, A0A1S3B4D0, A0A1S3B536, A0A1S3B5U5, A0A1S3B6L4, A0A1S3B4A8, A0A1S3BTL3, A0A1S4DST5, A0A1S3B4B3, A0A1S3B516, A0A1S3B4F1, A0AIS4DZ25, A0AIS3C0Q9, A0AIS4DSE8, A0AIS3CSN1, A0AIS3BC50, A0AIS4DSS6, A0A1S3B7B8, A0AIS3CF32, A0A1S3C207, A0A1S3B530, A0AIS3BTI5, A0AIS4DSE6, A0AIS4DSS8, A0AIS3CR70, A0A1S3B575, A0A0A0LS73, A0A0A0LS68, A0A0A0LUY8, A0A0A0LX93, A0A0A0LUE6, A0A0A0LRR4, A0A0A0KRF4, A0A0A0K287, A0A0A0LEC1, A0A0A0LUZ3, A0A0A0LDL3, A0A0A0LUF1, A0A0A0LAF2, A0A0A0LRR1, A0A0A0KK10, A0A0A0LBA0, A0A0A0LX89, A0A0A0KC43, A0A0A0L6H7, A0A0A0K3N8, A0A0A0LUZ5, A0A0A0LFV8, A0A0A0LUY4, A0A0A0LII6, A0A0A0LXA0, A0A0A0LD19, A0A0A0LX86, A0A0A0KC37, A0A0A0LRR8, A0A0A0K8W5, A0A0A0L7U1, A0A0A0LRS1, A0A0A0KWZI, A0A0A0LS76, A0A0A0LUD8, A0A0A0KSR7, A0A0A0LZJ7, A0A0A0K7B0, A0A1B6H4Y5, A0A1B6G4G8, A0AIB6EYW8, A0AIB6FLH2, A0AIB6FYJ9, A0AIB6GHV9, A0A1B6H2T7, A0AIB6FML8, A0AIB6GMC1, A0AIB6FMH3, A0AIB6FYA0, A0A1B6H1R8, B0VZB8, B0WCW6, B0XHR5, B0X861, B0WCW5, B0X859, B0WBN2, B0W214, A0A1S4J279, A0A1S4JBK4, B0WCW7, A0A1Q3FSKO, A0A1Q3FS43, A0A336M006, A0A336LTK1, A0A336LRS2, A0A336N522, A0A336LVN5, A0A336MHF2, A0A336MGD3, A0A336MI32, A0A336MYJ1, A0A336LUN7, A0A336MT58, A0A336MRX6, A0A336LTL3, A0A336LMK2, A0A328DVR3, A0A328D129, A0A328D3T7, A0A328D453, A0A328DBU8, A0A328D973, A0A328DIK3, A0A328DIS6, A0A2S6CT90, A0A2T1F9F0, A0A3B8Y4A2, A0AID3CT75, A0A1D3D2G3, A0AID3CZG8, A0A1D3D562, A0AID3CXV2, A0AID3CZF4, A0A1D3D4P8, A0A1D3D927, A0A1D3D792, A0AID3CRX6, A0AID3CZB5, A0AID3CRI2, A0A1D3D067, A0A3P6QZS2, A0A3P6QGH9, A0A3P6T344, A0A3P6R246, A0A3P6S9T1, A0A103YIH9, A0A124PXP6, A0A103XS92, A0A103XY72, A0A103Y5E6, A0A103XZ10, A0A103XC99, A0A124S064, A0A103Y815, A0A103XCM9, A0A118JT41, A0A118JSJ8, A0A103XF71, A0A118JU86, A0A124SCT9, A0A124SHJ7, A0A103YJR9, A0A103XQ49, A0A118K316, A0A103XC93, A0A103XYY7, A0A103Y467, A0A118K2V5, A0A124SHS0, A0A103Y6D6, A0A118HIH7, A0A103XU24, A0A103Y212, A0A103Y1Z5, A0A103YD45, A0A103YJV2, A0A103Y7Z6, A0A124SHR9, A0A103XQ54, A0A124SDG2, A0A103XWT3, A0A103YIJ1, A0A124SHK2, A0A103XF58, A0A118GC55, A0A103XQI2, A0A103Y7Z7, A0A103XBG4, A0A103XU62, A0A118JTB4, A0A103XZ33, A0A103MM20, A0A103Y9H4, A0A103Y652, A0A103Y6D3, A0A118JTV8, A0A103YDC6, A0A103Y6C0, A0A103VJ56, A0A103Y9I0, A0A103Y446, A0A103XWW8, A0A103YCS4, A0A103XIZ2, A0A103XDQ3, A0A3P8VG89, A0A3P8UTK9, A0A3P8VDI4, A0A3P8X2L6, A0A3P8X460, A0A3P8V360, A0A3P8VG76, A0A3P8X4M6, W2RU83, A0A195CFB6, A0A151INZ7, A0A195CUA0, A0A195CJ60, A0A151IBX2, A0A195CDF0, A0A3Q2D697, A0A3Q2FWU5, A0A3Q2D220, A0A3Q2FWW5, A0A3Q2E8M8, A0A3Q2D1Q5, A0A3Q2D1Y0, A0A3Q2E737, A0A3Q2D1Q1, A0A3Q2FR25, A0A3Q2DW28, A0A0U2S2V6, A0A250JIW8, A0A2C6KJH5, A0A2C6KQQ7, A0A2C6KGJ2, A0A2C6KJZ8, A0A2C6KRS7, A0A2C6KBF6, A0A2C6KGB3, A0A2C6L9R7, A0A2C6KLD1, A0A2C6LDQ6, A0A2C6KWX7, A0A2C6KJN3, A0A2C6KC22, A0A2C6KHJ6, A0A2C6KXQ4, A0A2C6LH90, A0A2C6KQM3, A0A2C6KV00, A0A2C6KD09, A0A2C6KJE6, A0A2C6KGE6, A0A2C6KQL0, A0A2C6KFU7, A0A2C6KW92, A0A2C6KGC7, A0A2C6KGB5, A0A2C6L6S4, A0A2C6L490, A0A423VS70, A0A423W0P4, A0A423WTP3, A0A423X084, A0A223PK27, S8C318, S8ASF1, S8BMH2, S8A7Q6, S8A4U1, S8BSD4, A0A1Y2XBG1, A0A1Y2WWT8, A0A1Y2XCF1, A0A1Y2WXC0, A0A212F9C1, A0A212FKU3, A0A212FKV4, A0A212F3I1, A0A212EVI3, A0A212FAB7, Q6JAN0, FiREK1, F1Q890, F6PA78, A4IGA7, Q5XFY1, Q90ZN6, E9QGF2, Q5RGG3, Q6PBA6, FlQEZi, A0A0P5WPN5, A0A0P5HGT8, A0A0P5T1U8, A0A0N8E7S9, A0A0P6AL09, A0A0P5Y462, A0A0P5L4W8, A0A0P5S420, A0A0P5TKN5, A0A0P6A674, A0A0P5YJR7, A0A0P5BMJ7, A0A0P5U7V1, A0A0P6C7R9, A0A0P5M729, A0A0P5DS66, A0A0P5C4X4, A0A0P5D7A2, A0A0P6ATK6, A0A0P5EBG6, A0A0P5XUR1, A0A0P6CTV5, A0A0P5NLP7, A0A0P5E9S4, A0A0P5JUZ4, A0A0P6CI84, A0A0P6C183, A0A0P5TSU1, A0A0P6J986, A0A0P5TJC9, A0A0P5ZKH9, A0A0N8CLP0, A0A0P5BT62, A0A0P6CKZ9, A0A0P5RCJ4, A0A0P6HJG1, A0A0P4XKS6, A0A0P5XUU9, A0A0P5LNA4, A0A0P5NMM3, A0A0P6D731, A0A0N8BA13, A0A0P6B472, A0A0N7ZWW0, A0A0N8DMK8, A0A0P5S7M2, A0A0P5CWX9, A0A0P5NCC6, A0A0P6GJS2, A0A0P5PR06, A0A0P6H6H4, A0A0P6GE29, A0A0N8EJE4, A0A0P5DGB0, A0A0P5XV46, A0A0P5F055, A0A0P6I548, A0A0P6IIP3, A0A0P5T6R7, A0A0P6HQE1, A0A0P4YGM2, A0A0P5BIZ1, A0A0N8DGI8, A0A0N8CBW6, A0A0P5DBP6, A0A0P5HLY6, A0A0P5T4T8, A0A0P5TZY1, A0A0P6E4B8, A0A0P6CID3, A0A0P5REG3, A0A0N8CB13, A0A0N8CAL2, A0A0P5SEKO, A0A0N8AN73, A0A0P5VJT5, A0A0P5H9B6, A0A0P4ZH74, A0A0P5HXM7, A0A0P6FTT5, A0A0P5VB25, A0A0P5Q0M1, A0A0P5PUQ2, A0A0P5Y2H7, A0A0P5CZZ0, A0A0P5BX45, A0A0P5N855, A0A0P5HF26, A0A0P5K8Q2, A0A0N8AQK2, A0A0P5KWZ5, A0A0P5TVC1, A0A0P6GSX1, A0A162QLH9, A0A0P5WUS7, A0A0P5ID96, A0A0P5F0V1, A0A0P5TUD8, A0A0P5JTA5, A0A0P5GZR4, A0A0P6G736, A0A0P5RD98, A0A0P5Y4G8, A0A0P6DIA9, A0A0P4Z167, A0A0N8DVY1, A0A0P5ZUY7, A0A0P4XS98, A0A0P5WA39, A0A0P5LT30, A0A0P5PEL1, A0A0P5YIS8, A0A0P6J1P8, A0A0P4XVA4, A0A0P5GQ84, A0A0P5VSK6, A0A0N8E8I5, A0A0P5Q8K8, A0A0P4YLV2, A0A0P5CFI1, A0A0P5FCE1, A0A0P5J8K4, A0A0P5N364, A0A0P5LSH1, A0A0P5JKT7, A0A0P5T0H1, A0A0P5YD20, A0A0P4XEQ9, A0A0P5D8R1, A0A0P5HZD6, A0A0P5BFL0, A0A0P5X5U9, A0A0P5NCN9, A0A0P4ZBT1, A0A0P5C3E7, A0A0P5J8A5, A0A0P5N689, A0A0N8A5F9, A0A0P5UOB1, A0A0P4XKJ3, A0A0N8A4V3, A0A0P5XZ51, A0A0P4YXW8, A0A0P5AIQ7, A0A0P6GA05, A0A0P6CWH0, A0A0P5VVF0, A0A0N8BSP2, A0A0P4XN82, A0A0P5AEQ6, A0A0P4ZZ55, A0A0P5EPQ8, A0A0P4ZPB2, A0A0N8ATY9, A0A0P5TTT7, A0A0P5RHI5, A0A0P5UDV1, A0A0N8EHV2, A0A0P6DW53, A0A0P4Y8F4, A0A0P5CPG0, A0A0P5SGL3, A0A0P4YDX7, A0A0P5C3L8, A0A0P5HRA4, A0A0P5QFT9, A0A0P6EVG4, A0A0P6AEB8, A0A0P6GFW0, A0A0P5BTL7, A0A0N8E1L0, A0A0P5YUR1, A0A0P4YWK0, A0A0P5PESO, A0A162RSS1, A0A0N7ZQ97, A0A0P5H5A9, A0A0P5W725, A0A0P5PKS1, A0A0P6IBH1, A0A0P5KMN8, A0A0P5N3S5, A0A0N8EJ07, A0A0P5MQE4, A0A0P5PNX2, A0A0P5RVR9, A0A0P6I9Y3, A0A0P6APJ6, A0A0P6CI42, A0A0P5AX29, A0A0P6BQC9, A0A0P6E3F3, A0A0P6CUC0, A0A0P5RW36, A0A0N7ZLQ2, A0A0P5TUC3, A0A164NEQ9, A0A0P5YU72, A0A0P5MRU3, A0A0P4XHH6, A0A0P5SP99, A0A0P5EW58, A0A0P5LF22, A0A0P5QI00, A0A165ADR2, A0A0N8BQK6, A0A0P5N9W7, A0A0P5CL62, A0A0P5X9Q5, A0A0P5NF07, A0A0P5NQH2, A0A0N8ACN6, A0A0P5UDC3, A0A0P5IA39, A0A0P5YW13, A0A0P5D6K9, A0A0P5WU76, A0A0P5ADU9, A0A0P5HLP5, A0A0P5PX75, A0A0P5WMQ3, A0A0P5WK98, A0A0P6DWY3, A0A0N8CYE6, A0A0N8DAF5, A0A0P5U6W6, A0A0P5UHUO, A0A0P5P0S3, A0A164WWC9, A0A0P5TQ92, A0A0P5YKC6, A0A0P5ECYO, A0A0N8DNH8, A0A0P6EUA0, A0A0P5RUF3, A0A0P5YP87, A0A0P4XUA1, A0A0P6FIA5, A0A0P5TWW9, A0A0N8BSD6, A0A0P4Z326, A0A0P5B454, A0A164XEY4, A0A0P5WQJ7, A0A0P4YXX0, A0A0P5RVL4, A0A0N8AUG9, A0A0P5ZPMO, A0A0P4YEX3, A0A0P5BTY4, A0A0P6CM30, A0A0P4WR04, A0A0P5BT21, A0A0P6GTG9, A0A0P6H7K9, A0A0P5DXB5, A0A0P5DIS4, A0A0P4Y9G9, A0A164ZHL4, A0A0P6ETE1, A0A0P5ZR54, A0A0P5G8Z5, A0A0P5PKW0, A0A0P5DCI3, A0A0P6DUY5, A0A0P5LVB4, A0A0P6I8Z3, A0A0P5QEZ7, A0A0P5CK22, A0A0P5U9K2, A0A0P5E2Z0, A0A0P4YWG8, A0A164VXV0, A0A0P5N8V1, A0A0P5YVP4, A0A0P4WYW8, A0A0P4Y5I5, A0A164XQP6, A0A0P6HVX9, A0A0P5UDG3, A0A0P6DTI4, A0A0N8AR21, A0A0P5HVL3, A0A0P4ZTX3, A0A0P5NV81, A0A0N8AXC3, A0A0N8BWQ2, A0A0P4Y6W6, A0A0P5CI46, A0A0P5GCD4, A0A0P5DAL0, A0A0N8DA56, A0A0P6AXE9, A0A0P5Q6F7, A0A0P5Q4A7, A0A0P5ARZ3, A0A0P5IZW7, A0A0P5V3V8, A0A0P5N7S0, A0A0N8A3D1, A0A0P5WMW8, A0A0P5IZ39, A0A0P5MIM1, A0A0P5RMT5, A0A0P5DZF1, A0A0P5LWH8, A0A0P5C3K9, A0A0N8CEH7, A0A0P5DSL3, A0A0P6EMYO, A0A0P5YSJ8, A0A0P5WJZ4, A0A0P5WSP3, A0A0P6F1X9, A0A0N8AZ22, A0A0P6IMA8, A0A0P5E5X7, A0A0P6EFJ2, A0A0P5UL87, A0A0P4Y7F0, A0A0P5HFI7, A0A0P5DIM6, A0A0P5PZV2, A0A0N8BZ24, A0A0P6IB26, A0A0N8EFF2, A0A0P5WLD1, A0A0P5U1N0, A0A0P5NZP1, A0A0N8BVC5, A0A0P5LU81, A0A0P4Z4U7, A0A0P5TAQ3, A0A0P5R736, A0A162RDZ5, A0A0P5G8N3, A0A0N8B914, A0A0N8CTUO, A0A0P5W8C4, A0A0P6I7V1, A0A0P5Y7U6, A0A0P6GKX2, A0A0P6CI97, A0A0P6HZU7, A0A0P5YP46, A0A0P5MTF8, A0A0P5W4R4, A0A0P6EJL6, A0A0P5WKU1, A0A0P5WJY7, A0A0P5Y923, A0A0P6CL50, A0A0P5YP12, A0A0P6JTG3, A0A0N8AZB0, A0A0P5RFE3, A0A0P5MR11, A0A0P5T4E7, E9FZF1, E9GUF9, E9HG92, E9H7K0, E9FZT3, E9H7J5, E9GC79, E9HGF3, E9GXM8, E9HXN3, E9HL86, E9HHQ3, E9GB65, E9GDUO, E9FZB7, E9FXUO, E9HD48, E9HD47, E9GMC8, E9GGB7, A0A164VCM7, A0A161YC10, A0A166G9N2, A0A161X5W6, A0A166F2H9, A0A166IMD7, A0A161ZJQ8, A0A165ZEG6, A0A164SJH2, A0A162AHQ4, A0A175YMP0, A0A165Z528, A0A166EUZ9, A0A175YJR0, A0A175YJB0, A0A166HZ24, A0A162AF38, A0A165WAR5, A0A164ZED3, A0A164X3K8, A0A165ZWM7, A0A164T5C8, A0A166GYV2, A0A164ZG59, A0A162ADMO, A0A166IUK9, A0A175YL17, A0A162AE67, A0A164Y320, A0A164TNL8, A0A175YLD1, A0A166IME5, A0A166AH33, A0A166I4U3, A0A164TNM5, A0A165Z541, A0A164SJF4, A0A165WAT6, A0A175YLF9, A0A166GYR4, A0A175YLB5, A0A175YLR0, A0A166IUK0, A0A175YLW6, A0A161ZVN8, A0A161ZTW6, A0A166GYN3, A0A166GYM4, A0A161ZKU6, A0A175YNJ8, A0A164TNT1, A0A166GV23, A0A164VUU7, A0A175YQ77, A0A166G9M3, A0A175YK13, A0A166GBE6, A0A162ADK4, A0A166I4V5, A0A175YNL1, A0A175YN80, A0A162B3S5, A0A162AD34, A0A175YKE6, A0A165Z6J7, A0A164W8A9, A0A175YLN5, A0A166AAK1, A0A164SJM1, A0A164ZI33, A0A165ZED0, A0A166JCX2, A0A166F2G3, A0A175YMV8, A0A166AH48, A0A175YM77, A0A166F590, A0A175YN69, A0A165ZEF7, A0A161ZHE5, A0A165Z522, A0A161Y240, A0A166IX90, A0A166CK63, A0A166AH68, A0A175YM10, A0A164WIW8, A0A175YMW5, A0A164W8B8, A0A166GYQ3, A0A166AH60, Q6I6Y6, D3P9U3, A0A159Z548, A0A2R8BKZ6, A0A2S8S490, A0A2W5GAR1, A0A2Y9M5R2, A0A2Y9N707, A0A2Y9N2B0, A0A2Y9LKQ9, A0A2Y9MXX5, A0A2Y9M328, A0A2Y9LK73, A0A2M7PDD6, A0A2M7JKL0, A0A2H0AN95, A0A0T6AER9, A0A2N2GCD1, A0AIF9CSM5, A0A2E7UQW9, A0A2D5SNG4, A0A2D6MP68, A0A359C885, A0A2I0WMU7, A0A2I0XFC1, A0A2I0VZ05, A0A2I0VZ03, A0A2I0WD68, A0A2I0VCZ8, A0A2I0VY27, A0A2I0WAD1, A0A2I0VN57, A0A2I0VZ11, A0A2I0WY89, A0A2I0WQII, A0A2I0WKE7, A0A2I0VZ09, A0A2I0XCU2, A0A2I0WR68, A0A2I0X2L0, A0A2I0VTF8, A0A2I0WD76, A0A2I0WMM2, A0A2I0VDJ3, A0A2I0V982, A0A2I0V8Q2, A0A2I0XIC7, A0A2I0VPC9, A0A2I0WAG8, A0A2I0WMK7, A0A2I0WUZ0, A0A2I0VZ10, A0A2I0V9B4, U4UF05, U4UJ61, U4UHH1, U4UAS2, U4UHR8, U4U368, U4UMJ6, U4UC62, N6TCY6, N6T5M3, U4U5U9, N6TNG4, N6TYW1, N6SW87, N6SZ63, A0A1J5E4F1, A0A351CCD8, A0A3N5QE56, M1NBW9, A0AII7N2V7, A0A0F5QJQ8, A0A1E5XPL7, A0A1E5XM33, A0A0F5LGC2, A0A0F5Q359, A0A087M5P3, A0A087M6Z6, A0A0F5L306, A0A087LR20, A0A1M3KU89, A0A1Q3Z4X7, A0A024KDE1, A0A024KC27, A0A0KOTS75, A0A2L1S3I6, A0A085EUA2, A0A085FE05, A0A0Q4PK00, A0A447I8R2, A0A0Q6PUQ7, A0A0Q7E2E7, A0A0Q7I7H2, A0A0T2MN01, A0A0Q8IC41, A0AIH9CCU2, A0AIH8ZG73, A0A3Q0JEG8, A0A3Q0J9Q0, A0A3Q0JCD1, A0A3Q0ISE2, A0A3Q0J8K4, A0A1S3D587, A0A0G2HYC6, A0A0G2FQS8, A0A0G2HKP0, A0A0G2FYA3, A0A0G2FNE9, A0A0G2HTH8, A0A2P5HKQ7, A0A2P5HFP5, A0A2P5HKH9, A0A2P5HW67, A0A2P5HMQ7, A0A2P5HTN7, A0A2P5HMX3, A0A2P5I8D4, A0AIE5V5N8, A0AIE5WE31, A0AIE5VW96, A0AIE5VZA9, A0AIE5VG01, A0AIE5VSM3, A0AIE5W1E8, A0AIE5VU03, A0AIE5UWE4, A0AIE5VYJ8, A0AIE5VM23, A0AIE5URK1, A0AIE5WF62, A0AIE5VK53, A0AIE5UP39, A0AIE5VK38, A0AIE5V886, A0AIE5W3D1, A0AIE5WE45, A0AIE5VDA3, A0AIE5WHY6, A0AIE5VK85, A0AIE5UVP0, A0AIE5UR82, A0AIE5ULJ0, A0AIE5VWU8, A0AIE5V9T7, A0AIE5V139, A0A1E5ULP8, A0A1E5VY77, A0A1E5WKA8, A0A1E5VIX5, A0A1E5VIW0, A0AIE5V1A3, A0AIE5VVS7, A0AIE5UY38, A0AIE5VM04, A0AIE5V8G9, A0AIE5VVN3, A0AIE5VKB6, A0AIE5WFT2, A0AIE5W4R6, A0AIE5WAP0, A0AIE5VM26, A0AIE5WME9, A0AIE5UVF0, A0AIE5VMMO, A0AIE5VU80, A0AIE5VS42, A0AIE5UV74, A0AIE5VIT8, A0AIE5VU74, A0AIE5VIF0, A0AIE5VW42, A0AIE5W8Y3, A0AIE5VS52, A0AIE5WIF4, A0AIE5ULC1, A0AIE5UQP5, A0AIE5VIJ8, A0AIE5WCL7, A0AIE5UW57, A0A0D8XPX3, A0A0D8X9K2, A0A0D8XPW4, A0A0D8XME1, A0A0D8X7W5, A0A0D8Y1F7, A0A0D8YBC7, A0A0D8XDB9, A0A0D8XCM5, A0A0D8XNV8, A0A0D8XSU8, A0A0D8Y821, A0A0D8Y118, A0A0D8XQ45, A0A0D8Y7C0, A0A0D8Y6I0, A0A0D8XYW9, A0A0D8Y6A7, A0A0D8XWZ8, A0A0D8XEJ7, A0A0D8XTU3, Q54TD6, F0ZKZ0, A0A163FB68, A0A163C076, A0A162ZDC7, A0A162ZPJ8, A0A163F3P6, A0A162WAZ1, A0A163J5K2, A0A2D8TLG9, A0A443QH82, A0A443QVN9, A0A3S4RFD9, A0A443Q9A2, A0A3S4Q503, A0A443Q760, A0A3S3QB39, A0A443RFM8, A0A3S3S9I9, A0A443R8P4, A0A2H3FTD0, A0A2H3F312, A0A1J9S7L6, A0A1J9R5G2, A0AIJ9RDT5, A0AIJ9RSV2, A0A1J9R6F7, A0A1J9QKG2, A0A1J9SJL7, A0A0G2GEA1, A0A0G2E8Z2, A0A0G2DVZ6, A0A0G2GU77, A0AIS8BAS3, A0A2A2JU22, A0A2A2L628, A0A2A2JTT9, A0A2A2L657, A0A2A2J1X0, A0A2A2K5D7, A0A2A2JJI2, A0A2A2JIU6, A0A2A2K5L0, A0A2A2KWG8, A0A2A2JS95, A0A2A2LN52, A0A2A2L620, A0A2A2J5B9, A0A2A2KVUO, A0A2A2L6B7, A0A2A2J211, A0A2A2LYR2, A0A2A2L669, A0A2A2L5U1, A0A2A2K927, A0A2A2L660, A0A2A2KCF6, A0A2A2KWT1, A0A2A2JR35, A0A2A2JRM6, A0A2A2K944, A0A2A2L6G5, A0A2A2JTJ2, A0A2A2J269, A0A2A2L631, A0A2A2JJ18, A0A2A2JW46, A0A2A2KN03, A0A2A2J7C6, A0A2A2LFJ4, A0A2A2JK27, A0A2A2LSI3, A0A2A2K984, A0A2A2J737, A0A2A2JVG0, A0A2A2LHE2, A0A2A2J9M8, A0A2A2LZT7, A0A2A2LK42, A0A2A2L5A9, A0A2A2L5Z3, A0A2A2K562, A0A2A2JIY3, A0A2A2JB04, A0A2A2JVQ1, A0A2A2M1Y0, A0A2A2L2G3, A0A2A2L2E8, A0A2A2L668, A0A2A2KEI4, Q8LP56, Q8LP55, A0AIS3FYB6, A0AIS3FZS6, A0A1S3G699, A0AIS3FYB4, A0AIS3FW29, A0A1S3G403, A0AIS3FU82, A0AIS3FX05, A0AIM4MWK1, M1WLK5, M1WM21, M1WJ15, M1WKU1, M1WQZ1, M1WJ16, A0A2Z7CXC8, A0A2Z7D6Z2, A0A2Z7AFH7, A0A2Z7D8Q9, A0A2Z7CER6, A0A2Z7B0U4, A0A2Z7CJB8, A0A2Z7A354, A0A2Z7BCF0, A0A2Z6ZZX4, A0A2Z7D973, A0A2Z7AG07, A0A2Z7B1U4, A0A2Z7B0H7, A0A2Z7CYS7, A0A2Z7A3I7, M2YM64, N1PXS3, N1PBL6, M2WMG3, N1PFX2, A0A3P7PPB3, A0A3P7Q930, A0A0N4U4X9, A0A3P7PQ65, A0A0N4UGI5, A0A0N4UGV7, A0A0N4UKQ2, A0A0N4UOP8, A0A0N4UIB0, A0A0N4UGG1, A0A0N4UDY3, A0A0N4UNZ5, A0A0N4UEW0, A0A0N4U2K8, A0A0N4U723, A0A0N4U3H3, A0A158Q4C2, A0A0N4U9B2, A0A0N4ULW1, A0A151GIG1, W712Z9, B3MYN7, B3MF92, B3MFW4, B3LWY2, A0A0P8Y1T9, B3M3A3, B3M3A4, B3LV56, A0A0M4ESS7, A0A0M4EKR6, A0A0M4EV08, A0A0M5J364, A0A0M4EXF3, A0A0M4EEP6, A0A0M4EME8, A0A0M4ECI2, B3P534, B3NSI7, A0A0Q5WP35, A0A0Q5WL19, B3N8M9, B3P5C3, B3P535, B3P944, B3P7G9, A0A1W4V338, A0AIW4UYM8, A0A1W4W708, A0A1W4UGH8, A0A1W4UYB4, A0A1W4U5N1, A0A1W4UGL8, A0A1W4U500, A0AIW4V9D3, A0AIW4VW08, A0AIW4UR70, B4JTG4, B4JSG6, B4J4M5, B4J8W1, B4JRW8, B4JMU5, B4J4M4, B4JRW7, B4JG50, A0A3B0KC09, A0A3B0KS22, A0A3B0JG58, A0A3B0JKN7, A0A3B0K9B5, A0A3B0KB54, A0A3B0JFB9, A0A3B0K860, A0A3B0JT97, A0A3B0JFL0, A0A3B0JKK1, Q9BMD4, Q86P35, A0A0B4LF37, A0A0B4LEX9, Q9BMD5, Q8IGS2, A1Z7G0, Q1WWD1, B6IDK6, Q9W5Z0, B5X535, Q32KF1, A1Z7G1, Q8MS37, Q9VCR4, Q9VAG2, Q9V9X0, Q9V9X1, A1Z8J3, A1Z8J4, A0A0Q9X7G2, B4KR75, A0A0Q9X8J5, B4K8A2, B4KT11, B4K8A0, B4K4N1, B4KT12, B4KMN4, B4L2D3, B4GBV0, B4GP76, B4GYU7, B4GYU9, B4GGH2, B4GBV1, B4GNR1, B4H315, B5E0A1, Q29BN3, B5DYT0, B5DYS9, A0A0R3NKK5, Q293D9, B5DKG8, B4HRZ3, B41M31, B4HNF3, B4HRZ2, B4HZF8, B4IJ01, B4I033, B4HFA6, B4QFU2, A0A0J9TYZ5, A0A0J9TWC5, B4QSN2, B4QB78, B4NSA9, B4QSN1, B4R0T6, A0A0J9R8B2, B4R722, A0A0J9RAB3, B4LMYO, B4MEP6, A0A0Q9WGQ1, B4LZT0, B4LMX9, B4LX01, B4LMZ8, B4MEC9, B4LX02, B4NIE3, B4NMW3, B4NA23, B4MYB6, B4N9U9, B4NMW2, B4NIE6, B4NCY5, B4P7G4, B4PPJ2, A0A0R1E8R0, B4PWJ2, B4P2V3, B4PNE6, B4PNE5, A0A0R1E9F3, B4PN38, A0A0R1DN71, A0A0RIEAK2, A0A0R1E956, A0A093GPD4, A0A093GWQ7, A0A437ACB8, A0A436ZUE2, A0A437A9N2, A0A154NWA8, A0A154PP16, A0A154PDD4, A0A154P8R1, A0A154PGX2, A0A154PF39, A0A2I6QP38, W6UHM1, A0A068WP17, W6UV63, W6UEB0, A0A068WL63, A0A068WDY5, A0A068X3L0, A0A068Y252, A0A068Y2J7, A0A068Y2T5, A0A087VY50, A0A183AP36, D8LQ47, D7FKT5, A0A091JLUO, A0A091JD63, A0A091J4H6, A0A091JSL3, U6GJ11, U6GCD3, U6GPI2, U6GEH8, U6GV22, U6GQW4, U6LNL3, U6M125, U6LQZ6, U6LPX3, U6LG02, U6LEP7, U6M8Q0, G0LEU7, U6M1Y7, U6MAY8, U6LYQ2, U6KE00, U6N3N3, U6N256, U6MUL8, U6MIM2, U6MTA8, U6MUE5, U6MZX5, B0FLX7, U6N5K1, U6H1N4, U6GYZ4, A0AIS6JVG4, U6KPE6, U6L5A4, U6KPH4, A9Q278, U6KZX4, U6KR47, U6L0S8, H9B9Q2, Q9U966, A0A0R3S3W6, A0A0R3RMT7, A0A0R3S175, A0A158Q7L5, A0A0R3S6Y6, A0A0R3RSG8, A0A0R3RQS7, A0A158Q8Q1, A0A0R3RJF9, A0A0R3SOT9, A0A0R3RLA6, A0A158Q7Z5, A0A0R3RNM3, A0A0R3RQF1, A0A0R3RND4, A0A2P7ZU16, A0A2P8A0K2, A0A2P7ZTR3, A0A2P7Z2I6, A0A2P8A4U8, A0A2P8AJH4, A0A2P8A0V9, A0A2P7ZE80, A0A2P7ZEA0, A0A2P7YG32, A0A2P8A7V0, A0A2P7ZAA3, A0A2P7Z3V7, A0A2P8AIG7, A0A2P8A7U5, A0A2P7Z292, A0A2P8ABP9, A0A2P7ZCS8, A0A2P7ZQ48, A0A2P7Z2J1, A0A2P8AE22, A0A2P8A8C3, A0A2P7ZQ26, A0A433T326, A0A3SiBH73, A0A3SOZKA2, A0A433TIN4, A0A3SOZE69, A0A3SiBVS8, A0A433SIG5, A0A433U408, A0A433TGA1, A0A433TX18, A0A433U537, A0A433TXC9, A0A3S1HNN2, A0A3S1BNP3, A0A3S1BSL7, A0A3SiBG98, A0A3SiAGJ4, A0A433UC51, A0A3SiCCH7, G8DGE3, R1EI28, R1BAM4, R1B683, R1C548, RiC7G4, RiCY05, R1DNH1, R1F3X8, R1BID0, R1B9P9, R1FUQ2, R1E328, R1BF00, R1D562, A0A0D3I431, R1DXZ7, R1FC05, R1G197, R1F3B4, R1BP77, RiEXU6, R1FN73, R1EEU0, RiC4W0, R1D1K2, UlGDJ7, UlHN00, U1HVD3, A0A2Y9J0Yi, A0A2Y9J0V1, A0A2Y9J9R1, A0A2Y9KTY8, A0A2Y9KI03, A0A2Y9KPG6, A0A2Y9IZ91, A0A2Y9J4D9, A0A444ETX7, A0A444FD73, A0A427BAD6, A0A444E0U3, A0A445MMM3, A0A444DKE0, A0A426Y944, A0A444DNP9, A0A444CQP6, A0A426ZAP5, A0A444DGM2, A0A426XMX6, A0A444DJ88, A0A444FQB9, A0A444EPL5, A0A444DYW9, A0A426Z8Y2, A0A427A7Z6, A0A427ADX1, A0A427A375, A0A426ZAK1, A0A426Y7Y5, A0A444F0S5, A0A426Z0W7, A0A426XTUO, A0A426X112, A0A445MI72, A0A426Y1Q2, A0A426XZ16, A0A444FSB7, A0A444DA88, A0A444CS48, A0A426YR82, A0A426YBE7, A0A426WZY4, A0A444EBR5, A0A444G7H0, A0A444FF33, A0A427BBYO, A0A426YCT9, A0A426X001, A0A426XKV3, A0A426YHE0, A0A444FQX5, A0A444FXT6, A0A444G8Y1, A0A444E0K6, A0A444FSD3, A0A444C1A8, A0A444EZB8, A0A444EYD8, A0A444FT20, A0A426YPX6, A0A444E275, A0A444FI43, A0A426YFG1, A0A427B9L9, A0A445MGF6, A0A426Z6T2, A0A426XGC5, A0A444GI94, A0A444C1E0, A0A444GAN1, A0A444C1T9, A0A444GIB6, A0A444G7E9, W8IAH2, A0A0L8BYH5, A0A2V4WJQ1, A0A285XA72, D1CSF5, A0A1E3VH17, A0A178XXR5, A0A249PID2, A0A2V4ZAK9, A0A2V4ZCS8, A0AIC0SLC3, A0A1C0SWS9, A0AiS1T063, A0A395L2C9, A0A2N8JDI4, A0A2T3DA21, A0A0Q9EJQ9, A0AIG9QSV3, A0A0N4UYF4, A0A0N4VB29, A0A0N4VKQ5, A0A158Q9W4, A0A0N4V3E1, A0A0N4V9C5, A0A0N4VBZ0, A0A0N4V6Q2, A0A0N4V4P5, A0A0N4USF4, A0A0N4UVL6, A0A0N4UTY5, A0A0N4VH10, A0A0N4V8H6, A0A0N4UVC2, A0A158QAV3, A0A158QB01, A0A0N4VNF8, A0A0N4VFH7, A0A0N4UVC1, A0A0N4V445, A0A3P6INW5, A0A1H2U8J0, A0A1Y2LTI9, A0A1Y2M3K0, A0A1Y2LUZ8, A0A1Y2LKS8, A0A1Y2LT57, A0A1Y2M6T7, A0A1Y2MOF2, A0A1Y2LRR1, A0A1Y2M3W5, A0A1Y2LUZ6, A0A1Y2M927, A0A1Y2LUE0, A0A1Y2LJN4, A0A1Y2LMN7, G4WG37, F6USP9, F6ZWI6, F6PR90, F7BK76, A0A3Q2H7K1, A0A3Q2HUE1, F6T3E4, A0A3Q2L7R0, A0A3Q2L1V8, A0AIS3WHJ7, A0AIS2ZDKO, A0AIS3WHE5, A0A1S3A071, A0A1S3W4L1, A0A1S3W4J1, A0AIS2ZMK4, A0A1S3A1S4, A0AIS2ZDJ8, Q29485, Q8LP54, Q8LP53, Q8LP52, Q8LP51, 080355, A0A022QTQ7, A0A022PT68, A0A022R2X9, A0A022QF32, A0A022R014, A0A022RNY5, A0A022QYC6, A0A022QY85, A0A022RTU8, A0A022QN22, A0A022QIR8, A0A022QN16, A0A022R8V5, A0A022Q9S0, A0A022QRE8, A0A022PWI2, A0A022QGP2, A0A022QI21, A0A022R1Z8, A0A022QJW6, A0A022RSP1, A0A022R428, A0A022QNQ9, A0A022QUF4, A0A022R0W8, A0A022QJX3, A0A022QY81, A0A022QT32, A0A022QZT5, A0A022QZQ9, A0A022QW47, A0A022RTP0, A0A022R1U4, A0A022QEX0, A0A022R559, A0A022R2X4, A0A022QPW6, A0A022R1U7, A0A022R4G1, A0A022RYE8, A0A022RS75, A0A022QX25, A0A022QZ29, A0A022QGM9, A0A022QFN7, A0A022R018, A0A022QKT8, A0A022R8M0, A0A074M1L8, A0A0N8GK13, A0A2F0AWT5, A0A3P9A6E8, A0A3P9AKF4, A0A3P8ZCM4, A0A3P8YY57, A0A3P8YIZ1, A0A3P8YIY1, A0A3P8Z4D8, A0A3P8XUT0, A0A3P8X7I2, A0A058ZUL1, A0A059C9G5, A0A059C6C8, A0A058ZUX1, A0A059CAD5, A0A059C8D0, A0A059C8W2, A0A059BMB1, A0A059CF25, A0A059BJE7, A0A059A1V7, A0A059CAF8, A0A059D2A0, A0A058ZTJ3, A0A058ZS00, A0A059BMY5, A0A059C9N1, A0A059CAG3, A0A059BJZ0, A0A059BVD6, A0A059CD96, A0A059D0P0, A0A059CDS6, A0A059C900, A0A059CDJ8, A0A059C9K6, A0A059BCC2, A0A059C904, A0A059C2G3, A0A059C908, A0A059CF20, A0A058ZUYO, A0A059BX54, A0A059BMJ5, A0A059DHG7, A0A059C975, A0A059DG99, A0A059CL14, A0A059CDT6, A0A059BNH3, A0A059C9N8, A0A058ZSM4, A0A059CDK9, A0A059BLH2, A0A059DCZ0, A0A059CZX4, A0A059BAQ1, A0A059CAB5, A0A059CAX2, A0A059BVE0, A0A059A785, A0A059C1G2, A0A059A615, A0A059A075, A0A059C9A3, A0A059C8U8, A0A059B9B7, A0A059BMI0, A0A059BRV2, A0A059BKX0, A0A059BNH7, A0A059A6I8, A0A059C945, A0A059C6U7, A0A059BVV6, A0A059AXJ7, A0A059DF87, A0A059C9T6, A0A059DFY6, A0A059BN16, A0A059CF13, A0A058ZSA5, A0A059D643, A0A059DISO, A0A059D249, A0A059BVR8, A0A058ZTY8, A0A059BTYO, A0A059BVQ8, A0A059CDF2, A0A059BWS9, A0A059DGY3, A0A058ZUE3, A0A059BR54, A0A059CDJ4, A0A059CE26, A0A059BLY4, A0A059CE51, A0A059BB89, A0A059DGF5, A0A059BUD2, A0A059BLZ5, A0A059CUD3, A0A059C982, A0A059C9H8, A0A059C958, A0A059BB94, A0A059CAH7, A0A059CXX9, A0A059C8V4, A0A059CE41, A0A059BLY9, A0A059CDR9, A0A059CAB0, A0A059DGX5, A0A059BJD9, A0A059BMJ2, A0A059BNG0, A0A059BM31, A0A058ZQZ8, A0A059CPC6, A0A059CF29, A0A058ZSW8, A0A059CEE1, A0A059BM22, A0A059CAC4, A0A059A163, A0A059A230, A0A059BVD2, A0A059CEG0, A0A059CBT7, A0A059C9B7, A0A059C114, A0A059BUV4, A0A059D2V1, A0A059D2V0, A0A059C631, A0A059CD83, A0A059CDT5, A0A059B8N1, A0A059C5L8, A0A059BMX2, A0A059A0X2, A0A059BMX6, A0A059AAQ7, A0A059CDH3, A0A059CE67, A0A059BMA5, A0A059DGF0, A0A059BMG9, A0A059AB79, A0A059CSN4, A0A059CED3, A0A059C9F2, A0A059CI09, A0A059BMW7, A0A059BLI8, A0A059CE37, A0A059A790, A0A058ZU59, A0A059BSQ0, A0A059D582, A0A059CEP6, A0A059C965, A0A059CB94, A0A059BEM4, A0A059CD78, A0A059CE46, A0A059BLG8, A0A059DG00, A0A059CF15, A0A059DDF4, A0A059BJQ8, A0A059B892, A0A059BUD7, A0A059BAR2, A0A059BMY1, A0A059AWY3, A0A059B897, A0A059BJX1, A0A058ZSV0, A0A059BNF8, A0A059A0R6, A0A059A823, A0A059BKW4, A0A059BDD1, A0A059C6B4, A0A059DFF0, A0A059DFE3, A0A059BMB6, A0A059BBN7, A0A059D2I7, A0A059C9R7, A0A059BTKO, A0A059BBA1, A0A058ZW23, A0A059A1Z3, A0A059BM37, A0A059C9W0, A0A059CF08, A0A059BMH9, A0A059A6J2, A0A059BM34, A0A059BLW8, A0A059AKP5, A0A059CAE7, A0A058ZV14, A0A059DFZ6, A0A059C153, A0A059B9A1, A0A059DFZ4, A0A059A113, A0A059A235, A0A059C9M3, A0A059BMI4, A0A059DGY9, A0A058ZVI2, A0A059BMB9, A0A059CB42, A0A059BLZ9, A0A059BUZ3, A0A059C536, A0A059CDUO, A0A059C8X4, A0A059B232, A0A059AB02, A0A058ZZN8, A0A059CBU2, A0A059C1M6, A0A059DGG0, A0A059D5Y0, A0A059CAC9, A0A059C9Q5, A0A059C9M7, A0A059BVV1, A0A058ZZL0, A0A059DG97, A0A059CDS9, A0A059BJX7, A0A059CDG7, A0A059CAI8, A0A059CDS1, A0A059BLH9, A0A059C8X9, A0A059C976, A0A059CEE5, A0A059CAJ3, A0A059BNG6, A0A058ZV47, A0A059BR56, A0A059BVR3, A0A059BUQ9, A0A059BVR0, A0A059C7M3, A0A059BB21, A0A059DFD9, A0A059BRU7, A0A059C951, A0A059CEQ0, A0A059A7T8, A0A059CAX8, A0A058ZQT7, A0A059C0W1, A0A059C9H1, A0A059D1S5, A0A058ZXR1, A0A059CF34, A0A059BNE6, A0A059DGE5, A0A059C970, A0A059CDU4, A0A058ZVT6, A0A059D0Q2, A0A059C9F8, A0A059DG91, A0A059CA18, A0A059BLV9, A0A059CAP5, A0A059A0T8, A0A058ZRN7, A0A059BB75, A0A059CDK1, A0A059BVR4, A0A059CAE0, A0A059BM19, A0A058ZSW3, A0A059DG93, A0A059CE63, A0A058ZVN1, A0A059D240, A0A059CAE9, A0A059BCB2, A0A059CE56, A0A059BWP5, A0A059C9H4, A0A059BRU8, A0A059BAZ9, A0A059CDH8, A0A059C9M0, A0A059BC92, A0A059CED7, A0A058ZTK4, A0A059CDF8, A0A059BMZ2, A0A059CFL3, A0A059C947, A0A310S483, A0A310SBN6, A0A310SWL1, A0A310SLW1, A0AIY3B584, A0AIY3AU99, A0AIY3AXF6, A0AIY3B164, A0AIY3BLQ2, A0AIY3AUF8, A0AIY3BLR8, A0AIY3AZZ7, A0AIY3AWF6, A0AIY3BKE0, A0A2E4CYV5, A0A093IZQ0, V4MUM3, V4L7H3, V4KWJ2, V4KQS5, V4KY09, V4MJV0, V4LNJ7, V4KDR5, V4L028, V4MC52, V4L095, V4L2I0, V4KJ04, V4KNB2, V4KHG6, V4M7D2, V4MED4, V4MBM7, V4LX42, V4LB40, V4MDH2, V4K896, V4KDS9, V4P511, V4LVA5, V4KWJ7, V4LD36, V4KC43, V4KHG0, V4L090, V4KDS4, V4L8J2, V4KPI9, V4JV72, V4MIC9, V4KPJ9, V4JTX9, V4NUT8, V4NAM5, V4MH01, V4KFK6, H6C4W5, A0A0D1Y6D0, A0A438NHT6, A0A0D2AIX8, A0A0D2DN60, A0A0D1X4X0, A0A0D1YE49, A0A0D1WNZ7, A0A0D1YQP5, A0A0D1Z1V9, A0A0D2BF00, A0A0D2CZ88, A0A0D2CUE6, A0A0D2D105, A0A2N9F471, A0A2N9G5U5, A0A2N9ID93, A0A2N9I6Q1, A0A2N9IVV1, A0A2N9F4G1, A0A2N9F6W7, A0A2N9F3H1, A0A2N9FT24, A0A2N9ERT8, A0A2N9G5U0, A0A2N9EZKO, A0A2N9I7P8, A0A2N9EYQ2, A0A2N9HI25, A0A2N9I8I7, A0A2N9H741, A0A2N9H8W4, A0A2N9F7R0, A0A2N9H6F3, A0A2N9H2X2, A0A2N9J533, A0A2N9G8Z2, A0A2N9HS19, A0A2N9HL38, A0A2N9IBZ3, A0A2N9G5W6, A0A2N9HCP4, A0A2N9H5Z2, A0A2N9FDE0, A0A2N9F3U1, A0A2N9IVM7, A0A2N9FCL3, A0A2N9HX85, A0A2N9FNS6, A0A2N9FA80, A0A2N9F6Y2, A0A2N9EZI6, A0A2N9HDA4, A0A2N9I2U2, A0A2N9IY45, A0A2N9EYP9, A0A2N9HL99, A0A2N9H4B2, A0A2N9HRU4, A0A2N9HI04, A0A2N9FE92, A0A2N9HPG2, A0A2N9FX35, A0A2N9H9S5, A0A2N9IUZ4, A0A2N9IV03, A0A2N9I632, A0A2N9J6T9, A0A2N9EYX2, A0A2N9FCP0, A0A2N9F9Y2, A0A2N9HTG9, A0A2N9H672, A0A2N9GTW2, A0A2N9IZ71, A0A2N9HNC1, A0A2N9HE29, A0A2N9FRE3, A0A2N9HZG0, A0A2N9FDT8, A0A2N9GA03, A0A2N9HIJ0, A0A2N9ICZ8, A0A2N9GJI4, A0A2N9IM33, A0A2N9HTB4, A0A2N9GJM5, A0A2N9HL23, A0A2N9FCP6, A0A2N9G696, A0A2N9IPQ1, A0A2N9GAM8, A0A2N9J640, A0A2N9FCQ2, A0A2N9EM72, A0A2N9EE92, A0A2N9ED67, A0A2N9G5P0, A0A2N9I9D1, A0A2N9HYD5, A0A2N9F021, A0A2N9H5L7, A0A2N9IG02, A0A2N9GLI0, A0A2N9ELE2, A0A2N9ER54, A0A2N9GLH4, A0A2N9F7N7, A0A2N9HKW5, A0A2N9I6X0, A0A2N9I1I5, A0A2N9HHV9, A0A2N9I2Z7, A0A2N9EM92, A0A2N9G9P1, A0A2N9EYP4, A0A2N9H6F1, A0A2N9GWJ5, A0A2N9HS70, A0A2N9HAB2, A0A2N9FAP6, A0A2N9HI47, A0A2N9EZA0, A0A2N9H5Z0, A0A2N9FCU8, A0A2N9GXA5, A0A2N9FZ00, A0A2N9ER32, A0A2N9H0J5, A0A2N9IT13, A0A2N9HLN1, A0A2N9GJ59, A0A2N9F736, A0A2N9EI38, A0A2N9HRI8, A0A2N9FLN9, A0A2N9I3I6, A0A2N9G737, A0A2N9J704, A0A2N9I596, A0A2N9FCQ0, A0A2N9HWH5, A0A2N9GWS3, A0A2N9IMS6, A0A2N9I1K9, A0A2N9J9T5, A0A2N9HRW8, A0A2N9HQ34, A0A2N9GA49, A0A2N9GID3, A0A2N9EE63, A0A2N9HNV5, A0A2N9IY81, A0A2N9EYQ0, A0A2N9IVN9, A0A2N9EME6, A0A2N9H6H0, A0A2N9ERJ4, A0A2N9F311, A0A2N9IBN6, A0A2N9HKN1, A0A2N9EYQ9, A0A2N9IGP9, A0A2N9H563, A0A2N9I8M7, A0A2N9I612, A0A2N9HB53, A0A2N9HHE3, A0A2N9FNV9, A0A2N9GFL6, A0A2N9GUQ4, A0A2N9EYQ8, A0A2N9HFF6, A0A2N9GAZ3, A0A2N9GZD3, A0A2N9IE26, A0A2N9HWE4, A0A2N9EZ06, A0A2N9EE70, A0A2N9IH59, A0A2N9G6C0, A0A2N9IC01, A0A2N9HTA6, A0A2N9HEY7, A0A2N9EM85, A0A2N9FTC8, A0A2N9J971, A0A2N9FSD1, A0A2N9HLB3, A0A2N9HHG3, A0A2N9I338, A0A2N9FD43, A0A2N9HYX6, A0A2N9HM72, A0A2N9IGI8, A0A2N9IVH2, A0A2N9HDT5, A0A2N9FXK4, A0A2N9GE58, A0A2N9F4K3, A0A2N9G9R4, A0A2N9FW96, A0A2N9GXP3, A0A2N9FYD6, A0A2N9IAR8, A0A2N9F9M6, A0A2N9GCZ4, A0A2N9G2I6, A0A2N9EYU2, A0A2N9G9Q1, A0A2N9IX44, A0A2N9IHR5, A0A2N9H6L7, A0A2N9HT54, A0A2N9EEA4, A0A2N9I6X5, A0A2N9GG82, A0A2N9FAN8, A0A2N9G0E5, A0A2N9HDUO, A0A2N9IY90, A0A2N9JBS8, A0A2N9HW38, A0A2N9HRB8, A0A2N9GE76, A0A2N9HGR5, A0A2N9HQP2, A0A2N9F979, A0A2N9JBR8, A0A2N9I7X6, A0A2N9IAJ3, A0A2N9EZS8, A0A2N9H640, A0A2N9G6V6, A0A2N9HI57, A0A2N9H894, A0A2N9IAS3, A0A2N9F3L2, A0A2N9HEM6, A0A2N9G7C0, A0A2N9F1Q0, A0A2N9EYP0, A0A2N9J7F4, A0A2N9H1C2, A0A2N9ERP4, A0A2N9HZ89, A0A2N9IA84, A0A2N9IH48, A0A2N9FXU7, A0A2N9H6E0, A0A2N9I4V8, A0A2N9IER8, A0A2N9IVP2, A0A2N9IHP1, A0A2N9I4B6, A0A2N9H6F8, A0A2N9IGJ1, A0A2N9IFD7, A0A2N9J390, A0A2N9HZI8, A0A2N9IGJ3, A0A2N9FUZ8, A0A2N9G2R4, A0A2N9ERG2, A0A2N9GAN0, A0A364JW94, A0A2H1C6Y1, A0A2H1C2I6, Q9BH09, A0A2I2UAP2, M3WZJ1, M3VWV2, A0A2K5TIV6, M3X3T9, U3K7I1, U3K2Y8, U3KBUO, U3KBT4, U3KCE5, A0A238LAN8, A0A238LBM3, A0A2A4M1B2, A0A2D5LP88, A0A0S7BYF0, A0A0K8PA51, A0A226E742, A0A226EIX0, A0A226EJL6, A0A226DUU9, A0A226ERX6, A0A226EKR3, A0A226EXA3, A0A226CZ90, A0A226E675, A0A226D8L3, A0A226DA03, A0A226E8X0, A0A226ES37, A0A226E4B6, A0A226DYP9, A0A226F3C7, A0A226DMW5, A0A177EZP3, A0A0D2JU71, A0A0D2JHMO, A0A178BR81, A0A178BZ24, A0A178BL33, A0A178CTQ1, A0A0D2E424, A0A0C9PLF2, A0A0C9Q7Z9, A0A0C9Q6E7, A0A0C9RT62, A0A0C9RVW4, A0A0C9QIV9, A0A0C9RYR6, A0A0C9QF47, A0A0C9RAM8, A0A0C9R7C8, W8PA20, A0A091DZJ9, A0A091DXE1, A0A091DVM4, A0A091CMD3, A0A093I9I3, A0A093ILG6, A0A093IZP1, A0A1W2EWK8, A0A1W2D4E0, Q0G412, A0A2E1H9K8, A0A146MU99, A0A146YM67, A0A146XF35, A0A146SKJ1, A0A146NDC0, A0A146SAR9, A0A3Q2P288, A0A3Q2U5Q0, A0A146UV90, A0A3Q2PXJ3, A0A147AF21, A0A146NFQ6, A0A146MVK4, A0A146NM15, A0A3Q2NV28, A0A146NKH6, A0A3Q2QG44, A0A3Q2NUN0, A0A146MX16, A0A146NV94, A0A146MUA7, A0A3Q2NRI0, A0A146UVF0, A0A146ML14, A0A3Q2P3A5, A0A146NEU9, A0A146NDC3, A0A146Y0F0, A0A146MMW4, A0A146R9E1, A0A146YKS9, A0A146NEK3, A0A147AX70, A0A3Q2P247, A0A146YWT4, A0A146NEL7, A0A3Q2STI2, A0A146YKR1, A0A146ML01, A0A3Q2NUL5, A0A0S6XK74, A0A0S6X4V7, A0A0C9MAW8, A0A0S6XHR7, A0A0S6XII5, A0A0S6XQ97, A0A0S6XL07, A0A0C9LMV7, A0A0S6XI06, A0A0S6XTD5, A0A0S6XM18, A0A0S6XLT8, A0A0S6XMF5, A0A0C9ME04, A0A0S6XI16, A0A0S6XNW0, A0AlViT8U0, A0AlV1T9B7, A0A1V1TBD7, A0A428UEU2, A0A428TVT7, A0A428UJD4, A0A428UW00, A0A428U053, A0A428U9S6, A0A428TW22, A0A428T8Z1, A0A428USM7, A0A428RYB6, A0A428U1S8, A0A428TDI1, A0A428U0L8, A0A428TDN9, A0A428TDI8, A0A428UAR9, A0A2T4GF04, A0A2T4GEI2, A0A2T4GM20, A0A2T4GQ39, A0A2T4GZI7, A0A2T4HAV6, A0A2T4H2K6, A0A2T4GU55, A0A2T4GS35, A0A2T4GCH5, A0A2T4GFN6, A0A2T4GL46, A0A2T4GYZ7, A0A2T4GVC4, A0A2T4GS29, A0A2T4GYX5, A0A2T4GET1, A0A2T4H3Y6, A0A2T4GRL8, A0A430M7X9, A0A430KY02, A0A430LYZ3, A0A430L5I8, A0A430LMK1, A0A430LAE0, A0A430M9V2, A0A430LTZ7, A0A430LXM6, A0A430LXM7, A0A430L0Q7, A0A430LT59, A0A430L7B2, A0A430LDB8, A0A430LIS3, A0A430M2H2, A0A430LBT6, A0A430M5A4, A0A430LG97, A0A3M2RQX3, A0A3M2SJ30, A0A3M2S4P6, A0A3M2RRC6, A0A3M2RNA0, A0A3M2S004, A0A3M2SGY5, A0A3M2RVM1, A0A3M2RP84, A0A3M2SAI4, A0A3M2RVN5, A0A3M2SMUO, A0A3M2SDA7, A0A3M2SDH3, A0A0N0V5S0, A0A0M9EXB1, A0A0N0DCT1, A0A0N0V8P4, A0A0M9ETX5, A0A0M9ERF9, A0A0M9EKX4, A0A0M9ETU9, A0A0N0DBP2, A0A0M9EQI9, A0A0M9F2P3, A0A395SMB0, A0A395SU16, A0A395T394, A0A395TAS6, A0A395RLH4, A0A395TAR6, A0A395RR81, A0A395T1Z9, A0A395T8X0, A0A395T8W6, A0A395T7Q5, A0A395RWV1, A0A395T6G5, A0A395SJK3, A0A395T9N8, A0A395TBU9, A0A395SWW9, A0A395SJJ1, A0A395T993, A0A395RYQ6, A0A395TAA6, A0A395RI56, A0A1L7T250, A0AIL7SNI5, A0AIL7SY16, A0A1L7U4Q3, A0AIL7SQ32, A0AIL7TU10, A0AIL7UDI1, A0AIL7UAA5, A0AIL7TG43, A0AIL7TZT8, A0AIL7SRG8, A0AIL7UDC6, A0AIL7TI59, A0A1L7TU11, A0A1L7ST20, A0A1L7TSY6, A0A1L7T2L2, A0A1L7TN71, A0AIL7UJW9, A0AIL7UHL6, A0AIL7STR4, F9F0W5, F9FQB6, F9FN55, F9G9P5, F9FRR0, F9FNV4, F9F3M4, F9F4N5, F9G352, F9G5P5, F9G3D6, F9F676, F9GDQ2, F9FWK6, F9FLK6, F9G1R4, F9G1R3, F9GBZ7, A0A3L6P746, A0A3L6NN57, A0A3L6NQQ4, A0A3L6MW91, A0A3L6NQT5, A0A3L6NPD4, A0A3L6NTI7, A0A3L6NEX4, A0A3L6N5S7, A0A3L6N706, A0A3L6NWN1, A0A3L6NBP9, A0A3L6MWX0, A0A3L6N4Z1, A0A3L6NXP5, A0A3L6N0T9, A0A3L6NTQ6, A0A3L6NQ88, A0A3L6MUU1, X0HDS5, X0ISO2, X0HCS8, X0HRY6, X0HVQI, X0HNT3, X0HXU3, X0HEE6, X0HT53, X0HE86, X0IFF6, X0I6Z5, X0HQT8, X0HZM6, X0HS18, X0HBE0, X0GNF1, X0HVP9, X0HB55, X0H868, X0HRQ2, N4TVA0, N4UWB4, N4UQ87, N4TTM6, N4U5D0, N4U486, N4TXC1, N4TPR8, N4UXC7, N4UER4, N4U8I0, N4V0A9, N4UMR2, N4UFQ1, N4UFD6, N4U504, N4US39, N4TMJ2, N1S2M4, N1RYI2, N1RQ64, N1RQ00, N1S7G0, N1RYM7, N1SAV4, NiSAD8, N1RGZ6, N1S5R5, N1RB09, N1R8K0, N1SB76, N1RPT0, N1RPN7, N1S4B5, N1R9G0, N1RV49, N1RC32, N1S7H6, X0KKH1, X0JD64, X0J7Z6, X0JRR5, X0IXV2, X0K4Y4, X0LFY3, X0IRL6, X0K6S9, X0J8E8, X0J8L0, X0KS72, X0JS17, X0KGE6, X0J5F8, X0KBP3, X0IQZ7, X0LJG6, X0K565, X0K4R2, X0JMF5, A0A0D2XFR8, A0A0D2XY32, A0A0D2Y321, A0A0D2XC34, A0A0D2X9W2, A0A0D2XKS9, A0A0D2XG03, A0A0D2YBX0, A0A0C4DIY3, A0A0J9W4W2, A0A0D2YOC7, A0A0J9WPH9, A0A0J9VTQ2, A0A0J9VIA4, A0A0J9UPV1, A0A0J9WAX7, A0A0J9WCE8, A0A0J9UUH4, A0A0D2Y340, A0A0J9W359, A0A0D2Y341, A0A0J9WQB9, A0A0D2YGJ8, A0A0J9VU72, X0AFU5, W9Z4V9, W9ZBD0, X0AIL8, X0ARG8, X0BEV5, X0ATQ4, X0AKC9, X0AP22, W9ZFM9, X0AH58, X0B6E5, X0AJE3, X0AC84, X0B5X6, W9ZBV2, X0A7A2, W9ZGB5, X0A725, W9NLV3, W9PM75, W9NVE9, W9PM48, W9NUL9, W9NXE1, W9NJT9, W9P038, W9PVA4, W9PIK8, W9NWZ0, W9NMUO, W9QB84, W9P2D0, W9PWG0, W9NHW9, W9Q8W3, W9NIE4, W9Q072, W9P3J9, W9PEW3, A0A2H3G3H1, A0A2H3GP16, A0A2H3GPR0, A0A2H3GI26, A0A2H3G3A7, A0A2H3GT41, A0A2H3HFX7, A0A2H3H582, A0A2H3HWB5, A0A2H3G8B5, A0A2H3H7J7, A0A2H3HDQ9, A0A2H3HBA8, A0A2H3HUG8, A0A2H3GTZ9, A0A2H3H1W4, A0A2H3H7B8, A0A2H3GR19, A0A2H3HIX3, X0CBT2, X0CJZ6, X0CA20, X0C0K3, X0BLK2, X0BYZ9, X0BHR3, X0D8W0, X0D9D6, X0BNE2, X0CCJ8, X0D7P5, X0C5I2, X0BWY5, X0BK94, X0C4F8, X0BU43, X0CH27, X0CCJ4, X0BT77, X0BGL4, X0BSM4, X0CBV1, X0KM84, X0KL93, X0LI06, X0LM24, X0L461, X0N5D7, X0MNF5, X0LVS2, X0LLL6, X0KZC8, X0MQ25, X0LJZ4, X0LYG6, X0MWI5, X0MGF6, X0MLD1, X0MQJ9, W9JZF8, W9K3B2, W9K2I6, W9KHK3, W9JKU5, W9KL95, W9KMV3, W9KB71, W9LDU1, W9K179, W9JPG0, W9KU91, W9KWA8, W9K2L5, W9JY72, W9JLD1, W9JH38, W9JK71, A0A420PSH8, A0A420PDU2, A0A2H3SXG2, A0A420PMW4, A0A2H3TNB9, A0A2H3TUT4, A0A2H3TT16, A0A2H3TGN6, A0A420MLC5, A0A420SEB3, A0A2H3TL83, A0A420SIY6, A0A420QWS3, A0A420PGL0, A0A420NGZ2, A0A420R9X9, A0A420QHW6, A0A420SOP8, A0A420TXI9, A0A420UC75, A0A420MBT8, A0A420PHU3, A0A420QP35, A0A420P6T6, A0A420QIK4, A0A420PYP6, A0A2H3T198, A0A420QQV1, A0A420NBX5, A0A420NZN8, A0A420QQT6, A0A420RGE4, A0A420PWD7, A0A2H3TZQ8, A0A420NEW3, A0A2H3SMM6, A0A420RVH4, A0A420NNM5, A0A2H3T9F1, A0A2H3TSH4, A0A420RN85, A0A420N964, A0A420MDX2, A0A420NZR8, A0A2H3TND6, A0A2H3SPH1, A0A2H3TLK4, A0A420R6D6, A0A420QGY8, A0A420P5A3, A0A420NJK8, A0A2H3TLA9, A0A420MLJ4, A0A2H3U2W3, A0A420R1M2, A0A420UEN2, A0A420SA71, A0A420PG81, A0A420NEU8, A0A2H3T2A7, A0A420U4W6, A0A420PC63, A0A420Q8J1, A0A420U9L0, A0A420N211, A0A2H3T2B5, A0A420SKN9, A0A420RIQ3, A0A2H3T2H0, A0A420MQK5, A0A420S1Y5, A0A2H3SKQ3, A0A420RSV8, A0A420QNM7, A0A420U9X9, A0A420QBG3, A0AIB8AI36, A0AIB8AIV6, A0AIB8AQ51, A0AIB8AHP8, A0A1B8B148, A0AIB8AZG1, A0AIB8AZ43, A0A1B8B687, A0AIB8AHJ0, A0AIB8ARR3, A0A1B8B037, A0AIB8ARV5, A0AIB8AQS5, A0AIB8ADY5, A0AIB8AE77, A0AIB8AH23, A0AIL7VUI1, A0AIL7VQT1, A0AIL7VTN8, A0AIL7VQ64, A0AIL7VRM1, A0A1L7W234, A0A1L7W7F8, A0A1L7W3Q1, A0AIL7VZE6, A0A1L7W7T0, A0A1L7W068, A0AIL7VBG9, A0AIL7VJ89, A0A1L7V6L5, A0AIL7VLJ1, A0A1L7W6T5, A0A1L7W2B6, A0A1L7W3Q7, A0AIL7VAZ6, A0A1L7W9S2, A0AIL7VG50, A0A1L7V558, A0AIL7VE71, A0AIL7VBJ5, K3V6C2, K3VNW8, K3UVA9, K3VI86, K3VD86, K3VSF8, K3VZG0, K3W149, K3UVV0, K3VF03, K3VSH8, K3V8J7, K3W1U8, K3UBY1, K3VV23, K3V722, K3UTP7, K3VPR6, K3VW92, K3VVL6, A0A096PED2, A0A428RK34, A0A428SD27, A0A428RDV5, A0A428RQ68, A0A428SN67, A0A428Q5F5, A0A428SNN1, A0A428S0J4, A0A428RZR3, A0A428RSR8, A0A428RXQ1, A0A428RX08, A0A428RZJ5, A0A428SEZ3, A0A428RPS7, A0A428RNL1, A0A428RDW7, A0A428UIA3, A0A428SWL5, A0A428U3R5, A0A428T156, A0A428U3Z4, A0A428TXX9, A0A428SFD7, A0A428TWR6, A0A428UIJ1, A0A428SQQ5, A0A428U227, A0A428UCR8, A0A428THD3, A0A428S8Z0, A0A428UDE6, A0A428SKX5, A0A428PKQ2, A0A428QUF4, A0A428P7J0, A0A428P7H3, A0A428NDT7, A0A428PH59, A0A428Q7Z0, A0A428QDG1, A0A428RAS8, A0A428NYA2, A0A428NM38, A0A428Q0Y4, A0A428PKP7, A0A428PTI2, A0A428NUP9, A0A428PR63, A0A428P8A4, A0A428R4H3, A0A428NIV0, A0A428NZ95, A0A428PCW5, A0A428Q8W1, A0A428NR66, A0A428NN65, A0A428QD28, A0A428QWP2, A0A428PBY7, A0A428PA97, A0A428QKE9, A0A428P7T0, A0A428NXE6, A0A428P4U5, A0A428QQ80, A0A428PCY9, A0A428NSA9, A0A428PX74, A0A428NS70, A0A395MGQ2, A0A395MGA8, A0A395M7J9, A0A395MAG0, A0A395MLX9, A0A395N4U8, A0A395MHX2, A0A395N159, A0A395M5H5, A0A395MHI4, A0A395MIF4, A0A395MBX0, A0A395N2D5, A0A395N4Y5, A0A395N3C1, A0A395MVX4, A0A395MI62, A0A395MVQ6, A0A395MCF6, A0A395N1D8, A0A395N4X6, A0A395MZD0, A0A366S714, A0A366QWA4, A0A366QY56, A0A366S516, A0A366R509, A0A366S9E0, A0A366S648, A0A366S7J1, A0A366S6E2, A0A366RU34, A0A366SAJ5, A0A366S5H5, A0A366RLX0, A0A366RZB1, A0A366RSR3, A0A366S6U1, A0A366SA36, A0A366S608, A0A366R259, A0A090N5T3, A0A090MBD1, A0A090N5N0, W9I3C2, W9HY89, W9IHK5, W9HQZ2, W9IYB6, W9I981, W9IXH4, W9IFQ1, W9HPW8, W9HU54, W9HPA2, W9J633, W9IFB4, W9J1V2, W9IC02, W9HM81, A0A395SCF9, A0A395SN15, A0A395SBE5, A0A395S6D9, A0A395SH08, A0A395SBA6, A0A395RVT2, A0A395RRP8, A0A395SJC5, A0A395SIC0, A0A395RMJ7, A0A395RYB3, A0A395RVS6, A0A395SNH5, A0A395SDT1, A0A395S6D1, A0A395SDH6, A0A2L2U374, A0A2L2TJ64, A0A2L2U3G9, A0A2L2TQW0, A0A2L2TFB0, A0A2L2TKB7, A0A2L2TJF0, A0A2L2TLG9, A0A2L2TPW8, A0A2L2U3E5, A0A2L2T6R1, A0A2L2TZ15, A0A2L2TR51, A0A2L2T472, A0A2L2SR66, A0A2L2TU58, A0A2L2T569, A0A2L2U3K6, A0A2L2SSM8, A0A2L2TMX3, A0A2L2TCF7, A0A3G4ZYX2, J3PC95, A0A2M7N8H7, Q90978, Q90865, A0AID5PAR6, F1NWX6, R4GMH5, E1C8A1, F1NUN5, A0A3Q2U5D1, Q788Q2, A0A315UVV8, A0A315UQL6, A0A315VVX5, A0A315UYF8, A0A315UVQ5, A0AIG0KYN1, A0A2D5I3I9, A0A2D5PM94, A0A2E1JMT7, G3N792, G3NR28, G3Q8D9, G3NR40, G3NX83, G3NR48, G3NX96, G3N8R7, G3PGI4, A0A093F083, A0A1H7ZTR7, A0A2T6B2Z8, A0A3S3UUA3, A0A451GG88, A0AlN7QCJ4, A0A3P3DPT3, A0A398BPI3, A0A398BSF3, A0A358B3Q1, S8CSG8, S8BXM1, S8ECB0, A0A0J9XCZ4, SOE9M1, SOEAW3, SOE050, SODSB2, SOElP0, SODJL6, SOECMO, SODSY7, SOE5X7, SOEAP9, SODSC6, SOEMX3, SOE357, SOE2Q5, SOECL2, SOE6U9, SODJP2, SODZF4, SOEFD2, SOE497, A0A0I9Z138, A0A0I9YAH3, A0A0I9XHA9, A0A0I9Y8Q9, A0A0I9YM17, A0A0I9XFP3, A0A0J0BUYO, A0A2H3RKI8, A0A2H3SN28, A0A0J0CHN8, A0A2H3RR38, A0A2H3SWB6, A0A0I9X760, A0A2H3S7W2, A0A0J0CQW4, A0A2H3S359, A0A2H3SM63, A0A2H3RNJ8, A0A2H3RMB7, A0A2H3SWM1, A0A0J0CT57, A0A2H3RRY9, A0A0I9Y942, A0A2H3RGR6, A0A0I9XEV3, A0A2H3RUA2, A0A0I9XAZ3, A0A0J0BYF6, A0A2H3RVF2, A0A2H3RW12, A0A0I9XY78, A0A0I9YHM8, A0A2H3RR57, A0A0I9X911, A0A0I9XJR0, A0A0J0C0013, A0A2H3RVE0, A0A2H3SQE6, A0A0I9XAJ6, A0A2H3SHG6, A0A2H3R980, A0A0I9XDN8, A0A420SS68, A0A420TP29, A0A420UB76, A0A365NIV2, A0A365N6M1, A0A365MMV3, A0A365N4F2, A0A365NNN5, A0A365ND98, A0A420T6U9, A0A365NJW8, A0A420SEE3, A0A365N416, A0A365NIH4, A0A420SZ33, A0A365MQF6, A0A420UA77, A0A365MQ14, A0A365MMJ0, A0A420SDB1, A0A365MPJ1, A0A365N099, A0A365NDF6, A0A420SSE2, A0A420T6Q6, A0A420U9Y0, A0A365N1D1, A0A420T9A6, A0A365N275, A0A365MYB1, A0A420TU20, A0A420TTT9, A0A365MRF5, A0A365MKN4, A0A420SGE2, A0A420TM01, A0A420SQ44, A0A365N043, A0A365NGM1, A0A420SEZ4, A0A420SPD6, A0A420SWC9, A0A365MMK9, W7MOV8, W7MQW8, W7LIK3, W7N541, A0A139YBX2, W7MTU5, W7NCD1, W7N6Q6, W7MUN6, W7ME54, W7MSX0, W7MR98, W7MZZ1, W7N269, W7N008, W7LSV5, W7N837, W7LEM9, W7MXJ6, A0A139YBM8, W7M9D1, W7MZW8, A0A366Q8Q7, A0A366Q909, A0A366R737, A0A366S001, A0A366QHY9, A0A366PSC3, A0A366R685, A0A366NS06, A0A366NR83, A0A366RE72, A0A366P026, A0A366P298, A0A366Q4J4, A0A366QZP0, A0A366Q9V0, A0A366PGF9, A0A366RYF1, A0A366RSA3, A0A366QJB0, A0A366RE50, A0A366QC02, A0A366PJS9, A0A366PTB2, A0A366Q4I7, A0A366QQ61, A0A366Q3H7, A0A366Q2F5, A0A366RV94, A0A366RKF4, A0A366PYT4, A0A366PSP3, A0A366R173, A0A366RE58, A0A366QPK3, A0A366PGR0, A0A366R225, A0A2KOW5D0, A0A2KOUTI6, A0A2KOW931, A0A2KOWC09, A0A2KOUTH2, A0A2KOWV95, A0A2KOWIM1, A0A2KOW2B0, A0A2KOW2K6, A0A2KOVVG2, A0A2KOW1N5, A0A2KOUTG7, A0A2KOW8T1, A0A2K0W365, A0A2KOVVT0, A0A2KOWAU9, A0A2KOW3Y3, I1S305, A0A098DHT5, A0A098DJ50, I1RTL3, I1S3P5, A0A098DZ54, I1RLE4, I1S5T9, I1RTD2, I1RV98, I1S3P4, A0A098D7I4, A0A098DJX2, A0A098DZM5, I1RA55, I1RXMO, I1R9J0, I1RKL8, I1S2Z6, A0AIC3YKN6, A0A098E223, A0AIC3YHH6, A0AIC3YKD6, A0AIC3YKB1, A0A2H3HMC5, A0A2H3GRJ6, A0A2H3HSA2, A0A2H3GYF5, A0A2H3FZY4, A0A2H3HDH3, A0A2H3G941, A0A2H3H4N8, A0A2H3GNB0, A0A2H3GIG1, A0A2H3FML8, A0A2H3G768, A0A2H3H9N3, A0A2H3HFP0, A0A2H3GGM8, A0A2H3FHF7, A0A2H3GHH8, A0A397VR36, A0A397W4J7, A0A397VYK2, S3D022, S3CIG6, S3D8B4, S3DNA8, H0EQA9, K3WFA2, K3WFA4, K3WN51, K3WWN0, K3WCW4, K3W5W5, K3WZR8, K3WJD0, K3WPX5, K3WPX2, K3WZD7, K3WD79, K3XBK6, K3WN48, K3WWN3, K3WI68, K3WRX0, K3WI73, K3WCV9, K3WY68, K3WCW1, K3WPX3, K3X667, K3WHY8, K3WGZ7, K3WCW6, K3WI69, K3WI70, K3WRY9, K3XDL0, K3WCV8, K3WP93, K3WPX4, K3WGZ2, K3WCW3, K3WCU2, K3WCU9, K3WK48, K3WFA3, A0A183BI04, A0A183CL17, A0A183BNL9, A0A183CQJ0, A0A183CDV8, A0A183CRD4, A0A183CBM9, A0A183BUJ7, A0A183BWP9, A0A183CDW0, A0A183CBA0, A0A183BWW1, A0A1A9VE33, A0A1A9UM99, A0A1A9UMI6, A0A1A9UK14, A0A1A9UT06, A0A1A9UM92, A0A1A9UV32, A0A1A9WZX2, A0A1A9WYV2, A0A1A9X167, A0A1A9X2J2, A0A1A9WP94, A0A1A9X5N1, A0A1A9X170, A0A1A9W7X4, A0A1A9XI74, A0A1A9XI75, A0A1A9YJ04, A0A1A9XI83, A0A1A9XZJ7, A0A1A9XDF9, A0A1A9XLY9, A0A1A9Y8K0, A0A1B0FQZ0, A0A1B0G5Z0, A0A1B0FMH8, A0AIB0F9A8, A0AIB0FN27, A0AIB0FJ31, A0AIB0FF31, A0AIB0ACN4, A0A1A9ZN42, A0AIB0AFV3, A0A1A9Z7T1, A0AIB0ACN9, A0A1A9Z0F1, A0AIB0ACN0, A0AIB0AAP1, A0AIB0AYB5, A0AIB0B913, A0AIB0BQV5, A0AIB0BRF1, A0AIB0AMM5, A0AIB0B615, A0AIB0BSL7, C6ZRQ3, C6ZS29, K7L2L4, I1L742, A0A0R0EVN8, I1KE73, I1LSR9, I1LSR6, A0A0R0KG24, K7M8Z4, K7KXE1, I1LSX3, I1MIG9, A0A0R0HEP2, K7KXI5, A0A0R0H4D6, I1ME78, K7KXH0, K7LCV5, I1MI78, K7M2T9, I1LST1, A0A0R0L9K9, K7M1S0, K7K3A5, I1KZA4, K7M1R9, K7KBW2, A0A0R0FWY9, K7LU18, A0A0R0IPR8, IlKE80, A0A0R0JLUI, K7K3A6, I1K7Y3, K7LU19, K7LUX6, I1M2F1, A0A0R0IHT4, K7K9A9, I1KEB9, A0A0R0GW83, A0A0R0JNK4, K7LUW1, K7KXF2, IlLSYl, A0A0R0KJ29, K7KXI4, I1LGT5, K7MZ69, A0A0R0HDR1, I1KEC5, I1KE67, K7M2F6, C6ZRW7, A0A0R0IRG6, K7M2E9, A0A0R0H8A8, I1KE60, A0A0R0JLS9, K7M8T0, K7MCU5, K7KXH6, A0A0R0HHK1, A0A0R0H240, K7L050, I1M2E7, K7LAQ9, A0A0R0HUZ3, A0A0R0HHN2, A0A0R0H5B6, K7LAS7, I1KEA9, A0A0R0HHS7, A0A0R0KYQ5, A0A0R0JT19, K7LUV4, K7KXC8, A0A0R0HFB3, K7M046, A0A0R0HDU4, I1MKV1, A0A0R0JM62, A0A0R0JM46, K7M1R8, I1MI79, K7M8Z5, A0A0R0H590, A0A0R0EG65, K7MCU6, A0A0R0J530, A0A0R0HEH1, A0A0R0JRN4, K7LUW3, I1JTI1, K7MCU7, A0A0R0JRK8, K7M2F1, A0A0R0INR7, A0A0R0JLL7, K7K3A1, A0A0R0FWT7, A0A0R0H6A2, K7KXD7, K7LQJ4, I1KZA6, A0A0R0EHM4, K7LCV6, A0A0R0JLQ4, A0A0R0FXR9, I1LSY2, K7KXH7, A0A0R0H7L6, K7LUX4, I1M2F0, A0A0R0H5I9, A0A0R0K7P2, A0A0R0GTK5, K7KBW3, I1KE90, K7KXI2, A0A0R0H043, K7M1R3, I1KF87, K7KDF6, K7KXH2, A0A0R0JLC9, I1M3F1, A0A0R0JVL9, A0A0R0HEE2, A0A0R0HFQ6, A0A0R0G425, K7KXF3, A0A0R0H539, IlKE88, I1KE68, A0A0R0J260, I1LSW7, I1M5U0, K7KI11, I1KE70, I1LAB6, A0A0R0J0N7, K7LAS8, I1KKX7, K7KXE2, A0A0R0KD14, K7MCT9, I1LSX8, K7LAQ6, A0A0R0G5B0, K7KXH9, A0A0R0JLZ5, A0A0R0H884, K7L8G9, A0A0R0FXA7, I1KZ76, K7LUV3, A0A0R0IC73, K7LUY2, K7KXH5, K7LVZ6, I1KZ77, K7M2F0, I1JNE6, A0A0R0EWT5, K7K3A2, I1MNZ4, K7M1R5, A0A0R0JTU4, I1M2E9, K7MYD3, I1JPX6, K7LVZ4, A0A0R0H5T0, I1LSR5, IlKEB0, K7LUW9, K7M1R6, I1KQR8, A0A0R0H5Y2, I1M2E5, K7LVZ3, K7LUSO, I1LLA6, K7KXD3, A0A0R0JLM5, K7KD19, C6ZRU8, K7MG77, I1LSY4, A0A0R0GV60, IlKEB1, A0A0R0H7N3, A0A0R0G5X7, A0A0R0H529, A0A0R0HEQ7, K7LUV6, A0A0R0JRU4, I1KEB7, A0A0R0H5H4, A0A0R0L9J9, A0A0R0H871, A0A0R0FNK4, K7LUV7, A0A0R0HHZ0, A0A0R0JLR9, K7KD21, K7MIR7, A0A0R0GTN5, K7KXI0, K7MCU9, K7M1R2, I1KE64, A0A0R0LIX3, K7KT18, K7LUW8, I1KEA7, A0A0R0H5K4, A0A0R0HFT7, A0A0R0JT32, A0A368UHR2, K7KXH1, A0A445IKH7, A0A445K4J5, A0A445IBB4, A0A445IC32, A0A445IBG5, A0A445KEL5, A0A445I9N1, A0A445IA22, A0A445KEC4, A0A0B2R8A6, A0A0B2RTJ3, A0A0B2R410, A0A0B2RYE8, A0A0B2R3Z9, A0A0B2NQG4, A0A0B2SR89, A0A0B2SHQ9, A0A0B2Q0P0, A0A0B2QFX4, A0A445LDU1, A0A0B2PJD4, A0A0B2PLC0, A0A0B2R8Z3, A0A0B2P2R6, A0A0B2SNF0, A0A0B2QYMO, A0A445KEJ3, A0A0B2SG46, A0A0B2SPZ0, A0A0B2NXR2, A0A0B2S035, A0A0B2S336, A0A0B2SSB0, A0A445GWF0, A0A445IFF3, A0A445HSY4, A0A445IB72, A0A0B2RZB0, A0A445IBT0, A0A0B2SIU2, A0A0B2PEM2, A0A0B2PA07, A0A0B2RQ81, A0A0B2QDD9, A0A0B2NSG5, A0A0B2PTL8, A0A0B2PCY8, A0A0B2QIS7, A0A0B2PHY5, A0A0B2QXQ7, A0A0B2SRB5, A0A0B2QH24, A0A0B2QEU6, A0A0B2SLN0, A0A0B2NSK2, A0A0B2PEM8, A0A0B2PD35, A0A0B2R1I6, A0A445L056, A0A445IBD0, A0A445KEK2, A0A0B2R2T4, A0A445JAL8, A0A445HQ61, A0A445JPE3, A0A445FGF3, A0A445M1W2, A0A445HN76, A0A445IA45, A0A445HQC5, A0A445HQE3, A0A445JV37, A0A445HPY3, A0A445KEI0, A0A445IB85, A0A445KE92, A0A445HPU3, A0A445HQB7, A0A445KEQ7, A0A445KVH1, A0A445KEH0, A0A0B2RLN3, A0A445KEG9, A0A445KEI1, A0A445L055, A0A445I607, A0A445HPX9, A0A445JAP1, A0A0B2QGK7, A0A445KF02, A0A445GWE9, A0A445JAK5, A0A445KV70, A0A445GGC1, A0A445L8H0, A0A445HSS9, A0A445KZL6, A0A445IZ56, A0A445KF53, A0A0B2PCH0, A0A0B2QFX9, A0A0B2P0P0, A0A0B2RPE1, A0A0B2NX34, A0A0B2P9T3, A0A0B2S2G1, A0A445I3H0, A0A445HXA0, A0A445KEJ2, A0A0B2RKG5, A0A0B2QGL4, A0A0B2P4X3, A0A445IZ50, A0A445IZ45, A0A445HPU8, A0A445IA11, A0A445KED2, A0A445KEI5, A0A445I9L6, A0A445IBA1, A0A445L054, A0A445GQ74, A0A445HQD2, A0A445GQ03, A0A445HQD8, A0A445HQC1, A0A445HQK7, A0A445HST5, A0A445KF01, A0A445HPX5, A0A445JPF3, A0A445KEN4, A0A445KEU4, A0A445IB73, A0A445FIR5, A0A445IBH5, A0A445GWZ7, A0A445I9N6, A0A445JPH7, A0A445HQ82, A0A445KET6, A0A445KVD1, A0A445L5S4, A0A445IBB3, A0A445LQP4, A0A0B2PDS8, A0A0B2QBU1, A0A445KEMO, A0A445HQA5, A0A445K4C6, A0A445I9M8, A0A445HQG0, A0A445JPL0, A0A445JAK4, A0A445KEF7, A0A445JPB5, A0A0B2RX25, A0A0B2SJF3, A0A0B2SL58, A0A0B2QH31, A0A445FNQ5, A0A445HPS5, A0A445KEW3, A0A445I3X8, A0A445FNS9, A0A445LBW9, A0A445L5L3, A0A445HQ49, A0A445HQ40, A0A445HPZ2, A0A445KED1, A0A445HQK9, A0A445HSU7, A0A445IB74, A0A445GQ81, A0A445KEK1, A0A445KEA4, A0A445HSU4, A0A445HN83, A0A0B2R9H1, A0A0B2PX39, A0A0B2PQ09, A0A0B2R9G6, A0A0B2NXL9, A0A0B2RS26, A0A0B2QYI7, A0A0B2QVJ9, A0A0B2NXT7, A0A0B2QCR2, A0A0B2PX80, A0A445KE89, A0A445HQ13, A0A445GQ70, A0A445KES8, A0A445HQG2, A0A0B2SMR2, A0A0B2R2T0, A0A445JAN3, A0A445KED5, A0A0B2RR49, A0A445JPM6, A0A445HQ19, A0A445IC34, A0A445M1S7, A0A445I3D7, A0A445JJ41, A0A0B2PNA6, A0A0B2R151, A0A445HT18, A0A445JPC2, A0A445IC64, A0A445FF01, A0A0B2QCR6, A0A445IB88, A0A445KEM6, A0A445KEJ0, A0A445F251, A0A445GJ58, A0A445HSW1, A0A445HPQ4, A0A445JPA6, A0A445HSU3, A0A445L7J1, A0A0B2PE82, A0A445HPU5, A0A445J754, A0A445HPW8, A0A445KEB3, A0A445GQ56, A0A445HPX8, A0A445HSUO, A0A445I9M0, A0A445HPV7, A0A445HQK5, A0A445HGW0, A0A445HQ03, A0A445K7Z2, A0A445KF30, A0A445I9P9, A0A445HPY4, A0A445JP99, A0A445JYL2, A0A445KEJ4, A0A445I9P3, A0A445L7J0, A0A445JB06, A0A445KEB9, A0A445IB86, A0A445HQF8, A0A445GWL4, A0A445L9S1, A0A445HSV5, A0A445KEE3, A0A445KEX3, A0A445JTF8, A0A445JB16, A0A445KET4, A0A445JXZ8, A0A0B2SV99, A0A0B2PTA3, A0A445GQ60, A0A445HPZ9, A0A445HQE5, A0A445JPB6, A0A445JPB2, A0A445KGH5, A0A445HQD5, A0A445JYL7, A0A445KEK3, A0A0B2RV84, A0A445IZ62, A0A445HQ72, A0A0B2NU47, A0A445M242, A0A0B2PI23, A0A0B2NVK2, A0A0B2NXP7, A0A445JPA5, A0A445KEY2, A0A445GWI7, A0A0B2RRS1, A0A445GWF9, A0A445KEJ8, A0A0B2QVY8, A0A445HPR4, A0A0B2PU27, A0A445M229, A0A0B2SLA2, A0A445GM55, A0A0B2PGJ9, A0A445GMK7, A0A0B2R815, A0A445GWF8, A0A445IC59, A0A445GM53, A0A445KEG8, A0A445HQK6, A0A445I9S8, A0A0B2R405, A0A445KEG0, A0A139AN84, A0A139AWG3, A0A3P7LQ98, A0A3P6SB28, A0A183E3M5, A0A183DSQ9, A0A183D9Z8, A0A3P6S9V6, A0A3P7MR12, A0A183D9N7, A0A183CUV3, A0A183CZ86, A0A183CVQ5, A0A183E7Y0, A0A183D9A1, A0A183CUC2, A0A183DYS1, A0A183D4T0, A0A183CZ38, A0A183EWR6, A0A183D6N3, A0A183DDG5, A0A183DJR2, A0A183D2S8, A0A150G4C6, A0A150G869, A0A150GQC4, A0A150GM43, A0A150GGI9, A0A150FY45, A0A150G7V9, A0A150G6Z0, A0A150GXZ1, A0A150GXR5, A0A150GAR7, A0A150G2N9, A0A150G968, A0A150FX17, A0A150G2Z2, A0A150GY18, A0A150FX43, A0A150G717, A0A150GGC8, A0A150GQP5, A0A150GGE4, A0A150G565, A0A150G8B8, A0A150FU74, A0A452H3F3, A0A452H966, A0A452H952, G3RXF7, G3R4W6, G3RNU9, G3R2X1, A0A2I2ZIP3, A0A2I2YSL6, G3RZ73, A0A2I2ZYB7, G3QLL7, A0A0B0N1R7, A0A0B0PPF2, A0A0B0NU75, A0A0B0MYX5, A0A0B0MTX0, A0A0B0MXU6, A0A0B0MZL4, A0A0B0NP12, A0A0B0N0U6, A0A0B0N855, A0A0B0PYF7, A0A0B0N5G9, A0A2P5QME1, A0A2P5WSV9, A0A2P5R530, A0A2P5QAQ1, A0A2P5RJB5, A0A2P5PX52, A0A2P5R799, A0A2P5XQ00, A0A2P5R525, A0A2P5YJR7, A0A2P5WUP4, A0A2P5XQE1, A0A2P5W537, A0A2P5QYR4, A0A2P5RJD9, A0A2P5SAL6, A0A2P5R4Z5, A0A2P5RMX0, A0A2P5Y4V6, A0A2P5XRT3, A0A2P5RJC7, A0A2P5XLL9, A0A2P5QP15, A0A2P5YQK4, A0A2P5Q2I3, A0A2P5W0S5, A0A2P5RX37, A0A2P5RFB1, A0A2P5XRP7, A0A2P5YMC7, A0A2P5XPY8, A0A2P5W117, A0A2P5Q5R1, A0A2P5QZB7, A0A2P5Y5I5, A0A2P5SRC9, A0A2P5RY44, A0A2P5WP31, A0A2P5RWS6, A0A2P5RMT2, A0A2P5Y6U2, A0A2P5R4X6, A0A2P5YX52, A0A2P5XNK6, A0A2P5WUK6, A0A2P5XT89, A0A2P5SBC1, A0A2P5SP55, A0A2P5W510, A0A2P5RJE1, A0A2P5QIM8, A0A2P5SJY1, A0A2P5QJX4, A0A2P5S011, A0A2P5QSI7, A0A2P5WKV8, A0A2P5RN97, A0A2P5RZU3, A0A2P5XM03, A0A2P5S017, A0A2P5WUK4, A0A2P5QAP8, A0A2P5W0S1, A0A2P5RMG9, A0A2P5VYB6, A0A2P5Y509, A0A2P5W461, A0A2P5Y5H9, A0A2P5X2H7, A0A2P5XQF5, A0A2P5QC40, A0A2P5WKV0, A0A2P5R7M3, A0A2P5WII7, A0A2P5XRR5, A0A2P5S3Y3, A0A2P5PX26, A0A2P5XT09, A0A2P5Y4V2, A0A2P5XYY9, A0A2P5YJS7, A0A2P5RWY7, A0A2P5SP77, A0A2P5RWV6, A0A2P5R4Z6, A0A2P5R7C6, A0A2P5WUM4, A0A2P5R7B5, A0A2P5YX45, A0A2P5R500, A0A2P5W2X4, A0A2P5QPE5, A0A2P5SP93, A0A2P5RMJ4, A0A2P5Q5Q4, A0A2P5Y5L5, A0A2P5R4Y1, A0A2P5Y9P7, A0A2P5RZT0, A0A2P5XRP9, A0A2P5RUA4, A0A2P5Y109, A0A2P5R7B6, A0A2P5QAB2, A0A2P5QJY8, A0A2P5SV42, A0A2P5WSV7, A0A2P5RKW8, A0A2P5WKX2, A0A2P5QJM8, A0A2P5SE83, A0A2P5SP86, A0A2P5R7B3, A0A2P5Q4C5, A0A2P5SE70, A0A2P5RMS7, A0A2P5YQL9, A0A2P5WKU3, A0A2P5R5J1, A0A2P5RN93, A0A2P5SP50, A0A2P5XRU3, A0A2P5VNQ9, A0A2P5PXQ4, A0A2P5R7A8, A0A2P5RMU3, A0A2P5RU57, A0A2P5RLV9, A0A2P5XRW6, A0A2P5XRW3, A0A2P5R7B4, A0A2P5WKW0, A0A2P5VXE2, A0A2P5VQP7, A0A2P5QAS2, A0AIU8LTN4, A0AIU8IY86, A0AIU8JWT7, A0A1U8MSL2, A0A1U8NIT3, A0A1U8JW83, A0A1U8M088, A0A1U8PII4, A0A1U8MW63, A0A1U8PIK8, A0A1U8PIH7, A0A1U8NKW8, A0A1U8KUL9, A0A1U8MSI7, A0A1U8MQ96, A0A1U8MJ01, A0A1U8INSO, A0A1U8J3Z6, A0A1U8MZ75, A0A1U8J8V8, A0A1U8K7I4, A0A1U8HRC6, A0A1U8JD70, A0A1U8K7G9, A0A1U8MW53, A0A1U8M3G9, A0A1U8PSO2, A0A1U8MWK7, A0A1U8IQI9, A0A1U8IQ79, A0A1U8J5M7, A0A1U8PL10, A0A1U8PJ03, A0A1U8KPI2, A0A1U8KDH7, A0A1U8KPN3, A0A1U8NGI9, A0A1U8MQB6, A0A1U8N4R6, A0A1U8KIK7, A0A1U8NU95, A0A1U8K568, A0A1U8JUG2, A0A1U8KP21, A0A1U8HJH7, A0A1U8MU48, A0A1U8J417, A0A1U8MTB7, A0A1U8IZ76, A0A1U8MSL8, A0A1U8KFJ8, A0A1U8IIC3, A0A1U8PRU7, I0B675, A0AIU8NVM4, A0AIU8N3Z5, A0AIU8J436, A0AIU8MWV1, A0A1U8L9V7, A0A1U8MU65, A0A1U8K7H3, A0A1U8J5E0, A0A1U8NTW1, A0A1U8J456, A0A1U8LQA8, A0A1U8LU33, A0A1U8MJ39, A0A1U8KJM4, A0A1U8N710, A0A1U8IIU3, A0A1U8HZZ8, A0A1U8JME3, A0A1U8MSV8, A0A1U8LQ71, A0A1U8KI27, A0A1U8N0I2, A0A1U8NVW2, A0A1U8JTJ3, A0A1U8MU44, A0A1U8HXT9, A0A1U8KN26, A0A1U8NAH6, A0A1U8MPV7, A0A1U8NWF2, A0A1U8JA35, A0A1U8MWB9, A0A1U8NWR2, A0A1U8MU69, A0A1U8N0D3, A0A1U8MXQ1, A0A1U8KM96, A0A1U8NTW9, A0A1U8KM40, A0A1U8KUG4, A0A1U8MSS6, A0A1U8HR08, A0A2P0JA21, A0A1U8N932, A0A1U8LW85, A0A1U8NVG7, A0A1U8MT98, A0A1U8L9U6, A0A1U8NWQ7, A0A1U8JLD2, A0A1U8NWF7, A0A1U8JPV5, A0A1U8PJ85, A0A1U8IIP2, A0A1U8NU86, A0AIU8IJ22, A0A0D2NHR6, A0A0D2S9W7, A0A0D2SHQ6, A0A0D2R293, A0A0D2UMW3, A0A0D2U925, A0A0D2UF09, A0A0D2R2A3, A0A0D2N3S2, A0A0D2Q7B3, A0A0D2NJM3, A0A0D2Q7C6, A0A0D2U4N2, A0A0D2U7M3, A0A0D2VH44, A0A0D2R1G8, A0A0D2U3X9, A0A0D2NYP5, A0A0D2NJL3, A0A0D2S4R5, A0A0D2RIS5, A0A0D2SLX3, A0A0D2W2H3, A0A0D2Q066, A0A0D2R018, A0A0D2VAH9, A0A0D2NJN0, A0A0D2VJL8, A0A0D2T102, A0A0D2Q7A2, A0A0D2U3Z0, A0A0D2VAL8, A0A0D2V8Y9, A0A0D2Q2C4, A0A0D2TKF7, A0A0D2NHP9, A0A0D2S2B9, A0A0D2TEH9, A0A0D2ST81, A0A0D2PWM1, A0A0D2SZD9, A0A0D2M695, A0A0D2RIT8, A0A0D2Q9G7, A0A0D2QW76, A0A0D2NG46, A0A0D2RFL5, A0A0D2U430, A0A0D2Q063, A0A0D2R2E2, A0A0D2QHX8, A0A0D2V1I8, A0A0D2PWL4, A0A0D2NHQ3, A0A0D2R874, A0A0D2SKU2, A0A0D2NJR3, A0A0D2NHR3, A0A0D2S5T3, A0A0D2ST84, A0A0D2VA22, A0A0D2T0Z5, A0A0D2R008, A0A0D2Q078, A0A0D2NHS1, A0A0D2TRL9, A0A0D2R2W6, A0A0D2TF92, A0A0D2RIT4, A0A0D2M871, A0A0D2T212, A0A0D2NTC5, A0A0D2R013, A0A0D2R877, A0A0D2TUH1, A0A0D2TTI3, A0A0D2Q2G3, A0A0D2RSA1, A0A0D2TQ92, A0A0D2VUT2, A0A0D2M7Q0, A0A0D2TWT0, A0A0D2RAR6, A0A0D2Q9E3, A0A0D2UOY6, A0A0D2M841, A0A0D2UGZ9, A0A0D2Q059, A0A0D2PQT1, A0A0D2SUB8, A0A0D2UNZ7, A0A0D2V1J1, A0A0D2Q081, A0A0D2R033, A0A0D2RQB0, A0A0D2M672, A0A0D2UF05, A0A0D2UUZ6, A0A0D2W712, A0A0D2UPR4, A0A0D2UF15, A0A0D2SYV9, A0A0D2V121, A0A0D2Q2D6, A0A0D2NIU8, A0A0D2UCR1, A0A0D2QA06, A0A0D2Q2G4, A0A0D2RIU4, A0A1B6MBH5, A0A1B6MT83, A0A1B6KHB7, A0A1B6LPX0, A0A1B6MRL1, A0A1B6KWB9, A0A1B6KTZ0, A0A023AXR7, A0A023AYU7, A0A023B052, A0A023BAV3, A0A023B0X7, A0A023B381, A0A023B0X6, A0A023AXP8, A0A023B1C3, A0A023B0P6, L1JJD2, L1JJX0, L1JTS3, L1JKL7, L1JCN2, L1IX89, LIK 113, L IIWJ0, LIIHZ2, LIJCP1, A0A3P4KMI5, A0A3P4RU50, A0A3P4RW44, A0A3P4RAU2, A0A3P4RWP0, A0A0L7QLL2, A0A0L7R1I8, A0A0L7RF61, A0A0L7R6V2, A0A0L7R538, A0A0L7RFI2, A0A212ACR4, A0A086XX93, A0A212AJW7, W6NGT9, W6NNJ2, A0A158QM82, A0A0N4W5U3, A0A158QK55, A0A3P7UET5, A0A3P7TV91, A0A3P7XBC6, A0A3P7TQB2, A0A0N4WUQ0, A0A0N4W8T5, A0A158QNJ5, A0A0N4WA48, A0A0N4X5R8, A0A0N4WPF3, A0A0N4X266, A0A0N4WAZ2, A0A158QLJ4, A0A158QPL6, A0A158QR36, A0A158QKJ2, A0A0N4WU40, A0A0N4WJV0, A0A0N4WKR9, A0A0N4WC06, A0A158QRG0, A0A0N4W2R6, A0A0N4WAT5, A0A0N4WNL0, A0A0N4WCR5, A0A0N4WS57, A0A0N4WAG3, A0A091PRS1, A0A091PH99, A0A091PGF6, A0A091PJK8, A0A126UY30, A0A1M7I344, W1NCY1, A0A074SYS7, A0A074SUJ0, A0A074TKE0, A0A074SSB4, A0A074T9T4, A0A074SY06, A0A074SSP2, A0A074SRW4, A0A074T002, A0A074SV30, A0A074SUT3, A0A074SME0, A0A074T415, A0A074T0Q6, A0A074TPN2, A0A074SMC6, A0A2G9HBA5, A0A2G9I607, A0A2G9GS19, A0A2G9HFW2, A0A2G9G3H0, A0A2G9GBC9, A0A2G9H9N3, A0A2G9FWW5, A0A2G9G020, A0A2G9H900, A0A2G9G8U4, A0A2G9H5E2, A0A2G9G0T8, A0A2G9G7H6, A0A2G9G7F0, A0A2G9HQH7, A0A2G9GLL5, A0A2G9I602, A0A2G9HMW9, A0A2G9H7Y4, A0A2G9HVS1, A0A2G9H5H4, A0A2G9FW99, A0A2G9G9M9, A0A2G9FX93, A0A2G9G0T2, A0A2G9GFB5, A0A2G9HQJ0, A0A2G9I4A0, A0A2G9G0G1, A0A2G9HNZ9, A0A2G9GUR0, A0A2G9H3N1, A0A2G9FY38, A0A2G9G8A6, A0A2G9GBD2, A0A2G9HQ39, A0A2G9HP33, A0A2G9G948, A0A2G9G8Y1, A0A2G9GK44, A0A2G9G7D7, A0A2G9IBJ2, A0A2G9G5U6, A0A2G9HBA6, A0A2G9G7K3, A0A2G9G7Z8, A0A2G9G482, A0A2G9GNC7, A0A2G9HBD0, A0A2G9GA17, A0A2G9GNR7, A0A2G9GGQ5, A0A2G9I619, A0A2G9G874, A0A2G9GY40, A0A2G9HZV9, A0A2G9G7A6, A0A2G9GZU4, A0A2G9GC79, A0A2G9G877, A0A2G9G5N8, A0A2G9I606, A0A2G9HM51, A0A2G9GUP3, A0A2G9GEQ2, A0A2G9HNL0, A0A2G9H7Q3, A0A2G9G4X6, A0A2G9IBI1, A0A2G9GJX1, A0A2G9I6C9, A0A2G9G5B6, A0A2G9GB96, A0A2G9HHD9, A0A2G9GFY4, A0A2G9H5D1, A0A2G9GFA7, A0A2G9GBC7, A0A2G9H719, A0A2G9H716, A0A2G9IAYO, A0A2G9GX58, A0A2G9GA47, A0A2G9GKN2, A0A2G9FXC4, A0A2G9FY42, A0A2G9FXI3, A0A2G9H718, A0A2G9HP44, A0A2G9GMC6, A0A2G9HMZ3, A0A2G9I5Y5, A0A2G9G1J5, A0A2G9H762, A0A2G9GKG3, A0A2G9IBT8, A0A2G9GT09, A0A2G9G6Z8, A0A2G9I585, A0A2G9HHH7, A0A2G9HTZ3, A0A2G9HHL8, A0A2G9H5I0, A0A2G9I2J4, A0A2G9HP00, A0A2G9GSG6, A0A2G9FYE8, A0A2G9H932, A0A2G9G8L4, A0A3Q2WKE2, A0A3Q2VWA3, A0A3Q2VMX2, A0A3Q2W714, A0A3Q2V9X5, A0A3Q3BK39, A0A3Q3CJW9, E2BZU8, E2BV16, E2C0I8, E2C2K1, E2B4Q6, E2B676, A0A2C9D802, A0A251VNS3, A0A251T9D7, A0A251S5U8, A0A251RZC7, A0A251URM3, A0A251U4N7, A0A251SPB8, A0A251U419, A0A251TB44, A0A251VQC9, A0A251TTA7, A0A251T9Z3, A0A251T8T6, A0A251S5S5, A0A251USV7, A0A251V9P5, A0A251SNMO, A0A251RSS9, A0A251ST77, A0A251U4U7, A0A251URK0, A0A251V8P7, A0A251V4L5, A0A251T8Z6, A0A251V3F7, A0A251SGZ7, A0A251UQR4, A0A251S8P2, A0A251SC29, A0A251ULP0, A0A251T9K9, A0A251T9M7, A0A251TAJ6, A0A251T967, A0A251U5M7, A0A251UIR2, A0A251RT61, A0A251TNE9, A0A251T9J2, A0A251THT3, A0A251TZ72, A0A251U550, A0A251U473, A0A251TNN4, A0A251UQL0, A0A251RSW7, A0A251U760, A0A251VDL9, A0A251UDJ8, A0A251U3Z2, A0A251T939, A0A251V5W8, A0A251TDT0, A0A251SDU7, A0A251T4J5, A0A251SMJ5, A0A251TA85, A0A251S960, A0A251T8Z8, A0A251UJMO, A0A251T4T1, A0A251VPZ1, A0A251TCA5, A0A251TAB3, A0A251SW79, A0A251RXKO, A0A251S8G7, A0A251TSR7, A0A251U418, A0A251T909, A0A251TAA8, A0A1Y3BZF9, A0A251T8Y8, A0A251RSP8, A0A251SSD8, A0A251T949, A0A251RMY8, A0A251TA21, A0A251T2S5, A0A251RN23, A0A251T906, A0A251UL16, A0A251RV73, A0A251RTF5, A0A251S990, A0A251STX9, A0A251S8Y4, A0A251URH5, A0A251SOK1, A0A251UEQ1, A0A251RNY5, A0A251TL03, A0A251SS56, A0A251TN10, A0A251THY3, A0A251S995, A0A251TA25, A0A251T5L3, A0A251V227, A0A251T959, A0A251TL38, A0A251TAD2, A0A251SXL9, A0A251U5K9, A0A251T266, A0A251U403, A0A251V611, A0A251THH7, A0A251T9X4, A0A251TV70, A0A251TAX5, A0A251RS35, A0A251RS51, A0A251T7B5, A0A251T1U1, A0A251RNF0, A0A251VBF9, A0A251UHX2, A0A251U6B1, A0A251ULD7, A0A251T3N2, A0A251TA13, A0A251T908, A0A251U4V0, A0A251UAKO, A0A251TAL5, A0A251VCQ1, A0A251SMN4, A0A251U509, A0A251U5G8, A0A251SMR2, A0A251UHY6, A0A251TY69, A0A251TCD7, A0A251U4T1, A0A251TJD6, A0A251U476, A0A251UPA1, A0A251T447, A0A251T2R7, A0A251U4S3, A0A251VPX0, A0A251T8U9, A0A251TiW6, A0A251T929, A0A251T9Q8, A0A251T2N3, A0A251ULK0, A0A251TA08, A0A251T9E6, A0A251U5H8, A0A251T585, A0A251UNM9, A0A251SVM1, A0A251SOB8, A0A251TAB8, A0A251U4T7, A0A251VUB1, A0A251U519, A0A251UJH6, A0A251VLZ7, A0A251SM06, A0A251TiY3, A0A251T2F8, A0A251SA55, A0A251TAM7, A0A251RTF9, A0A251RTR7, A0A251U5H1, A0A251UTN4, A0A251UTW0, A0A251SU98, A0A251UQN1, A0A251SNN8, A0A251U3Z3, A0A251USS8, A0A251TMP9, A0A251UTL3, A0A251SS58, A0A251S4B6, A0A251SKT0, A0A251T8U8, A0A251U463, A0A251U4V7, A0A251U422, A0A251TASO, A0A251RMW7, A0A251TAE1, A0A251U8J7, A0A251VPQ8, A0A251U8C6, A0A251SMR8, A0A251TA09, A0A251SS64, A0A251VRM9, A0A251U6A1, A0A251VHM6, A0A251U4T8, A0A251T3G0, A0A2B7WMP5, A0A2B7XNZ2, A0A059LJS3, A0A059LQC7, A0A183GCJ4, A0A183FDS9, A0A183GQL5, A0A183FNG0, A0A183F5N0, A0A183F1S4, A0A183G9U4, A0A183FAM8, A0A183GCA0, A0A183GSN8, A0A183FC10, A0A183FLN4, A0A183GR87, A0A183FV70, A0A183FLD7, A0A183GDB3, A0A183FE94, A0A183FLN5, A0A183GEW6, A0A3P7TAF8, A0A3P7XS44, A0A3P7UBD8, A0A3P7YBC3, A0A3P8CJY4, A0A3P8B899, A0A3P8BPW5, A0A3P7XWA8, A0A3P8FFK8, A0A3P8BG03, A0A3P7Y3L6, A0A3P8AS32, A0A3P8DWK6, A0A3P8CGZ0, A0A3P7YEL9, A0A3P8A3U8, A0A3P7XYB2, A0A3P8C2W2, A0A3P8BGP5, A0A3P8BSP4, A0A3P8E3J7, A0A3P7X235, A0A3P8DNT7, A0A3P7WBJ4, A0A2A4KOR0, A0A2A4J5Y3, A0A2A4J736, A0A2A4IUN6, A0A2A4JF45, A0A2A4JFF2, A0A2A4K3D6, A0A2A4JED9, A0A2A4IXP5, T1FFM4, T1F4T5, T1FEH2, T1F969, T1FKL0, T1FFM5, T1EQR6, T1F4U5, T1F4U0, T1FB09, T1EQR5, T1F1E0, T1FEJ2, TiFUN0, T1F4U7, T1FJS5, T1FFA4, T1F854, T1FVJ6, TIF4U6, TIF4U3, TIFVK2, TIF5M2, TIFEF5, TIFPY2, TIFUT1, TIETQ4, TIFVK1, T1F970, T1EVQ0, T1F9B8, T1FR64, T1FBN8, T1FCJ6, T1FJA3, T1F4T4, T1FVKO, T1F4T9, T1FDP6, T1EQR7, TiFUN1, T1EP89, T1ET71, T1FFD2, T1FDN8, T1FEJ8, T1F4T6, T1EQP1, T1F4U4, T1FPT1, T1F960, T1FLN5, T1FB69, T1F954, T1EZ95, T1EQP5, T1FV32, T1FKK7, T1F659, T1FLC6, T1FJL9, T1FDA4, T1F4U8, T1F916, T1F973, T1FHL6, TiFJN5, T1FUI3, T1FLN6, T1FJM1, T1EQP4, T1F4T3, T1FBM8, T1FS89, T1EZ93, T1FPY5, T1FF45, TiFEG8, T1FPZ1, T1EQN9, T1FVV8, T1EPB3, T1FLR7, T1F975, T1FFM6, T1FFG1, T1FBA6, T1FDR4, T1FBP2, T1F682, T1FPY3, TIEQP3, TIF4U2, TIFBN9, TIF974, TIFG81, TIF4V1, TIFBM7, TIF4T7, TIFG80, T1FH28, TiFUI8, TiFB01, T1ETA4, T1FB02, T1F4T8, T1EQR0, T1F174, A0A399RHT4, A0A399RLR9, A0A399RSL7, A0A399RMI5, A0A2D8YFK7, A0A0P6JB42, G5BZW9, G5BNV2, G5BNV1, G5B0R1, A0A0P6IYV4, A0A0P6JIW8, A0A0P6J2P0, G5BC53, A0AII7W716, A0AII7XH77, A0A1I7WWV5, A0A1I7XSR7, A0A1I7XAF8, A0A1I7XHQ1, A0A1I7X146, A0A1I7XM37, A0A1I7X745, A0A1I7XHZ8, A0A1I7XII8, A0AII7X5P0, A0AII7WM55, F2LUA4, A0A3Q2XCJ9, A0A3Q3D5K9, A0A3Q2YZJ7, A0A3Q2YUT9, A0A3Q2YLY3, A0A2P2ICY2, Q9SEB5, Q9SEB2, Q9SEB3, Q9SEB6, Q9SEB4, Q9SEC5, A0A0F7ZU04, V5L2N5, V5L3N5, V5L3U0, A0A3MOJH56, A0A3MOKB79, A0A3MOKPT5, A0A286IFF5, A0A317PT30, A9D968, A0A098TDW6, A0A0F2PZU5, A0A0F7PKQ6, A0A2G2DJB6, A0A1B6IND1, A0A1B6JBA6, A0A1B6IQ37, A0A1B6HRP0, P03951, P00747, P03952, P26927, P14210, Q02325, Q15195, Q2TV78, C9WSJ4, G3XAK1, B7Z3P3, A0A0F7G8J1, H0Y596, B4DMX2, C9J075, A8K6K9, C9JS80, B2R7F8, Q59H59, Q9UEG0, C9JCT1, C9JDP4, F8WCD6, Q9UE70, Q53QL7, E9PBC5, B4DPN1, B4DPQ8, A0A2R8YEI1, A6PVI2, B7Z538, A8K9A9, H0YAC1, D6RB32, Q5TEH5, C9WSJ3, B7Z250, Q13208, Q53GN8, A0A2R5GHI4, A0A2R5GZ90, A0A2R5G055, A0A2R5G2W7, A0A2R5GHT1, A0A2R5GPJ4, A0A2R5GQ72, A0A2R5GFH2, A0A2R5GZ49, A0A2R5GMR6, A0A2R5FCZ9, A0A2R5GLX6, A0A2R5GNL7, A0A2R5GI37, A0A2R5GR48, A0A2R5GP36, A0A2R5GCC8, A0A2R5GTZ6, A0A2R5GIQ6, A0A2R5GBG8, A0A2R5GIT4, A0A2R5G3T7, A0A2R5GCH5, A0A2R5GJ87, A0A2R5GGB6, A0A2R5G7Y5, A0A2R5GWX3, A0A2R5GQR5, A0A2R5GUR1, A0A2R5GIX0, A0A2R5GVR7, A0A2R5G8F7, A0A2R5GQH2, A0A2R5GRQ0, A0A2R5GDB9, F2CSY2, MOXR46, A0A287JPN5, F2CQZ9, MOZ8K2, A0A287JKX2, A0A287IUF4, F2CZD5, A0A287KLF9, MOYWF3, MOY2Z8, A0A287JLH6, A0A287N5Q1, A0A287Q5F5, MOX4U6, A0A287JL69, A0A287LS65, MOXR48, A0A287JKB3, A0A287V5S3, F2E6E0, A0A287VXI1, A0A287KLC6, F2E5G0, A0A287VXR9, A0A287KLB4, A0A287JL74, A0A287JLD4, MOX5T3, A0A287JKR9, MOX4U1, MOWTQ2, A0A287JL26, A0A287S531, MOX4U2, A0A287LSB4, MOXH34, A0A287N5X8, A0A287DZN4, MOUVT7, MOY3I5, A0A287JKM2, A0A287SWW6, MOY2Q7, A0A287JKJ1, MOY9R6, A0A287MK73, M0W648, MOX5T4, F2DE34, A0A287KLB0, A0A287JKS4, A0A287LS98, A0A287S544, MOX4U8, A0A287XZA8, MOVMR6, A0A287JPW4, A0A287KLD9, MOVIJ8, A0A287JPJ2, A0A287JPN9, A0A287JKU9, MOYRS9, MOW0Il, A0A287Q137, M0X645, MOVB75, A0A287XIX9, A0A287JVX3, MOVXQ3, MOX4U9, A0A287S793, A0A287KLA1, A0A287HTP8, A0A287JKA6, A0A287XAT4, A0A287LSA0, A0A287HU15, A0A287LS94, A0A287ULQ4, A0A287G0I2, A0A287I9B0, A0A287X4W8, A0A287V603, A0A287JKW2, A0A287VXJ1, A0A287S581, MOUSE4, A0A287G0I0, MOYWF2, MOYRS8, A0A287EG31, A0A287G0I1, A0A287JPP7, A0A287X536, A0A287Q497, A0A287JPV5, F2DPF7, A0A287JKP7, A0A287H0I3, F2E9C6, A0A287SY82, A0A287H0K6, A0A287JKX4, A0A287LSB2, A0A287JPY8, A0A287G0J3, A0A287VSN4, A0A287JKP4, F2E736, A0A287E1S1, A0A287JKX3, A0A287JVM6, A0A287IUE8, A0A287NM49, MOWIJ2, F2D8U5, A0A287MNZ7, A0A2R9IZN9, F2CR46, A0A287IUJ8, A0A287S537, A0A287S520, A0A287VS95, A0A287XZG6, A0A287JKV6, A0A287JK91, MOUW74, A0A287H0K5, A0A287XMV2, A0A287ETX1, A0A287EJ56, MOVKE6, A0A287Q400, A0A287IUH9, A0A287H787, A0A287Q1D6, A0A287VSA5, A0A287S509, A0A287H719, A0A287JKP2, F2DZW7, A0A287SK29, A0A287JL87, A0A287LSA4, A0A287IGH2, A0A287EE31, F2DVH7, A0A287XS45, A0A287XIR7, A0A287IGL1, A0A287I9A0, A0A287DZP5, A0A287EG67, MOXKB6, A0A287MK90, A0A287JPN4, A0A287XJ9, A0A287JKI2, A0A287JKU7, A0A287X8T7, A0A287JKL9, MOUS74, A0A287H0Q6, A0A287HTT3, A0A287XAZ9, F2DVP4, A0A287IGC1, A0A287LSA9, A0A287JKZ7, A0A287JDY8, A0A287EXW8, A0A287JPK4, A0A287XJ32, A0A287XS09, A0A287H0I2, A0A287GWW0, A0A287JKA0, A0A287GYF4, A0A287JKJ3, A0A287HU10, A0A287JKV4, A0A287JLG8, A0A287JDY3, MOYMH8, A0A287JKH8, A0A287HTI0, MOWAI6, A0A287I9D8, A0A287LSE2, A0A287LSE8, F2DDM1, A0A287Q5G8, A0A287JKQ6, A0A287LSE1, A0A287LSD9, A0A287S577, A0A287JDM9, A0A287XIY2, MOW0I0, A0A287V653, A0A287JKI8, A0A287EJ83, A0A287JKY5, A0A287JKY7, A0A287H0I8, A0A287JL01, A0A287N0H0, MOW0I5, F2DZS5, MOUIL6, A0A287JL57, A0A287EDZ4, MOUVT6, A0A287LSB9, A0A287H0H7, A0A287LS90, A0A287LSA1, A0A287JKZ5, A0A287JKQ5, A0A287IGK1, MOUDYO, MOWBF9, A0A287JL20, A0A287XIT5, A0A287H0J8, A0A287MK96, A0A287GX15, A0A287JKZ9, A0A287L9W8, A0A287G0I8, A0A287LSA5, A0A287Q632, A0A287JKZ4, A0A287KQ24, A0A287M8A3, A0A287LS92, A0A287S515, A0A287JDV3, A0A287HU21, A0A287XS50, A0A287E1N3, A0A287N0G0, A0A287M896, A0A287JKZ2, MOXZ56, A0A287JKM9, A0A287V7H0, A0A287JKR1, A0A287L9U3, A0A287V5K1, A0A287HU00, F2DV22, A0A287S4V9, A0A287JL54, A0A287NLW9, A0A287LS76, A0A287EGH8, A0A287JKU5, A0A287XU8, A0A287EG37, A0A287E1N0, F2DZG8, F2DWF4, A0A287JDQ7, A0A287KH06, A0A287VXV7, A0A287GFZ2, A0A287MK60, A0A287H0J7, A0A287H0H8, A0A287E1M5, A0A287EJ40, A0A287G0I5, A0A287I9D4, F2D0V2, A0A287HTP3, A0A287XIZ1, F2E792, A0A287H0N6, A0A287JPZ0, A0A287S554, A0A287JLI4, A0A287LS59, A0A287U4M0, A0A287MK62, MOX8T3, F2D0M2, A0A287ETW5, A0A287JKR4, A0A287G0I7, A0A287EG04, A0A287S504, A0A287XAP8, MOUQL7, MOX1P9, A0A287JKQ4, A0A287JKG2, F2DJ24, A0A287JKD6, A0A287T4N7, F2D6A9, A0A287S550, A0A287S507, A0A287JKY8, F2D857, F2E0N1, F2D0S6, A0A287HTS3, A0A287HU22, A0A287N5T2, F2E071, A0A287LSD5, A0A287SWR5, F2DYG0, A0A287E1M9, A0A287TTW0, F2E424, A0A287T1C1, A0A287IGI8, A0A287LSC1, F2DGX1, A0A287N5Y3, A0A287LSD3, A0A287XS04, A0A287HTT9, A0A287S5B0, A0A287LSI8, A0A287JKK0, A0A287TU03, F2DJ90, A0A287S5A2, A0A287JE19, A0A287JKSO, A0A287H785, A0A287G0H8, F2EEQ7, MOX5F1, A0A287LSD4, F2CSL7, A0A287LSF0, A0A287N5T8, A0A287XZF8, MOY1V6, A0A287G0H7, A0A287JK96, A0A287T4Y6, A0A287G0I6, A0A287XSE7, F2DBV1, A0A287LSC8, A0A287JKV9, A0A287KLG4, A0A287JPP4, A0A287JKI4, A0A287JLH1, A0A287JL30, A0A287LS79, A0A287JKX1, A0A287SWV1, A0A287XMW2, A0A287LS85, A0A287SX07, A0A287VXG8, A0A287V5Z3, A0A287JGN6, A0A287S4X9, A0A287JKT0, A0A287XIUO, A0A287IGE2, A0A287JPV4, A0A287JL84, A0A287H0R0, F2DSN1, A0A287IGB2, A0A287SYA0, A0A287L9W9, A0A287SWSO, A0A287VDJ7, M0V508, A0A287XAQ3, F2ED78, A0A287JLE3, A0A287JKK2, A0A287H0L7, A0A287XIX4, A0A287JKY3, A0A287V5G7, A0A287NLN4, A0A287JVK7, MOUR24, A0A287E1L3, F2D4F6, F2DZY6, A0A287JKH3, MOV5N5, MOXKB5, A0A287H0P5, A0A287S580, A0A287JK95, MOYRS5, A0A287S4W4, A0A287H0I7, MOYDWI, F2E6M1, A0A2R9IZN3, F2CXU1, MOUFX9, A0A287MK66, A0A287KLC4, A0A287FZA9, A0A287H0L1, A0A287L9W2, A0A287N0F8, A0A287M890, A0A287JKR0, A0A287S5E8, A0A287JL81, A0A287JL40, A0A287SY15, A0A287V5G1, A0A287Q1Y2, A0A287I9D7, A0A287JKD0, A0A287JKQ0, A0A287EJ45, A0A287JGA6, A0A287HTT1, MOYMI0, A0A287IGH4, A0A287HTR9, A0A287XIR2, A0A287IUF8, A0A287XS55, A0A287X4U2, A0A287KGI7, A0A287JKN7, A0A287LS81, A0A287V5I8, A0A287Q176, A0A287JKE5, A0A287GG05, A0A287JKL1, A0A287IUE9, A0A287IUL0, MOYMH9, A0A287JL90, A0A287XAW7, A0A287H0P0, A0A287JKK7, F2DQ07, F2DVF8, A0A287EXV5, A0A287EXX5, A0A287EG25, A0A287XZG3, A0A287XBL1, A0A287JKV7, A0A287G0I4, A0A287V5J4, A0A287X4U7, A0A287S5B5, A0A287EG62, A0A287JDT8, A0A287JPY5, A0A287EG71, A0A287JKX7, A0A287JLI6, A0A287N0F4, A0A287HU27, A0A287G0J5, A0A287MK69, F2DP44, A0A287Q173, A0A287I9A6, A0A287ULB9, A0A287XSC3, A0A287JKF5, A0A287XRW5, A0A287N5W5, A0A287HU05, A0A287Q638, F2DDU9, F2EJV9, A0A287UVQ5, A0A287LS87, A0A287LS64, A0A287E1P0, A0A287I9C6, A0A287KLC0, A0A287MUT0, F2DUT1, M0W647, A0A287JGC0, MOX5T2, A0A287KLF4, F2DGU3, MOXSP1, MOW3T9, A0A287I9C1, A0A287JL89, A0A287H0J6, A0A287EFT8, A0A287K4Z4, A0A287FZB8, A0A287XIW0, A0A287KGP2, A0A287NLR1, A0A287SYB4, A0A287H0L5, A0A287HU06, A0A287HTJ9, A0A287KLD6, A0A287V5F6, A0A287LSC4, A0A287EG21, A0A287VST9, A0A287LSA6, A0A287S505, A0A287H0K2, A0A287JKK1, A0A287HU32, A0A287IUK4, A0A287JGI4, MOYSV2, A0A287G0I9, A0A287GY94, A0A287JK87, A0A287JKX9, MOXCH9, A0A287LS99, A0A287S571, A0A1Z5T7T5, A0A1Z5T1G6, A0AIZ5SZU9, A0AIZ5TRS5, A0AIZ5SNU5, A0A1Z5T2F6, A0AIZ5TKS3, A0A1Z5T220, A0A1Z5T860, A0A3M6XEF2, A0A3M6YRP2, A0A3M7B8I9, A0A3M7BD57, A0A3M7EN81, A0A3M7IHZ9, A0A3M7F5J7, A0A3M7CY13, A0A3M7IF39, A0A3M7IKN2, A0A3M7F3Z2, A0A3M7J989, A0A3M7DTD4, A0A3M6X1R5, A0A3M7B9I1, A0A3M7HRW2, A0A3M6ZPM6, A0A3M7A6V9, A0A3M7CW34, A0A3M7FE79, A0A3M6Z7L5, A0A3M6WLT3, A0A3M7IN62, A0A3M7AEZ4, A0A3M7D1M2, A0A3M7GWG3, A0A3M6YTF6, A0A3M7FEQ6, A0A3M7GDI6, A0A3M7GKN4, A0A3M7F5S1, A0A3M7H9H8, A0A3M7E7K4, A0A3M7BMV2, A0A3M6YFN9, A0A3M7HTY7, A0A3M7C6P1, A0A3M6Y8Q0, A0A3M7GBS4, A0A3M7FVY9, A0A3M7AI77, A0A3M7H9D0, A0A3M7F5D2, A0A3M7EZS7, A0A3M6Y2F1, A0A3M7EXQ6, A0A3M7CWK5, A0A3M7FQH4, A0A3M7FKN6, A0A3M7CZJ1, A0A3M7HXS6, A0A3M7ITW6, A0A3M7DCL5, A0A3M7FY57, A0A3M7AY00, A0A3M7HF76, A0A3M6XNG0, A0A3M7IR78, A0A3M7D168, A0A3M7EZ41, A0A3M7E391, A0A3M7GVU9, A0A3M7FYN0, A0A3M7BRN4, A0A3M6X571, A0A3M6XIS4, A0A3M7IC91, A0A3M7F1K5, A0A3M6WR01, A0A3M6WED0, A0A3M7BI24, A0A3M7BYP9, A0A3M7EB66, A0A3M7ETI4, A0A3M7HLN2, A0A3M7DQ18, A0A3M6X7W9, A0A3M7C0U4, A0A3M7AI42, A0A3M7DXT2, A0A3M7CVC3, A0A3M7EYQ9, A0A3M7DQ84, A0A3M7I235, A0A3M7IA13, A0A085W504, M4B6K3, M4B6K2, A0A2J6RSW5, A0A2J6RMZ2, A0A2J6RSV8, A0A2J6RGD2, A0A2J6RVZ8, A0A2J6S4F4, A0A2J6R4R6, A0A2J6RYD0, A0A0R3WZ01, A8SOU4, A0A2M7IBC9, A0A3B0T141, A0A170PTA1, A0A3B0T4U7, A0A3B0S6G0, A0A3B0YDW4, A0A3B0RX13, A0A158QEB2, A0A3P7BEC2, A0A158QBS7, A0A1I7NHV8, A0A2P5KXM8, A0A2P5KRL9, A0A2S5PBC1, A0A2S5PC10, A0A2S5P4Z9, A0A0F2QD64, A0AIZ9YBG4, A0A2D8ZSJ2, A0A2E3AXJ3, A0A2E4SOJ7, A0A2D8ZV35, A0A069E6T4, A0A3B9H2G8, A0A059DX53, A0A3B9KYM9, A0A062U7B1, A0A062UCV7, A0A059FLM4, A0A059G005, A0A059F7I7, A0A059FSSO, Q0BYL9, Q0C462, A0A059G8G7, A0A062VJB5, A0A258BI69, A0A259LR84, A0A0F2RG41, A0A328JPZ2, A0A344WD66, A0A059DSX7, A0A059EES6, A0A062TZP2, A0A2D8W5J1, A0A3D2LBT6, A0A350ZTY8, A0A2D8ER08, A0A2E7B2U2, A0A2E3JXR2, G9P8Z6, G9NQ37, A0A024SGL6, A0A024SLI6, G0RDV7, G0RFA4, G9MYE6, G9NAA5, G9NBF0, G9MT46, A0AIY2W191, A0AIY2VQD0, A0AIY2VLV2, A0AIY2UVW5, A0AIY2V838, A0A1Y2VII2, A0A1Y2TBN0, A0A1Y2U492, A0A1Y2TUW0, A0A1Y2TMZ5, A0AIW0X097, A0AIW0WIH9, A0AIW0WWV0, A0AIW0X6K9, A0AIW0X4X0, A0A1W0WI98, A0A1W0WIM8, A0A1W0WIA3, A0A1W0WQ69, A0A1W0WPS6, A0AIW0WNF7, A0AIW0X6A8, A0AIW0X3K6, A0AIW0WF75, A0AIW0XCI5, A0A1W0WSL7, A0A1W0X0I9, A0A1W0WBY5, A0A1W0X277, A0A1W0X910, A0A1W0WDI1, A0A1W0WXQ4, A0A1W0X8W3, A0A1W0WR27, A0A1W0WFY4, A0A1W0WII5, A0A1W0WII2, A0A1W0WLH6, A0A1W0WG72, A0A1W0WBT5, A0A1A7YAK1, A0A1A7WJK9, A0A2D0SOY9, W5UJ51, A0A2D0PYF3, A0A2D0PVZ8, A0A2D0PZW9, A0A2D0SOB2, A0A2D0PX86, A0A2D0PN50, A0A2D0PUK1, A0A2D0PXQ6, A0A2D0PYM6, A0A2D0PW18, W5UM75, W5UH86, I3ME70, I3NFV2, I3M1N9, I3MLI1, I3ME62, W9VHP2, A0A177B984, A0A177B686, Q40096, Q40100, Q40099, Q40098, V5HBD1, V5H4U1, V5HJ86, A0A147BGK7, A0A131YAQ2, A0A131XWE4, A0AIC8FEE7, V5HEL7, A0A147BMG4, V5ICT5, B7QCV3, B7Q2N4, B7QM85, B7P1S6, B7P6R1, B7QDUO, B7PWP4, B7PQI9, B7Q7Z0, B7QM84, B7QM83, B7PM04, B7PQI8, A0A316V1R6, A0AII3UGX8, A0A2Y9B448, Q28VT1, A0AIB6YPR7, A0AIB6Z0H5, A0A067LHY6, A0A067KUJ8, A0A067L0Z2, A0A067L5M7, A0A067JDL7, A0A067L394, A0A067JQ57, A0A067JDL1, A0A067LB03, A0A067KQJ4, A0A067JMW2, A0A067K6D5, A0A067KR73, A0A067JMV5, A0A067KZS2, A0A067KVG4, A0A067KS71, A0A067KMS7, A0A067JLG1, A0A067KWS2, A0A067JLG5, A0A067KVF4, A0A067JMV9, A0A067L3A3, A0A067LB09, A0A067KXT3, A0A067JDL4, A0A067JFW9, A0A067KRX7, A0A067LDD5, A0A067L3A8, A0A067KRY8, A0A067JLG8, A0A067L4G0, A0A067JAL4, A0A067JQM4, A0A067KEY5, A0A067KZQ0, A0A067JQ92, A0A067JII4, A0A067K362, A0A067JAK6, A0A067L4F6, A0A067L4H1, A0A067KVG0, A0A067JAQ2, A0A067L398, A0A067L3B7, A0A067LDW4, A0A067JAQ7, A0A067JWU9, A0A067L392, A0A067K7M8, A0A067KS66, A0A067KRX3, A0A067KVG9, A0A067KB60, A0A067LBK7, A0A067KS82, A0A067K9W6, A0A067KF77, A0AID9BCC6, A0A2I4GK62, A0A2I4G4M2, A0A2I4H823, A0A2I4FLZ3, A0A2I4FU95, A0A2I4DI82, A0A2I4EPU3, A0A2I4E0M1, A0A2I4HI12, A0A2I4H7B3, A0A2I4GAB4, A0A2I4E0L2, A0A2I4FZE8, A0A2I4HNL1, A0A2I4FFM6, A0A2I4EKA5, A0A2I4ERC6, A0A2I4H782, A0A2I4H784, A0A2I4HSV7, A0A2I4EKB9, A0A2I4G4H1, A0A2I4HV68, A0A2I4H7D7, A0A2I4EPT2, A0A2I4DYI9, A0A2I4DYZ0, A0A2I4GFL8, A0A2I4E0L5, A0A2I4GBN2, A0A2I4FA92, A0A2I4DU39, A0A2I4E0L6, A0A2I4FU89, A0A2I4GBS2, A0A2I4HLF8, A0A2I4DYZ5, A0A2I4EGC9, A0A2I4DU26, A0A2I4DGG7, A0A2I4GV20, A0A2I4E0K7, A0A2I4H7H3, A0A2I4HHT8, A0A2I4DYJ2, A0A2I4FLN8, A0A2I4H7E7, A0A2I4GV14, A0A2I4GT05, A0A2I4DGF8, A0A2I4FFQ2, A0A2I4DKP2, A0A2I4GV12, A0A2I4FFG4, A0A2I4FIE0, A0A2I4GGL6, A0A2I4FZE7, A0A2I4ERD8, A0A2I4GT07, A0A2I4DKP0, A0A2I4FJE4, A0A2I4HTL0, A0A2I4HHJ4, A0A2I4GV23, A0A2I4EKK6, A0A2I4HNL3, A0A2I4GA68, A0A2I4HEF2, A0A2I4HLF5, A0A2I4H4Y0, A0A2I4GAY6, A0A2I4ERB9, A0A2I4FU46, A0A2I4HTX2, A0A2I4DQG1, A0A2I4ERD4, A0A2I4H6J2, A0A2I4HP53, A0A2I4GAU2, A0A2I4EGB5, A0A2I4DEN2, A0A2I4ERC8, A0A2I4FIB5, A0A2I4FLY6, A0A2I4G9T9, A0A2I4ERD7, A0A2I4H7C7, A0A2I4FFP8, A0A2I4HHI9, A0A2I4FIE8, A0A2I4H4L2, A0A2I4GG21, A0A2I4G912, A0A2I4GBS9, A0A2I4HPJ3, A0A2I4GAA0, A0A2I4EKJ5, A0A2I4HSV3, A0A2I4HTK6, A0A2I4FIL5, A0A2I4EK97, A0A2I4FFQ0, A0A2I4GV28, A0A2I4HIN1, A0A2I4EMX6, A0A2I4HV91, A0A2I4ERD1, A0A2I4DU21, A0A2I4EKU1, A0A2I4E0L9, A0A2I4GBS6, A0A2I4HP69, A0A2I4GWB0, A0A2I4GAW7, A0A2I4DET7, A0A2I4G4J0, A0A2I4GV30, A0A2I4HV71, A0A2I4FKE4, A0A2I4E7G7, A0A2I4GBT4, A0A2I4DGG1, A0A2I4DU29, A0A2I4FFP3, A0A2I4DN46, A0A2I4HAL5, A0A2I4FLV5, A0A2I4FID4, A0A2I4HV84, A0A2I4GBV7, A0A2I4E8G1, A0A2I4E8H5, A0A2I4E6A6, A0A2I4HNL4, A0A2I4HEG9, A0A2I4E3B8, A0A2I4EWD4, A0A2I4GBR4, A0A2I4EG75, A0A2I4GAA7, A0A2I4EG66, A0A2I4HG78, A0A2I4HSV9, A0A2I4DTA7, A0A2I4HNR2, A0A2I4H926, A0A2I4EG64, A0A2I4DGG9, A0A2I4DEM3, A0A2I4EN07, A0A2I4FU87, A0A2I4FIJ8, A0A2I4GBT2, A0A2I4EGC4, A0A2S5U2E7, A0A1M4XZE6, A0A1E4CVK7, A0A1E4C319, A0A1Y1I6D8, A0A1Y1IA49, A0A1V0SHW7, A0A2W5CL52, A0A3Q2Z9K7, A0A3Q3B1Z9, A0A3Q3B902, A0A3Q3F291, A0A3Q3BH26, A0A3Q3B198, A0A1B9IXV1, A0NVX3, A0NMA0, A0A0M6YIA7, A0A0M6Y878, A0A0M7AJT2, A0A0M6YXQ5, A0A0M6ZWY2, A0A2S3UK78, A0A2T7H985, U7G8P7, U7GAT6, A0A163UN31, A0A163WYR8, A0A222F1C4, A0A222FCV3, A0A1M7CSY2, A0A3Q3N8L8, A0A3Q3GBJ6, A0A3Q3FDA4, A0A3Q3GEZ8, A0A3Q3GFE0, A0A3Q3N8X5, A0A3Q3GFJ4, A0A3Q3N8N2, A0A3Q3GFZ5, A0A3Q3GB54, A0A3Q3GFB9, A0A2J6K9H8, A0A2J6M9S4, A0A2J6LQZ4, A0A2J6JXN1, A0A2J6KN62, A0A2J6KKT3, A0A2J6KC77, A0A2J6MH90, A0A2J6LXZ5, A0A2J6JZ56, A0A2J6LVI0, A0A2J6K3Q9, A0A2J6L847, A0A2J6L7A5, A0A2J6K925, A0A2J6K8G7, A0A2J6MDS4, A0A2J6M338, A0A2J6LCC7, A0A2J6MGQ2, A0A2J6M6H7, A0A2J6KQ34, A0A2J6MB10, A0A2J6KSB6, A0A2J6M2X5, A0A2J6KQ62, A0A2J6KD61, A0A2J6KDN2, A0A2J6KlB8, A0A2J6K3R7, A0A2J6MI63, A0A2J6MGT2, A0A2J6JXJ5, A0A2J6M5A7, A0A2J6KQC0, A0A2J6JL93, A0A2J6KKU2, A0A2J6JT61, A0A2J6JYI5, A0A2J6KK13, A0A2J6M2T7, A0A2J6KQKO, A0A2J6KS85, A0A2J6LUT0, A0A2J6KKG9, A0A2J6JXL9, A0A2J6LRN0, A0A2J6M2U7, A0A2J6K992, A0A2J6JM53, A0A2J6KI92, A0A2J6MGS8, A0A2J6MH59, A0A2J6JU53, A0A2J6KQ13, A0A2J6MH39, A0A2J6KDQ7, A0A2J6JXP1, A0A2J6JXMO, A0A2J6K937, A0A2J6JXM4, A0A2J6LRI2, A0A2J6K9T5, A0A2J6JU38, A0A2J6L6J2, A0A2J6LFL5, A0A2J6KI16, A0A2J6KQR3, A0A2J6KQ10, A0A2J6LRK6, A0A2J6M2U5, A0A2J6MFF9, A0A2J6MFK8, A0A2J6LRQ0, A0A2J6JHP7, A0A2J6L1L1, A0A2J6KKP5, A0A2J6LL57, A0A2J6K8G6, A0A2J6L780, A0A2J6K927, A0A2J6JX67, A0A2J6JXK7, A0A2J6JPF2, A0A2J6LV17, A0A2J6LRI5, A0A2J6M2Y3, A0A2J6MGY5, A0A2J6L1K0, A0A2J6LVM5, A0A2J6M6F0, A0A2J6LUM7, A0A2J6MGS4, A0A2J6KZL2, A0A2J6M316, A0A2J6JXN5, A0A2J6JXL3, A0A2J6KFI6, A0A2J6KDB6, A0A2J6LRT0, A0A2J6JM37, A0A2J6KQI6, A0A2J6L1H5, A0A2J6JXA5, A0A2J6JXK1, A0A2J6KJY7, A0A2J6JW82, A0A2J6KQH9, A0A2J6M327, A0A2J6M2S7, A0A2J6K9D7, A0A2J6K917, A0A2J6KQ59, A0A2J6KJX6, A0A2J6KI82, A0A2J6L1I8, A0A2J6L7M7, A0A2J6KJV7, A0A2J6MGS5, A0A2J6M8C4, A0A2J6M2U1, A0A2J6L6Y1, A0A2J6KQ32, A0A2J6L9N2, W0G9U1, W0G5H4, A0A0F8CNM7, A0A0F8ASU5, A0A0J7NTZ9, A0A0J7KV86, A0A0J7KKG3, A0A0J7NL95, A0A0J7NBH3, A0A0J7KUZ9, A0A0J7KRYO, A0A0J7L2K3, M3XJC9, M3XGZ2, H3ACN6, H3AXI3, M3XKU7, E7D1R2, B0ZRK1, B0ZRN7, B0ZRK3, B0ZRK7, B0ZRK0, B0ZRK2, B0ZRJ7, B0ZRM2, B0ZRM5, B0ZRI5, B0ZRP4, B0ZRI9, B0ZRK4, B0ZRP3, B0ZRM6, IlVlS2, B0ZRL1, B0ZRM7, B0ZRL9, A0A2Z4HJD9, B0ZRI6, R9S7T9, B0ZRJ2, B0ZRM8, B0ZRI4, B0ZRK6, B0ZRJ6, B0ZRN9, B0ZRN2, B0ZRJ1, B0ZRM9, B0ZRN6, B0ZRK9, B0ZRM1, B0ZRJ3, B0ZRL7, B0ZRI2, R9S7P5, B0ZRI8, B0ZRL6, B0ZRL5, B0ZRN3, R9S7H9, B0ZRN5, B0ZRL0, R9S9P4, B0ZRJ4, B0ZRI3, B0ZRL8, B0ZRI7, B0ZRN4, B0ZRL4, B0ZRL3, B0ZRJ8, B0ZRM4, B0ZRK8, I3QHF6, B0ZRJ5, B0ZRN0, B0ZRK5, R9SA46, B0ZRM0, B0ZRP0, A0A2Z4HJM8, R9S9V7, B0ZRJ9, B0ZRN1, A0A0D9W6W8, A0A0D9XWL1, A0A0D9W161, A0A0D9WRU9, A0A0D9V8W7, A0A0D9XNSO, A0A0D9WA06, A0A0D9V3Y6, A0A0D9W9ZI, A0A0D9V8T3, A0A0D9V8U6, A0A0D9V8Q3, A0A0D9XGF3, A0A0D9WXV1, A0A0D9W4W1, A0A0D9W9Y8, A0A0D9X052, A0A0D9XGI1, A0A0D9WD80, A0A0D9X057, A0A0D9W9Z0, A0A0D9WTD1, A0A0D9VVN7, A0A0D9V8T2, A0A0D9XB99, A0A0D9W2T7, A0A0D9X065, A0A0D9W9Z8, A0A0D9UY17, A0A0D9XZW4, A0A0D9XGF7, A0A0D9XIR5, A0A0D9WA00, A0A0D9W4V1, A0A0D9WR41, A0A0D9XGF4, A0A0D9W9Y9, A0A0D9V3F7, A0A0D9WA08, A0A0D9VVD3, A0A0D9WRG7, A0A0D9X056, A0A0D9WA07, A0A0D9WA13, A0A0D9V6J6, A0A0D9WIB2, A0A0D9UWX5, A0A0D9XGF8, A0A0D9WGJ8, A0A0D9XWK7, A0A0D9V3Y5, A0A0D9XWK5, A0A0D9W4V4, A0A0D9XGI2, A0A0D9V3F8, A0A0D9XZV4, A0A0D9W1R5, A0A0D9V6K0, A0A0D9W4V9, A0A0D9V6J7, A0A0D9UYF7, A0A0D9VRC5, A0A0D9XGF5, A0A0D9W4V2, A0A0D9XNR9, A0A0D9V6J2, A0A0D9V3Y7, A0A0D9W4V3, A0A0D9W3A9, A0A0D9WA14, A0A0D9W4V7, A0A0D9V7B0, A0A0D9V3Y8, A0A0D9V1T5, A0A0D9WQ05, A0A0D9VAIl, A0A0D9UYF9, A0A0D9V6J9, A0A0D9W3B3, A0A0D9W9Z3, A0A0D9WD66, A0A0D9W2T8, A0A0D9V8T6, A0A0D9WRU8, A0A0D9UYF8, A0A0D9WA03, A0A0D9WAH9, A0A0D9XZV3, A0A0D9XQ93, A0A0D9WA05, A0A0D9WR42, A0A0D9X053, A0A0D9W9Z9, A0A0D9W9Z6, A0A0D9WGJ7, A0A0D9XH63, A0A0D9V6J3, A0A0D9W9Y7, A0A0D9X503, A0A0D9W3B1, A0A0D9W4V6, A0A0D9W4V5, A0A3P3YXB7, A0A3P3YXB3, A4H3V1, A0A3S5H596, A0A1ElINT0, A0A381M993, E9AK14, A0A088RHV5, A0A0P1H8K4, V9VZT4, A0AIH3GY41, A0A0K2TCD8, A0A0K2V009, A0A0K2T1Y7, A0A0K2TPD6, A0A0K2UTX9, A0A0K2TTX1, A0A0K2U3D2, A0A0K2T870, A0A0K2U346, A0A0K2TlX1, A0A0K2SXW0, A0A0K2UNM9, A0A0K2TDI0, A0A0K2TFU3, A0A0K2TBQ4, A0A0K2UUX8, A0A0K2SZ08, A0A0K2U2F7, A0A0K2UVK3, A0A0K2TZM7, A0A0K2UP87, A0A0K2USH8, A0A0K2UOI6, A0A0K2UUV6, A0A0K2UUZ9, A0A0K2UTJ0, A0A0K2TZZ8, A0A0K2UVW1, A0A0K2UU23, A0A0K2UVG2, A0A0K2UWI6, W5NER0, W5M199, W5NEQ8, W5NFF8, W5NAB0, A0A3M9ZBN0, A0A2U3YDU7, A0A2U3X6Q7, A0A2U3YLJ6, A0A2U3YE66, A0A2U3YDS5, A0A2U3YDS8, A0A2U3XSF8, A0A2U3XSB8, A0A2U3X6P8, A0A091PSC7, A0A091PQK1, A0A091Q2P9, E4ZGL2, E5AD62, E5A991, A0A443SF82, A0A443RUF8, A0A443SUSO, A0A443SE45, A0A443RVB4, A0A443SM33, A0A443SQ21, A0A443SF48, A0A443SHP2, A0A443SQl1, A0A443S6A4, A0A443SH42, A0A443SI69, A0A443SN48, A0A2Z5XDQ4, A0A2Z5XDQ7, A0A1Y2FYL8, A0A2I0UCB4, A0A2I0U654, A0A2I0THH2, A0A2I0U125, A0A1S3KCR3, A0A1S3I3B4, A0A2R2MTN1, A0A1S3JBH9, A0AIS3HNX8, A0AIS3HFI9, A0A1S3I1V9, A0AIS3JR18, A0AIS3IEH2, A0A1S3J1T8, A0A1S3H9T3, A0A1S3HBYO, A0A1S3K773, A0A2R2MTZ4, A0A1S3INU8, A0A1S3IE57, A0A1S3JU66, A0A1S3I9N3, A0A1S3JZ08, A0A1S3K775, A0AIS3JZ64, A0AIS3JQD4, A0A1S3J5V4, A0AIS3GZQ5, A0A1S3IAW8, A0A1S3K770, A0AIS3HKV5, A0A1S3IIA6, A0AIS3JFE9, A0A1S3IIY2, A0AIS3IQU7, A0AIS3KBV0, A0AIS3KC90, A0AIS3INP9, A0AIS3KEU6, A0A1S3H081, A0A1S3K6K4, A0AIS3KOC7, A0A1S3I3C2, A0AIS3KFD1, A0AIS3KEE4, A0AIS3KBC6, A0A1S3H208, A0A2R2MSR5, A0A1S3K4Q7, A0AIS3JEN3, A0AIS3GZU4, A0A1S3I1W2, A0AIS3JBKO, A0AIS3KHB8, A0A1S3INY3, A0A2R2MLU4, A0A1S3H7Z1, A0A1S3HI84, A0A1S3HZL4, A0AIS3KCC7, A0A1S3ISSO, A0A1S3IMR2, A0AIS3HER8, A0A2R2MJK4, A0AIS3KEG7, A0AIS3HEB9, A0A1S3K901, A0AIS3KOQ7, A0A1S3K7Q8, A0AIS3KA03, A0AIS3JR32, A0A1S3H2K1, A0A1S3I4L8, A0AIS3HD07, A0AIS3KAP7, A0AIS3J0J2, A0A1S3J4U5, A0AIS3JX74, A0A1S3KiK5, A0AIS3IC24, A0AIS3JXG0, A0A1S3K6B9, A0AIS3J163, A0A2R2MRD9, A0AIS3HR32, A0AIS3JYB9, A0AIS3KBX3, A0AIS3JYD2, A0AIS3HYX9, A0AIS3KFK1, A0A1S3I1T9, A0AIS3ILB6, A0AIS3KOU4, A0AIS3JHR9, A0AIS3JBP0, A0AIS3JSJ3, A0A1S3ICN0, A0AIS3JR04, A0AIS3HBMO, A0A1S3J6C4, A0A1S3ID77, A0AIS3JZB8, A0AIS3IZ38, A0AIS3JW52, A0A1S3J693, A0A2R2MIB3, A0A1S3I2T0, A0A1S3K221, A0AIS3KBT6, A0AIS3HVT4, A0AIS3IHR4, A0AIS3KD35, A0AIS3GZL9, A0A2R2MJM7, A0A1S3K756, A0A2R2MRG8, A0AIS3MIG5, A0AIS3JYC8, A0A1S3H9K2, A0AIS3JQN5, A0A1S3JZA1, A0A2R2MNI0, A0A1S3KB21, A0A340XF42, A0A340XZU6, A0A340WGH7, A0A340Y6H1, A0A340Y5U1, A0A340X8C1, A0A340X7S9, A0A340X8B6, A0A340XEK1, A0A340XC28, A0A340XC32, A0A3P7K4H3, A0A3P6TYE8, A0A3P7M9S7, A0A3P7M8L9, A0A3P6TRE5, A0A3P6V4Q9, A0A3P6S6Z6, A0A3P6T8W6, A0A3P6USV6, A0A3P6UER0, A0A3P6U602, A0A3P6U3U8, A0A3P6UAP6, A0A3P6SQ54, A0A3P6SHG7, A0A3P6V945, A0A3P6RZY6, A0A1H2WL46, S9QDQ2, A0A1M5BZJ4, A0AII6G0Y4, A0AIH9DXX5, A0A420WEA0, A0A420WFF1, A0A420WKP0, A0A0A7RPZ1, A0A0A7RPW5, A0A0A7RPU1, A0A1I7VDG7, A0A1I7VW75, A0AISOU4V4, A0A1I7VW66, A0A1I7W211, A0A1I7VX34, A0AISOU2W5, A0AISOU290, A0A1SOTQ84, A0A1SOU4B4, A0A1I7V595, A0A1SOTXG2, A0A1SOTGL3, A0A1I7VEP2, A0AII7W1P7, A0AII7VM68, A0A1SOU513, A0A1I7V7Z8, A0AISOTYRI, A0AII7VP24, A0AII7W1K7, A0AISOU521, A0AII7VEI3, A0A1SOTM76, A0A1SOTVKO, A0A1I7V913, A0A1SOU7X7, A0A1SOTNT1, A0A1SOTXD9, A0A1SOU5P5, A0A1I7VER6, A0A117VJ23, A0A1SOTNL5, A0AISOTP23, A0A366W4W6, A0A2N3N2R9, A0A218UQI0, A0A218V7U2, A0A2A8CVC7, B3XXB9, V4ASX7, V4CNX9, V4AQ34, V4A085, V3ZGG1, V3ZBJ5, V4AP13, V4A568, V4B215, V3ZQU5, V3ZUA7, V4AXR6, V4AG04, V4BSL1, V4A7Y, V4BB69, V4BDZ0, V4A5J1, V3ZJU6, V4B1H4, V4AGQ9, V4BAA4, V4BQJ5, V4AWC8, V4CGE2, V4A396, V3ZYP3, V3ZKE9, V4BQJ9, V4B0J4, V4CE61, V3ZTQ2, V4AZ96, V4ACW6, V3ZKU5, V4ADH8, V4BC73, V4A6L5, V3ZFI8, V4ANB3, V3ZGM4, V4C4P7, V4AFG6, V4AAJ8, V3ZJQ9, V4B893, V4A878, V4AAG6, V3ZVU7, V4A5C0, V4AF63, V3ZP38, V4ANZ3, V4BCE9, V4A5K9, V4BEG6, V4ADA5, V4BFS5, V4ABU2, V4AGS3, V4B189, V4A0F5, V4BZ59, V4C8K6, V4ANZ0, V4AAL1, V4BV31, V4AM58, V4BSL7, V4AJ20, V3ZA19, V4BF63, V4AA83, V3ZC53, V4AZU1, V4A6E5, V4A9B0, V4AZF9, V3ZRI2, V3ZE05, V4BHI1, V4CFZ0, V4AAD3, V4BND1, V4A8U5, V4AAP8, V4A8U0, V4AQF9, V3Z3B9, V3Z9D7, V4BXB3, V3ZKT4, V4B8Q8, V4A2F6, V4BGT4, V3ZJF4, V4ATM4, V4BAE3, V4ARJ8, V4BB58, V4AZ26, V4AJG9, V4B9R7, V3Z371, V4AGU9, V4ALG6, V3Z819, V4AXG5, V3ZP70, V4A0V8, V4AZ89, V4BDK1, V3ZNC3, V4A9Q6, V3ZHP8, V4AWY7, V4C4C1, V4B8W2, V4B493, V4BY76, V3ZM12, V4C7E0, V4AUH2, V4CL71, V3ZP26, V3ZV91, Q70128, B0BLA2, Q70130, Q8GSP1, B0BLA4, Q70129, Q8GSP2, I3SEG1, Q8GSP0, G3TJ67, G3SSI7, G3U701, G3TJ74, G3U7P9, A0A0L0CF27, A0A0L0BZD3, A0A0L0CF53, A0A0L0CB36, A0A0L0CRG8, A0A0L0C513, A0A0L0BPA5, A0A0L0CB71, A0A0L0BLS4, Q8LP50, A0A1J7H6R4, A0A1J7GA64, A0A1J7HQD3, A0A394DIC4, A0A1J7I619, A0A1J7H3V7, A0A1J7GFK8, A0A1J7GZ77, A0A1J7HH30, A0A1J7HEQ0, A0A1J7GDB9, A0A1J7GNM1, A0A1J7I6S6, A0A1J7HD03, A0A1J7FWA6, V4RQY9, A0AIH0GW80, A0AIB0CHS8, A0AIB0CDT4, A0AIB0CQS2, A0AIB0CJ91, A0A1B0CLB1, A0A146LQ91, A0A146M6Y4, A0A146KQT6, A0A0K8SEA4, A0A0A9ZGD8, A0A0A9XMZ2, A0A0A9X4C5, A0A0A9ZB53, A0A0A9VYR8, A0A0A9YIK1, A0A0K8T345, A0A0A9VV93, A0A0A9X899, A0A0A9XR44, A0A0K8SWZ6, A0A0K8SQ51, A0A146L1N9, A0A146LN03, A0A0A9YAH9, A0A0S2G5W4, A0A2K5VVK2, G7PKF4, A0A2K5WCZ4, G7PKF5, A0A2K5WGT6, G7P478, A0A2K5VTJ9, C9E9X5, A2V9Z1, A0A2K5WGT9, A0A2K5TJJ3, A0A2K5WH02, A0A2K5VTQ7, C9E9X6, G8F4N5, A0A2K5TJJ6, F7A5G3, F6PJA8, F6PJA1, G7MSF8, F7CC91, A0AID5RGB5, H9FEQ2, G7ML90, G7MSF7, F6PJ95, F7A5F6, A0A1D5Q7U7, G7MM01, P12545, A0A2K6DQD1, A0A2K6DDC3, A0A2K6DDA5, A0A2K6DD94, A0A2K6DCN9, A0A2K6DH51, A0A2K6E3X7, A0A2K6E3W9, A0A2K6DQE6, A0A200QXH0, A0A200RDT1, A0A200Q4Y2, A0A200QXM5, A0A200Q8X0, A0A200QRV5, A0A200QQN4, A0A200Q130, A0A200QBX7, A0A200R1B1, A0A200QTR7, A0A200QXK1, A0A200QXI1, A0A200QQN7, A0A200Q8Y1, A0A200Q8J1, A0A200Q0W5, A0A200QXH4, A0A200QIT5, A0A200Q0X7, A0A200QXF5, A0A200QRS4, A0A200Q8V5, A0A200Q2G3, A0A200Q0W0, A0A200QXP0, K2RH51, K2RS30, K2RCW8, K2RNM7, K2S8A1, K2RUQ8, 018783, A0A1I8GHF8, A0A1I8FVR7, A0A1I8H365, A0A1I8J167, A0A267EWK4, A0A1I8IBQ6, A0A1I8JEV8, A0A1I8FXH1, A0A1I8GNZ2, A0A267GQH1, A0A267GGW3, A0AII8GL54, A0A1I8HG15, A0A1I8IV72, A0A267G2E7, A0AII8G1P9, A0A267EA31, A0A267GUN8, A0AII8IW00, A0A1I8IVY1, A0A1I8IRT6, A0AII8H8D5, A0AII8G8D6, A0AII8HC33, A0A1I8IMI9, A0A267FXX8, A0A1I8J0U1, A0A1I8GHG0, A0A1I8HE61, A0A1I8J1U2, A0A1I8JJX3, A0A1I8GHR2, A0A1I8JKN1, A0A1I8FX03, A0A1I8J0K0, A0A1I8GPQ5, A0A267FEP6, A0A1I8GLQ8, A0A1I8J5S5, A0A1I8GNC3, A0A1I8GF89, A0A267DMG9, A0A267H2N9, A0AII8JF80, A0AII8GE13, A0A267DXV5, A0A1I8H2N3, A0A1I8FHG8, A0A1I8GCM3, A0A1I8FWJ3, A0A1I8GNW6, A0AII8IZ00, A0A267F306, A0A267EXC8, A0A267GX64, A0AII8GFR2, A0AII8I316, A0A1I8ID15, A0A1I8G103, A0A267E9Q2, A0A267DNT5, A0A1I8G9H2, A0A1I8J5P8, A0A267E0M5, A0A267G2S7, A0AII8FV01, A0A1I8HV10, A0A1I8J8F5, A0A1I8GJX9, A0A1I8H4R7, A0A1I8J9R2, A0A1I8JG79, A0A1I8GZN4, A0A1I8J542, A0A1I8JFP1, A0A1I8HVW8, A0A1I8J222, A0A1I8G6K2, A0A1I8GEP5, A0A1I8G513, A0A1I8I3M0, A0A1I8J5N9, A0A1I8FTM3, A0A1I8HQT8, A0A1I8GI38, A0AII8GEN9, A0AII8G9F7, A0AII8IHH8, A0A267H9R8, A0A267GB32, A0A267GHE2, A0A267EFJ6, A0A1I8FYE4, A0A267F4N8, A0A1I8FY63, A0A1I8FMH1, A0A1I8GYH3, A0A1I8IU21, A0A1I8GI06, A0A1I8F694, A0A1I8GHZ2, A0A1I8GIV2, A0A1I8FT25, A0A267F4Y9, A0A1I8JK67, A0A1I8GKV5, A0AII8GC08, A0A1I8GBL5, A0A1I8GKD7, A0A1I8HSV8, A0A267FJ10, A0A1I8J628, A0A1I8HKD5, A0A1I8IZJ9, A0A1I8HM08, A0A267GBV5, A0A1I8J8D6, A0A267GVU8, A0A267GP16, A0A1I8JFH6, A0A1I8J7E2, A0A1I8GC74, A0A1I8H460, A0A267E6R9, A0A1I8FRS9, A0A267G961, A0A267DRT5, A0A1I8HUT3, A0A1I8FU86, A0A267EFS8, A0A1I8HYI4, A0A1I8IQU1, A0A267ETV5, A0A267E615, A0A1I8FYK6, A0A1I8FWD9, A0A267ETH5, A0A1I8J0A4, A0A1I8FYP1, A0A1I8GUN9, A0A267FDY5, A0A1I8IHJ7, A0A175WH97, A0A175W7N1, A0A175WB07, G4MZI7, Q2KGYO, G4MXA4, G4MZU5, L7J520, L7JDZ1, L7JG00, L7HS16, L7I490, A0A0C4EBJ2, A0A2E6X7J5, A0A2E6X7K3, A0A238JQ45, A0A0B3RUU6, A0A0B3S7C3, A0A2E9I414, A0A2E9I2Q1, A0A2E9I2P9, A0A093QGB8, A0A093Q5H9, A0A2K5ZUK4, A0A2K5Z4M0, A0A2K6AE30, A0A2K5Z4L5, A0A2K5Z4N9, A0A2K6ADW4, A0A2K5YTU2, A0A2K5Y8K1, A0A2K5ZUK1, A0A2K5Z4U2, A0A2C9UZL6, A0A2C9UQY7, A0A2C9U9R2, A0A2C9W6L4, A0A2C9WFX9, A0A2C9VN71, A0A2C9V805, A0A199UBF6, A0A2C9U5I8, A0A2C9U8X9, A0A2C9U847, A0A2C9V7G1, A0A2C9VAX0, A0A2C9VBF0, A0A2C9VNX9, A0A2C9VLQ8, A0A2C9U446, A0A2C9VAL0, A0A2C9U4D2, A0A2C9U801, A0A2C9U4I0, A0A2C9U805, A0A2C9U7Y6, A0A199UAQ8, A0A2C9UYI9, A0A199UBH6, A0A2C9U8Y9, A0A2C9UPX0, A0A2C9UFH3, A0A2C9U3X3, A0A2C9W8T4, A0A2C9U4C4, A0A2C9UF19, A0A2C9W6W4, A0A2C9U642, A0A2C9VNX3, A0A2C9UK30, A0A2C9VP96, A0A2C9W6N6, A0A199U9M8, A0A2C9UUL6, A0A2C9URP0, A0A2C9UZM3, A0A2C9WC49, A0A2C9UX56, A0A2C9U928, A0A2C9UMD6, A0A2C9UFX9, A0A2C9U9Q2, A0A2C9W8S6, A0A199UC06, A0A2C9UF31, A0A2C9U4L0, A0A2C9U5B5, A0A2C9U487, A0A2C9UDV6, A0A199UC16, A0A2C9VLR9, A0A2C9UOH2, A0A2C9VN68, A0A2C9UPX3, A0A2C9UPE4, A0A2C9UY52, A0A199UBJ1, A0A2C9W426, A0A2C9WEY1, A0A2C9W6P6, A0A2C9W2Y5, A0A2C9W6Q6, A0A2C9VLT1, A0A2C9U478, A0A2C9U869, A0A2C9UZ81, A0A2C9WFM5, A0A2C9W6P5, A0A2C9U8V8, A0A2C9U4K0, A0A2C9UFD3, A0A2C9UUW9, A0A2C9VNC9, A0A2C9VF19, A0A2C9UEZ1, A0A2C9UAW2, A0A2C9UYH0, A0A199UBF1, A0A2C9VN80, A0A2C9U9K7, A0A2C9VTQ5, A0A2C9U5A6, A0A2C9UPQ0, A0A2C9U8W8, A0A199UAZ0, A0A2C9VNE5, A0A2C9UPZ5, A0A2C9U839, A0A2C9UFW8, A0A2C9VB28, A0A2C9U816, A0A2C9U5B6, A0A2C9ULZ6, A0A2C9W6L3, A0A2C9VLU2, A0A199UBJ8, A0A2C9U935, A0A2C9VAT4, A0A2C9U7Q9, A0A2C9VLX7, A0A2C9W6M3, A0A2C9U632, A0A2C9VMC6, A0A2C9UEM3, A0A2C9UFH5, A0A2C9UON6, A0A199UBH1, A0A199UC12, A0A2C9VLU8, A0A2C9VLS2, A0A2C9VP90, A0A2C9WEW1, A0A2C9U537, A0A2C9U4I6, A0A2C9VE98, A0A251IUP5, A0A199UBE6, A0A251JEQ5, A0A176VVC5, A0A176VSU9, A0A2R6WIUO, A0A2R6WIQ4, Q0AR81, A0A1G9Q3S7, A0AIQ8FK24, A0A2E2XTA6, A0A2D7Y7C9, A0A161Q3D4, W0EIA6, A0A3M8NI56, A0A0F9HEY4, A0A225NEP4, A0A2U2BRH1, A0A2U2BSN0, A0A0H4LDC5, A3VM18, A0AIX7NU32, A0A2D9YV70, A0A2D8PDZ1, K1XNZ5, K1WYE2, K1XJ48, A0A218YS99, A0A218Z724, A0A2T0JV19, A0A3Q3MRJ9, A0A3Q3LF07, A0A3Q3MRQ7, A0A3Q3MWE6, A0A3Q3S4P9, A0A3Q3LIA0, A0A3Q3MRT6, A0A3Q3MXL6, A0A3Q3SHD3, A0A3Q3RXI1, A0A3P9CUC6, A0A3P9CVH7, A0A3P9CXJ8, A0A3P9BY20, A0A3P9BCB7, A0A3P9CDB0, A0A3P9CW19, G7ITJ8, G7IK65, A0A072TZ18, A0A072UMY1, A0A072TMX7, G7LFV0, G7IK59, G7LFU9, A0A072UY36, A0A072ULK1, A0A072UP57, G7LFV1, G7JF50, G7JSW6, G7IK51, G7JF44, G7LFU8, G7IK53, G7IVB0, A0A072VB47, G7IRL5, G7IK61, A0A072UA49, A0A072ULW3, A0A072V563, G7LFU6, A0A072UZ61, G7L503, G7K610, G7J0P4, A0A072V398, G7IK50, A0A072TZS6, A0A072UUL6, A0A072UTW4, G7IZ17, G7K613, A0A072UFT0, G7IK52, A0A072U488, A0A072UVG0, A0A072UN27, G7IVA0, A0A072TZ03, G7IZ18, G7IK54, G7J0Q4, A0A072VB61, A0A072VAM1, Q2HTU4, G7JF49, G7J0Q7, A0A072TPH7, G7L990, G7LDQ6, G7LA23, A0A072UA25, A0A072UUL9, A0A072TYW2, A0A072UOS7, G7IYY4, A0A072TZR6, G7L9B9, A0A072THU9, A0A072UA87, A0A072TZY0, G7J708, G7L199, A0A072TQM4, G7IYX7, A0A072TQ44, A0A072TZ15, G7LA28, G7IH64, A0A072UA68, G7IYX3, A0A072UNR6, G7KZ40, G7LA34, G7L986, A0A072UA82, G7IYY1, A0A072TZ68, A0A072UR75, A0A072THC7, A0A072TZY9, G7JZD9, A0A072V9X1, G7J0Q3, A0A072U1F5, A0A072TPK7, A0A072TYZ8, A0A072V4F9, A0A072TQ73, G7LBP9, A0A072TLA3, G7ITK1, A0A072V4F3, A0A072UN78, A0A072VL26, G7IYX6, A0A072U1H2, A0A072V3X0, A0A072U1E3, A0A072TS26, A0A072UOS1, G7KKY8, G7LA08, A0A072TZ74, A0A072UON7, A0A072UP51, A0A072VBN5, A0A072TTD3, G7J0Q1, A0A072UP20, A0A072TZ09, A0A072TZ31, G7IRK8, A0A072UVG3, A0A072VAL5, G7IK58, A0A072TQ29, G7JXF4, G7IZ14, A0A072UTW1, A0A072TQF1, A0A072UTA1, G7LA16, G7L9C7, A0A072TQ50, G7JXG0, A0A072TZ10, A0A072UTV6, G7JSW4, G7JSV8, A0A072UKC3, A0A072UN82, A0A072V8H5, A0A396GBYO, A0A396IB06, A0A396I8P5, A0A396I8N0, A0A396II14, A0A396GIR1, A0A396IJL1, A0A396IAC3, A0A396GKB0, A0A396GKM1, A0A396GID7, A0A396GXQ7, A0A396IPQ6, A0A396H464, A0A396J1V4, A0A396GHQ0, A0A396IJS7, A0A396GXP2, A0A396IJV7, A0A396IFP3, A0A396GZH7, A0A396J1V8, A0A396GIN5, A0A396IKZ3, A0A396IEA5, A0A396GIL7, A0A396GJK4, A0A396H410, A0A396J0V6, A0A396GXZ2, A0A396H073, A0A396IH94, A0A396IJQ8, A0A396JG02, A0A396ICL0, A0A396H452, A0A396IB36, A0A396J5K8, A0A396H4I4, A0A396GKI2, A0A396GAD8, A0A396JBU6, A0A396I3J8, A0A396GIN1, A0A396IQ10, A0A396I8N1, A0A396GC28, A0A396GHT6, A0A396H0A3, A0A396IJM8, A0A396IDT3, A0A396H2F4, A0A396J9F7, A0A396J198, A0A396IGJ9, A0A396IMY4, A0A396HUI6, A0A396GXR8, A0A396GJX9, A0A396GMB1, A0A396IQZ7, A0A396JBT7, A0A072TFN9, A2Q3L3, G7JSW9, G7JF45, A0A072TZ95, G7L1A0, A0A072TZR2, A0A072TLV0, G7L2A0, A0A072UDX2, G7IXM3, G7L299, G7L994, G7IRM2, A0A072UTA5, G7JZE3, A0A072U7C9, G7IVB2, A0A072TQ06, A0A072UQ11, G7L9B8, A0A072VJ29, G7J0P5, A0A072ULW9, A0A072TR17, A0A072U021, G7J0P6, A0A072USP0, G7JSX3, A0A072UF01, G7L2A2, A0A072U1F1, G7IYX8, G7IK56, G7IZ19, G7IK62, G7ITK2, G7JSX1, G7JSW5, A0A072UM50, A0A072VE90, A0A072TGW7, A0A072VAH2, A0A072VGD0, A0A396HUX6, A0A396GF10, A0A396J9G3, A0A396GLE7, A0A396J1B2, A0A396GK01, A0A396II24, A0A396H0B4, A0A396IAC8, A0A396GKN1, A0A396H9W1, A0A396GKK2, A0A396ICL7, A0A396GQP4, A0A396GIS2, A0A396GZH2, A0A396GZ32, A0A396J4V5, A0A396IIB1, A0A396GY25, A0A396IPU4, TiGV96, TiGCN7, G5CS05, G5CSX9, G5CSYO, K7YVH7, K7Y8W4, K7YGR5, H2EAU8, H2EAV0, H2EAE1, L7Y445, L7Y5I8, L7XXX5, A0A2P1EIL3, A0A2P1EIK7, A0A2P1EI82, A0A2H8TNX4, A0A2H8TQD0, G1N219, G3UPW8, G3USW9, GiNG08, G1NIF9, G1MVV6, A0A2J6SGV1, A0A2J6SNC2, A0A2J6T0X6, A0A2J6TAR8, A0A0M8ZTA1, A0A0N1IT07, A0A0M9AA61, A0A0N0BEK1, A0A0N0BFS3, A0A0N1IU30, A0A0N0BKD6, A0A250ING7, A0AII8B4B8, A0AII8BK90, A0AII8AXB8, A0A1I8AXZ1, A0A1I8C0R8, A0AII8BY25, A0A1I8BIP0, A0A1I8B5R6, A0A1I8BYM1, A0A1I8BQ11, A0A1I8B4D5, A0A1I8BG52, A0A1I8AY49, A0AII8BQG6, A0AII8BBQ9, A0AII8BCA3, A0A091QTC3, A0A091QC15, A0A091QSV6, A0A091QSR7, A0A091QUC8, A0A091RMA7, A0A0R3U559, A0A1U7Q8Z9, A0A1U7R772, A0A1U8C9R2, A0A3Q0CLL1, A0A1U7QLW9, A0A3Q0CJX2, A0AIU7R4K2, A0A3Q0CDV3, A0AIU8BGC2, A0AIU8BFL9, A0AIU8BRY1, A0AII3VIU9, A0AII4EVC7, H0HTQ8, A0A212DNS9, G6YBH1, A0A2W5AVW2, A0A2W4ZIG5, L0KNN6, A0AIX7PA47, A0AIX7PI50, E8TE24, A0A143NC21, A0A2P9AXN5, A0A2P7SR23, A0A330I171, A0A330H340, A0A2T4IQ86, A0A068DAZ6, A0A068D8X3, Q982A2, Q982A0, Q983Q7, Q98K29, Q982A3, A0A3M9XHZ6, A0A3A5KV12, A0A2W7C7Z2, A0A2W7CT51, A0A271L788, M5F7L2, A0AIG9IZK6, A0AIL3SUG4, F7Y3J3, F7YDM6, A0A090DAC7, A0A090GF48, A0A090GJ86, A0A090EAQ0, A0A0K2VPY4, A0A1R3VHN0, A0A1R3VG59, A0A1G5YRE1, A0A2A6F891, A0A2P7SMF1, A0A1Q3LTY8, A0A1Q4AJJ6, A0A1A5T9T0, A0A395KH22, A0A3R9Y594, A0A2N7SIF6, A6N7X9, A6N7X6, A6N7X7, A6N7X5, A0A330GWG4, A0A3A3HUH9, X6KLP5, X6K883, X6KA10, V7HDL0, X6JHB4, X6JBU3, X6IKL6, X6HND0, V7H8P5, X6FM84, X6GLA2, A0A371XGB0, A0A1S1SGU3, A0A1S1S606, X6EAG6, X6DBJ5, X6DP76, X6EWU4, X6F6P8, X6EVU3, X6CWC0, X6C4J0, X6DDI2, V7G7Z9, X5Y7Z1, A0A0E2NHH7, V7FGSO, X5V9Q4, X5V974, X5UDZ6, X5RA25, X5PRZ7, X5QT36, X5QMX6, X5QIF0, A0A436RRF8, A0A3S2R3M1, A0A437Z7D9, A0A3S2YUX2, A0A3S3C863, A0A3S3YBG7, A0A437YW26, A0A3S3A539, A0A437ZX63, A0A437VVF2, A0A3S2YVR6, A0A437WTZ3, A0A437VGM9, A0A434THQ7, A0A434SHC2, A0A434NS16, A0A434P1D2, A0A434MM88, A0A3SiILE2, A0A434PMQ0, A0A434L3Y6, A0A3Q8YPJ5, A0A3S9DBX0, A0A3S9DC85, A0A3S9DBY6, A0A3S9D2V1, A0A3S2KY76, A0A437WND3, A0A3S9FK01, A0A3Q9AI45, A0A3S3LC86, A0A443GJA2, A0A3S2I1J4, A0A436SS41, A0A3Q8YI66, A0A3SiKJ31, A0A434SWL3, A0A3S2KH58, A0A3S1NX24, A0A444KCUO, A0A3S3R3Y8, A0A444K8W2, A0A435D6W3, A0A3SiQJG7, A0A3S9BTR5, A0A436QSC2, A0A436SLUO, A0A436SBV8, A0A3S9DJ20, A0A3Q8YYA6, A0A3S9DRS6, A0A434VEP9, A0A3SiE0H8, A0A436Y6Z9, A0A436VBS5, A0A437ZLF5, A0A436P9R0, A0A436PHM8, A0A3S2IWX9, A0A3S2YQZ9, A0A437X9T7, A0A437X6J9, A0A436WL48, A0A436SR86, A0A3S2IF88, A0A3S2KLT7, A0A436SRB7, A0A436YYKO, A0A435B6A1, A0A3S2QL44, A0A434RXG0, A0A436UW61, A0A436WQQ7, A0A3S2LXT0, A0A3S1MLP3, A0A3Q8ZQ16, A0A436RI15, A0A3S3QWG9, A0A434MRM8, A0A3SiHQV7, A0A434MG50, A0A434JX21, A0A3SiHYB4, A0A434EHK3, A0A434CMJ8, A0A454WRI7, A0A3S2FRB0, A0A436KR71, A0A436KVN4, A0A3S2F8X3, A0A3SiVTV1, A0A435QXV3, A0A3SiV2H9, A0A435NFG8, A0A435K6G4, A0A3SiV0K8, A0A436HLD5, A0A3S2ERE3, A0A3S2AZW4, A0A3S2LEV5, A0A3S2DZD0, A0A436BIE9, A0A3S2FP85, A0A435P576, A0A3SiU647, A0A3SiTF94, A0A3SiUAZ5, A0A3S2DDW8, A0A436BS52, A0A3S1VGD1, A0A435KAI1, A0A3SiVM62, A0A3SiT8V1, A0A3SiTXYO, A0A435UXY3, A0A435U085, A0A3SiX503, A0A435QVB4, A0A436GBI0, A0A436GCM6, A0A436HTL9, A0A3S2HVT7, A0A436KG24, A0A435W6B2, A0A3SiZTB0, A0A3SiU3E5, A0A435BZ93, A0A3S2DEI1, A0A435APW1, A0A3SlQT55, A0A3SiWAI0, A0A3SiVV68, A0A3S2G7B5, A0A435MSE4, A0A3S2E2D1, A0A3S2FII5, A0A435FX83, A0A3S2I3S2, A0A436GZ56, A0A435RZZ7, A0A3S2PD80, A0A3S2ASJ9, A0A3S2CHW9, A0A435H1J4, A0A3S2K8R7, A0A434HTC2, A0A434L053, A0A3S2B6C9, A0A435W3M5, A0A3SiRN73, A0A3SiTEX9, A0A435WUH2, A0A3SlYYJ5, A0A3SiZ5H9, A0A3SiP7C5, A0A3SiTWK1, A0A3SlYOD5, A0A436CQA8, A0A3S2H9U8, A0A435PLS9, A0A434UUY9, A0A3SiLTZ1, A0A3SiNPV1, A0A3SiNFM4, A0A3SlQ0N3, A0A3SiPV14, A0A3T0JEF6, A0A434H439, A0A3SiHEH3, A0A434C2T4, A0A3S1S2I8, A0A3S2A461, A0A3Q8ZK90, A0A3Q8ZHB5, A0A3Q8ZIL9, A0A3S9EAD3, A0A3SiMPI8, A0A434UBI8, A0A434XFT3, A0A436XWS3, A0A3S9F3K3, A0A3SiLKL0, A0A3Q8XZG9, A0AII5G550, A0A090DJ28, A0A090DM34, A0A090DBY7, A0AISITYY4, A0A3S1DJG9, A0A434DDZ6, A0A0Q6P090, A0A0Q8AAI4, A0A0Q8AHI2, A0A0Q8KRL3, A0A0Q7WFD1, A0A0Q7WHK9, A0AIE2TiC8, A0A090EMI5, M5FEZ9, A0AIC2DYW0, A0A3SiCC63, A0A1W6X7T5, A0A2A3E108, A0A2A3D8Z1, A0A2A3DCN7, A0A2A3CRF1, A0A2A3B4T1, A0AIA5S3H4, A0A2A3C913, A0A2A3BMA6, A0A2A3BJT5, A0A2A3BJX8, A0A2A3BT00, A0A2A3APY2, A0A3F2UDB7, A0AII7B871, A0A440WS30, A0A441BU16, A0A3S4GRJ1, A0A441KJB7, A0A441X4E6, A0A440BP34, A0A442P447, A0A440DU90, A0A440FRF7, A0A439HIJ1, A0A440UWQ5, A0A440VCK8, A0A3S4JKR1, A0A439H2W3, A0A442F213, A0A3S3MKL6, A0A440LVW5, A0A439LX41, A0A443D0J6, A0A439SGK3, A0A441U7J5, A0A439Y445, A0A3S3FIG. 9, A0A442J3G7, A0A439J7B3, A0A3S3JNQ5, A0A440S8T8, A0A3S3FVM8, A0A442U2E3, A0A3S4EUE8, A0A439VKN8, A0A439QVQ7, A0A442LAV1, A0A3S3ENK4, A0A440VEQ9, A0A441HPM6, A0A439V159, A0A442JPW1, A0A439P052, A0A442WYU6, A0A3S3DCE8, A0A3S3F5U4, A0A3S3DDR4, A0A3S4EHK5, A0A442ZIC7, A0A442Y373, A0A442Y1R2, A0A440J505, A0A442JQC4, A0A442SPV3, A0A442VU88, A0A439PZL8, A0A440GZD4, A0A442S531, A0A440KF60, A0A442GQ66, A0A442QWA3, A0A441U254, A0A440N3Y3, A0A442MNF5, A0A3S3F8A3, A0A3S4BUS5, A0A3S3FRUO, A0A3S3K008, A0A3S4KZN4, A0A3S3JXW3, A0A443AW77, A0A439U373, A0A442M396, A0A440I076, A0A443ER01, A0A440CPT0, A0A442G6N0, A0A442JQB2, A0A441T6H6, A0A439IC62, A0A439ZLB8, A0A439FL51, A0A440L807, A0A442XI98, A0A440NAA5, A0A442XL36, A0A442Q031, A0A440TiP1, A0A441CKQ3, A0A439V3E1, A0A441SU59, A0A442YNW8, A0A442HP09, A0A439GTQ9, A0A439Z2H0, A0A440JZN6, A0A442YRX4, A0A442GXF5, A0A439MT07, A0A443G159, A0A442NGT0, A0A442JPV2, A0A439IIB9, A0A443A4B0, A0A439LUJ8, A0A440CVJ3, A0A440GVE4, A0A440JFB0, A0A441TTA0, A0A443EMA7, A0A442IY47, A0A3S3EEL7, A0A443APX6, A0A441V9P3, A0A442J2Z3, A0A442UTB2, A0A440IBK9, A0A440EGN0, A0A439XJM6, A0A443G1J9, A0A442JQC7, A0A442WB73, A0A442ZRR9, A0A440EB87, A0A442I508, A0A442B2D2, A0A439RGV3, A0A442XE59, A0A441XQ43, A0A439KBP8, A0A443FHR0, A0A442V753, A0A439XT51, A0A439KGC3, A0A439FUH3, A0A442VDM9, A0A440FQR3, A0A443CHW7, A0A443CDR9, A0A443E755, A0A442YE60, A0A442QCW7, A0A443E034, A0A443BMA7, A0A440JYY7, A0A442NIN0, A0A3P3G863, A0A271LPN7, A0A3A5KAS4, A0A3A5L844, A0A271KG65, E9DV60, A0A0B2WJX9, A0A0B4GVG0, A0A0B4GKK8, A0A0B4IAF8, A0A0B4G2X5, A0A167AMX9, A0A014N1B6, A0A3N5N557, A0A1V4V301, A0A1V5AGG2, A0AIV5AWY6, Q2FN86, A0A2V2MVB0, A0A3R7NX32, A0A2R4WGM2, A0AIE3GZU6, A0AIE3H0I6, B8EJN0, A0A395CUD5, A0A177P9W2, A0A2U1SNI8, A0A2D2CXA0, A0A2P6V6K0, A0A2P6VA87, A0A2P6VCG7, A0A2P6V942, A0A2P6VI36, A0A2P6VHH0, A0A2P6VA39, A0A2P6V3M9, A0A2P6V2A4, A0A2P6VB87, A0A2P6VES9, A0A2P6VPE2, A0A2P6VH52, A0A2P6VES2, A0A2P6VML2, A0A2P6V2M3, A0A2P6VH58, A0A2P6VK95, A0A2P6VMI8, A0A2P6VBH1, A0A2P6V9Q5, A0A2P6VI19, A0A2P6VGM6, A0A238FKR1, A0A349JEI0, A0A136J612, A0A136IJV1, A0A136J606, A0A136J3L2, A0A136ITG3, C1EAS9, C1E8G6, C1E8N1, C1FJX3, C1E192, C1MHF5, C1MZS3, C1MT37, A0A086MKH3, A0A159ZQX3, A0A159ZSK6, A0A165XJE2, A0A3S8UXA9, A0A3Q8U814, A0A3S8UYS9, A0AIL6LTM2, A0A246H318, A0A2G9CDV5, A0A1I6IYG6, A0A210Q014, A0A210Q865, A0A210QCM8, A0A210QRE0, A0A210PFV8, A0A210Q3Z0, A0A210QIL1, A0A210QAM1, A0A210R204, A0A210Q475, A0A210PED4, A0A210Q439, A0A210QTF2, A0A210R6V4, A0A210PET2, A0A210QAL4, A0A210QH12, A0A210PTJ6, A0A210QDU9, A0A210PKW3, A0A210PYD7, A0A210PEI4, A0A210Q2X6, A0A210QH27, A0A210Q9G7, A0A210QMZ3, A0A210PNY2, A0A210PKZ8, A0A210QAL2, A0A210QQF1, A0A210Q9Y6, A0A3Q3W1J6, A0A3Q3WBY9, A0A3Q3W970, A0A3Q4B8Q9, A0A3Q3WI21, A0A3Q3XDI1, A0A3Q3VSX4, A0A395J3G5, A0A395J2C4, E2L5Y0, V2YJ73, A0A0W0G933, F7G1K1, F7F243, F7FKH6, F6YOF0, F7FL16, A0A3Q3IFF7, A0A3Q3Q2I1, A0A3Q3Q2G6, A0A3Q3QBW9, A0A3Q3JMK3, A0A3Q3ILB0, A0A3Q3IFK2, A0A3Q3KJG2, A0A0D2MPA4, A0A0D2LGW3, A0A0D2N2N7, A0A0D2J9H2, A0A0D2M8B4, A9V705, A9V3K4, A9V0V7, A9VCQ3, A9VCI2, A9V610, A9VDX2, A9UWN2, A9VAX1, A9UWV1, A9UYMO, A9V068, A9V646, A9V0Z5, A9UNM7, A9UTMO, A9UVN3, Q8LP49, W9S1W6, W9SC56, W9S2J3, W9RMV6, W9R2F2, W9SEU6, W9RS41, W9RZT6, W9RGD7, W9SVD8, W9S9U6, W9SAT2, W9SGF4, W9SFY6, W9R8I4, W9QN88, W9RRQ7, W9S089, W9QYJ9, W9SJF4, W9R8B3, W9RV46, W9SEU1, W9SHC4, W9QI76, W9RLJ1, W9RUW2, W9S558, W9S6F9, W9QMK7, W9S5M0, W9S6T4, W9RG28, W9SB99, W9RS35, W9SIC7, W9S774, W9R087, W9S4J8, W9QCV1, W9QMB2, W9QMB6, W9RTS6, W9QJS9, W9RP60, W9S6W6, W9R543, A0A2P1EL58, A0A2P1ELG5, A0A2P1ELE8, M1NNC1, M1PN76, M1PXF7, H2EEU0, H2EF15, H2EET9, A0A371HKR4, A0A371E8L2, A0A371HQJ5, A0A371F8L2, A0A371HH60, A0A371E8Q5, A0A371GAP6, A0A371I1M6, A0A371HCN9, A0A371HKR5, A0A371GHE4, A0A371FRB0, A0A371H143, A0A371IEP8, A0A371HMC9, A0A371G0L4, A0A371H7G4, A0A371F5G3, A0A371EQX7, A0A371HK74, A0A371HCI3, A0A371F802, A0A371GT85, A0A371E352, A0A371H7E8, A0A371EKZ3, A0A371EVC7, A0A371HGU5, A0A371GDQ1, A0A371H1M0, A0A371G1Y0, A0A371EKI3, A0A371HKT8, A0A371HDB0, A0A371GTF3, A0A371G807, A0A371G1X9, A0A371F145, A0A371GCZ5, A0A371HJ92, A0A371HMD7, A0A371GXN7, A0A371FRUO, A0A371HB49, A0A371HDD5, A0A371HKS1, A0A371IER3, A0A371EZ33, A0A371HH99, A0A371F945, A0A371IEM8, A0A371E4N2, A0A371EL02, A0A371HBI0, A0A371F936, A0A371HME2, A0A371FJ78, A0A371GQ72, A0A371HCE8, A0A371EN60, A0A371GC08, A0A371E6H1, A0A371HKB0, A0A371FWJ5, A0A371HMF4, A0A371GXU7, A0A371HHMY2, A0A371HHME4, A0A371ECI5, A0A371EGW9, A0A371HMM7, A0A371HK49, A0A371H7E5, A0A371EBC1, A0A371EB07, A0A371ECG7, A0A371FNR9, A0A371HAZ0, A0A371EUQ1, A0A371E529, A0A371HCF5, A0A371EGY1, A0A371ET34, A0A371FNL4, A0A371E3B1, A0A371EG56, A0A371HB25, A0A371HB07, A0A371I1M7, A0A371ER06, P20918, Q08048, P26928, P26262, Q3V1T9, Q6TCI0, Q8C4E2, E0CXN0, H9H9R5, Q3UZ05, Q91XG8, Q8C9G5, Q91Y47, MOSRJ9, MOSUC6, MOU4G9, MORY71, MOSRJ7, MOTMOO, MOS383, MORH24, MOTF78, MOTDY2, MOU372, M0TB77, MOT6Z8, MOSRJ8, MORVH0, MOTDY4, MOTlW0, MOS810, MOU587, MOTCZ2, MORY70, MOTNE6, MOUAY4, MOSP19, MOTVR6, MOS240, MOS243, MOTZMO, MOT308, MOU8W4, MOTLZ6, A0A1I8MY63, A0A1I8M6T0, A0A1I8M8N5, A0A1I8N6V2, A0AII8NAG5, A0AII8MV57, A0AII8NEV1, G9K488, M3YFX5, M3Z3L5, M3YFW6, M3YQ99, M3XZU6, G2QGY9, A0A2T5B5R9, A0A2T5BAW0, S7PRA1, S7QDN3, S7MS05, S7NFT0, L5LR27, L5LGA9, L5M976, G1PGD5, G1PSR3, G1Q6A3, A0A409V789, A0A3L5TVE3, A0A3L5TQ46, A0A3R5Q2Z7, A0A2E8RW71, A0A2D5EZ81, A0A0F7E4G9, A0AII0BCH0, L7UOL6, Q68BK5, K7J454, K7IRC8, K7J148, K7J2B0, K7J4P8, W2TTT0, W2TUD4, W2TVZ2, W2TY26, W2SM98, W2TYP1, W2U1I9, W2TDU4, W2TCG9, W2T3P4, W2TVN9, W2TK17, W2SPF8, W2TZT5, W2SJP5, W2T4Y4, W2TP33, W2TVL3, W2TNE7, W2T4H9, W2T7H4, W2T2S3, W2TRM4, C7YKV9, C7ZLU7, C7YHL5, C7ZI7, C7ZNF6, C7Z7I1, C7YTH9, C7ZD94, C7ZLD5, C7ZBG8, C7YSG4, C7ZEX1, C7Z173, C7ZLD0, SuboUtiue Spec Clean C7YXE5, A0A1U8Q9H4, A0A1U7ZHR0, A0A1U8AZI5, A0A1U8QBE6, A0A1U7ZR51, A0A1U7ZR95, A0A1U7YTF2, A0A1U8PYZ2, A0A1U7ZWW8, A0AIU8A5W5, A0AIU8B247, A0AIU8A090, A0AIU8AYK2, A0AIU8Q8H4, A0A1U8B4B8, A0A1U8A4R2, A0A1U7Z5Y2, A0A1U7Z9J8, A0A1U7ZJI1, A0A1U8A7C2, A0A1U8B095, A0A1U7ZYN8, A0A1U8QBD9, A0A1U7ZYY7, A0AIU8Q7Y9, A0AIU7ZSA9, A0AIU8ARE9, A0AIU8Q7X5, A0AIU8BCK6, A0A1U7ZLL7, A0A1U7Z475, A0A1U8QCS1, A0A1U7ZID0, A0A1U8B0A1, A0A1U7YTS6, A0A1U8AM32, A0A1U8AN45, A0A1U8B9J8, A0A1U8AIZ5, A0A1U8Q0W3, A0A1U7ZY78, A0A1U8AZI0, A0A1U8BI59, A0A1U8ABD3, A0A1U8AY95, A0A1U7ZHQ5, A0A1U8QAY8, A0A1U7ZFF0, A0A1U7ZRU9, A0AIU7Z1L5, A0AIU8A603, A0AIU7Z5Y7, A0AIU8ARI0, A0AIU8Q9G1, A0AIU8ALH3, A0AIU7Z9W7, A0AIU8Q6V5, A0AIU8BCG2, A0AIU7Z2V6, A0A1U7Z4C3, A0A1U7Z3J0, A0A1U8A7U0, A0A1U8AN31, A0A1U8Q7F4, A0AIU8Q349, A0AIU8BA38, A0AIU8BGB5, A0AIU8AM80, A0AIU7ZY31, A7SZH1, A7SIZ3, A7SRX6, A7SBL4, A7RSM4, A7RNE4, A7RMZ7, A7S8E8, A7RX77, A7RFP1, A7RRUO, A7SBM9, A7RQ47, A7SHY9, A7S6R4, A8DUZ5, A7RU88, A7T3J8, A7SHH8, A7S968, A7SRE8, A7RPSO, A7SM86, A7SEE3, A7S366, A7T6F6, A7RWW1, A7S6Y4, A7S6U4, A7SAJ9, A7S7H3, A7SFL5, A7S6T9, A7RNV4, A7SDI1, A7S941, A7RKH7, A7S6Y5, A7T2L8, A7SEM5, A7SGJ1, A7SRX5, A7RU08, A7S942, A7SRE3, A7T7G4, A7SCA6, A7RIK4, A7RWB2, A7T1F4, A7RQ57, A7SI46, A7SBD5, A7RHX3, A7RQH4, A7T3Q6, A7SFU7, A7RSX6, A7RWB4, A7RUJ4, A7SVL2, A7RVC4, A7SXQ3, A7SIB1, A7SCH3, A7S3Q4, A7RWZ4, A7S293, A7SX86, A7SSU6, A7S547, A7SR11, A7S6D1, A7SDG5, A7RJD0, A7RQE2, A7RP92, A7S3L4, A7T9M2, A7T780, A7T8U0, A7SE24, A7RYZ8, A7SD38, A7SC13, A7S6L2, A7SJ07, A7SN79, A7TCJ6, A7RTC0, A7T1Z2, A7RWV8, A7S6U0, A7SL04, A7RJN3, A7SNB6, A7RM74, A7SK70, A7S6Y3, A0AIY2BK59, A0AIY2A5T7, A0A3Q4MLG0, A0A3Q4I4H3, A0A3Q4I627, A0A3Q4H1X3, A0A3Q4HU08, A0A3Q4H1Q8, A0A3Q4GWT9, A0A3Q4IEG5, A0A1U7LM76, A0A2Y9H1Q2, A0A2Y9HDN4, A0A2Y9HJV5, A0A2Y9HGI1, A0A2Y9HK72, A0A2Y9HH66, A0A2Y9GTJ0, A0A2Y9HJV1, A0A2Y9H6M8, A0A0P7B6P7, A0A0P7BBW4, A0A0P7BTD6, A0A0N8H811, A0A0P7BG58, A0A0P7BD71, A0A0P7ALI4, A0A0P7BIY5, A0A0P7B5T9, A0A0P7B6I9, A0A0P7BLF3, A0A0P7AUR2, A0A0P7AKI4, A0A0P7BL89, A0A0P7BSA0, A0A341BPU6, A0A341BNE4, A0A341C1A0, A0A341BM03, A0A341ATW5, A0A341C3X0, A0A341BNF2, A0A341CM99, A0A341BLW0, A0A068TC39, A0A0T7H3C9, A0A0T7FB36, A0A0T7FXT8, A0A068SW47, A0A0T7HIV6, A0A386E2E6, A0A346YQL0, AiDN01, Q4WBJ3, B0YAB7, B0XSS6, A0A229XX59, A0A229X3Y5, F0V833, F0V892, F0VKE6, F0VBW4, F0V9H8, F0JAV0, F0VJW9, F0VDC1, F0V7W6, F0VJW8, F0VI90, F0VKF4, A0A0F7UGU6, A0A0F7UM27, F0VKE8, A0A0F7UH55, F0VQW3, F0V8T4, A0A0F7UIB5, A0A0F7U8D9, A0A0F7U4M4, F0V8K8, F0VKE7, A0A0F7UQW1, F0VCD3, F0VJW7, A0A0H4LVB4, A0A0H4LYS9, A0A0H4MAT4, Q2KHJ3, A0A1A6FUU5, A0A1A6FTR8, A0A1A6GY88, A0A1A6HIS2, A0A1A6FW21, U6CR68, A0A2P6L9E4, A0A2P6L9F6, A0A2P6LI56, A0A2P6L9G3, A0A2P6KM95, A0A2P6K685, A0A2P6L9F0, A0A2P6K678, A0A2P6KT77, A0A2P6K695, D6PR18, D6PR67, A0A091S6P5, A0A091SBL5, A0A091T5V5, A0A091S1J9, A7UVW4, Q7S109, V5ILM8, Q7SFE8, V5ILY1, A0A0B0DW84, Q96U11, A0A0B0E396, A0A0B0E9R9, A0A0B0E9P7, F8N082, F8N4S9, F8MEC8, F8MC30, G4U7X4, G4UFH6, G4UD81, G4U9Q3, A0AIJ6HWP7, A0A314L2N5, A0A314KMT1, A0A1J6IQT9, A0A314KMZ2, A0A314KWS7, A0A314LDT0, A0A1J6J3K8, A0A1J6KDN9, A0A1J6ISD8, A0A1J6I486, A0A1J6ICK1, A0A1J6KII4, A0A1J6JDG5, A0A314KM80, A0AIJ6IQM4, A0A314L5L5, A0A314KNT5, A0A314L814, A0A1J6I9A0, A0A314L5W9, A0A1J6INQ4, A0A1J6IB07, A0A1J6IG63, A0A1J6JYUO, A0A1J6KUR6, A0A314L5M3, A0A1J6I9G8, A0A1J6KXS8, A0A1J6L9X1, A0A1J6IAJ9, A0A314KLH0, A0A314KHP6, A0A1J6K8T5, A0A1J6KOY6, A0A1J6KY06, A0A1J6I3J3, A0A1J6I7I0, A0A1J6J380, A0A314L474, A0A1J6HU55, A0AIJ6KMP9, A0AIJ6JV58, A0A314L438, A0AIJ6UN2, A0A314L6V5, G4XXY5, A0A1J6J9K7, A0A1J6KSS8, A0A1J6IF17, A0A1J6ID41, A0A314L7K3, A0A1J6L0B3, A0AIJ6IWL3, A0AIJ6IGU9, B9A9C5, B6EB06, A0AIU7VDG6, A0A1U7VUJ3, A0A1U7X9V3, A0A1U7WCZ7, A0A1U7XDD8, A0A1U7VEB0, A0A1U7Y2A8, A0A1U7XQK4, A0A1U7WXX2, A0A1U7WDB0, A0A1U7YLM9, A0A1U7WKL4, A0AIU7WLL2, A0AIU7XQZ3, A0AIU7W2G6, A0AIU7WAK5, A0AIU7W505, A0A1U7WKF6, A0A1U7VZG2, A0A1U7WH46, A0A1U7Y9T1, A0A1U7WED3, A0A1U7WV83, A0A1U7VL33, A0A1U7XGW4, A0A1U7VQE3, A0A1U7YRY2, A0A1U7W1W1, A0A1U7XXL7, A0A1U7Z1C5, A0A1U7YL89, A0A1U7WMH9, A0AIU7VPX9, A0AIU7X9V8, A0AIU7V5P9, A0AIU7XLP8, A0AIU7WT92, A0AIU7V2M1, A0A1U7WD36, A0AIU7VZ90, A0AIU7XDE3, A0AIU7V541, A0AIU7VJP5, A0AIU7W2Y1, A0AIU7VKV2, A0AIU7W5R6, A0AIU7WW15, A0AIU7W0I9, A0AIU7Y7Q9, A0AIU7VN54, A0AIU7VUC4, A0AIU7WBT7, A0A1U7XL68, A0A1U7W973, A0A1U7X5P3, A0A1U7YK15, A0A1U7XTF4, A0A1U7WPB7, A0A1U7UUN4, A0A1U7WCX4, A0A1U7XPV3, A0A1U7Y3E7, A0A1U7XGV9, A0A1U7Y6J9, A0A1U7VQF3, A0A1U7XPM9, A0A1U7WVB8, A0A1U7W731, A0A1U7XYW4, A0A1U7VPS9, A0A1U7WLD5, A0A1U7XC49, A0A1U7XLK4, A0A1U7W910, A0A1U7WKM8, A0A1U7Y8N9, A0A1U7VET8, A0AIU7WMI3, A0AIU7VC33, A0AIU7XCN9, A0AIU7W9Z4, A0AIU7X4E1, A0AIU7VW07, A0AIU7W8N5, A0AIU7VNJ5, A0AIU7VUJ7, A0AIU7XS99, A0A1S4A8G6, A0A1S3Z9W1, A0A1S3ZKW9, A0A1S3YVQ2, A0A1S3ZKG6, A0A1S3ZPQ4, A0A1S3XZW1, A0A1S4AIL3, A0A1S4AMR9, A0A1S4DLY3, A0AIS4AQV3, A0A1S4A3C8, A0AIS4BVH7, A0AIS3YT81, A0AIS4DJR1, A0AIS4BG39, A0AIS4AAV5, A0AIS3YCL1, A0AIS3YGV9, A0AIS4AS71, A0A1S4CBP5, A0A1S4AIV1, A0A1S3YGK9, A0A1S4C8A5, A0A1S3ZLY6, A0AIS3XKI4, A0AIS4BVI6, A0AIS3YS52, A0AIS3XGG9, A0A1S4C2V4, A0A1S4C7V6, A0AIS3YX32, A0A1S3X9D4, A0A1S4D768, A0AIS4CCG0, A0A1S4C7R5, A0A1S4AAW3, A0A1S3ZFI4, A0A1S4C0I7, A0A1S3Z379, A0A1S4CT07, A0A1S4BZ07, A0A1S4CBP9, A0A1S3YCE1, A0A1S3YOA5, A0AIS4DI92, A0AIS4ASV5, A0AIS3XFC6, A0A1S3X1W3, A0A1S4D7F2, A0A1S4D8R5, A0A1S4C899, A0AIS4AB13, A0AIS3YI37, A0AIS4BCW1, A0AIS3ZBM9, A0AIS3ZWG5, A0AIS3YH70, A0AIS4DEP7, A0AIS3YCZ3, A0A1S4BQF7, A0A1S3Z2M2, A0A1S4DMO5, A0A1S4DN45, A0A1S4BD11, A0A1S3Z7R3, A0A1S3ZF88, A0A1S3ZBH4, A0A1S3ZK91, A0A1S3XKN5, A0AIS4DEYO, A0AIS4DPZ0, A0AIS3YCZ7, A0AIS4BJE9, A0A1S4B6P0, A0A1S4DER9, A0A1S4A3U1, A0A1S3ZG58, A0A1S4BX98, A0A1S3YZW8, A0A1S4AAQ4, A0A1S3Y6Q2, A0A1S3Y621, A0A1S4BVJ0, A0A1S4DI53, A0A1S4AGM7, A0A1S3ZWE8, A0A1S4C7S1, A0A1S4C2N1, A0A1S4BVM4, A0A1S4AHG2, A0A1S4BIZ9, A0A1S3Z1I1, A0A1S4BD90, A0A1S3ZKX2, A0A1S4B4B4, A0A1S3Z7T1, A0A1S3ZXB1, A0A1S3YHU7, B9A9C4, A0A1S4BF74, A0A1S4A7H4, A0AIS3YH89, A0AIS3ZFQ4, A0AIS3XJQ9, A0AIS4AV00, A0A1S3ZWC2, A0A1S4AJE1, A0A1S4C3P0, A0A1S3Z2H5, A0A1S3Z761, A0AIS3ZGF4, A0AIS4ARU2, A0AIS4AAC7, A0AIS3ZBB3, A0AIS3ZWF8, A0A1S3ZIS3, A0A1S4BU26, A0A1S3ZIZ8, A0A1S4CTL2, A0A1S3Y6I1, A0AIS4BXG7, A0AIS3WZV5, A0AIS4BFA7, A0AIS3YUE5, A0AIS3ZFP6, A0AIS4BBN7, A0AIS4AAQ8, A0A1S4A7H2, A0AIS3YHW7, A0AIS4BF06, A0A1S4B7P0, A0A1S4A3X6, A0A1S4BF18, A0A1S4DQJ7, A0A1S4C7S6, A0AIS4DI64, A0AIS3YNY7, A0AIS3YZ96, E5D7E5, A0AIS4BTQ9, A0AIS4AAV8, A0A1S4BVJ1, A0A1S4CI20, A0A1S3YL50, A0A1S4AHU4, A0A1S4AGG2, A0A1S4C7S0, A0AIS4AGE4, A0A1S4C7G1, A0A1S4C1I5, A0A1S4C7F1, A0AIS4DEL0, A0A1S4C4Q3, A0AIS4BTX0, A0AIS4CUC0, A0A0U2UIV4, A0A0U2TUD0, A0A0U2K3G9, A0A091VZYO, A0A091W0K6, A0A091UP08, A0A091ULT1, A0A0N4XE48, A0A3P7CNB1, A0A0N4YIX3, A0A3P7A1Y9, A0A3P7AQW4, A0A0N4Y5X9, A0A158R0D4, A0A0N4XTQ9, A0A3P7B0Z6, A0A0N4YDL0, A0A0N4Y521, A0A0N4XTZ4, A0A0N4YX44, A0A158QWT4, A0A158R0F4, A0A0N4YRN0, A0A0N4XCL2, A0A158R0A2, A0A0N4XP57, A0A0N4YJU3, A0A158QZV0, A0A0N4XMB6, A0A158R2Z3, A0A0N4YT44, A0A0N4XW83, A0A158R099, A0A0N4XDZ8, A0A0N4XLC5, A0A0N4YJQ3, A0A0N4YT73, A0A0N4XKU9, A0A158QZH2, A0A158QX22, A0A0N4YVS7, A0A0N4XWH8, A0A0N4XTR1, A0A0N4YUM8, A0A0N4YSD3, K2PNL4, K2P3G0, K2LLY9, A0A1H4K6F6, A0A2P8ND91, A0A2E0JHX2, Q1QRJ6, A0AIQ3JTV4, A0A1M3C7Q1, A3WSN9, A0A1V4I335, Q3SMU6, A0AIH8BAS7, A0AII0D0D5, A0A1H9S965, A0A358JLT2, A0A2N6HD53, A0AIJ3FP03, A0AIJ3HMF5, A0AIJ3HW34, A0AIJ3GB39, A0AIJ3JNB8, A0AIJ3JZS2, A0AIJ3ISG7, A0AIJ3DXH5, A0A1J3E580, A0AIJ3GBL6, A0A1J3K818, A0A1J3H057, A0AIJ3GQN5, A0AIJ3HRL3, A0AIJ3DRG8, A0AIJ3GNA1, A0A1J3J3Z7, A0AIJ3JRE5, A0AIJ3FL37, A0AIJ3JQL6, A0AIJ3GRF7, A0AIJ3JX24, A0AIJ3GGC1, A0A1J3E7G3, A0A1J3F3A5, A0AIJ3JPT9, A0AIJ3DU93, A0AIJ3DTR9, A0AIJ3CXR2, A0AIJ3JBY9, A0A1J3E9F3, A0AIJ3FSR4, A0AIJ3JJ88, A0A1J3H7F2, A0AIJ3JTS5, A0AIJ3FCK6, A0AIJ3FKJ0, A0AIJ3DSR5, A0AIJ3GCV7, A0AIJ3FI92, A0A1J3G1G3, A0AIJ3HR35, A0AIJ3DHA0, A0A1J3E845, A0AIJ3FS72, A0A1J3G1T7, A0A1J3H5P9, A0AIJ3CQE8, A0A1J3IWX7, A0A1J3G0P7, A0A1J3IM74, A0A1J3DTR0, A0A1J3JQL3, A0A1J3E7J0, A0AIJ3DTZ1, A0A1J3J3Q6, A0AIJ3CCB9, A0AIJ3FIV7, A0AIJ3EAN5, A0AIJ3IVU3, A0AIJ3GZI8, A0AIJ3HK52, A0AIJ3HAN9, A0A1J3JM42, A0A1J3DI79, A0A1J3DSA9, A0A1J3DX70, A0A1J3HPH3, A0AIJ3DW17, A0AIJ3DSG0, A0AIJ3FWI3, A0AIJ3IK70, A0AIJ3IIK4, A0AIJ3JKH6, A0AIJ3CFM4, A0AIJ3EV57, A0AIJ3IX87, A0A1J3I886, A0A1J3J875, A0AIJ3EEU1, A0AIJ3ES29, A0AIJ3JTY3, A0A1J3I7Z8, A0AIJ3EFY5, A0AIJ3CL94, A0AIJ3I0U3, A0AIJ3IVB8, A0A1J3D311, A0AIJ3EEH9, A0AIJ3DSO4, A0A1J3D5T0, A0AIJ3DME5, A0AIJ3IH96, A0A1J3F2S3, A0AIJ3JRD9, A0AIJ3GZM1, A0A1J3FB28, A0A1J3EN22, A0A1J3HDE8, A0A1J3CMT3, A0A1J3EEN0, A0A1J3FFP7, A0A1J3JUT5, A0A1J3D2I7, A0A1J3H709, A0A1J3DF21, A0A1J3J0E4, A0A1J3H499, A0AIJ3GMB9, A0AIJ3GVE5, A0AIJ3DWJ7, A0AIJ3JHE1, A0AIJ3DMMO, A0AIJ3JS61, A0AIJ3IP86, A0AIJ3FSR0, A0A1J3J1W0, A0A1J3GG27, A0A1J3EMF4, A0A1J3G4X8, A0A1J3ELH1, A0A1J3EPX9, A0AIJ3GZP2, A0A1J3F8K9, A0A2I3GPI4, G1RKX8, M3ZBA9, G1RLC0, A0A2I3G9T1, G1RXL8, A0A2I3HH53, G1RLE8, G1R5A0, A0A2R5FFP8, A0A2D0H9I4, A0A318AZE0, A0A1A8UHT3, A0A1A8U2N8, A0A1A8UGE6, A0A1A8AMP3, A0A1A8UYV9, A0A1A8DKN8, A0A1A8CX61, A0A1A8F858, A0A1A8KU64, A0A1A8J1X1, A0A1A8HP15, A0A1A8Q0T0, A0A1A8M3J2, A0A1A8N5R8, A0A1A8S3A7, A0A3D1P0S1, A0A3D1P0P9, A0A2T4ZM68, A0A2D9MLX3, A0A2E5UVY4, Q2CK54, A0AlN6H695, A0A254R8C3, K2I7J2, A6X4J9, A0A01l UJA9, A0A137XJ12, A0A3R8ZDT7, A0A1J6HPX3, A0A256FNR5, A0A397M124, A0A2P9HHH8, U4V6A2, C4WPE7, M5JM46, A0A256GVM1, A0A2S7J3N1, A0A256G407, A0A1A9FPL5, A0A256F053, A0A2M9SAC5, A0A2M9RTW1, A0A2M9RPN7, A0A248UL68, A0A248UDK1, A0A421BXV1, U1XL45, A0A3N0BIYI, A0A2S8YAJ0, A0A2S8Y7R3, A0A2S8TZD4, A0AIV3YWR0, A0A317LE59, A0A292GL07, A0A256FB23, A0A0L8HR65, A0A0L8G008, A0A0L8GIC5, A0A0L8G5H6, A0A0L8HWE5, A0A0L8HQP4, A0A0L8HYY9, A0A0L8G081, A0A0L8HPA3, A0A0L8HXI7, A0A0L8HS26, A0A0L8FY82, A0A0L8HWF0, A0A0L8HQ38, A0A0L8G444, A0A0L8IDR0, A0A0L8G3A8, A0A0L8HMG8, A0A0L8HW99, A0A0L8HWE2, A0A0L8H6T5, A0A0L8HQN8, A0A0L8HP85, A0A0L8HDL0, A0A0L8FNP5, A0A0L8FJ32, A0A0L8HW19, A0A0L8GXG5, A0A0L8GT95, A0A0L8GXD9, A0A0L8HWB9, A0A0L8GSV1, A0A0L8GYH4, A0A0L8FUE7, A0A0L8HNN0, A0A0L8H7N3, A0A0L8GWJ9, A0A0L8G1Z8, A0A0L8HP77, A0A0L8G183, A0A0L8FRC1, A0A0L8I283, A0A0L8HWA4, A0A0L8I485, A0A0L8GWC4, A0A0L8H6J5, A0A0L8HZ03, A0A0L8HPE9, A0A0L8FRF9, A0A2U3VG05, A0A2U3VGE7, A0A2U3VVF2, A0A2U3X506, A0A2U3W015, A0A2U3VZS1, A0A2U3ZHH0, A0A2U3X5D6, A0A2U3X502, A0A2U3X508, A0A0B1SMS5, A0A0B1SZF3, A0A0B1T7G4, A0A0B1TNZ7, A0A0B1SVQ8, A0A0B1TSF3, A0A0B1THL2, A0A0B1RPJ3, A0A0B1TVI4, A0A0B1T7X3, A0A0B1TLL5, A0A0B1SJV9, A0A0B1SZQ3, A0A0B1TJJ8, A0A0B1TQU4, A0A0B1SPE9, A0A0B1TRM5, A0A0B1TM49, A0A0B1TP05, A0A0B1TLV5, A0A0B1SLD5, A0A0B1T699, A0A0B1TQQ2, A0A0BISIU2, A0A0C3GVM9, A0A0C3GHZ3, A0A0C3GYJ2, A0A0C3C6A0, A0A0C3GWZ0, E4YAT7, E4X175, E4Z726, E4XPL0, E4YKT2, E4WX62, E4WXA9, E4X236, E4XM60, E4X025, E4YGB1, E4YEZ4, E4Y3D2, A0A3N6PYR2, D7RJY6, D7RJY4, B6JDB0, A0A238BQ40, A0A183HYH0, A0A183HKI7, A0A183H9Q1, A0A183HZR7, A0A183HM98, A0A183I5Y8, A0A183H621, A0A183HW79, A0A183HZQ7, A0A183HBN7, A0A183HGE2, A0A183I2D1, A0A183I087, A0A183GZD3, A0A238CiL1, A0A238C0I5, A0A238BZT1, A0A238CiR8, A0A3P7VC50, A0A183HF33, A0A238BHJ2, A0A238C1C5, A0A238BUI0, A0A238BPJ5, A0A3P7KGB4, A0A182ERB1, A0A3P7JTR2, A0A182EUC0, A0A182E9R2, A0A182E294, A0A3P6SEL8, A0A3P6STP6, A0A182EK23, A0A182EGU8, A0A182EBS9, A0A182ELR8, A0A182E8N8, A0A182E853, A0A182E148, A0A182E7K9, A0A182DWJ9, A0A182E5V3, A0A044TXH1, A0A044TB24, A0A044V6J3, A0A044SAK8, A0A044T6C9, A0A044TQE7, A0A044VG28, A0A044TXN9, A0A044QW86, A0A044TQV4, A0A044TS10, A0A044VIE3, A0A044SXT1, A0A2K6W0F3, A0A2K6VJT9, A0A2R7VUY6, A0A2R7WMN5, A0A2R7X3S8, A0A2R7VTV5, A0A2R7WIU5, A0A2R7WIV9, A0A2R7WJA5, A0A060X7U1, A0A060XRA8, A0A060XME5, A0A060XFN8, A0A060W8B1, A0A060WHF0, Q5DVP8, A0A060XU84, A0A026W7N3, A0A3L8E197, A0A026VZ91, A0A026WBB7, A0A3L8DVC2, A0A3L8DB52, A0A026W4C2, A0A3L8DPD8, A0A026X1V9, A0A3L8EIU8, A0A3L8DP99, A0A026W2Z5, A0A3L8DFM8, A0A0L7LTQ8, A0A0L7L2P1, A0A0L7LM72, A0A0L7L0G5, A0A0L7L335, A0A0L7L2H0, A0A0L7LA69, T5ADX7, T5AAZ9, A0A2A9PMS4, A0A2A9PLL3, V8NK09, V8PHB4, S3D177, S3C2I7, A0A091WHX0, A0A091XY89, A0A091WJ34, A0A091WK45, A0A1S8WUG3, A0A074ZV38, A0A075A3I4, A0A1S8X8S4, A0A1S8WQZ1, A0A074Z2H8, A0A074Z9I0, A0A1S8WPP4, A0A074ZMS3, A0A2E9RXB9, A0A1D2NBH8, A0A1D2MZY1, A0A1D2NJJ1, A0AID2MYU3, A0AID2ND60, I3K037, I3KFD6, I3K036, I3KFB8, I3KCE9, I3JAQ5, I3JAR3, I3KCF0, I3KFB7, I3J849, I3J1D2, A0A0P6YFR3, A0A293M3U3, A0A293LHY5, F7CM56, F7BQ99, F7FSH7, F7CM40, F7FSI1, 080354, A0A0T6BGW1, A0A0T6B7H3, A0A0T6B598, Q95ME7, D0PTV6, G1U9Q9, G1SSQ6, G1T127, G1SMZ3, G1SFS6, D0PPI8, C9E9X4, E0CW66, E0CW57, B9V0G8, A0A0D3HR23, A0A0D3HAE3, A0A0D3EL74, A0A0D3G020, A0A0D3HR22, A0A0D3EX91, A0A0D3H9S4, A0A0D3GD84, A0A0D3ESA3, A0A0D3EUU8, A0A0D3FJY4, A0A0D3GS78, A0A0D3EUU6, A0A0D3G016, A0A0D3EUU9, A0A0D3GS89, A0A0D3FKE2, A0A0D3ESB1, A0A0D3H9S5, A0A0D3FU00, A0A0D3EUU5, A0A0D3GD82, A0A0D3EUU4, A0A0D3EUV0, A0A0D3FU04, A0A0D3ESA2, A0A0D3EWT3, A0A0D3GI36, A0A0D3HI98, A0A0D3EQN3, A0A0D3FR49, A0A0D3EL79, A0A0D3FXQ4, A0A0D3G018, A0A0D3H9R8, A0A0D3FTZ3, A0A0D3G022, A0A0D3GHQ2, A0A0D3EX86, A0A0D3EWT4, A0A0D3FVT7, A0A0D3HHT7, A0A0D3GS79, A0A0D3G017, A0A0D3G132, A0A0D3EL82, A0A0D3FVUO, A0A0D3H9S1, A0A0D3FVT5, A0A0D3HCH4, A0A0D3EL84, A0A0D3FU01, A0A0D3EUU3, A0A0D3HS10, A0A0D3HV71, A0A0D3G021, A0A0D3H9R9, A0A0D3EUV2, A0A0D3ESB4, A0A0D3FFK8, A0A0D3EX49, A0A0D3HSZ8, A0A0D3HX90, A0A0D3H9S2, A0A0D3GIF2, A0A0D3G9H3, A0A0D3HJF3, A0A0D3G019, A0A0D3FVT4, A0A0D3G3N6, A0A0D3FUK4, A0A0D3HHT5, A0A0D3G011, A0A0D3GS82, A0A0D3GS90, A0A0D3GI35, A0A0D3G012, A0A0D3G7P0, A0A0D3G013, A0A0D3G550, A0A0D3G024, A0A0D3FVS9, A0A0D3EL80, A0A0D3HCH7, A0A0D3EX87, A0A0D3FVT0, A0A0D3GI32, A0A0D3HHT9, A0A0D3G3N8, A0A0D3FRT4, A0A0D3ESB0, A0A0D3FT93, A0A0D3HHU2, A0A0D3G023, A0A0D3FUM6, A0A0D3ESA7, A0A0D3GS81, A0A0D3FVS8, A0A0D3HJF4, A0A0D3G3N7, A0A0D3G3P0, A0A0D3EJR8, A0A0D3G131, A0A0D3EL81, A0A0D3ERR0, A0A0D3G9G2, A0A0D3FAT2, A0A0D3EUU7, A0A0D3HV70, A0A0D3EL83, A0A0D3FU02, J3L4R2, J3L0V7, J3MLV8, J3LUL2, J3L5D6, J3LLH5, B9V0I5, J3MZX1, J3LPV8, J3L2Q4, J3NBU7, A0A0UIWXR2, J3M4A9, G2XM38, J3L2R2, J3MFKO, J3N1W7, J3L2A1, J3LX34, J3KXUO, J3M263, J3MZW5, J3M1Q6, J3MLV6, J3LQ96, J3MZW8, J3NB41, J3KXT4, J3L6M2, J3MQB8, J3KWJ2, J3LXT3, J3M4A7, J3MID2, J3M1Q3, J3L4R1, J3KXU3, J3L2Q7, J3MEX9, J3MLV9, J3M4A8, J3MF85, G2XM40, J3LVJ9, J3LXT4, J3L6J2, J3LWL4, J3MQW0, J3KUK1, J3M1Q9, J3M265, J3M1Q4, J3MEX8, J3LXT1, J3KW69, J3L2Q8, J3L809, J3M264, J3NFE2, J3MF84, J3L4Q9, J3LXT5, J3LW49, J3KXU2, J3LWL5, J3L2Q3, J3MLW9, J3L2Q5, J3M780, J3M7G4, J3MGY9, J3LXT6, J3N0F9, I1NQI2, I1NQI4, I1PZU2, I1NKC7, I1PL71, I1QTK2, I1NU21, I1P904, I1R3R0, I1NTS1, I1NQI7, I1PL76, I1NQI3, I1R3R2, I1R4I3, I1PCQ7, I1QS23, G2XMM6, I1PL69, I1Q381, I1PJJ4, G2XMM7, G2XMM5, I1PPM3, I1QR22, I1PTT5, I1R3R1, G2XMM4, I1PPM4, I1PIJ8, I1QR27, I1PL68, I1QR20, I1PQE8, I1PL73, I1PST0, I1NU52, I1PL72, I1QR24, I1PX09, I1PZT8, I1PPM7, I1NQI6, G2XMV1, I1PST2, I1PMV0, I1QR21, I1R8A7, I1R3Q9, I1PL77, I1NTS0, I1QYI5, I1NLF0, I1QR28, I1PPM6, I1NS91, I1PK04, I1QXD1, I1PL79, I1P0W9, I1PCD1, I1P4I6, I1PPL9, I1NU57, I1PK06, I1Q383, I1R5B8, I1NQI9, I1NLF3, I1QXD4, I1PK03, I1QBG0, I1QBF9, I1QR19, I1PPM5, I1QBG7, I1PH64, I1QBF7, I1QYI6, I1NQI5, G2XLC0, I1PPMO, I1Q380, I1PQE7, I1PIJ7, I1PSR4, I1PL75, I1NLE2, I1PPM2, I1PL74, I1PSS6, I1PSS8, I1NTS2, I1QTK4, I1Q3F8, I1NLF2, I1NS88, A0A0D9YK84, A0A0D9YCX9, A0A0D9ZG94, A0A0D9ZKN7, A0A0D9ZJI1, A0A0D9YIH2, A0A0D9ZKN8, A0A0D9ZR94, A0A0E0B677, A0A0D9YFW0, A0A0D9Y5E5, A0A0E0AL88, A0A0D9YI07, A0A0E0ARK9, A0A0D9Y5D1, A0A0D9ZMW8, A0A0D9Y5E7, A0A0D9Z436, A0A0D9ZR74, A0A0E0AL85, A0A0D9ZIM8, A0A0D9YFW3, A0A0E0A5I8, A0A0D9Y5C8, A0A0D9ZUP3, A0A0E0BEX9, A0A0E0AL72, A0A0E0AL76, A0A0D9ZR97, A0A0E0BFC3, A0A0E0B678, A0A0D9ZIN0, A0A0D9ZFJ1, A0A0D9ZRA5, A0A0D9YFW4, A0A0D9ZRA4, A0A0D9ZR98, A0A0D9ZRA7, A0A0D9ZR81, A0A0E0AIR6, A0A0D9YA73, A0A0E0BNH0, A0A0D9YIC5, A0A0E0BNG7, A0A0D9ZG93, A0A0D9ZZ82, A0A0D9ZJF8, A0A0E0A5J0, A0A0D9Z9I2, A0A0D9ZRS7, A0A0D9Y5C9, A0A0D9ZIM7, A0A0E0AL74, A0A0D9YC92, A0A0D9Y4Z1, A0A0D9YCY7, A0A0D9ZRA6, A0A0D9ZR95, A0A0D9Y5E4, A0A0D9Y5E6, A0A0D9ZR83, A0A0D9Y3F4, A0A0E0BGJ9, A0A0E0BR07, A0A0D9YFX1, A0A0D9ZR90, A0A0D9YCY3, A0A0D9YI02, A0A0D9ZIN2, A0A0E0B670, A0A0E0B668, A0A0D9ZR80, A0A0E0BNG4, A0A0D9YYI7, A0A0E0A5J1, A0A0E0BR04, A0A0E0B667, A0A0E0B671, A0A0D9YI05, A0A0D9YCY4, A0A0D9ZUP5, A0A0D9Y5D0, A0A0D9ZR79, A0A0D9YIG5, A0A0E0ARL0, A0A0E0ABJ9, A0A0D9ZKP7, A0A0E0BPR9, A0A0D9Z920, A0A0D9YIH3, A0A0D9Y5E8, A0A0E0B705, A0A0D9Y5D3, A0A0D9YFW1, A0A0E0ARL1, A0A0E0BVT0, A0A0E0A194, A0A0D9ZRA3, A0A0D9YFW2, A0A0D9ZUP4, A0A0D9ZR96, A0A0D9Y5D2, A0A0E0B985, A0A0E0B664, A0A0D9ZRS6, A0A0D9ZKN9, A0A0E0B982, A0A0D9YT30, A0A0D9YCY1, A0A0D9YFX0, A0A0D9YIG8, A0A0D9ZG92, A0A0D9ZR82, A0A0D9YFW8, A0A0E0ARK8, A0A0D9ZR78, A0A0E0AIE6, A0A0D9ZIM6, A0A0D9YFW7, A0A0D9ZKP8, A0A0D9YI03, A0A0E0BTG8, A0A0D9ZRA2, A0A0E0DCW6, A0A0E0C8L4, A0A0E0DFW2, A0A0E0EUL2, A0A0E0DE62, A0A0E0CAW3, A0A0E0DNX0, A0A0E0EUM2, A0A0E0CLE9, A0A0E0EUK8, A0A0E0BXB3, A0A0E0DE59, A0A0E0DC72, A0A0E0EUK7, A0A0E0DIT6, A0A0E0C5U5, A0A0E0ECX2, A0A0E0DXL5, A0A0E0DIT9, A0A0E0DSYI, A0A0E0EWS8, A0A0E0EUL1, A0A0E0FCQ8, A0A0E0FA77, A0A0E0EUL0, A0A0E0EUM1, A0A0E0C5U3, A0A0E0CBD2, A0A0E0DE49, A0A0E0DE58, A0A0E0CBC9, A0A0E0E351, A0A0E0CL32, A0A0E0F268, A0A0E0CAW4, A0A0E0DC71, A0A0E0C599, A0A0E0DIU3, A0A0E0E354, A0A0E0DC70, A0A0E0ECX0, A0A0E0BZ76, A0A0E0FAG5, A0A0E0F2L6, A0A0E0DE64, A0A0E0C5U8, A0A0E0DXL9, A0A0E0F269, A0A0E0CB81, A0A0E0E2S8, A0A0E0EWS6, A0A0E0C5U2, A0A0E0C5U7, A0A0E0DML9, A0A0E0DC67, A0A0E0DCZ3, A0A0E0E3L6, A0A0E0DE54, A0A0E0FA73, A0A0E0D9W7, A0A0E0BZ80, A0A0E0DIS8, A0A0E0FA68, A0A0E0BZ68, A0A0E0DC68, A0A0E0C3C6, A0A0E0D9B1, A0A0E0DBN8, A0A0E0ECW5, A0A0E0EUK6, A0A0E0D2Q4, A0A0E0F3G5, A0A0E0FAZ3, A0A0E0DIUO, A0A0E0DIS6, A0A0E0DE56, A0A0E0FA37, A0A0E0DJC4, A0A0E0FA69, A0A0E0ECX4, A0A0E0EIN0, A0A0E0DIS7, A0A0E0DE65, A0A0E0DML8, A0A0E0EA66, A0A0E0BZ79, A0A0E0FDQ6, A0A0E0DIS5, A0A0E0C8L7, A0A0E0ECW7, A0A0E0E264, A0A0E0FDQ5, B9V0L1, E0CW78, A0A0E0GKI4, A0A0E0FIN1, A0A0E0FIP8, A0A0UIWYS9, A0A0E0HGH4, A0A0E0J8N9, A0A0E0INR6, A0A0E0H0N8, A0A0E0FQS6, A0A0E0GKI6, A0A0E0GKI2, A0A0E0HXK8, A0A0E0J697, A0A0E0GX03, A0A0E0FQR8, A0A0E0HSU5, A0A0E0FQ49, A0A0E0H0P1, A0A0E0GX02, A0A0E0HJW4, A0A0E0HS89, A0A0E0GKI7, A0A0E0I7U3, A0A0E0H0P8, A0A0E0FGF6, A0A0E0J8N8, A0A0E0INB5, A0A0E0HUM6, A0A0E0H0N9, A0A0E0FWA9, A0A0E0GDA8, B9V0Q1, A0A0E0H2X3, A0A0E0HY24, A0A0E0G7M5, A0A0E0FIM7, A0A0E0FQR3, A0A0E0H6W8, A0A0E0GXL4, A0A0E0IXT2, A0A0E0FWUI, A0A0E0FQR6, A0A0E0H0P7, A0A0E0JB34, A0A0E0GW59, A0A0E0HGH5, A0A0E0IZH0, A0A0E0H0P6, A0A0E0INB6, A0A0E0FIN2, A0A0E0HR40, A0A0E0GKJ1, A0A0E0FIP9, A0A0E0INC5, A0A0E0H0N5, A0A0E0HY06, A0A0E0FQR7, A0A0E0GKJ2, A0A0E0GKH3, A0A0E0I273, A0A0E0INB4, A0A0E0IZH1, A0A0E0FIN0, A0A0E0HGG1, A0A0E0HS95, A0A0E0GKI5, A0A0E0H0P0, A0A0E0J2D2, A0A0E0GXK3, A0A0E0J696, A0A0E0I250, A0A0E0GKI3, A0A0E0FQS1, A0A0E0HLM1, A0A0E0GXR3, A0A0E0FWQ0, A0A0E0J8N4, A0A0E0HS91, A0A0E0HLM4, A0A0E0IZG9, A0A0E0I254, A0A0E0INC4, A0A0E0FU79, A0A0E0GYT0, A0A0E0J9E6, A0A0E0FLY3, A0A0E0FIP7, A0A0E0GPL9, A0A0E0HST7, A0A0E0J7G5, A0A0E0FI93, A0A0E0FWT5, A0A0E0I249, A0A0E0I0E2, A0A0E0H0N6, A0A0E0GP69, A0A0E0H0N7, A0A0E0IGG9, A0A0E0IXT5, A0A0E0IXT1, A0A0E0I252, A0A0E0FU81, A0A0E0GKJ0, A0A0E0JCW6, A0A0E0JB33, A0A0E0GP70, A0A0E0GKI9, A0A0E0HSU3, A0A0E0FIN9, A0A0E0J698, A0A0E0H6W7, A0A0E0FNQ7, A0A0E0GKJ3, A0A0E0FWT8, A0A0E0J2D0, A0A0E0HT73, A0A0E0HS88, A0A0E0FIN8, A0A0E0INB8, A0A0E0FU76, A0A0E0FIN7, B9V0Q0, A0A0E0FIN4, A0A0E0KQI0, A0A0E0KF64, A0A0E0KVP1, A0A0E0MCH2, A0A0E0M481, A0A0E0KM14, A0A0E0LRX0, A0A0E0JS11, A0A0E0JFS6, A0A0E0KQJ0, A0A0E0KVP4, A0A0E0M493, B9V0S7, A0A0E0KNA2, A0A0E0LM11, A0A0E0KVP5, A0A0E0LRW9, A0A0E0JPL8, A0A0E0KYW2, A0A0E0JFS5, A0A0E0MC44, A0A0E0JPM2, A0A0E0LCW7, A0A0E0JQG4, A0A0E0LDK4, A0A0E0MJ94, A0A0E0KVP2, A0A0E0JPL6, A0A0E0LHZ7, A0A0E0LBV1, A0A0E0L4C9, A0A0E0JPM1, A0A0E0KQI6, A0A0E0M482, A0A0E0M4V9, A0A0E0LZP4, A0A0E0JFQ8, A0A0E0LYC7, A0A0E0MJ93, A0A0E0MQ50, A0A0E0M487, A0A0E0LRW7, A0A0E0KML7, A0A0E0LM19, A0A0E0KW47, A0A0E0KVN9, A0A0E0M6P1, A0A0E0M6P2, A0A0UIWXQ2, A0A0E0LZM8, A0A0E0KSK1, A0A0E0KQI4, A0A0E0JE78, A0A0E0KOK1, A0A0E0KAM8, A0A0E0MKA3, A0A0E0KQI5, A0A0E0L2K7, A0A0E0JFS2, A0A0E0JRX9, A0A0E0KFK8, A0A0E0KVN4, A0A0E0KQI8, A0A0E0JM75, A0A0E0LCW4, A0A0E0KQI3, A0A0E0LD93, A0A0E0LCX3, A0A0E0M492, A0A0E0KNT6, A0A0E0K5I5, A0A0E0JLN9, A0A0E0KVP3, A0A0E0KRU6, A0A0E0JM78, A0A0E0KYV9, A0A0E0KLA8, A0A0E0JS10, A0A0E0LM06, A0A0E0MC39, A0A0E0KNA3, A0A0E0JRK2, A0A0E0KYY4, A0A0E0LM08, A0A0E0JRK4, A0A0E0JLN5, A0A0E0L8B2, A0A0E0MDQ4, A0A0E0KVN6, A0A0E0JM73, A0A0E0KQI2, A0A0E0JPL9, A0A0E0JM82, A0A0E0LYG7, A0A0E0KNT9, A0A0E0LET8, A0A0E0JSO9, A0A0E0LZM7, A0A0E0KNU2, A0A0E0JS12, A0A0E0LRW8, A0A0E0JM79, A0A0E0JRX7, A0A0E0KYY5, A0A0E0LZM9, A0A0E0JTU7, E0CWC2, A0A0E0P929, A0A0E0QUS7, A0A0E0Q9Q2, A0A0E0R5M3, A0A0E0R5M2, A0A0E0N0I2, A0A0E0PPL5, A0A0E0N0I7, A0A0E0N0II, A0A0E0PEJ2, A0A0E0QUS6, A0A0E0PEJ4, A0A0E0PY75, A0A0E0R492, A0A0E0N0I4, A0A0E0PI15, A0A0E0NWN9, A0A0E0QF46, A0A0E0R5M4, A0A0E0PTZ3, A0A0E0MRI6, A0A0E0PEI3, A0A0E0PYZ1, A0A0E0R5M1, A0A0E0PEJ6, A0A0E0P930, A0A0E0R3S3, A0A0E0RKL1, A0A0E0Q9N8, A0A0E0MYM4, A0A0E0RI41, A0A0E0N0H4, A0A0E0Q9P1, A0A0E0PZV4, A0A0E0PEJ5, A0A0E0PZG7, A0A0E0QF44, A0A0E0P3N3, A0A0E0P717, A0A0E0PZ15, A0A0E0Q9Q1, A0A0E0P724, A0A0E0P936, A0A0E0REI6, A0A0E0N628, A0A0E0NRR4, A0A0E0QUS8, A0A0E0MS90, A0A0E0QVH0, A0A0E0RDA8, A0A0E0P672, A0A0E0N0H5, A0A0E0PEI8, A0A0E0PEJ3, A0A0E0N633, A0A0E0MT99, A0A0E0QF47, A0A0E0QF43, A0A0E0PZG2, A0A0E0P571, A0A0E0PF17, A0A0E0MT98, A0A0E0PEI6, A0A0E0NL99, A0A0E0QXV0, A0A0E0MTA1, A0A0E0N631, A0A0E0N5M2, A0A0E0N3D9, A0A0E0Q505, A0A0E0PI17, A0A0E0P556, A0A0E0P7R6, A0A0E0Q6Z5, A0A0E0P4C6, A0A0E0PF18, A0A0E0QUT5, A0A0E0N3E2, A0A0E0P934, A0A0E0P723, A0A0E0MSU8, A0A0E0RDA9, A0A0E0N0J0, A0A0E0N5M3, A0A0E0QUT6, A0A0E0P935, A0A0E0NFS2, A0A0E0QXU8, A0A0E0MT87, A0A0E0P933, A0A0E0N5Z2, A0A0E0RFL8, A0A0E0PZG5, A0A0E0PEJ1, A0A0E0P726, A0A0E0PBB3, A0A0E0NX59, A0A0E0PTY5, A0A0E0Q9N9, A0A0E0N3D6, A2YMH7, B8AP81, B8A9Q7, B8B3W2, A2YDY8, A2XYE9, B8AUV8, B8BFX5, A2XPF2, B8B7X0, B8AUV4, B8AAG4, A2XRP3, B8B3K4, B8AAN9, B8A6L2, B8BIX3, B8B784, B8A6Z2, B8BE63, A2X9W0, B8BBD1, B8AYF8, A2Z666, A2Y9R2, B8A7D5, B8ASQ4, B8A779, A2ZIL9, A2XRP6, B8AUV7, A2YEC0, B8AAP0, A2WVT4, B8BM83, A2XTD8, B8B2E5, A2ZBG9, B8AUV2, B8AS67, B8BEE8, A2YMH8, A2Y2G0, A2YMG6, B8ASF5, A2ZCH9, A2XTE2, B8ATQ3, B8BE64, A2XTD2, B8ATQ2, B8AS68, B8ATQ5, B8A780, A2WM87, B8BLW7, B8AK50, A2YMG7, A2XTE5, B8AUZ5, A2WTM1, B8AUV3, A2Z664, A2Z3T6, A2WXE7, B8AB24, A2YER5, B8AAN5, A2WXT2, B8AUW7, B8BE67, A2WVT7, A2XVC1, A2YTP0, B8BLW6, B8A783, A2XTD1, A2XTD0, B9V0N4, B8ATQ4, A2ZB52, B8BEE9, A5JS87, A2ZB38, A2WTM3, A2Y9Q7, B8AZQ7, B8A782, B8AUW5, A2XE17, A2ZN71, A2WRY3, A2WKV0, A2XTD4, B8A7B9, A2YOR8, B8AUV5, B8ATY7, A2XII0, A2XTE4, A2X5J7, A2YOQ5, B8BE62, B8AVL9, A2ZB35, A2WT48, A2XTE3, B8AUV6, A2XNUO, A2XXZ2, B8AUW6, B8BE66, B8AUV1, B8B3W0, B8BP34, A2ZHT0, A2ZCI0, Q0JDYO, Q69MP3, B9FEC4, B9FBW5, B9EYI9, Q0JD96, B9FZD1, B9FCW6, B9GBP9, Q5N835, A0A0P0Y6H8, B9G4Y0, B9FMN5, A0A0P0WFW5, B9ETD6, Q60D67, A3CFG6, A0A0P0UZX5, A0A0P0XQJ7, Q0J9S4, A0A0P0WIC4, Q5Z607, Q0J9S9, A0A0P0WSZI, Q2R920, A0A0N7KR84, A0A0P0XQY3, A0A0P0V6A7, Q5NB60, Q0J9S8, Q5QNL1, Q69MN9, A0A0N7KJ20, A3C195, Q7X6C7, Q0JEB3, A0A0P0YOI8, B9FCQ2, Q2QY66, B9FMM3, B9FEC2, B9FCW7, A0A0P0XQ09, A0A0P0XQ04, A0A0P0WXZ2, B9FCQ0, C7J132, A0A0P0XRC3, Q7XQT3, Q0JH14, A3BL04, C7J1M6, Q2RAY8, Q5QLM8, A3AVE3, A3BCZ7, A0A0P0Y6V4, A3AXR8, Q5JQW1, A0A0P0WF90, A0A0P0WF87, B9F816, B9FCQ5, A0A0P0XYU6, C7J3E6, B9FXT7, Q0IZT3, A0A0P0Y7N1, A0A0P0X5S1, A0A0P0VAV4, A0A0P0WG14, A0A0P0XQ32, A0A0P0WIA4, A0A0P0WAJ2, A2ZYH9, Q0J9T9, Q0JEB1, Q2QWY4, Q7XL24, B9FEC1, B9GE14, A3AS58, A0A0P0VBI4, B9G4X2, A0A0P0V6F2, A0A0P0W6Q5, Q0J9T4, B9FCP9, A0A0P0XYD6, B9EUW0, Q0J9S6, Q8H353, A0A0P0V6E9, B9FCP8, Q7XQU2, A0A0P0VBE8, B9EU56, A0A0N7KHG5, Q7XMH7, B9F999, Q0J9T3, Q0ITYO, B9G985, Q0E0R8, Q7XL27, B9FCQ4, B9EU61, A3A0E3, B9EUW2, B9EVA3, A0A0P0Y700, A0A0P0XPZ9, A0A0P0W9H2, A0A0P0WF77, A0A0P0WF71, A0A0N7KDE3, A0A0N7KJSO, Q0JEPI, Q7XQU6, B9FKZ6, A0A0P0X7Q1, A0A0P0WAE2, A0A0P0XRC1, B9EYJ0, B9FCQ1, B9ETD5, B9GCM6, A3B8R2, Q6ATL4, Q7XQUO, A3C9L3, B9FD08, B9G4X9, A3A014, B9FCQ3, B9FMN4, B9G4X5, A3CEN4, B9FF59, A0A0P0WFH6, Q7XJ40, A0A0P0W6K3, A0A0P0WAN9, B9FXT6, B9FF58, A0A0P0WF66, Q7XNW1, A0A0P0WF95, A3A0E0, Q7XQT7, Q8W054, Q69MN2, Q7XI25, Q5NAQ9, Q8LMB0, Q2QPI9, Q7XHV3, Q2R917, Q5QLR7, Q5QLN0, Q9LE23, Q5ZAL3, Q0JH38, Q53PX8, Q0IUV4, Q8W3C9, Q5N9Y5, Q7XMR2, Q0JKJ3, Q7XXJ5, Q5ZAK8, Q7XQU3, Q2QLI8, Q851M7, Q60D69, Q7EZ32, Q60EJ2, Q5ZBQ1, Q8H821, Q6ZF83, Q8SOR2, Q7XQT8, Q5Z604, Q7XTD6, Q7X6I1, Q2RAY6, Q5VRR9, Q84MF7, Q6Z6H3, Q5Z601, Q7XTD7, Q9LDT8, Q2RAY2, Q7XMH4, Q5Z932, Q69XK0, Q2QUP5, Q0J9T7, Q69MM4, Q0JD95, Q2QY70, Q5SMQ2, Q2QY68, Q7XMG8, Q7XMG6, Q2RAY4, Q2R918, Q0D733, Q10JE9, Q5NB61, Q6H6Q2, Q8LMB2, Q7XQT9, B7F353, Q7XMH8, Q7XMG5, Q7XMC0, Q5QLM9, Q0J9T2, Q7EZ33, A2ZWE1, Q8LQN6, Q8W059, Q8LQH1, B9FEC3, Q60D78, Q60D68, Q0JIQI, Q5ZAV5, B9FJP0, Q7Y1P1, Q2QY71, Q943Q1, Q7XMH0, Q7XUM4, Q7XQX5, Q69MM9, Q0JPG7, Q7XSF1, Q7XMH5, Q7XMH1, Q25A54, Q01KU2, Q01J05, Q25A55, Q8LLI2, Q25A21, Q01MD3, Q8LLI3, Q01K00, Q25AB4, Q01KM2, Q01MD6, Q25A60, Q8LLI4, Q01K01, Q01K07, Q9LLN5, Q01MD7, Q01LZ8, Q01N33, Q01K06, Q01N32, Q25A58, Q25A57, Q01MD8, Q01K02, KOJBC6, Q25AB6, Q01M86, Q8LLI1, Q25A00, Q01M00, Q259Z7, Q25A01, A0A437CU31, A0A3S2U5L5, A0A3S2MCZ5, A0A437DAI2, A0A3S2MFYO, A0A437DEQ2, A0A3P9ICR8, Q50LG6, A0A3B3I1H2, A0A3P9MP48, A0A3P9K011, A0A3P9I2I7, H2LF02, A0A3P9JAV4, A0A3P9LI60, A0A3P9J614, H2MEZ4, A0A3P9MAE3, A0A3P9LHP9, H2MEU2, H2MOV9, A0A3P9HMN6, A0A3P9K000, A0A3P9JJS7, A0A3P9LIA1, A0A3B3D7P0, I1SRG1, A0A3B3D5Q6, A0A3B3BZR1, A0A3B3CM08, I1SRH8, A0A3B3DHX8, C1BLZ6, A4S6G3, A0AIY5IBM2, A0A096P7Z7, A0A090N4R5, A0A1Y5I2L6, A0A1Y5IDE4, A0A1Y5I7I8, A0A090LXV4, A0A096P9I0, A0AIY5I593, A0A096P7B4, A0A090MCI5, H0XRY6, H0WTA2, H0WJV6, H0X5J0, H0WJV3, W5PEMO, W5P3R3, W5PEI4, W5PUG1, W5Q5H9, A0AIX6WFX5, A0AIX6WF83, A0A086XR69, A0A2R9CEQ2, A0A2R9B5R5, A0A2R9CAN4, A0A2R9B8J6, A0A2R9BIY7, A0A2R9CER0, A0A2R9CESO, A0A2R9CL99, A0A2R9BBJ0, A0A2J8L7X0, A0A2J8JZZ2, A0A2J8L7Y1, A0A2J8L7X6, A0A2J8L807, A0A2I3RJ89, A0A2I3S7A8, A0A2J8L7W7, A0A2J8L7X2, A0A2J8LGB0, A0A2I3S7L0, A0A2I3SM29, A0A2J8JZY8, H2QIA7, A0A2J8K5V2, A0A2J8LGB6, A0A2J8LGB4, A0A2I3S5X5, K7CWX4, A0A2J8L7V9, H2QUU8, A0A2J8K020, H2QUO6, A0A2I3TJS6, A0A2I3T423, A0A2I3REN2, H2QQJ4, A0A2I3TKS5, A0A2I3SRY8, K7BAC9, K7AMD5, A0A2J8K5U6, H2R8A3, A0A409YP06, A0A409YU19, A0A2T7D0I5, A0A2T7EVKO, A0A2T7EDS2, A0A2T7E6P0, A0A2T7DT33, A0A2T7CS41, A0A2T7CTI1, A0A2T7E553, A0A2T7E643, A0A2T7ET21, A0A2T7ER75, A0A2T7DIN6, A0A2T7DDD3, A0A2T7CBD2, A0A2T7EVJ0, A0A2T7D0G2, A0A2T7CT36, A0A2T7E6C1, A0A2T7C4S3, A0A2T7DFU5, A0A2T7DFU8, A0A2T7CMR2, A0A2T7EF41, A0A2T7EWK1, A0A2T7BYK9, A0A2T7DLQ8, A0A2T7DJ77, A0A2T7D0E9, A0A2T7C3A3, A0A2T7DDF0, A0A2T7EW50, A0A2T7CBC7, A0A2T7CL36, A0A2T7EX72, A0A2T7EIN3, A0A2T7D0E7, A0A2T7D509, A0A2T7CFW6, A0A2T7DT37, A0A2T7CUW6, A0A2T7D0D5, A0A2T7DLD3, A0A2T7F513, A0A2T7E7E6, A0A2T7F551, A0A2T7F5F3, A0A2T7D0D9, A0A2T7F5F1, A0A2T7CUW0, A0A2T7D494, A0A2T7EBW9, A0A2T7D0E0, A0A2T7E7B7, A0A2T7DFW0, A0A2T7F436, A0A2T7EVJ2, A0A2T7CUW5, A0A2T7CUZ3, A0A2T7CMN6, A0A2T7E7C7, A0A2T7CB30, A0A2T7EX73, A0A2T7EX64, A0A2T7ER93, A0A2T7D0F4, A0A2T7EVJ5, A0A2T7ERX6, A0A2T7EX66, A0A2T7DDD0, A0A2T7D0E4, A0A2T7CBK9, A0A2T7E0A7, A0A2T7D0F3, A0A2T7D0D8, A0A2T7DV28, A0A2T7DT28, A0A2T7DIM9, A0A2T7DX83, A0A2T7DIM2, A0A2T7F574, A0A2T7F5H7, A0A2T7D0J0, A0A2T7DFV2, A0A2T7EVK4, A0A2T7CUW4, A0A2T7DX94, A0A2T7D0Q9, A0A2T7EVI9, A0A2T7EJU9, A0A2T7EBW4, A0A2T7CB26, A0A2T7EBW3, A0A2T7EVI4, A0A2T7DFU4, A0A2T7C4G3, A0A2T7DJ67, A0A2T7D0F2, A0A2T7DFW9, A0A2T7E6H9, A0A2T7DLC8, A0A2T7BYV2, A0A2T7ENB4, A0A2T7CUV7, A0A2T7D0Y3, A0A2T7DLE0, A0A2T7CUW3, A0A2T7DRA7, A0A2T7DDA9, A0A2T7D0K1, A0A2T7DLD0, A0A2T7D0Q0, A0A2T7EBV8, A0A2T7D0H3, A0A2T7E7B6, A0A270R6Y3, A0A2S3HXW3, A0A2T8IPC6, A0A2T8KJ78, A0A2T8ICA6, A0A2S3HTA8, A0A2S3IAP2, A0A2T8I4D3, A0A2T8KV97, A0A2S3H0F9, A0A2S3IAP8, A0A2S3H1N8, A0A2S3HIG5, A0A2S3IAR5, A0A2T8KQB7, A0A2S3I5P1, A0A2T8IE12, A0A2T8KRJ5, A0A2S3H2E7, A0A2S3HGY6, A0A2S3IKM6, A0A2S3IAM3, A0A2S3H186, A0A2S3I0N4, A0A2T8I4D7, A0A2S3H205, A0A2S3H7F5, A0A2T8IJZ0, A0A2S3H1U7, A0A2T8KUQ8, A0A2T8IC99, A0A2T8I0T0, A0A270R6C5, A0A2T8IE65, A0A2S3HTE7, A0A2T8KRKO, A0A2T8IE16, A0A2S3HAQ3, A0A2S3IGG3, A0A2S3HPQ6, A0A2S3HR11, A0A2T8IDZ9, A0A2S3ID39, A0A2T8IE34, A0A2S3H3A8, A0A2S3GXJ4, A0A2S3H396, A0A2T8KM77, A0A2S3HUX0, A0A2S3IAS7, A0A2S3H762, A0A2T8IE17, A0A2T8IE14, A0A2S3IAP0, A0A2T8KS60, A0A2S3IS50, A0A2S3HP70, A0A2S3HKO5, A0A2S3IQN4, A0A2S3IPM5, A0A2S3HAQ6, A0A2S3HR06, A0A2T8KNP2, A0A2T8KRH6, A0A2S3IGI2, A0A2S3HR12, A0A2S3HD01, A0A2S3H5Z0, A0A2S3IE86, A0A2S3HT43, A0A2S3IQM7, A0A2T8IJ38, A0A2S3IIE5, A0A2S3IB91, A0A2T8JCF9, A0A2S3IAQ3, A0A2T8IE28, A0A2S3I6M3, A0A2T8KV35, A0A2S3IBD2, A0A2S3IPQ8, A0A2T8KHH4, A0A2S3H6Z9, A0A2S3IAP1, A0A2S3HV18, A0A2S3HBZ4, A0A2S3HRI0, A0A2S3I6M7, A0A2S3GP95, A0A2S3HUW3, A0A2T8KRL1, A0A2S3HJZ4, A0A2S3HP69, A0A2T8I1B4, A0A2S3IAN4, A0A2T8IAS4, A0A2T811S3, A0A2T8IDZ2, A0A2S3H370, A0A2S3HW47, A0A2S3IAP4, A0A2T8IE05, A0A2T8KRJ0, A0A2S3HGB8, A0A2S3HZ22, A0A2S3GNM4, A0A2S3HFW8, A0A2S3H7E9, A0A2S3HIN5, A0A2T8KLZ9, A0A2S3I6H4, A0A2S3IAN6, A0A2T8I8I3, A0A2T8IBV6, A0A2S3I506, A0A2T8IJW5, A0A2S3IAP7, A0A2S3HAP8, A0A2T8KHP9, A0A2S3I6I3, A0A2S3GTT4, A0A2T8I1Q5, A0A2S3IST7, A0A2T8KVD3, A0A2T8IE22, A0A2T8IJV0, A0A2T8IE03, A0A2S3GYI1, A0A2S3IAT7, A0A2S3HZR6, A0A2S3IAN7, A0A2T8IKX9, A0A3L6TCR6, A0A3L6PZS7, A0A3L6S5J8, A0A3L6PDC5, A0A3L6QHJ2, A0A3L6PX19, A0A3L6PCT4, A0A3L6PL39, A0A3L6PX91, A0A3L6S5N0, A0A3L6QA61, A0A3L6PG20, A0A3L6SUD9, A0A3L6RDD5, A0A3L6Q4M6, A0A3L6PCG6, A0A3L6SC35, A0A3L6QNE5, A0A3L6PWM5, A0A3L6SS66, A0A3L6S1T6, A0A3L6SHN4, A0A3L6PTZ7, A0A3L6TP99, A0A3L6SU86, A0A3L6R9E9, A0A3L6SL66, A0A3L6QSF1, A0A3L6Q3C3, A0A3L6TLT5, A0A3L6PTZ2, A0A3L6Q8B0, A0A3L6QWW8, A0A3L6RTV3, A0A3L6SY93, A0A3L6THN0, A0A3L6S034, A0A3L6SRC3, A0A3L6QXJ3, A0A3L6T0D2, A0A3L6T329, A0A3L6R3K6, A0A3L6RCL0, A0A3L6T8M5, A0A3L6QUJ7, A0A3L6SPW4, A0A3L6T7S6, A0A3L6QCA5, A0A3L6RXE5, A0A3L6T8Z5, A0A3L6RSYO, A0A3L6SVG3, A0A3L6R496, A0A3L6RL96, A0A3L6PWI7, A0A3L6T589, A0A3L6T3C1, A0A3L6R910, A0A3L6QPB7, A0A3L6SAN9, A0A3L6Q7C9, A0A3L6T2B5, A0A3L6SZ69, A0A3L6R088, A0A3L6PV23, A0A3L6R2W0, A0A3L6TPL9, A0A3L6S8F1, A0A3L6QW70, A0A3L6PE95, A0A3L6R0F3, A0A3L6RH98, A0A3L6SDA3, A0A3L6P9T3, A0A3L6PX52, A0A3L6RQD8, A0A3L6S172, A0A3L6TFE8, A0A3L6SSN5, A0A3L6S7G8, A0A3L6SBX7, A0A3L6QI59, A0A3L6SGX8, A0A3L6PSV3, A0A3L6PB20, A0A3L6SW57, A0A3L6PS89, A0A3L6T996, A0A3L6QZB1, A0A3L6Q8B5, A0A3L6RFQ9, A0A3L6R701, A0A3L6QDT6, A0A3L6SN21, A0A3L6T804, A0A3L6QYH1, A0A3L6SXL6, A0A3L6TUW6, A0A3L6STY3, A0A3L6TQD8, A0A3L6QWJ0, A0A3L6T346, A0A0K6HYJ7, A0A378ZUH8, A0A0U3PCH3, A0A224XG17, A0A224XJC5, A0A224XLL6, A0A224XI83, A0A194RNJ8, A0A194R138, A0A0N1PJMO, A0A194RDJ8, A0A194R2I6, I4DM08, A0A0N114I2, A0A194PVK1, A0A194PQ35, I4DIJ2, A0A194QJF1, A0A194PTA1, A0A194PXM5, A0A2I3MER4, A0A096NQZ2, A0A2I3MEH4, A0A096MT27, A0A096NZY9, A0A2I3LKX1, A0A096MR85, A0A2I3M8D7, A0A096NC95, A0A1H6MTW0, S5XSK4, A0A3D9XH37, A0AIG9LDL1, A0AIW6CWQ5, A1B4L0, A0A2W5A1K7, A0A099F544, A0A368YRU1, A0A1K1YUK1, A0A454NRD8, A0A1I5KJ26, A0A2A2GLC7, A0A1N7M497, A0A099GIU4, A0AIH2UIH5, A0A418SQD8, A0A2K9F7T9, A0A3N7HKP6, A0A1X7K5P3, A0A3S4DYQ8, A0A0V0Q0U8, A0A1N6QLJ3, A0A3E0CAT2, A0A369U330, A0A386UNW4, A0A2D2C4W0, A0AIV0GXG8, A0A2H5F0H5, D1M702, A0A135HYP7, A0A3B3T1V7, A0A3B3T2D5, A0A3B3QTI7, A0A3B3T216, A0A3B3R816, A0A3B3T3Q0, A0A3B3QMB5, A0A3B3T389, A0A3B3T3H8, A0A3B3T3U8, A0A177CZE2, A0A177CRA0, A0A177CUG9, A0A177C382, A0A177C6N6, A0A177CID1, A0A1C7P136, A0A2P5BJA4, A0A2P5BW50, A0A2P5B4R0, A0A2P5BW53, A0A2P5BW51, A0A2P5BM82, A0A2P5A6J9, A0A2P5BW67, A0A2P5E4M6, A0A2P5D441, A0A2P5BW21, A0A2P5BKD8, A0A2P5E4L5, A0A2P5BMA8, A0A2P5BKI9, A0A2P5AA39, A0A2P5BMB4, A0A2P5B4Q4, A0A2P5BM80, A0A2P5DYR7, A0A2P5AHJ9, A0A2P5E4N0, A0A2P5D258, A0A2P5BW45, A0A2P5BKH5, A0A2P5E4Q5, A0A2P5A6G1, A0A2P5BM96, A0A2P5B4Q6, A0A2P5BW66, A0A2P5BM92, A0A2P5BN11, A0A2P5BMD0, A0A2P5CS31, A0A2P5BMA5, A0A2P5E4N1, A0A2P5B4P1, A0A2P5CSJ2, A0A2P5BM72, A0A2P5E4Q3, A0A2P5BH32, A0A2P5B4S4, A0A2P5CFJ1, A0A2P5E4M4, A0A2P5DP24, A0A2P5BIW1, A0A2P5CC51, A0A2P5BM84, A0A2P5BM71, A0A2P5BJC8, A0A2P5B4T3, A0A2P5BKJ2, A0A2P5AA49, A0A2P5BW30, A0A2P5BW41, A0A2P5BW35, A0A2P5B1T6, A0A2P5E4R8, A0A2P5B1R8, A0A2P5DGX2, A0A2P5B4R2, A0A2P5B4Q1, A0A2P5B4S3, A0A2P5BJA2, A0A2P5BW77, A0A2P5BW40, A0A2P5B4Q8, A0A2P5DYQ3, A0A2P5CA29, A0A2P5AIJ3, A0A2P5BM74, A0A2P5E4J2, A0A2P5BJC9, A0A2P5BM50, A0A2P5A6F1, A0A2P5BW89, A0A2P5BW37, A0A2P5BW20, A0A2P5E4P8, A0A2P5BW58, A0A2P5E4M9, A0A2P5E4P4, A0A2P5E4N5, A0A2P5B4R9, A0A2P5A6G8, A0A2P5DYT0, A0A2P5BW31, A0A2P5AEM1, A0A2P5D7Y5, A0A2P5BW71, A0A2P5BMA2, A0A2P5AH20, A0A2P5B4S8, A0A2P5BMC0, A0A2P5BXQ1, A0A2P5BVZ5, A0A2P5BKL3, A0A2L2YD42, A0A2L2Z1W7, A0A2L2Z8H1, A0A2L2ZA53, A0A2L2Z530, A0A2L2YWS1, A0A0N4ZHI4, A0A0N5A0H4, A0A0N4ZPS9, A0A0N5A3M1, A0A0N4ZRF2, A0A0N4Z938, A0A0N4Z4J1, A0A0N4ZEA2, A0A0N4Z1R8, A0A0N4Z6U2, A0A0N4ZP70, A0A0N5A5G9, A0A0N4ZHI3, A0A0N4ZNC4, A0A0N4ZKC3, A0A0N4ZPV9, A0A0N4Z312, A0A0N4ZIU0, A0A0N4Z5S0, A0A0N4ZS52, A0A402ESB7, A0A402FAU3, A0A402FMT0, A0A402EEMO, A0A2D8XC50, A0A1V4K051, A0A1V4JKY3, A0A1V4JG98, A0A1V4JKX2, A0A1V4K8H7, A0A1V4K1P3, A0A1V4K8P3, A0A1E1WSI0, A0AIE1WED2, E0VCL9, E0VHE3, E0VC84, E0VX62, E0VX61, E0VJE4, A0A1P8UUY1, Q0FI61, A0A2D6BAV5, A0A1E4DW35, A0A1E4FC43, A0AIE4GBYO, A0AIE4GLE6, A0AIY5RQR3, A0A2U2CX27, A0A238KIZ4, A0A2T7G6G5, A0A091SM44, A0A091TMT4, A0A091TWU6, A0A091STZ2, E5RLR1, K7GFR6, K7GGY8, K7FLF2, A0A347ZT03, A0A3R7LS22, A0A423SJ34, A0A423TTP3, A0A3R7M4Q0, A0A423SF89, A0A3R7MK76, A0A3R7P3K5, A0A423U6F6, A0A423TME0, A0A423TTP4, A0A3R7M498, A0A3R7MRL0, A0A3R7P0E8, A0A3R7PNR3, A0A3R7PF83, A0A1L9S4X6, A0AIL9SBG1, A0AIV6PCL2, A0AIF5LG55, A0AIF5LFH2, A0AIF5LBV1, A0A1F5L7N0, A0A1F5LFH7, A0A1F5L4M3, A0A0G4PQR1, A0A1V6UT76, A0A1V6URX4, A0AIV6USL6, A0AIV6PHF0, A0A0A2ITH3, A0AIV6SGC7, A0A0A2KKT0, A0AIV6WU75, A0A1V6Z413, S8B8Z7, S7ZRP3, S7ZGN1, S7ZMP6, A0A135LCH0, A0A135LCH3, A0A135LCQ1, W6Q172, B6HSU8, A0AIV6QSF1, A0AIV6QSE8, A0A2H3I1I4, A0A2H3IC68, A0AIV6SRD0, A0A1Q5T8E6, A0AIQ5TYZ6, A0A1V6RF18, A0A1V6RE75, A0A1V6RG25, A0A1V6S597, A0A1V6RFI7, F1C726, F1C7D0, A0A2V1DWV5, A0A2V1D2D5, A0A2V1DIZ4, A0A2V1DJW4, A0A2V1DLD9, A0A2V1D8T0, A0A3B3ZPQ5, A0A3B3ZY43, A0A3B3ZXI2, A0A3B3ZP74, A0A3B3ZYU4, A0A3B4AGU9, A0A3B3ZP80, A0A3B3ZYS8, A0A0L1L095, A0A3M6VL27, A0A3M6VAW8, A0A425CJM8, A0A3R7Y325, A0A3M6V6Z7, W3XET2, W3X3J5, W3XMF5, W3XPT6, W3XC43, W3WRT1, W3XNX9, A0A2J6QKC3, A0A366X020, A0AIB0ZTK3, A0A0PIGVW2, A0A0P1IHK9, B7G5U0, A0A0G2E5S8, Q0UMP0, Q0U636, Q0V168, Q0V5G3, Q0V4N1, Q0TZX6, Q0UPL0, Q0UWY3, Q0UP20, A0A091ULD4, A0A091T5G0, A0A091TAK8, A0A091TBV2, A0A093QYY4, A0A093TDY9, A0A093QPS2, A0A093QSI2, A0A0L9TKR2, A0A0L9VGP0, A0A0L9UFV5, A0A0L9ULD5, A0A0L9TCV5, A0A0L9V3Z2, A0A0L9UKZ9, A0A0L9U8P2, A0A0L9U553, A0A0L9TLV1, A0A0L9U6Q9, A0A0L9UIH5, A0A0L9ULI3, A0A0L9UM53, A0A0L9U552, A0A0L9V319, A0A0L9UN60, A0A0L9TLV8, A0A0L9ULD1, A0A0L9V362, A0A0L9V8X3, A0A0L9V600, A0A0L9ULS9, A0A0L9T851, A0A0L9VUB8, A0A0L9TGQ7, A0A0L9U4V0, A0A0L9THP8, A0A0L9VUS9, A0A0L9V9B9, A0A0L9UIP6, A0A0L9UPU8, A0A0L9TKM5, A0A0L9V913, A0A0L9VTA1, A0A0L9V3F3, A0A0L9V376, A0A0L9UN55, A0A0L9V4V3, A0A0L9V1W9, A0A0L9V3H0, A0A0L9VFN3, A0A0L9UTN1, A0A0L9TGU5, A0A0L9V3V3, A0A0L9UPN2, A0A0L9TGG9, A0A0L9V486, A0A0L9UMB7, A0A0L9VG17, A0A0L9UJE5, A0A0L9TUI5, A0A0L9V464, A0A0L9VFUO, A0A0L9U5F8, A0A0L9U882, A0A0L9UJ28, A0A0L9VSL2, A0A0L9UMD9, A0A0L9UM48, A0A0L9VFN1, A0A0L9V5Z3, V7BQJ3, V7CQM2, V7A1N2, V7BX92, V7B6U7, V7AIF8, V7BXZ6, V7ALS3, V7B4W4, V7AHY7, V7AGA5, V7AIM9, V7AIL8, V7CPR4, V7BXT0, V7AIF2, V7AHM7, V7BUN3, V7ALT3, V7AMQ9, V7AIH2, V7AJU3, V7APL9, V7B7X1, V7ALI5, V7AHN7, V7BVD1, V7AHQ4, V7AMQ6, V7AJR9, V7BRW9, V7AE52, V7ALQ7, V7BV89, V7AJK3, V7D2F6, V7BZ49, V7AHK3, V7CPG4, V7B5J8, V7AID3, V7AIM3, V7BYK2, V7B3V1, V7AH65, V7BXM2, V7AJP5, V7BYJ9, V7BQM9, V7BZ42, V7B3X6, V7AJE9, V7AJS5, V7ATU1, V7CPG0, V7CM72, V7AST9, V7ALL6, V7CZ14, V7BTA1, V7AKL3, V7BXZ1, V7AHE0, V7ALR2, V7ALS5, V7AKL1, V7AVN9, V7C143, V7C7H2, V7CA41, V7ALS8, V7B451, V7D003, V7ANE1, V7AVN4, V7BUI7, V7AMN5, V7AHR0, V7BTA2, V7AMM7, V7AHK9, V7AM09, V7ALF8, V7BUL6, V7AGA9, V7AHL4, V7BWF6, A0A194X242, A0A194XBS6, A0A132BBB6, A0A194WTN9, A0A132B592, A0A194X7W7, A0A194X856, A0A194XR54, A0A194WSB8, A0A194WSX5, A0A194WTK6, A0A194XX66, A0A132BCG7, A0A132B964, A0AIL7XLE9, A0A1L7X859, A0AIL7WF44, A0AIL7XLL5, A0A1L7XTN8, A0A1L7X9R0, A0A1L7WMU3, A0A0N0NQR5, A0A0N1HGJ5, A0A0N0NQ51, A0A0N1GX61, A0A0N1H4B9, A0A0N1NZ06, A0A0N1HG31, A0A370TCU2, A0A370TYQ2, A0A370TJW0, A0A370TQF0, A0AIB0GNH6, A0A1B0D9P3, A0A1B0DGU9, A0A1B0DI86, A0A1B0GP61, A0A091U239, A0A091UAN5, A0A3Q0HVZ9, A0A3Q0HMY7, A0A3Q0I1K4, A0A2H3ZIZ6, A0A2H3ZAB6, A0A3Q0I914, A0A2H3X918, A0A3Q0HT42, A0A2H3ZS58, A0A3Q0IFF5, A0A3Q0HQF8, A0A3Q0HSM9, A0A3Q0HVI7, A0A3Q0HTF0, A0A2H3XGF9, A0A2H3X9J5, A0A2H3X0Q6, A0A2H3X3J0, A0A2H3YZ59, A0A2H3Y805, A0A2H3X8K1, A0A2H3XA64, A0A2H3WXV9, A0A2H3X7L6, A0A2H3ZCS3, A0A2H3ZIK9, A0A3Q0HR72, A0A2H3XQV7, A0A2H4A1V3, A0A2H3ZJF3, A0A3Q0HQX3, A0A3Q0HSL1, A0A2H3X0N3, A0A2H3YKV9, A0A2H3Z3E8, A0A3Q0HVZ4, A0A3Q0HTK9, A0A2H3XJR1, A0A3Q0I0R7, A0A2H3YI52, A0A2H3X0N6, A0A3Q0I4E3, A0A2H3Y849, A0A2H3ZQ89, A0A2H3Y8E0, A0A2H3ZT49, A0A3Q0IGU8, A0A3Q0HRL0, A0A2H3YUK0, A0A2H3X6E4, A0A3Q0IBE3, A0A3Q0I4N2, A0A2H3Z4R9, A0A2H3Y059, A0A2H3WYX2, A0A3Q0HQU7, A0A2H3ZI64, A0A2H3XQR6, A0A2H3ZW91, A0AIY1KHG4, A0AIY1MWD5, A0AIY1N456, A0AIY1LYJ5, A0AIY1K6Q9, A0AIY1KVK9, A0AIY1NDL0, A0AIY1MWC5, A0AIY1KDA6, A0AIY1N145, A0A1Y1N670, A0A1Y1MXX0, A0A2E1XX07, A0A368ZAV4, A0A368Z5S6, A0A2P7BX27, A0A2P7B2B9, A0A2P7B199, A0A318STX6, A0A2S9JB27, A0A368K297, A0A2P7BBS7, A0A2S9IZ33, A0AIH0QMP3, J3CCV4, A0AIH0UPJ4, A0A2N9VRL8, A9SNW9, A0A2K1K665, A0A2K1KEJ6, A0A2K1K664, A0A2K1IS53, A9T7Y0, A0A2K1JCS2, A0A2K1ISB0, A9TEC1, A9SP81, A0A2Y9FNN2, A0A2Y9FMM8, A0A2Y9S5B0, A0A2Y9FL53, A0A2Y9FNH7, A0A2Y9FVN1, A0A2Y9RZG7, A0A329R7W9, A0A329R9F3, A0A329RYD9, A0A329SSU7, A0A329RKN5, A0A329S4Z6, A0A329T4J1, A0A329SHI1, A0A329S9V0, A0A329SRY9, A0A329RNK1, A0A329SNY9, A0A329S7M0, A0A329S1Z2, A0A329SRE1, A0A329S1D7, A0A329SWT6, A0A329S4B3, A0A329SPJ8, A0A329RIU8, A0A329SPU9, A0A329RT73, D0MY50, D0NX36, D0NX35, D0NTA4, D0MY47, D0P016, D0NEY9, D0NYL6, D0NTB0, D0PlE8, D0N2D7, D0N7R8, D0MT29, D0NRT7, D0MRR0, D0MY49, D0P0T9, D0NUN5, D0NQ00, D0NZF4, D0N386, D0P028, Q2M441, B9VUU8, A0A421GDD8, A0A3R7K3Z6, A0A3F2RDE5, A0A421FHH8, A0A3R7KOA4, A0A3R7HDZ2, A0A421ETD0, A0A421FA97, A0A3F2RJX1, A0A3F2RE35, A0A3F2RF52, A0A3R7IQA6, A0A3F2RY02, A0A3F2RER3, A0A3F2RZC8, A0A3F2RR68, A0A3R7KUW5, A0A3R7JT10, A0A3R7J7R7, A0A3F2RF49, A0A3R7G004, A0A3R7HP88, A0A225WAC9, A0A225WNV7, A0A225VPW0, A0A225VX31, A0A225V0S2, A0A225WNJ4, A0A225VUT8, A0A225V861, A0A225VY03, A0A225VWU9, A0A225WU58, A0A225UTR0, A0A225US74, A0A225VHE2, A0A225WUN7, A0A225VTE7, A0A225WIB3, A0A225WKQ4, A0A0W8C7T1, A0A0W8CDF7, A0A0W8DMYO, A0A0W8DQSO, A0A0W8C6H3, A0A0W8DC43, A0A0W8CY53, A0A0W8C244, A0A0W8DC46, A0A0W8BQT9, A0A0W8BN24, A0A0W8C8H2, A0A0W8CP70, A0A0W8C0L1, A0A0W8CT29, A0A0W8DME7, A0A0W8D9R0, A0A0W8C2P6, A0A0W8CY27, A0A0W8CIP5, A0A0W8B357, A0A0W8AZV8, A0A2P4YEQ1, A0A2P4YER5, A0A2P4YOY6, A0A2P4XL73, A0A2P4XH46, A0A2P4XJK4, A0A2P4XVK1, A0A2P4Y6J7, A0A2P4X3D0, A0A2P4XLE8, A0A2P4X3F5, A0A2P4YFR7, A0A2P4Y2R6, A0A2P4YH90, A0A2P4XUW4, W2Q467, W2RHF8, W2QVF4, W2Q4M7, W2PJM9, W2PLH6, W2Q115, W2PKG5, W2QD00, W2QZF4, W2QTN8, W2Q634, W2Q9Y7, W2PJB5, W2R209, W2PFP4, W2PU68, W2QCZ4, W2REW3, W2PEG7, W2Q870, W2WEV6, W2X913, W2X1M9, W2X5D1, W2VX79, W2WUZ2, W2X348, W2XWF0, W2VZ24, W2WSU4, W2W607, W2W0N8, W2VXA4, W2XT69, W2WTF3, W2W2G5, W2XAA7, W2WHH5, W2VUZ5, W2XAB2, W3A0G8, W2Y5P0, W2ZM34, W3A6G4, W2ZDS8, W2ZHI4, W2YS73, W2Z3U6, W2YNN5, W2Y9I4, W2Z3S0, W2ZBQ5, W2YBW1, W2ZD75, W2ZHQ3, W2YGM9, W2Y8A6, W2YD26, W2Z356, W2YBY5, V9EY76, V9E3H4, V9DZZ5, V9FZZ4, V9FE93, V9E8N5, V9EY37, V9EX27, V9F9H0, V9E3Y2, V9ENS5, V9F7A9, V9F7C6, V9E3S0, V9FXZ9, V9FC98, V9EKG8, V9EI96, V9FDK5, V9EC42, V9E1H4, A0A081AYV0, A0A081A0S9, A0A081A8Z3, A0A080ZLK1, A0A081AB04, A0A081B3V1, A0A080Z747, A0A081A0T1, A0A080Z748, A0A080Z738, A0A080ZP81, A0A081AYU8, A0A080Z4Y8, A0A081AYU4, A0A081A1R2, A0A080Z2Z3, A0A081AB01, A0A081AH92, A0A080ZDR9, A0A080ZAI3, W2LFS8, W2HKN0, W2L0R5, W2P1G7, W2FR98, W2K4B4, W2J7J1, W2KMQ1, W2FPQ8, W2K6H5, W2K5X3, W2P694, W2L728, W2K2G5, W2MHW4, W2NET7, W2GUQ1, W2J9V7, W2GNC4, W2H193, 042830, W2HY18, W2K697, W2MTG5, W2MC44, W2KBB4, W2G7E9, W21G72, W2J2Z4, W2J2Z6, W2MA47, W2FRI0, W2M865, W2GWC8, W2GWD7, W2L8Q2, W21021, W2M2S0, W2N786, W2N694, W2G037, W2G9R5, W2JQ56, W215H9, W2HZQ8, W2MA56, W2ISN0, W2H2J5, W2NEV2, W2HNT1, W2JB43, W21254, W2MW48, W2NEU1, H3G593, H3H1Z2, H3GT31, H3HDS4, H3H1Y9, H3H2Y9, H3H2Y8, H3G5T4, H3H1Y8, B2ZU37, B2ZU68, B2ZU62, H3GQA8, B2ZU49, H3H1Y6, H3H1U8, H3GLB2, H3G5P9, H3H1Z1, B2ZU72, H3GQA7, B2ZU45, H3H2Z0, H3GT32, H3G5B0, H3H2Y7, H3GDG9, B2ZU71, H3H1Z0, B2ZU70, B2ZU32, A0A088CB98, B2ZU23, H3GVW9, B2ZU31, A0A2L0WUS4, AlEAQ0, G4Z5J6, G4ZN72, G4ZGF9, G4YIR5, G5ABJ2, G4Z8P9, G5AEI8, G4ZGF6, G4YRJ3, G4Z802, G5A0U5, G4YU93, G4YIR6, G4YTG2, G5AEI6, G4Z8P8, G4ZRE5, G4YIR7, G4YXF1, G4Z8Q8, G4Z5J7, G4YRE7, G4ZT51, G4Z5J8, G4Z8Q0, G5AEI5, G4YW39, G4ZTF9, B8LRA5, B8LRG3, A0A194ANQ8, L0N3W3, A0A1Y1VF86, A0A1Y1V4N4, A0A1Y3NGF1, A0A2D9PR14, A0A2P6NN51, A0A2P6NBD6, A0A2P6NS16, A0A2P6NFX0, A0A2P6NBF7, A0A2P6NM78, A0A2P6NZX6, A0A2P6NQW3, A0A2P6NJ93, A0A2P6NKK7, A0A2P6NXQ1, A0A2P6N084, A0A2P6N8A1, A0A2P6MQS5, A0A2P6NWD1, A0A2P6N6L8, A0A2P6NUU8, A0A2P6NYV0, A0A2P6MVG3, A0A2P6N8R1, A0A2P6N8J2, A0A2P6NNF7, A0A2P6N1U3, A0A2P6NZG2, A0A2P6NJU9, A0A2P6NU31, A0A2P6NNX6, A0A2P6MNV7, A0A2P6N4J7, A0A2P6P052, A0A2P6NPL0, A0A2P6N398, A0A2P6NVG2, A0A2P6MWF9, A0A2P6NLH5, A0A2P6N891, A0A2P6NKS8, A0A2P6NRU2, A0A2P6NI63, A0A2P6NPR4, A0A2P6NBF0, A0A2P6NL20, A0A2P6NRH4, A0A2P6NWM6, A0A2P6MUC2, A0A2P6MQR6, A0A2P6NL12, A0A2P6NVA7, A0A2P6NKZ3, A0A2P6MZC6, A0A2P6NII8, A0A2P6P0D1, A0A2P6NUJ5, A0A2P6NMA2, A0A2P6NGP7, A0A2P6MT35, A0A2P6MSH3, A0A2P6NK96, A0A2P6NR97, A0A0P1B1W3, A0A0P1A668, A0A0P1B457, A0A0PIALX1, D0VX36, D0VX37, A0A411F668, A0A2G9WWN9, A0AII4U8D5, A0A2Z6HWP3, A0A2Z6I0M2, A0A179F4S7, A0A179F441, A0A3M6TS79, A0A3M6USF2, A0A3M6U8L5, A0A3M6T8B8, A0A3M6UTQ7, A0A3M6V1M2, A0A3M6TMM1, A0A3M6T9H4, A0A3M6UUH4, A0A3M6UCX4, A0A3M6TR63, A0A3M6TQV1, A0A3M6UUS3, A0A3M6T9L3, A0A3M6UH21, A0A3M6UFJ0, A0A3M6TLA8, A0A3M6TXJ3, A0A3M6UNB9, A0A3M6TV38, A0A3M6UY98, A0A3M6UGR8, A0A3M6UHN8, A0A3M6TQC2, A0A3M6UZT4, A0A3M6TG14, A0A3M6UAH9, A0A3M6U389, A0A3M6T9B4, A0A3M6TK43, A0A3M6TKR9, A0A3M6UW14, A0A3M6USD4, A0A3M6TZG9, A0A3M6UIH2, A0A3M6UHX1, A0A3M6TJU1, A0A3M6TLI2, A0A3M6UQ55, A0A3M6UM67, A0A3M6T7C9, A0A3M6T879, A0A3M6TMQ8, A0A3M6U799, A0A3M6UYP3, A0A3M6UY17, A0A3M6V3Y0, A0A3M6UUQ2, A0A3M6TKU3, A0A3M6UYG1, A0A3M6TTJ5, A0A3M6TY63, A0A3M6TFR8, A0A3M6U3C3, A0A3M6U1C9, A0A3M6U041, A0A3M6U1S3, A0A3M6TKF4, A0A3M6TTSO, A0A3M6U9P4, A0A3M6TU17, A0A3M6V0G4, A0A3M6UKJ5, A0A3M6U9D2, A0A3M6UMI0, A0A3M6V1P4, A0A3M6TZE9, A0A3M6U9C4, A0A3M6UK43, A0A3M6TDQ0, A0A3M6UQ00, A0A3M6T9N0, A0A3M6TL38, A0A3M6UGQ2, A0A3M6T5E6, A0A3M6TYN4, A0A3M6UZA1, A0A3M6V499, A0A3M6TZ45, A0A3M6T7Z7, A0A3M6UQN2, A0A3M6V0A1, A0A3M6UAD8, A0A3M6TA22, A0A3M6UL69, A0A3M6V562, A0A3M6UHQ1, A0A3M6V6G0, A0A3M6U1J0, A0A3M6ULX9, A0A3M6TX33, A0A3M6V3R3, A0A3M6UG70, A0A3M6TFV1, A0A3M6TCE3, A0A3M6U1L3, A0A3M6USJ5, A0A3M6UV09, A0A3M6THX6, A0A3M6TSD4, A0A3M6UHD5, A0A3M6TIM9, A0A3M6V609, A0A3M6UMN1, A0A3M6T7T9, A0A3M6TRU4, A0A3M6UPF6, A0A3M6TA00, A0A3M6TTM9, A0A3M6T9S3, A0A3M6V4P3, A0A3M6URI8, A0A3M6T6W2, A0A3M6TGI7, A0A3M6U3G3, A0A3M6UL17, A0A3M6UCE1, A0A3M6USZ6, A0A3M6UMF9, A0A3M6TET4, A0A3M6TG44, A0A3M6U3J1, A0A3M6UXV8, A0A3M6TWK8, A0A3M6UV46, A0A3M6UL16, A0A3M6ULU8, A0A3M6T6A8, A0A3M6U377, A0A3M6TAL1, A0A3M6T5X9, A0A3M6V0S5, A0A3M6UZX3, A0A3M6V292, A0A3M6USE0, A0A3M6V1C4, A0A3M6TRT6, A0A3M6TGP2, A0A3M6TNZ6, A0A3M6U6B3, A0A3M6TYI9, A0A3M6U5N1, A0A3M6TZH8, A0A3M6TZB0, A0A3M6U1T5, A0A3M6UTP6, A0A3M6V3G9, A0A3M6UAU9, A0A3M6UVP6, A0A3M6UUS4, A0A3M6U1B0, A0A3M6U9H5, A0A3M6U252, A0A3M6T542, A0A3M6UJV7, A0A3M6TA55, A0A3M6T9T7, A0A3M6TWX2, A0A3M6V721, A0A3M6U9I9, A0A3M6U1G7, A0A3M6U527, A0A3M6U1R3, A0A3M6V1Z1, A0A3M6UQ36, A0A3M6TI45, A0A3M6UIZ7, A0A3M6UMS9, A0A3M6UY19, A0A3M6UPE3, A0A3M6TNQ1, A0A3M6UMUO, A0A3M6TGA7, A0A3M6TX72, A0A3M6V494, A0A3M6UG64, A0A3M6UK86, A0A3M6UYI3, A0A3M6V0I9, A0A3M6T8D1, A0A3M6TZSO, A0A3M6TCK3, A0A3M6UF72, A0A3M6URU5, A0A3M6U5L9, A0A3M6UQ23, A0A3M6U9G1, A0A3M6UGB6, A0A3M6UUP1, A0A3M6TGX0, A0A3M6UKV3, A0A3M6U1N8, A0A3M6V463, A0A3M6UKM5, A0A3M6TYF4, A0A3M6UON0, A0A3M6UMN0, A0A3M6U2G5, A0A3M6T9Q3, A0A3M6TY60, A0A3M6UCM1, A0A3M6TVJ0, A0A3M6U3I0, A0A3M6VBMO, A0A3M6TTN4, A0A3M6TST7, A0A3M6V143, A0A3M6TTD9, A0A3M6U218, A0A3M6U1Q8, A0A3M6T410, A0A3M6UKS4, A0A3M6T9Q5, A0A3M6US86, A0A3M6TYB9, A0A3M6TTW0, A0A3M6TCD6, A0A3M6UMC9, A0A3M6USH7, A0A3M6TD69, A0A3M6UK11, A0A3M6UVR5, A0A3M6U2Q5, A0A3M6V3V5, A0A3M6UBX9, A0A3M6UK88, A0A3M6UUI1, A0A3M6TKL2, A0A3M6V010, A0A3M6TK69, A0A3M6TDY5, A0A3M6TIX2, A0A3M6TYN3, A0A094L7R4, A0A094NEK3, A0A094KG51, A0A094LT96, B2B3F1, B2AEW5, B2ASY6, B2B631, A0A090CJ60, A0A447CBI5, A0A447CFK1, A0A447C1B4, A0A447CHJ2, A0A087YC08, A0A087YDJ9, A0A096M9X7, A0A096MHY9, A0A096LUY1, A0A087XAX0, A0A087YK42, A0A096M7R2, A0A087YBW9, A0A3B3V3Z1, A0A3B3TJZ9, A0A3B3V421, A0A3B3V1M0, A0A3B3UUE6, A0A3B3W2N5, A0A3B3VXR8, A0A3B3TK34, A0A3B3USD0, A0A3B3TX33, A0A3B3VD97, A0A3B3V427, A0A3B3TZ12, A0A3B3XYA2, A0A3B3YGN7, A0A3B3YU59, A0A3B3YUMO, A0A3B3XXC4, A0A3B3XJP2, A0A3B3XXG3, A0A3B3X0F3, A0A3B3XY03, A0A3B3YUR5, A0A3B3XRS1, A0A3B3Z3V6, A0A3B3WRD1, A0A3B3YVC7, A0A3P9Q3Q9, A0A3P9Q2T6, A0A3P9NA69, A0A3P9Q2U6, A0A3P9Q2Z7, A0A3P9Q320, A0A3P9Q335, A0A3P9Q431, A0A3P9Q3Q0, A0A3P9Q4J8, A0A3P9Q3N5, A0A3P9PH03, A0A3P9Q356, A0A0S7K5B4, A0A0S7J5R7, A0A0S7K6Q0, A0A0S7KT15, A0A0S7K5P7, A0A0S7M8P1, A0A0S7L542, A0A0S7ELW6, A0A0S7K6K9, A0A0S7K5V9, A0A0S7M7Y8, A0A0S7EN80, A0A0S7EV87, A0A125SL75, A0A125SL77, Q9Y1V3, F2IW28, A0A2B7YVT1, A0A2B7Y009, A0A2B7XRG7, A0A2T7PUF8, A0A2T7P4A6, A0A2T7P2C2, A0A2T7NW21, A0A2T7NVT2, A0A2T7P293, A0A2T7NRV8, A0A2T7P283, A0A2T7NUX7, A0A2T7NZW8, A0A2T7P282, A0A2T7NPR7, A0A2T7NP19, A0A2T7PDK6, A0A2T7PBMO, A0A2T7NH27, A0A2T7NGZ5, A0A2T7PVC4, A0A2T7P296, A0A2T7NPP6, A0A2T7PIV1, A0A2T7NT09, A0A2T7P290, A0A2T7P2A0, A0A2T7NT13, A0A2T7PXQ6, A0A2T7P2A4, A0A2T7P9Q0, A0A2T7NDL4, A0A2T7NMC9, A0A2T7PII0, A0A2T7Q143, A0A2T7P231, A0A2T7NAQ2, A0A2T7NUW9, A0A2T7PVQ3, A0A2T7PQ77, A0A2J8T957, K7EVW6, A0A2J8VLI2, A0A2J8VLJ4, A0A2J8T3S9, A0A2J8VLH8, A0A2J8T3R6, H2PKT3, A0A2J8T3Q1, A0A2J8VLJ0, A0A2J8TGX6, A0A2J8T3Q9, H2PAR3, A0A2J8T3Q0, A0A2J8VLK6, A0A2J8VLJ5, A0A2J8TGY3, H2PEX4, A0A2J8VLK7, A0A2J8T3Q5, A0A2J8T3T1, Q5R976, A0A2J8T3R5, Q5R502, H2PEX7, H2PN16, A0A2J8T950, Q5R8X6, A0AIR3WSMO, A0A2D9RCA7, A0A2D6GQ64, A0A2D7IW65, A0A2D9RE05, A0A0W7WIT4, A0A0W7WNN3, A0A0W7WKJ9, A0A0W7WED2, A0A385JHS5, A0A3S9LX70, A0A385JID9, A0A385JIC9, A0A385JIW5, A0A3S9LX83, A0A3S9LX72, A0A385JIF5, A0A3S9LX97, A0A1L6K4F6, A0A3S9LX71, A0A3S9LX61, A0A3S9LXA8, A0A3S9LX98, A0A3S9LXB3, A0A3S9LX57, A0A3S9LXA3, A9PJX6, A0A3N7FJX8, B9HYR9, A0A3N7FA41, A0A2K1Y5Z8, U5FEZ9, U7E2S0, A0A3N7FPW1, A0A2K2CBM7, A0A3N7H0W6, A0A3N7FQG9, B9I033, A0A2K1YMT8, A0A2K2CD15, A0A2K1YMD4, A0A2K1YF78, A0A2K1YF67, B9HYQ3, A0A2K2AP05, A0A2K2CBR0, A0A3N7G2Z5, U5GW32, A0A2K1YMF9, A0A2K1YMF8, A0A2K2CBV8, U7DXY3, A0A2K1YLY5, A0A3N7H7G1, B9HYS3, A0A2K1YJB9, U7E151, A0A2K1R9J9, A0A2K2CBS5, A0A2K1XDN9, A0A3N7FS13, U5G9S9, A0A2K1YF84, B9I035, B9NGV9, A0A3N7EEZ9, A0A3N7ETB6, A0A3N7GAS2, U7DV48, A0A3N7EEW7, A0A3N7G946, A0A2K1YMS1, A0A2K2AP11, A0A3N7GLP6, A0A3N7FPY6, A0A3N7FJT6, U7DVW0, A0A2K2BAE9, A0A3N7FRW4, A0A3N7GIT0, A0A2K1YF54, A9PFH0, A0A3N7FPB6, A0A2K2CBN3, A0A2K1YMF6, A0A2K1YF72, A0A2K1YF37, A0A3N7FK42, A0A2K1YJI6, A0A2K2CBUO, A0A3N7FQ27, A0A2K1YMF7, A0A2K1YMK1, A0A2K1R9K1, U7DZT8, A0A2K2CBV2, A0A2K1YRU7, U5FQT0, A0A2K1R435, A0A2K2CBU5, A0A3N7FKB6, A0A3N7EYY1, A0A3N7F931, A0A2KIR9M9, A0A3N7HDJ2, A0A3N7H721, A0A2K2CBR5, A0A2K1YMD5, U7DWR5, U7E0R0, A0A3N7G378, A0A3N7G0M4, A0A2K2CBS7, U5GSZ3, A0A2K1YF20, A0A2K1YCZ6, A0A2K2AP00, A0A2K2CBS4, A0A2K1R9L1, A0A2K1R9K8, A0A2K1YF88, A0A2K2CBR3, A0A3N7EYL9, A0A3N7FPG8, U7DYK7, A0A3N7EJM8, A0A2K1YMF0, B9IFY4, A0A2K1R9K3, A0A2K1R9L4, U7E198, A0A3N7FBU8, A0A3N7HE18, U7E213, A0A3N7FRQ4, A0A2K1R7A4, U7DZU3, A0A3N7FTM4, A0A2K1YJJ2, A0A2K2CAQ6, A0A3N7F920, A0A3N7FQE7, A0A2K2AAA5, B9HAE5, A0A2K1XB50, A0A2K1YMH4, A0A2K1YJA7, A0A2K1YMF3, U5FZN7, A0A2K1WRN2, A0A3N7EEZ8, A0A2K1R9I9, B9I048, A0A2K1R9K2, A0A2K2CBW8, A0A3N7FBT5, A0A2K1YF32, U5FP35, A0A3N7H774, A0A3N7FUN1, A9PF61, A0A2K1YJJ1, A0A2K2AQM5, A0A2K1YF42, A0A2K1R9J8, A0A3N7ERB7, A0A2K1ZA48, A0A3N7FRY4, A0A2K2AAA9, A0A3N7G2Z7, A0A2K1YF57, A0A3N7EBP4, A0A2K1R5I9, A0A3N7EXE8, B9INT4, B9MU91, A0A3N7EII0, A0A3N7HDF5, A0A2K1YF71, A0A2K2AAC8, A0A2K2CBV0, A0A2K1R9L6, A0A2K1YF62, A0A3N7EMG0, A0A3N7GEG5, A0A3N7FKUO, A0A2K2CC26, A0A2K1R9K7, A0A2K1R9L9, A0A3N7FQE2, A0A3N7FE30, B9IQL6, B9HYQ6, A0A2K2CBSO, A0A2K1Y5X3, A0A2K2CBN5, A0A3N7I1Z5, A0A2K1R9K4, U7E275, A0A2K2CBV3, A0A3N7GT81, A0A3N7FK00, A0A2K1YJB7, A0A3N7GCP1, A0A3N7FXT6, A0A3N7FWX1, A0A2K2CBR2, U7E2B5, A0A2K1YJJ4, A0A2K2CBQ6, A0A2K1Y5Y0, U7DTV7, A0A2K1R9J0, A0A2K1YJC0, A0A3N7EN77, A0A3N7HDH1, A0A2K2CBV5, A0A2K1YMS3, A0A3N7H760, A0A2K2CBU2, A0A3N7FMC7, A0A3N7FFN1, A0A2K1YII9, A0A2K1YFA5, A0A2K1YMG3, Q0ZCC5, A0A3N7F1A3, A0A2K1YMC5, A0A2K1R9L8, A0A2K2CBS9, A0A2K1YF89, A0A2K1YJB0, A0A3N7G5C0, B9I819, A0A3N7EZT8, B9H1V2, A0A2K1YF22, A0A2K1YKS5, A0A3N7FQ08, A0A2K2CBS8, A0A2K2AC56, A0A2K1YF85, A0A2K2AA68, A0A2K2CBS3, A0A2K1YF29, A0A2K1YF09, U7DZK5, A0A2K2AA71, A0A2K2CBQ2, A0A3N7F8U4, B9INUO, A0A2K2CD09, A0A2K2BQD8, A0A2K2CBR1, A0A2K1YME5, A0A3N7FHR5, A0A2K1YIJ7, U7DSM0, B9MUS9, B9H1U4, A0A3N7FQ25, A0A2K1YF75, A0A2K1YME2, A0A2K1YF38, A0A3N7G9D1, B9H9X2, B9I050, A0A2K1R9I3, A0A3N7F8V7, A0A2K1WUYO, A0A2K2AP04, A0A3N7FUP4, A0A2K2AP06, A0A3N7FF46, A0A3N7FTN5, A0A2K1YF58, A0A1X6NVT8, A0A1X6NQM4, A0A1X6PFD2, A0A1X6P8C6, A0A1X6NNT9, A0A1X6P5P1, A0A0N0JQI8, A0A167RBV0, A0A167R7I2, A0A167RBU3, A0A2R8AUKO, H3FLN2, H3FU64, A0A2A6CLV0, H3G269, H3DYL1, A0A2A6BKN6, A0A2A6CL96, H3F912, A0A2A6CKS7, H3DXL2, H3EPT2, A0A2A6BKX0, A0A2A6BRD4, H3EMR6, H3F239, H3FWN3, H3EMS5, H3E6Q3, A0A2A6CW20, H3G484, A0A2A6CWJ1, A0A2A6BQE5, H3G483, A0A2A6C8D4, A0A2A6CWL7, H3F6C9, A0A2A6BXL8, A0A2A6BTB7, H3E9C6, H3F0T6, A0A2A6CRJ5, H3FU41, A0A2A6CC90, A0A2A6CXB5, H3EUD8, H3EUB5, A0A2A6CVS9, A0A2A6C5D7, H3EYA4, A0A2A6CNUO, A0A2A6CPM8, H3FXU2, H3FXP1, H3FH55, H3FDUO, H3DRR5, H3F6D1, A0A2A6C0F3, A0A2A6CJP0, H3FUP4, H3EC28, A0A2A6C3M2, H3E6Q4, A0A2A6BWE1, A0A2A6CV10, A0A2A6C2I9, A0A2A6BTN5, H3FDT3, A0A2A6BRT5, H3EUB4, A0A2A6BRY8, H3EIL7, A0A2A6B687, H3F237, A0A2A6B8K1, A0A2A6CBV8, A0A2A6BIV2, A0A2A6BTB0, H3EA22, A0A2A6CPA6, A0A2A6D2U8, A0A2A6CLQ3, H3G4B7, A0A2A6BP16, H3FP67, A0A2A6B6P2, A0A454XRJ9, A0A2A6CS22, A0A2A6BMJ9, A0A2A6CVF2, A0A2A6C5B7, H3FKW8, H3E9C4, H3FLR8, A0A2A6CL26, A0A2A6CAV6, A0A2A6C0V0, H3DT54, A0A2A6C9M6, H3F624, A0A2A6CIU4, H3G421, H3EKC8, A0A2A6CYT9, A0A2A6CJH0, A0A2A6CTB6, H3FFI6, A0A2A6BY19, A0A2A6BF62, A0A2A6C2U7, A0A2A6C8U6, A0A2A6CM39, A0A2A6BTW0, A0A2A6B724, H3EWG7, A0A2A6CGU1, A0A2A6C3N2, A0A2A6BRI6, A0A2K6FYF0, A0A2K6FWB1, A0A2K6FWE4, A0A2K6GMV5, A0A2K6FWC7, A0A2K6GU92, A0A2K6FYE7, A0A2K6EIZ2, A0A0P6W8Z7, A0AIW9IFG2, A0A1W9I8T4, A0A2W4P8I4, A0A2W4P3J5, A0A3A0F8E5, A0A317HTN1, A0A448WDT9, A0A3S5A576, E6YCZ6, E6YCZ1, M5WXQ1, A0A251PZP6, A0A251Q7E1, A0A251PF29, A0A251PTU7, M5WCL9, A0A251Q445, M5VZY3, A0A251PKI1, A0A251Q478, A0A251P735, A0A251PF35, M5WJ95, A0A251Q7C8, A0A251PKC2, M5WMF2, Q6DU55, M5X5D7, M5WWL5, M5VUG0, A0A251PN29, A0A251PMZ2, A0A251RBW4, M5WM53, M5WZG6, A0A251Q443, M5XEL7, M5X9Q1, A0A251PMZ1, A0A251PN38, A0A251REA2, A0A251Q484, A0A251RGM7, M5X9P7, A0A251PF27, M5VTG3, M5WZA7, M5WZT3, A0A251PF21, M5WK87, A0A251PZD2, A0A251PLL1, A0A251NRG7, A0A251PID0, M5WZ40, A0A251NHZ5, A0A251PWJ2, M5WPH8, M5VZT1, A0A251PKJ4, A0A251Q4B5, A0A251PLJ6, A0A251PGK7, M5X1J1, M5WG45, M5WD24, A0A251PF18, A0A251PN12, A0A251PLYO, A0A251PMY4, A0A251Q434, M5X8S1, M5X455, A0A251PYL5, M5XQT3, M5X887, M5X3N3, M5Y1R6, M5W6E4, A0A251P734, M5WH13, M5Y3U1, M5X159, M5WK95, A0A251PF34, A0A251Q485, M5WPP2, A0A251NRF5, A0A251PF25, M5WLB2, A0A251Q454, M5XAQ5, A0A251PN32, M5WPSO, M5X3A2, A0A251PN76, A0A251PKI0, A0A251PQ97, M5WXJ1, A0A251PN13, A0A251PF23, M5WKA3, M5WM79, A0A251Q5C4, A0A251Q4D5, A0A251RGX7, M5X9S4, A0A251NRE5, M5WXU6, A0A251REE6, A0A251PN41, M5XL06, A0A251Q458, M5X216, M5WH09, A0A251Q463, M5X8Y5, A0A251Q479, M5XMQ7, M5XHF0, M5WQ28, M5X7U6, M5WVR7, M5WSH4, A0A251NDA6, A0A251PN25, A0A251PN53, A0A251Q7F0, A0A251Q464, A0A251NT57, M5X6Z5, A0A251PRA6, A0A251PLN9, M5WPY5, M5WPV3, A0A251NIW0, M5WJJ1, M5X3N0, A0A251Q190, M5X3I1, A0A251N2H7, A0A251PK13, M5WNI3, A0A251PN15, A0A251PPI2, A0A251PWI5, A0A251PEY5, A0A251NFQ2, A0A251Q5B2, A0A251PF13, A0A251PN44, A0A251Q436, A0A251PEZ5, M5WVG0, A0A251PN23, A0A251PF11, M5WPG0, A0A251PPF4, A0A251PWI0, A0A251Q435, M5WN54, M5WDJ6, M5X2T2, A0A251REB1, A0A251PPD9, M5X9G3, A0A251NKN9, A0A251PWL4, A0A251Q4A4, M5WZT2, M5W812, M5WX54, A0A251PKK4, M5X978, M5WZV5, M5WPYO, A0A251PF28, A0A251PN85, M5WWQ5, A0A251PN40, A0A251PWJ6, M5XIJ4, A0A251QKG7, M5WNK5, M5WM02, A0A251Q470, A0A251Q456, M5VVP2, A0A251PF31, A0A251PEZ9, A0A251PN33, M5WWU8, A0A251PEC6, A0A251PKE3, M5WPH5, A0A314U9M1, A0A314Y373, A0A314Z0A8, A0A314ZKH3, A0A314XG48, A0A314YPW4, A0A314XMH1, A0A314YT22, A0A314XP08, A0A314XQJ9, A0A314Y542, A0A314UQL0, A0A314YBU3, A0A314YQ99, A0A314UY39, A0A314YQW7, A0A314YYP7, A0A314YVF9, A0A314XGA1, A0A314UX31, A0A314Z5K6, A0A314Z001, A0A314YRN2, A0A314YT57, A0A314YBR0, A0A314XP39, A0A314Z720, A0A314Y5X9, A0A314UHG7, A0A314Z8Q0, A0A314Y699, A0A314YB32, A0A314UTP8, A0A314UPQ4, A0A314V0I2, A0A314ZFK4, A0A314YL52, A0A315AX60, A0A314YAA4, A0A314Z0M5, A0A314Z3J4, A0A314Y7M3, A0A314V0X4, A0A314YXT4, A0A314YUF3, A0A314UJA2, A0A315AGJ7, A0A314ZPW0, A0A314Y1L0, A0A314XZH9, A0A314ZSU2, A0A314UXP5, A0A314Y0Z2, A0A314YAA6, A0A314Y818, A0A314Y3J8, A0A314ZB68, A0A314ZP76, A0A314YUG3, A0A314Z802, A0A314UEM7, A0A314YRH3, A0A314Y8A2, A0A314UWI0, A0A315ARL6, A0A315A6R0, A0A314UJ27, A0A314YAF3, A0A314UEA8, A0A314Y3B8, A0A314ZHU3, A0A314YJP3, A0A314XSE1, A0A314YFP3, A0A314Y3T4, A0A314Y829, A0A314Z9C8, A0A314ZVV1, A0A314YLI7, A0A314ZJ97, A0A314YUI6, A0A314YBY2, A0A314XVQ6, A0A314XIP0, A0A314XP62, A0A314UP42, A0A314UV53, A0A314XIQ0, A0A314Z5Z0, A0A314YBQ1, A0A314YSN1, A0A314YKD5, A0A314UCK4, A0A314YAE2, A0A314YCB1, A0A314XVQ9, A0A314UXN9, A0A0L1JPX5, A0A1V8RTC8, A0A1V8RVSO, A0A316C775, A0A432VCJ6, A0A432UZB1, K9SMW0, A0A448Z8L7, A0A167HEYO, A0A0F6AB37, A0A161Z1S3, A0A1C0TWN0, A0A1Y6CLA4, A0A1Y6C4J4, A0A1Y6CJZ7, A0A139HR58, A0A139H9G6, A0A139HU89, A0A139HUE9, A0A139H0C0, A0A139HE34, A0A139HF24, M3AJY5, M3ABI7, N1QAP8, M3AM66, A0A139IDZ4, A0A139ISD7, A0A139IAH9, A0A139I9V6, A0A139IQ32, A0A139HLG1, A0A139I5S0, A0A139IE78, A0A139IRW9, A0A139I5G1, A0A139I8T4, A0A366DTY3, A0A0V0Q930, L8FX08, A0A177A5U9, A0A177AEB2, A0AIB8FQ74, A0AIB8FAM6, A0AIB8FAB7, A0AIB8EVM3, A0AIB8DV39, A0AIB8EGD8, A0A1B8DP42, A0A1B8DS74, A0A1B8E4V2, A0A1B8DQT9, A0A1B8D360, A0AIB8DGK9, A0A094EGR9, A0A094DWV3, A0A094E8W1, A0A094ECR8, A0A094F5X7, A0A093X015, A0A093Y9E1, A0A093ZSP4, A0A093XA42, A0A094AHX5, A0A093YEN8, A0A093YI19, A0A093ZRK7, A0A093ZUL1, A0A093YA26, A0A093Y8L0, A0A093XLF2, A0A093XX47, A0A093XB82, A0A093ZC45, A0A093ZL44, A0A093Z902, A0A094BF38, A0A094B572, A0A094B804, A0A094AQ42, A0A094AF07, A0A094CGQ0, A0A094AJK3, A0A094C0V4, A0A094AXJ9, A0A094C5H2, A0A094CCB3, A0A094DRK2, A0A094D2R0, A0A094DJC7, A0A094D600, A0A094CUY7, A0A094D6Z9, A0A094CLP7, A0A094FL50, A0A094DH72, A0A094FUT8, A0A094GG55, A0A094FUK7, A0A094G2V6, A0A094G2T3, A0A094F380, A0A094HAD8, A0A094FXE5, A0A094F3W2, A0A094IUK4, A0A094FUX7, A0A094GE65, A0A094IFS5, A0A094FC36, A0A094HXC7, A0A094HR56, A0A094JG43, A0A094HUW8, A0A094H2I0, A0A094GL04, A0A094IWA8, A0A094HPW1, A0A094I5T1, A0A094K6B1, A0A094H1N9, A0A094KPW5, A0A094HXM1, A0A094IA09, A0A094I941, A0A094HH70, A0A094H7Z8, A0A094HW58, A0AIB8CEM6, A0A1B8C167, A0AIB8BVH8, A0AIB8BVN1, A0AIB8CLH8, A0AIB8GFJ5, A0AIB8GIK4, A0A1B8G6U4, A0A1B8G8P5, A0AIB8GXU4, A0A371BDI6, A0A0Q7TDU5, A0A345ZXK6, A0AIY2DW08, A0AIY2D6M0, A0A1Y2E0V4, A0A1Y2DSV9, A0A1Y2D8N4, A0A1Y2E3R2, A0A1Y2E4X6, A0AIY2E8Q9, A0A2V4SND8, A0A2T5HEC1, A0A0A0EH55, A3U263, A0AIX6ZN84, A0AII1NTG6, A0A399J8R4, A0A2E3H3U4, A0A2E2PAW3, A0A1M6RS66, A0A081MMN6, A0A1H8C883, A0A433S634, A0A2M8MZT2, A0A257GRH4, A0AIW6ZZH9, A0A2T0RJD8, A0AIG8QA31, A0A1M7Z7T5, A0A081CEQ5, A0A093DR50, L5JNS7, L5KBX5, L5KUG3, A0A3B4GGZI, A0A3B4ER40, A0A3B4EPW7, A0A3B4EUN9, A0A3B4G794, A0A3B4GLL4, A0A3B4F895, A0A218X271, A0A2I0HMY7, A0A2I0J4H2, A0A218X161, A0A2I0UU8, A0A2I0J4I9, A0A218Y2C4, A0A2I0HIQ9, A0A2I0J9K2, A0A218Y341, A0A2I0IQT3, A0A2I0KMH9, A0A2I0U58, A0A2I0KZ71, A0A218X0P0, A0A218WZC1, A0A218WYH1, A0A218WXK1, A0A2I0JQJ0, A0A218WZ09, A0A2I0HYS2, A0A218WXG4, A0A218WUD1, A0A2I0HAP0, A0A218XP01, A0A218WER6, A0A2I0IQZ3, A0A2I0HSV7, A0A218Y426, A0A2I0HST7, A0A218Y3D0, A0A2I0HPH7, A0A218Y2B4, A0A218WC26, A0A218VVJ2, A0A2I0KRY6, A0A218Y294, A0A2I0I1Y2, A0A218WXR5, A0A218WY26, A0A2I0HEN9, A0A2I0KGT9, A0A218X1M6, A0A218X8E1, A0A2I0I1U5, A0A2I0K6N7, A0A218X1Y4, A0A218YOY4, A0A2I0HHU4, A0A2I0KWU4, A0A2I0IB26, A0A2I0KD33, A0A2I0JRD0, A0A218Y212, A0A218VZL6, A0A218X0N1, A0A218Y398, A0A218X6V7, A0A218W3Q4, A0A218X0A3, A0A2I0IHT5, A0A218VUN0, A0A218Y175, A0A218WY07, A0A2I0I1X9, A0A218YlX1, A0A218X956, A0A218Y478, A0A218XUG7, A0A2I0I3E5, A0A218WYF1, A0A2I0I1U6, A0A2I0KZ81, A0A218Y494, A0A218W9L6, A0A218XS02, A0A218Y227, A0A218X264, A0A218WXQ6, A0A2I0L0F4, A0A2I0J4I3, A0A2I0HKJ3, A0A2I0KZ88, A0A2I0KZ67, A0A2I0KYL9, A0A218X7K9, A0A218WXT2, A0A2I0KZ70, A0A218WY64, A0A2I0KW29, A0A2I0KZA1, A0A2I0J932, A0A2I0U71, A0A2I0KG46, A0A2I0I6U2, A0A2I0HSU7, A0A218WXY7, A0A218XFG8, A0A218XFR5, A0A2I0KN21, A0A2I0HKH6, A0A2I0I2C3, A0A218X1C5, A0A218Y271, A0A2I0KET6, A0A218X305, A0A218Y234, A0A218Y351, A0A2I0KX43, A0A218X2P8, A0A218Y2C0, A0A2I0IHV8, A0A2I0IPE4, A0A218WYC8, A0A2I0KFP7, A0A2I0IHT9, A0A218XW17, A0A218XFS5, A0A218Y0Y2, A0A2I0IQG0, A0A2I0KAW6, A0A2I0KC11, A0A2I0JYX5, A0A218W566, A0A218WF52, A0A218WZS3, A0A218Y3E1, A0A2I0HIE5, A0A2I0KWT0, A0A2I0HT88, A0A218X7K7, A0A218WXZ7, A0A2I0L4U1, A0A218WY16, A0A218WYD8, A0A218XUR4, A0A218WY96, A0A2I0KZ61, A0A218X3M6, A0A218XAX6, A0A218XDE2, A0A2G8RAW8, A0A238VTL4, A0A0J5QAI6, A0A179GCR8, A0A2U3DTP8, A0A2U3DSM1, A0A179G1G3, A0A2U3ECC8, A0A179HQR1, A0A179GK49, A0A3B4BXA6, A0A3B4DRA6, A0A3B4BXD6, A0A3B4D157, A0A3B4BXJ5, A0A3B4C326, A0A3B4C1E6, A0A3B4C446, A0A3B4DN28, A0A093P1B8, A0A093RLN1, A0A093QRK6, A0A093N8L5, A0A178E6Z5, A0A178EA49, A0A178EEX5, A0A178DK72, A0A178DY86, A0A3M7M5G6, A0A3M7MFK4, A0A3M7MG51, A0A3M7M128, A0A3M7MCE0, E3S6M1, E3S7C0, E3RU61, E3RPP7, B2WLR3, B2VUMO, B2WMT4, B2WBS3, A0A2W1GRF2, A0A2W1HFA3, A0A2W1HWY2, A0A2W1EGN9, A0A2W1ELB0, A0A2D4C6Q4, A0A2D4BUE8, A0A2D4CGH4, A0A2D4C0E0, A0A2D4C4K6, A0A2D4C7F2, A0A2D4BJS7, A0A2D4BHR4, A0A2D4BT34, A0A2D4BXK6, A0A2D4BRG2, A0A2D4BI32, A0A2D4C690, A0A2D4BGL9, A0A2D4C6A5, A0A2D4C3B2, A0A2D4C8R4, A0A2D4C6I3, A0AIV8SZ61, A0AIV8TRB4, A0A1V8T5T1, A0AIV8TF18, A0AIV8TEM5, A0A1V8SMS7, A0A1V8TN34, A0A1V8SPQ2, A0A1V8TVE0, A0A1V8ST60, A0AIV8TZH6, A0AIV8UTC1, A0AIV8V8Q1, A0AIV8UBK3, A0AIV8UW76, A0AIV8UON8, A0A1V8U6W6, A0AIV8TZA8, A0A1V8U585, A0AID1VG42, A0AID1UP37, A0AID1UTW6, A0AID1VPW1, A0AID1UMW3, A0A1D1V436, A0A1D1W957, A0AID1UIH8, A0AID1UU11, A0A1D1W7T4, A0AID1VJV1, A0A1D1VJT6, A0A1D1VAI5, A0A2D3V2X9, A0A2D3ULC3, A0A2D3UWU9, A0A2D3V3D6, A0A2D3V0K0, A0A2D3UT01, A0A2D3V4J3, Q9SBP5, Q9SBP1, Q9SBP4, Q9SBP3, Q9SBP2, Q9SBP0, A0A0D5XMK2, A0A0D5XNG1, Q8LP63, A0A0D5XNJ2, A0A3871HF6, 080347, 080351, Q6L8R0, A0A2D1CRR0, A0A3B1EZU5, 080350, A0A387IHI8, 080346, Q5QHE9, A0A387IHE2, D2I902, A0A387IHI2, A0A387IHE1, 080349, A0A3B1EZV1, Q6L8R6, Q5QHE7, Q940W8, A0A387IHI7, Q001P7, Q6L8R1, G4XN45, G4XN49, A0A387IHI6, Q6L8R4, A0A387IHH3, G4XN46, Q6L8Q9, Q6L8R2, A0A3B1EZU3, Q6L8R3, 080356, A0A387IHI4, Q940W6, A0A387IHH9, 080352, Q940X0, Q5QHE8, G4XN47, Q5QHE6, Q6L8R7, Q5QH04, A0A387IHF0, 080353, A0A2D1CRR9, 080348, G4XN48, Q6Q2Z5, A0FLG6, A0A387IHE8, A0A3BIEZU4, Q6L8R5, Q940W9, P93511, Q940W7, A0A2V0NKU4, A0A2V0PBY3, A0A2V0PFW8, A0A2V0P2D6, A0A2V0PQQ0, A0A2V0NRX2, A0A2V0NLP1, A0A2V0NLF4, Q01177, P17945, P14272, A0A0G2K4I9, F7FMSO, P70521, Q5FVS2, Q6TUF8, A0AIVIFYM3, A0AIVIFQA1, A0A1I8CMM6, A0AII8C8R0, A0A1I8CK66, A0A1I8CA14, A0AII8C8S3, A0AII8D6L7, A0A1I8CRQ9, A0A1I8C3E9, A0A1I8CQ74, A0A1I8CUY7, A0AII8CVM8, A0AII8D1P2, A0A1I8CH38, A0A1I8CBE6, A0A1I8CKM7, A0AII8CF77, A0AII8CLA6, A0AII8CV32, A0A2K6L170, A0A2K6MRUO, A0A2K6MWT8, A0A2K6N2E4, A0A2K6MN57, A0A2K6MWU2, A0A2K6MWT4, A0A2K6N2J7, A0A2K6L173, A0A2K6PRN4, A0A2K6NWB1, A0A2K6N799, A0A2K6RCW8, A0A2K6NTA1, A0A2K6N7A2, A0A2K6N796, A0A2K6NWB3, A0A2K6NWB7, A0A2K6PRM9, A0A2K6NTA8, Q8I6X5, A0A2D8K1E4, A0A1Q4C6M3, A0A1Q4DD97, A0A1M2Z0A0, A0A1Q3KY06, A0A2E4Z0V6, A0A2E2MCQ3, A0A2E2M8M6, A0A2N6AXD7, A0A2N6B0J3, A0A2A5G4Z8, A0A2N6B7F0, A0A2A4P7M7, A0A3A0EE51, A0A2A4R9T6, A0A349QK41, A0A2A4NQQ8, A0A0N1DQF6, A0A0N0LCX8, A0A1C3Y3G1, A0A2U2DQR8, A0A109K3K2, A0A3SOSF88, A0A246DZ15, Q2K005, B3Q100, B3PUG4, A0AIL5PF86, S5S373, A0A0A8GIJ8, F2AFE1, A0A327YDD0, W6RLA5, G9AGQ7, A0A2L0H058, A0A2A6LWB4, A0A2L0HFL1, A0A2A6LVY4, N6V882, A0A0B4XEY4, A0A1L5NUL5, S3H7T7, A0A370KJW2, A0A1C3WED0, A0A2A6KF27, A0A387FJYO, A0A1S9GBZ1, A0A1S9GD17, A0A072BW40, C6B7V3, C6B6X0, B6A442, W0IT72, J0KL73, J0L6R4, I9N4J7, I9NL44, A0AIC9HR08, A0AIC9HXI1, A0A3E1B354, A0A3E1AZA5, A0A3E1BI76, Q1M687, Q1M724, J0K300, J0VE20, A0A1Q8H6V5, A0A2L1CRP2, A0AIQ8HGY6, A0A222U8G4, A0A222U658, A0A444NFR8, A0A179BUW9, A0AIBICLA0, A0A154IRG5, A0AIL3ZLB7, A0A3S4Z848, A0A154IPB2, A0A444HN98, A0A444IGJ5, A0A444NC52, A0A3S3WH50, A0A2K9ZEW6, A0A2K9ZE91, A0A2Z4YEZ8, A0A3S4ZBC5, A0A2Z4YX29, A0A1G4TJ35, A0A1B4YZE1, A0A1B4YZG6, A0A1B4YZE6, A0A2N5AWF1, A0A1B4YGKO, A0A316CJ14, A0A117N3F6, A0A101KVC7, A0A1B4YE29, A0A1A5HXY1, A0A1A5QH69, A0A1A5R3P7, A0A1C3W6S0, Q92VA6, A0A2J0Z7E8, A0A437F0L4, A0A222GTF2, A0A437G4H5, A0A222K2F7, A0A3S2TJR4, A0A437E078, K0Q667, KOPTJ5, A0A1C3U1J6, A0AIC3WEX5, A0A376ASS4, A0A376AR20, A0A0D8KAH4, A0AIX7CLE5, A0AIQ9AYC7, A0AIQ8ZX36, A0AIW6GK72, A0AIW6GQX6, A0A0M3GJ47, A0A0M3GFH7, A0A192TDW1, A0A192TJG2, A0A2U3CMZ0, A0A3R9AP92, A0A1L9CN61, A0A1S9ESQ4, A0A176XJZ8, A0A0X8IY53, A0A0D0J656, A0A176X4G7, A0AIB9SXL8, A0A0D0KZ04, A0AIV2AH49, A0AIB9UR06, A0A2T7W9Z1, A0A2L2LHE9, A0A083ZGC6, A0A2C5Z3N8, A0A109VT16, A0A3G2DPM2, A0A420FYH9, A0A024IWY1, A0A3B8F8H3, A0A2T7W547, A0AIQ9ALQ8, A0AIQ9AGL9, A0A376ABJ2, A0A3R9CXJ4, A0A2A5KUS5, A0A248W2A8, A0A444LBM9, A0AIL8PLX4, A0A1M3P8H7, A0AIM2YPN5, A0A143ZFH2, A0A0N1AWY9, A0A0N1A5V6, A0A1B9RQK2, A0A1B9S8M0, A0A292ECMO, A0A292EB32, A0A420CH60, A0A420CND4, A0A285Y1D3, J2DBP0, J2WMP8, A0A432P6G8, A0A432NWE3, A0A3SOQ0E5, A0A2A6JF54, J5QAT9, A0A387GV30, W6WJL6, J2IQH9, J1T8K3, A0AIW6KW30, A0A2A6R2N3, A0A098RMU8, A0A060I507, U4Q372, A0A2A6K1I0, A0A2T3EJJ0, A0A2S6BBW7, A0A2A6ICH9, A0A2A6I5L3, A0A2A6HV73, A0A2A6HMS6, A0A2A6I066, A0A2A6KX61, A0A0M3B778, A0A0M3B5N6, A0AiS1T9A9, A0AiS1TD64, A0A0Q4WAW7, A0A0Q5D0H9, A0A0Q5EIT4, A0A0Q5W5J8, A0A0Q5WYK9, A0A0Q6H5F7, A0A2A6LEH7, A0A192M5Y9, A0A235AY41, A0A235AJ79, A0A235AZH9, A0A1G5HB02, A0A1H9WX89, A0A1I0ZNA8, A0A1H6GJQ1, A0A329AJB1, L0NJT2, A0AIW6PV42, A0AIW6PZX1, A0A2L0W608, A0A2L0W9E8, A0AIG8WJR3, H4EZX2, KOVNQ6, A0A2V4UN27, A0A2V4VIK9, A0A2V4UL45, A0A246SBW7, A0A246T4R1, A0A246T354, A0A246TWX7, A0A437NBSO, A0A0Q6QM12, A0A0Q6RH95, A0A0Q6S6H3, A0A0Q6VUN6, A0A0Q6UVB6, A0A0Q6ZH68, A0A0Q7XTZ6, A0A0TIWTZ2, A0A0Q7NJK2, A0A0Q7PCQ9, A0A0Q8C8S6, A0A0Q8BTC4, A0A0Q8GJX7, A0A0Q8NRH2, A0AIS1PS94, A0A1N6XKQ2, A0A1N6UY85, A0AIW2DVC3, A0A2T3GU97, A0A2N4XYT7, A0A2V5CDS1, A0A198YKG9, A0A198YI97, A0A2KOZQK3, A0A2KOZP10, A0A1E4YFI2, A0A095UEN9, A0A3D3BI31, A0A2W6XJX2, A0A2M8UlJ2, A0A353G6F7, A0A285U5F1, A0A2N0DBB1, A0A1Q9A5B5, A0A1H8WJ94, A0AIH8IHA7, L0LF24, A0A329Y7X4, A0A2T0P999, A0A2W4CTD1, A0A2W4B084, A0A432PRA9, A0A1Y2CKU5, A0A1Y2CE17, A0A1Y2B783, A0A1Y2AMS3, A0A1Y2C375, A0A1Y2C103, A0A1Y2CFU8, A0A1Y2BGJ3, A0A1Y2CP39, A0A1Y2BQKO, A0A1Y2CED3, A0A1Y2AE69, A0A1Y2CJV3, A0A1Y2CDM5, A0A1Y2C0V7, A0A1Y2BFL8, A0A1Y2CJS8, A0A1Y2AQ77, A0A1Y2BHM1, A0AIY2CL98, A0A2P2IMF7, A0A2P2IIC6, A0A2P2JRF0, A0A2P2II72, A0A2P2MSG9, A0A2P2MSG6, A0A2P2MQG3, A0A2P2MSF5, A0A2P2K922, A0A1V3P122, T1HW46, T1HKB1, T1HVY5, T1H815, T1I1K7, T1HB28, A0A1N7LNA9, A0A2T5JUG7, A0A2T4JF26, D5AST4, A0A0E2PIW9, A0A1G7E3U5, A0A330HGS9, A0A285SYE0, A0A239GLZ7, A0A285CQ45, A0A366K6J3, A0A1H4S7Q6, A0A421BL93, C8RWS1, A0A3D6CUW1, Q3IY48, A4WNG2, A3PFM8, B9KQL4, A0A370XYR7, A0A2W5U3H5, A0A3G6W6P9, A0A2T4J571, A0A2T4JJD8, A0A1N7KZF5, A0A318UiP8, A0A2G1CH11, A0A372EZL6, A0A327MOR4, A0A3N2R174, A0A347UC92, A0A3S3T2P1, A0A2M7EPC2, A0A1B6YLV4, A0A0P7W120, B6ATT1, A3JM87, A0A418SAV9, A0A3L9Y7X3, A0A257F196, A0A0B4E3A1, A0A372IHY4, A0A3D4DUJ2, A0A3D5PSG7, A0A2V5FZ51, A0A2D4V2U9, A0A2A4NME6, A0A2E3PJV4, A0A2E3FHN5, A0A356V427, A0A2E3PH72, A0A350AHD8, A0A2E3PP51, A0A2A4ZFU9, A0A2D5J967, A0A2V5G5E0, A0A2D5IXH4, A0A259DA28, A0A259LR21, A0AIQ4CVQ4, A0AIG3GSI9, B6B7T2, A0A2G6ISQ3, A0A2G6CFH3, A0A2G6I8H1, A0A2G6II81, A0A2G6IV12, A0A2A4S6F2, A0A355T6E9, A0A2Z4UG21, A0A2E5BRN7, A0A2A5EMY3, A0A327JMD5, A0A327KJ78, A0A327K3R9, A0A3S4B6R7, A0A327L3N5, A0A127F2Y1, A0A318TNH4, Q6NBK5, Q07TU8, Q212S6, Q21BD4, Q13CZ0, E6VBP2, Q2IR81, B3QEG9, A0A0D7EX63, A0A0D7F517, A0A323UFY3, A0A2R4GPL3, A0A418UYX9, A0A336JVI2, A0A1H8TZ49, A0A0N1C8P1, A0A365U4P5, A0A2E7W3G6, A0A2T5U026, A0A428K1H3, A0A366C229, A0A1V0I275, J5PI78, A0A2E2JWZ9, A0A1Q9QUC6, A0A1E1KPQ4, A0A1E1LMW4, A0AIE1KU29, A0AIE1KM25, A0AIE1JTK9, A0AIE1LGC2, A0AIE1K9E7, A0AIE1KXI0, A0AIE1KAU5, A0AIE1LP64, A0AIE1MDX2, A0AIE1M5W1, A0AIE1MFU2, A0AIE1M9F8, A0AIE1MTL8, A0AIE1MD61, A0AIE1M215, B9SSB2, B9STG0, B9SEN3, B9RXY2, B9SXC2, B9RQP9, B9RFM4, B9SIG2, B9SSB7, B9TAY3, B9R758, B9RHL2, B9SJQ1, B9T444, B9SV42, B9S7P6, B9SIQ7, B9SSB5, B9T7U7, B9RLS4, B9SIR0, B9R757, B9R755, B9SSA1, B9RXX5, B9R760, B9SNG5, B9RQP5, B9SSC0, B9R761, B9SOR9, B9SRH2, B9SFF3, B9RI65, B9S9P6, B9SFF2, B9SFX3, B9RXY0, B9RXY1, B9RVX8, B9SW97, B9SFX5, B9SFF5, B9SIR1, B9SZ96, B9RXX9, B9SFF1, B9RFM6, B9S858, B9SSB8, B9RV63, B9RLS2, B9T7U8, A0A2G2GCP5, A0A2G2R0P0, A0A2G1YWK1, A0A2G2HQX6, A0A2G2GH02, A0A2P6Q8G4, A0A2P6SHN7, A0A2P6SCY4, A0A2P6Q365, A0A2P6QUU6, A0A2P6Q342, A0A2P6S2Q9, A0A2P6QU66, A0A2P6PXU3, A0A2P6Q3D1, A0A2P6S898, A0A2P6SAU9, A0A2P6RJH7, A0A2P6Q908, A0A2P6QQ00, A0A2P6QVT9, A0A2P6QJ32, A0A2P6Q399, A0A2P6S8C4, A0A2P6Q341, A0A2P6RSA0, A0A2P6PFN5, A0A2P6Q3C0, A0A2P6RCT1, A0A2P6QB94, A0A2P6Q3C5, A0A2P6Q391, A0A2P6QHF8, A0A2P6Q8G0, A0A2P6S2T7, A0A2P6S756, A0A2P6RTY8, A0A2P6Q351, A0A2P6QBC3, A0A2P6QBG0, A0A2P6PXY7, A0A2P6QBF8, A0A2P6RCR1, A0A2P6QAY4, A0A2P6Q3C2, A0A2P6Q3E7, A0A2P6R3M6, A0A2P6Q0T1, A0A2P6RX36, A0A2P6SAB0, A0A2P6Q3A9, A0A2P6Q390, A0A2P6QB71, A0A2P6S6W6, A0A2P6S765, A0A2P6Q3D5, A0A2P6Q8T0, A0A2P6QBH7, A0A2P6Q3A8, A0A2P6Q3C6, A0A2P6Q8Z8, A0A2P6Q367, A0A2P6RX44, A0A2P6QU56, A0A2P6QE17, A0A2P6QE07, A0A2P6QVC9, A0A2P6QAX3, A0A2P6Q355, A0A2P6Q396, A0A2P6RRU2, A0A2P6Q2N8, A0A2P6RUA2, A0A2P6Q3A7, A0A2P6RRQ5, A0A2P6SCB0, A0A2P6RU56, A0A2P6RS89, A0A2P6Q372, A0A2P6QM19, A0A2P6PX37, A0A2P6Q374, A0A2P6Q0Q2, A0A2P6SGJ1, A0A2P6QR39, A0A2P6Q381, A0A2P6Q362, A0A2P6QBG3, A0A2P6S2K8, A0A2P6S2R0, A0A2P6Q3B2, A0A2P6QBH3, A0A2P6RBI2, A0A2P6S6X4, A0A2P6PD71, A0A2P6SD05, A0A2P6S6W4, A0A2P6RST1, A0A2P6QBL5, A0A2P6PXZ1, A0A2P6Q348, A0A2P6RVC0, A0A2P6RRR7, A0A2P6S2Q6, A0A2P6S2L9, A0A2P6Q397, A0A2P6Q8F1, A0A2P6S2P5, A0A2P6QBJ8, A0A2P6QVN2, A0A2P6Q8K4, A0A2P6QBA5, A0A2P6RU59, A0A2P6Q8F3, A0A2P6QR92, A0A2P6P691, A0A2P6Q8G5, A0A2P6PDR1, A0A2P6QB88, A0A2P6RRU7, A0A2P6Q394, A0A2P6SAD4, A0A2P6SAC0, A0A2P6Q3A3, A0A2P6Q8H0, A0A2P6S8D3, A0A2P6Q8G3, A0A2P6PDQ4, A0A2P6QM80, A0A2P6RCQ5, A0A2P6Q8U1, A0A2P6Q0P6, A0A2P6RVC4, A0A2P6Q3B1, A0A2P6SAY2, A0A2P6RUW8, A0A2P6Q8U4, A0A2P6QW50, A0A2P6R760, A0A2P6RCX2, A0A2P6RRU6, A0A2P6REX4, A0A2P6PDQ7, A0A2P6SLZ0, A0A2P6Q385, A0A2P6S6Y9, A0A2P6Q8L4, A0A2P6QQ26, A0A2P6RCS7, A0A2P6SD19, A0A2P6RU70, A0A2P6R9I7, A0A2P6S2N8, A0A2P6Q8V1, A0A2P6PXZ2, A0A2P6S8C8, A0A2P6QM44, A0A2P6QBI9, A0A2P6PAH1, A0A2P6QBF9, A0A2P6QBI2, A0A2P6QBK6, A0A2P6Q387, A0A2P6Q392, A0A2P6QBI6, A0A2P6REY3, A0A2P6Q8I8, A0A2P6QVT7, A0A2P6QA23, A0A2P6S790, A0A2P6QL63, A0A2P6RW66, A0A2P6S8G7, A0A2P6QR51, A0A2P6Q3C7, A0A2P6PXY8, A0A2P6Q3C3, A0A2P6Q3D6, A0A2P6Q398, A0A2P6QB80, A0A2P6S2F9, A0A2P6S2M5, A0A2P6PEP6, A0A2P6QBC9, A0A2P6RS63, A0A2P6RRT9, A0A2P6QBC1, A0A2P6Q388, A0A2P6S745, A0A2P6PXY5, A0A2P6RS64, A0A2P6Q8Y2, A0A2P6QP61, A0A2P6PD75, A0A2P6S2K2, A0A2P6QBA3, A0A2P6R9L9, A0A2P6RWS3, A0A2P6S2N2, A0A2P6S729, A0A2P6Q8F7, A0A2P6QBK3, A0A2P6S742, A0A2P6RS56, A0A2P6PDH3, A0A2P6Q393, A0A2P6QBH0, A0A2P6Q8Y0, A0A2P6Q378, A0A2P6PY16, A0A2P6S8D1, A0A2P6S8F6, A0A2P6Q8U6, A0A2P6Q3J3, A0A2P6PXZ6, A0A2P6R9M2, A0A2P6SB38, A0A2P6QBK4, A0A2P6QB92, A0A2P6QBL2, A0A2P6SCZ3, A0A2P6QUV7, A0A2P6QWZ3, A0A2P6Q389, A0A2P6QA32, A0A0P7W3J3, A0A3B0MGU5, W8RZV3, E2CPR9, A0A316GM60, A0AIY5S619, X7F4L9, A0AlN7JUR1, A0A2T6B3V4, A0AIIlU481, A0A1W2TSD6, A0AIW2TQM6, B7RMG1, A3X6K1, A4ET50, A0AIX7BTA5, A0AIX6YTQ2, A0A0T5NYW0, A0A0T5PEA0, A0AII4YX20, A0A1N7E9E7, A3SKX6, A0A348WF88, A0A176F0X4, A0A176FFA7, A0A3A8B3W9, A0A365TZC0, A0A2E2S3M6, A0A2D9CQ95, A0AIH8EWM5, A0A017HNB8, A0AIY5RCZ8, A0A1G7AEP1, A0A0X3TTM9, A0A2R8C330, Q5LWT4, A0A346SKI6, A0A2V3VSP3, A0AIE3D7V4, A0AIE3DCB0, A0A2S7PS49, A0A2S7PDR4, A0A2S7Q765, A0A2R4QNS3, A0A2K8WAW8, A3K2N2, A0A2K6UT93, A0A2K6SSN8, A0A2K6S9K0, A0A2K6S9I8, A0A2K6UT83, A0A2K6SSN3, A0A2K6SSP3, A0A2K6UTP6, A0A2K6UYV6, A0A2K6UYX4, A0A0E9NMM4, A0AIH8QTL5, A0A327Y1D3, A0A1G8I7T9, S9QTS7, A0A1U7D1X3, A0A1G7KG72, A0A1S3LE46, A0A1S3S8H0, A0A1S3SH05, A0A1S3MAB2, A0A1S3SH07, A0A1S3SH04, A0AIS3MLI3, A0AIS3SH06, A0A1S3N1I0, A0AIS3SH08, A0AIS3SI18, A0A1S3SIC4, A0A1S3KP78, A0A1S3SH12, A0A1S3LE48, A0A1S3P228, F2TWZ6, F2UF68, F2TWR8, F2UG85, F2UPS4, F2USW3, F2U618, F2UA60, F2UFK9, F2U2Y5, F2UGI4, F2U386, F2UIR6, F2UHB7, F2TVR9, F2UJ78, F2UIC5, F2UDJ0, F2U6I3, A0A140E138, W6GGU3, A0A140E0U4, A0A2E0TIG6, T0RAA8, T0RYA1, T0QTS1, T0Q639, T0RYL1, T0PVB7, T0RQN3, T0R976, T0RRW1, T0QUW1, T0QNZ2, T0RMV6, T0PNV2, T0RAF6, T0QYA6, T0S243, T0RG82, T0QXL6, T0RI62, T0RPR9, T0R5M4, T0RIQI, T0RBY8, T0QL36, T0PWZI, T0QQ07, T0RBY3, T0RH69, T0QGJ1, T0RJMI, T0PH14, T0R5A9, T0Q529, T0PVB2, T0RYU4, T0PU14, T0RJM4, T0QZF9, T0R762, T0SIC6, T0R8A8, T0Q4I6, T0QAA3, T0Q2C6, T0QMW7, T0QN46, T0RIN6, T0QI45, T0QJB1, T0QUH0, T0RQM9, T0Q4C3, A0A067BU55, A0A067BYE3, A0A067BYA7, A0A067BJE2, A0A067C8S2, A0A067CBB6, A0A067C0P6, A0A067BSE2, A0A067BIR1, A0A067C156, A0A067BJJ2, A0A067CR05, A0A067CK80, A0A067C126, A0A067CKM6, A0A067C128, A0A067BMR9, A0A067CL67, A0A067BJT0, A0A067CFL9, A0A067BLZ4, A0A067BU12, A0A067BRT0, A0A067CCE3, A0A067CGD2, A0A067BEG5, A0A067CYL5, A0A067CF87, A0A067C4Y2, A0A067C8W4, A0A067BUH1, A0A067D5R2, A0A067CQB6, A0A067CG93, A0A067CKH3, A0A067C9Y0, A0A067BWR1, A0A067BRT6, A0A067CC90, A0A067BYB2, A0A067C8G3, A0A067C2R2, A0A067D5R8, A0A067CDQ0, A0A067BZY9, A0A067C9M8, A0A067C9D7, A0A067C6I2, A0A067CHH5, A0A067BRS1, A0A067BXJ9, A0A067CJ02, J5KAB5, A0A368BEY8, Q08668, P81860, Q26539, G3WZ37, G3VTC0, G3WZ36, G3VQX8, G3VAC2, G3VAC3, G3WT27, G3VQX9, A0A131ZZD7, A0A132A4Q6, A0A132A395, A0A132A5Y6, A0A132A5S6, A0A131ZTE1, A0A131ZSZ0, A0A1S5V1F4, A0A1S5V1F6, A0A1S5V127, A0A084G238, A0A084G6H4, A0A084G8P5, A0A0X3PVM5, A0A430QM43, A0A430QQG0, A0A183JN66, A0A183JYW3, A0A095C245, A0A094ZN30, Q5DBB4, A0A3Q0KQV0, A0A183MF64, A0A183NRF4, A0A3P8BBP8, A0A183R1Q6, A0A2S2N7W5, A0A2S2NT68, A0A2S2P2Z5, A0A2S2NBX7, A0A2S2NWJ0, A0A2S2PP89, A0A2S2NVK1, A0A2S2NBX8, A0A2S2NQG9, A0A2S2NP55, A0AIW5APA3, A0A0P7VVQ1, A0AIW5APF2, A0A0P7XEA4, A0A0P7ZBC4, A0A0P7UFY3, A0A1W4Y2F3, A0A0P7ULB2, A0AIW4YBZ6, A0AIW4YNQ9, A0A1W5A615, A0A0P7UJJ9, A0AIW4ZAZ3, W9CB98, W9C6T6, A0A1D9Q902, A0AID9PTT0, A0AID9PV26, A7E7Z9, A7E800, A7F252, A0A2U9B2H8, A0A2U9BYQ0, A0A2U9CUU6, A0A2U9CZF4, A0A2U9BGH2, A0A2U9CZ16, A0A2U9CYT4, A0A2U9D1N9, A0A2U9CUM9, A0A2U9CYZ6, A0A401NHP4, A0A401NSN8, A0A401NY63, A0A401PJU3, A0A401P1Q0, A0A2Z5XDQ5, A0A0P4WNK8, A0A0P4WFH3, A0A0P4WR24, A0A0N7ZDF3, A0A0P4WF65, A0A0P4WMW9, A0A3E2GVD1, A0AII6XD87, A0A3T0N402, A0A3T0N638, D8R4F7, D8SC04, D8QZ24, D8TB11, D8T7A8, D8TF62, D8SlJ3, D8SDZ7, D8QTB9, D8RST4, D8S4Y9, D8QZK2, D8T4B9, D8SRG6, D8RA35, D8RHS8, D8S2E7, D8TG77, D8RWA6, D8SX02, D8SNE6, D8SC01, D8QTB8, D8S6U2, D8R007, D8R008, D8RVP2, Q5NJB1, R4G2C9, R4G7D8, A0A3B4TE38, A0A3B4T255, A0A3B4THQ6, A0A3B4TCE1, A0A3B4UM85, A0A3B4X7F7, A0A3B4WXD7, A0A3B4WCE8, A0A3B4WX70, A0A3B4X514, A0A3B4Y8L3, K4A2F5, K3Z304, A0A368R0J1, A0A368REB7, K3XQN4, A0A368RTU8, A0A368RZT1, A0A368RZL0, A0A368PG49, A0A368SB62, A0A368RZH4, K3XPE2, K3XSM1, K3Z530, A0A368R959, A0A368R9A6, K3ZF16, A0A368QBK5, A0A368QCH9, A0A368Q4U7, A0A368Q482, K3ZPX7, A0A368RTX5, A0A368RTT9, K3XPE3, K3YDB8, A0A368RS21, K3YDQ0, A0A368RU04, A0A368QK95, A0A368RIC1, A0A368QBY1, A0A368R0I2, A0A368QB50, A0A368R8H3, K4A740, A0A368RSJ9, A0A368QB49, A0A368QCI5, A0A368R1M3, A0A368Q1T4, A0A368RZS3, K3YDS3, K3YIY9, A0A368Q4R1, K3ZCG7, K3XFB5, A0A368RZL6, A0A368RZL1, A0A368RZK1, K4A6H6, A0A368RZK0, K3YPU6, K3YEW1, K3YGJ6, K3XSH2, A0A368Q490, A0A368SI17, A0A368RZG9, A0A368RZG3, K3XEF3, K3YD20, A0A368RBT8, K3ZQI9, K3Y585, A0A368Q289, A0A368R1M5, A0A368R0L6, K3Y129, A0A368SAN2, A0A368Q6K5, A0A368Q6G7, A0A368Q569, K3Y505, K3ZZR5, K3ZRF9, A0A368RZI0, A0A368RZJ2, A0A368Q4J9, A0A368QR38, A0A368QXB9, A0A368Q621, A0A368PRU5, A0A368S195, A0A368RZM3, K3YD40, A0A368RZK9, A0A368R030, A0A368PK67, A0A368Q4N4, A0A368RU08, A0A368RZE2, A0A368RBV9, A0A368RZK6, K3XVB7, K3XIC1, A0A368R502, K3XEI2, K4A357, A0A368R9C0, A0A368QYB1, A0A368Q4M4, A0A368QBF6, K3Y2F6, A0A368S2P0, A0A368R035, A0A368RVM9, A0A368RZF9, K3XEM4, K3Y572, A0A368SR44, A0A368QMF4, K3YCM4, K4ALT0, K4A323, A0A368QPX0, A0A368RZI2, A0A368Q570, K4A115, K4AMY1, A0A368R970, K3Y5N0, A0A368RBZ6, A0A368RE63, A0A368SH77, A0A368RZR9, A0A368Q474, K3YEX8, A0A368R046, K4A086, A0A368RZF3, A0A368RH05, K3ZLB6, A0A368RRV9, K3Y545, K3Y5K2, A0A368Q6F6, K3YDM6, K4A5Z2, A0A368QBG1, K3XVB8, A0A368QJJ5, A0A368RZN4, K3YCU3, K3Z037, A0A0Q3PQD3, A0A368RFZ5, A0A368SP74, A0A368R3U7, A0A368RZU1, A0A368SII8, A0A368QW32, A0A368S227, A0A368RG75, A0A368RAQ1, A0A368Q480, A0A368RZI6, A0A368QC37, K3Z3T5, A0A368STV8, A0A368RZM9, A0A368QCP0, A0A368SC73, A0A368R950, K4A5S5, K3XQB1, A0A368SB67, K3ZQM2, K3YDMO, A0A368RZMO, A0A368R4P0, A0A368SBE7, K3YDB1, K4AIK7, K3ZQP3, K3Y559, A0A368SP77, K3XEG7, A0A368R690, K3Z3U1, K3XEE4, K3Y247, K3YC50, K4A5Y3, A0A368R8Y6, K3Y179, K3ZQP4, K4A5Y7, R0IH14, R0JVZ7, R0K2V0, A0A2P8FBJ9, A0A1I4K541, A0AII4DH54, A0A0P1FE41, A0A0N0V3A4, A0A021XGD0, A0A1A9FVP4, A0A372NA60, A0A372NIR6, A0A2W6YNN0, A0A2W6XTN2, A0A2W6TGS4, C9D0Y9, U3M974, Q8LP48, Q8LP47, Q9SEC7, Q8LP46, Q9SEB7, Q9SEC6, A0A443L654, A0A443KHG8, A0A443IWD5, A0A443LU18, A0A443KBX3, A0A443JST8, A0A3N2DQJ3, A0AIL3LEX8, A0AIL3KYZ3, A0A2S3YKP1, A0A2S3YS27, A0AIL3LHN3, A0AIL3LX24, D1CSK6, C3KRM2, A0A2Z3G701, A0A2U8GC90, A0A249Q4C1, A0A0T6ZYX4, I3X127, I3XBU9, A0A0E0UPB7, F7XGE8, H0G729, A0A178Y5H3, A0A178YA01, A0AIV3R0M3, A0A2A6PA40, A0A249P2H9, A0A249NZ41, A0A0T6ZY03, A0A0T6YWU1, A0A226CRW2, A0A2N8JWB1, A0AIG5WW04, A0A2A6MGF3, A0A2A6MEJ3, A0AIB3MF54, A0AIB3MIS4, A0A0T6XVR8, A0A0T6Z662, A0A2S2R1U8, A0A2S2R590, A0A2S2PZKO, A0A2S2PXW4, A0A2S2R5W8, A0A2S2PY34, A0A2S2PWP9, A0A2S2Q0X7, R9S9Z3, R9SAI4, R9S9I9, A0A183IUB1, A0A183IUA7, A0A183IL94, A0A183J2V0, A0A183IH80, A0A183IL56, A0A183ISK1, A0A183J3L0, A0A1831HX2, A0A183IAW3, A0A183IZI2, A0A183IV49, A0A183IMAR6, A0A183IBD8, A0A183IAM1, A0A183IN87, A0A183IPU9, A0A183IMI5, A0A183J9K8, A0A183ITM5, A0A3P7ZKR8, A0A183IID0, Q17U57, A0A0V0GU91, A0A0V0IAL3, A0A0V0IL88, A0A0V0IT75, A0A0V0H401, A0A0V0GP59, A0A0V0IZC1, A0A0V0ITT0, A0A0V0ISB1, A0A0V0HK80, A0A0V0HB28, A0A0V0IWF8, A0A0V0I770, A0A0V0GWH3, A0A0V0HTT1, A0A0V0IQW6, A0A0V0ISZ6, A0A0V0HEP1, A0A0V0IW22, A0A0V0IQJ4, A0A0V0ITB2, A0A3Q7FRL4, A0A3Q7JRJ5, A0A3Q7IBI2, A0A3Q7GDZ4, A0A3Q7F4P0, K4CH55, K4B9J0, A0A3Q7FY38, A0A3Q7HF37, A0A3Q7HDB6, A0A3Q7G892, A0A3Q7G5Y6, A0A3Q7F4L9, A0A3Q7F595, A0A3Q7G6E0, A0A3Q7IAB2, A0A3Q7G8E1, A0A3Q7FWM1, A0A3Q7EL09, A0A3Q7J270, A0A3Q7F4Q9, A0A3Q7HBS1, A0A3Q7HEV3, A0A3Q7EWM9, A0A3Q7F4N0, A0A3Q7IAP8, A0A3Q7F4K4, A0A3Q7HYY7, A0A3Q7G5X9, A0A3Q7HTG3, A0A3Q7FFS6, A0A3Q7G8V9, A0A3Q7HF02, A0A3Q7GP81, A0A3Q7F2Q7, A0A3Q7HHX3, A0A3Q7FRL0, A0A3Q7HHD3, A0A3Q7FRP6, A0A3Q7F582, A0A3Q7G5Z5, A0A3Q7I091, K4BE95, A0A3Q7I1N1, A0A3Q7IBL7, A0A3Q7HQY4, A0A3Q7INM2, A0A3Q7G218, A0A3Q7F3P0, A0A3Q7I8J1, A0A3Q7IBE5, A0A3Q7I8I7, A0A0D4BTD1, A0A0D4BT35, A0A0D4BTY2, M1C323, M1BNV7, M1A4E2, M1AlMO, M1C8X1, MOZGV4, M1CGZ5, M1B352, M1C1G6, M1DW58, M1BYZ9, M1BIT6, MiD080, M1B369, M1CJN9, M1BGH7, MOZKJ6, M1A1J1, M1BZ01, M1BGB2, M1B373, M1BNW8, M1CVL9, M1BP27, M1BGH6, M1BP21, M1DRI9, MOZGV5, M1A1J0, M1AFX4, A0A0M3STW8, M1BIU3, M1CRP0, M1CVM0, M1CGZ4, M1BNV6, M1AFZ1, MiD081, M1CH47, MOZUR7, M1AII5, M1BGH3, M1CVL4, M1C1G5, M1ASH7, M1BGG2, M1BNW9, M1A1M2, M1AII7, M1C1G7, M1BP20, M1BNV8, M1BZ00, M1BNX0, M1AlI8, M1BGB0, M1DRP4, M1CGZ2, M1A226, M1CGZ3, M1AII9, M1AJH1, M1B367, M1B363, M1B357, M1BIU2, M1BGA3, M1CVM1, M1AJH2, M1BNV5, M1BIU4, M1B356, M1AFY9, M1BGA2, M1C322, M1CGZ7, M1AFZ0, M1B366, M1DZ63, M1BGA7, M1BGA4, M1BGG5, M1C8X2, M1CC72, M1BNV4, M1BNW1, MiCJP1, M1BIT7, M1CVL6, M1A4E5, M1CH44, M1B371, E9J8M2, E9IHS5, E9IN88, E9J669, E9J909, E9J910, E9IHS6, E9IN89, A0A150STA1, F7VYZ6, F7VR72, F7W2U3, A0A1Z5R405, A0A1W0W663, A0AIZ5RKG3, A0A1Z5R8K9, C5YTR3, A0A1B6Q1T4, A0A1B6P7F5, A0AIW0W011, C5XIK1, C5X8J9, A0AIW0W6A4, A0AIB6QDZ9, A0A1Z5R8L3, C5XNJ5, A0AIB6PQ00, A0A1B6Q6I4, A0AIW0W4G9, C5YRQ1, A0AIZ5RF80, C5XKC4, A0AIB6QBX0, C5XG13, A0AIB6QJV5, A0A194YPP4, C5YI80, A0A1Z5S8X7, A0A1B6PN86, A0A1B6Q0T3, A0AIZ5RF61, A0A1B6P674, A0A1W0VTKO, A0A194YKI9, A0A1B6QP87, A0A1B6QBJ7, A0A1B6PNF9, A0A194YKJ5, C5Z879, A0AIZ5RCUO, A0AIB6QDZ0, A0AIW0W4H9, C5YFX5, A0AIB6QEU8, C5YZF4, A0AIB6QE05, C5XAW9, A0AIB6QPL1, C5YGN7, A0AIZ5RCV0, C5YGM5, A0A1Z5R1H2, A0AIZ5RGB4, C5XAX0, A0AIW0VYV0, A0A1B6P8A7, A0AIZ5RF79, C5XNJ6, C5XL73, A0AIZ5RF74, C5X9Y1, A0A1B6Q255, A0AIZ5RFW8, A0A1B6Q7G8, A0AIZ5RIL9, C5WQK6, A0AIZ5RKG6, A0A1W0W6M4, A0A194YHF9, A0A1B6P5Y4, A0A1B6PNE9, A0AIW0VTM8, A0AIW0VTM2, C5XLS4, A0AIZ5RFC4, A0A1Z5R535, C5YT42, A0AIB6PNG4, C5Y4M3, C5X494, C5YV85, C5WV97, A0AIZ5RF78, C5XSW0, C5X2A2, C5YGP6, A0AIB6QE00, A0AIW0W6E0, A0AIB6QBX3, A0AIB6PNF5, A0A1Z5S3X9, A0A1W0W6U5, A0A1W0W672, A0A1B6Q6J1, C5YGP5, A0AIW0VSW8, A0AIB6QBW8, A0AIB6QBW6, C5YI84, Q8LJZ0, A0A2K1QVG0, A0A2K1QHN6, A0A2K1QKQ3, A0A2K1QK63, A0A2K1QSX4, A0A2K1QFH6, A0A2K1QKW0, A0A2K1QK44, A0A2K1QZH3, A0A2K1QY48, A0A2K1QX05, A0A2K1QSH2, A0A2K1R2J1, A0A2K1QRG3, A0A2K1QKB9, A0A2K1QGJ2, A0A2K1R3T9, M3AV53, M3BZM1, A0A349QUZ7, A0A354KFP2, A0A2T7WNY4, A0A2T7WSG0, A0AIL3JC18, A0AIL3JC23, A0AIL3JC30, A0A371B272, A0A371BHY4, A0A371BHT8, A0A0K9RR85, A0A0K9RDP3, A0A0K9QHY2, A0A0K9QR37, A0A0K9R7Z2, A0A0K9Q8S7, A0A0K9SOX2, A0A0K9QPL4, A0A0K9RF56, A0A0K9S213, A0A0K9RH17, A0A0K9RT23, A0A0K9RB42, A0A0K9QZ62, A0A0K9RVT8, A0A0K9R8M3, A0A0K9Q888, A0A0K9RMQ3, A0A0K9QW25, A0A0K9S082, A0A0K9R4K7, A0A0K9QRC1, A0A0L0HA63, A0A0L0H8G6, A0A2H1WV32, A0A2H1VNI1, A0A2H1VQJ4, A0A2H1WDX9, A0A2H1W4L3, A0A0H5QFI6, A0A0H5QHL0, A0A0C2ER25, A0A162MRJ9, A0A167PWC0, U7Q5P1, A0A0F2LUQ6, A0A084B8D0, A0A084AUC4, A0A084AH70, A0A084AW91, A0A084R5E6, A0A084R699, A0A084RSS0, A0A084R0N3, A0A084QS85, A0A084QWE9, A0A084QR64, A0A178AM66, A0A178B4D4, A0A178B8B6, A0A178AC45, A0A178AYG8, A0A178B8W6, A0A178AV13, A0A178B2D4, A0A285S9J4, A0A1Y1SPA5, A0A420AAV3, A0A1H2FDZ7, A0A2E6Y7I2, A0A3B4ZKK7, A0A3B5B6X3, A0A3B4ZPZ3, A0A3B4ZIA4, A0A3B5ADV9, A0A3B4ZH28, A0A3B4ZII7, A0A087T2K8, A0A087U8M8, A0A087UVI0, A0A087UVI1, A0A087UVH9, A0A087T2K7, A0A087TCV3, A0A087SWB4, A0A087UKG6, A0A087SVJ4, A0A087UKG5, A0A087UBD7, A0A087UAP8, A0A087TMG3, A0A087T9G3, A0A087T7G6, A0A087T2C9, A0A087T0G3, A0A087TEI6, A0A087UVX7, A0A087THX0, A0A1I8AEG0, A0AII7Y5R0, A0A1I8AKS5, A0A1I8AL78, A0A1I7Y544, A0AII8A6X3, A0A1I8ATW3, A0A1I8AU60, A0A1I7YBL2, A0A1I8A4V1, A0A1I7Z6J2, A0AII8A5G4, A0A1I7YSK9, A0A1I7YBL9, A0A1I7YON0, A0A1I7ZHS2, A0AII7Y6B7, A0A1I7YB77, A0A1I7ZQD0, A0A1I7ZLK9, A0A1I7ZW10, A0A1I7Y539, A0AII7Z2T9, A0A1I7YQG9, A0A1I7ZVA4, A0A1I7YYH6, A0AII8AAT9, A0AII8AAI7, A0AII7Z483, A0AII8ARD3, A0A364NG07, A0A364N8Y3, A0A364NBE1, A0A2G8KGD5, A0A2G8KPK3, A0A2G8KY26, A0A2G8JWF4, A0A2G8KY13, A0A2G8KMR0, A0A2G8KON1, A0A2G8LND1, A0A2G8LNU1, A0A2G8JX12, A0A2G8KPX2, A0A2G8KPE7, A0A2G8K4I4, A0A2G8K4G4, A0A2G8JWF7, A0A2G8L1A0, A0A2G8KPD9, A0A2G8KPE6, A0A2G8L837, A0A2G8LH26, E3FLQ8, Q098U7, A0AII8PQ32, A0AII8QFD3, A0A1I8PLI1, A0AII8NR90, A0A1I8QDM2, A0A1I8PQE0, A0AII8QFE6, A0AII8QFB9, A0AII8NPE1, T1JBA9, T1ILR6, T1IZB1, T1IYR6, T1J1D9, TlJiD8, TlJ1E1, T1J2Z9, T1IYR5, T1IUF2, T1IWK2, W4YFT8, W4XEH1, W4Y9R3, W4Y7C8, W4Y7X2, W4ZAY8, W4YX16, W4ZEP3, W4Z4Q9, W4YIM2, W4Z490, W4XV31, W4XT52, W4Y4J9, W4YJJ7, W4Y4H6, W4XV55, W4XRN9, W4YFP9, W4ZK20, W4YB28, W4ZB03, W4YM62, W4ZIR0, W4YUE3, W4Z6A9, W4Y9R4, W4Z5T2, W4ZD80, W4XET4, W4XV33, W4Y748, W4YH25, W4YVK8, W4Y2F2, W4YXL4, W4ZIA9, W4YQ09, W4XPT9, W4ZG23, W4Y9H1, W4XN66, W4Z197, W4ZKJ1, W4YMZ9, W4Z0S4, W4YI87, W4Y1N4, W4Z525, W4XT71, W4XAZ6, W4Y432, W4Z5T3, W4XGA6, W4Z430, W4XDS2, W4YJJ8, W4Y9R5, W4XX39, W4Z280, A0A0N5BIA2, A0A0N5CCT3, A0A0N5CGC3, A0A0N5BMA1, A0A0N5B5D0, A0A0N5CGC2, A0A0N5BQ06, A0A0N5B5E6, A0A0N5BCKO, A0A0N5B3M3, A0A0N5C548, A0A0N5C796, A0A0N5BRL1, A0A0N5BF41, A0A0N5C8M1, A0A0N5C2G3, A0A0N5C549, A0A0N5C5W7, A0A0N5BTC5, A0A090MZF3, A0A090LAM2, A0A090KSC6, A0A090KTL6, A0A090MTX8, A0A090L5S9, A0A090KXKO, A0A090KWR4, A0A090KR38, A0A090LBKO, A0A090KZM2, A0A090LMM7, A0A090LMB5, A0A090L0U9, A0A090L8Q0, A0A090LEE0, A0A090KR99, A0A090LS81, A0A090LH59, A0A090L2B1, A0A090KZU3, A0A0KOEQ10, A0A0KOEN25, A0A0KOENZ3, A0A0KOEPC1, A0A0KOE3P4, A0A0KOE4B4, A0A0KOEFB6, A0A0KOES05, A0A0KOEEE0, A0A0KOESE2, A0A0KOE6Z7, A0A0KODU24, A0A0KOEIP0, A0A0KOEGC5, A0A0KOEE54, A0A0KODX88, A0A0KODTQ3, A0A0KOEGC4, A0A0KOE8Q0, A0A0KOEJ96, A0A0KOE4B5, A0A0KOFAY7, A0A0KOFYJ7, A0A0KOF449, A0A0KOF450, A0A0KOF9B8, A0A0KOFA09, A0A0KOF2N2, A0A0KOG164, A0A0KOFJN2, A0A0KOFCQ4, A0A0KOG4U8, A0A0KOEWF5, A0A0KOF5J9, A0A0KOF3F0, A0A0KOEUB2, A0A0KOF008, A0A0KOFCQ5, A0A0KOEZ99, A0A0KOF0L9, A0A0KOFEV0, A0A3P7ILC4, A0A3P7I9T4, A0A3P7JE85, A0A3P7IEU2, A0A3P7KH13, A0A3P7IJW3, A0A3P7IZH6, A0A3P7I491, A0A3P7LAH1, A0A3P7J5F1, A0A3P7IRM1, A0A3P7JJX8, A0A3P7J7H7, A0A3P7IN45, A0A3P7JMN8, A0A3P7INY8, A0A3P7LGM1, A0A3P7J2L6, A0A3P7JB01, A0A3P7JA70, A0A3P7L1J1, A0A093KD36, A0A093HHU1, A0A093HGM9, A0A2B4SM39, A0A2B4RRZ6, A0A2B4SJV0, A0A2B4SAG5, A0A2B4SES4, A0A2B4RBI1, A0A2B4S2H8, A0A2B4S3A5, A0A2B4SNH9, A0A2B4S207, A0A2B4S6F9, A0A2B4R7J5, A0A2B4SFG4, A0A2B4S741, A0A2B4SJX2, A0A2B4RAJ9, A0A2B4SUX3, A0A2B4SH64, A0A2B4SU12, A0A2B4R4B9, A0A2B4S3B6, A0A2B4R832, A0A2B4SXX8, A0A2B4SUD0, A0A2B4R2R9, A0A2B4RFK9, A0A2B4RMT1, A0A2B4R623, A0A2B4R7E0, A0A2B4RD03, A0A2B4S9T7, A0A2B4RFP1, A0A2B4RPK6, A0A2B4R401, A0A2B4RAC0, A0A2B4RS71, A0A2B4RXZ8, A0A2B4T1E8, A0A2B4RAX9, A0A2B4R5W3, A0A2B4REV2, A0A2B4RBF8, A0A2B4SUP0, A0A2B4RLC2, A0A2B4T0K3, A0A2B4RYT3, A0A2B4RW39, A0A2B4RR37, A0A2B4RK16, A0A2B4SDB6, A0A2B4SD97, A0A2B4R0H8, A0A2B4SY37, A0A2B4RMM4, A0A2B4R9P6, A0A2B4SAP0, A0A2B4S7C1, A0A2B4T076, A0A2B4RQ27, A0A2B4RZ31, A0A2B4SW57, A0A2B4RQZ6, A0A2B4SW29, A0A2B4SXJ7, A0A2B4R394, A0A2B4REL3, A0A2B4SKC3, A0A2B4RCQ4, A0A2B4RCF9, A0A2B4SGL1, A0A2B4SKU4, A0A2B4S352, A0A2B4RR27, A0A2B4R5T8, A0A2B4RIF0, A0A2B4SI30, A0A2B4RF56, A0A2B4S048, A0A2B4S8S0, A0A2B4RCW0, A0A2B4S421, A0A2B4S3Y9, A0A2B4SWB5, A0A2B4RNZ9, A0A2B4RKY5, A0A2B4SYK2, A0A2B4RCU9, A0A2B4RG52, A0A2B4SQ47, A0A2B4SFY8, A0A2B4SEZ0, A0A2B4RQ71, A0A2B4RI82, A0A2B4RXE4, A0A2B4R8V7, A0A2B4SNN3, A0A2B4S8F7, A0A2B4R5T1, A0A2B4RA51, A0A2B4RNS9, A0A2B4RAH7, A0A2B4RTJ2, A0A2B4SKD6, A0A2B4RCL9, A0A2B4SOE1, A0A2B4SOY6, A0A2B4RJW9, A0A2B4SZUO, A0A2B4SRH6, A0A2B4T229, A0A2B4SOG9, A0A2B4S040, A0A2B4RDG4, A0A2B4R9R0, A0A2B4R5S5, A0A2B4SPN4, A0A2B4S8L9, A0A2B4SBF7, A0A2B4R907, A0A2B4RDE8, A0A2B4RFA7, A0A2B4S2N4, A0A2B4SVB4, A0A2B4SXM9, A0A2B4RIY2, A0A2B4RLZ3, A0A2B4SOI5, A0A2B4S7S6, A0A2B4RYB4, A0A2B4SE06, A0A2B4S470, A0A2B4R8S2, A0A2B4SYR5, A0A2B4RI65, A0A2B4RE37, A0A2B4RJT3, A0A2B4RHB2, A0A2B4S982, A0A2B4SI73, A0A2B4RI05, A0A2B4RX94, A0A2B4SNE8, A0A2B4RJP3, A0A2B4SBI8, A0A2B4R7I8, A0A2B4RED5, A0A2B4R819, A0A2B4RAT8, A0A2B4RDB8, A0A2B4RM65, A0A2B4SMY1, A0A2B4RXN2, A0A2B4SDQ3, A0A2B4S5V1, A0AII1VVN6, A0A073ILQ0, A0A196QUR2, A0A420DSB1, A0A061SV07, A0A2T6CEN1, A0A221K513, A0A1J0WMJ3, A0A196P088, A0A345Q6D2, P06867, FlSPS6, F1SB93, F1RZN7, A0A286ZVK1, 097506, A0A286ZR66, F1SB81, A0AIQ9ESG7, A0AIQ9CQI2, A0A1Q9C6C8, A0AIQ9CCQ0, A0AIQ9EEK4, A0A1Q9D1E5, A0A1Q9DKU8, A0A1Q9DRV2, A0A1Q9F029, A0A1Q9ESU8, A0A1Q9EQ68, A0A1Q9D2R1, A0A1Q9E4J1, A0AIQ9CW84, A0AIQ9CMK9, A0AIQ9CF00, A0A1Q9C5U3, A0A1Q9E8L2, A0A1Q9D4N5, A0A1Q9EKZ9, A0A1Q9C5H9, A0A1Q9E9H4, A0A1Q9C600, A0A1Q9CGB9, A0A1Q9BXP7, A0A1Q9CQY3, A0A1Q9CR00, A0A1Q9CAA1, A0A1Q9EMW5, A0A1Q9DB15, A0A1Q9CW09, A0A1Q9CR59, A0A1Q9ED26, A0A1Q9ERZ2, A0A1Q9CQZ9, A0A1Q9CZK1, A0A1Q9DNS4, A0A1Q9F1U4, A0A1Q9CT81, A0A1Q9D3Z6, A0A1Q9CQ66, A0AIQ9CSN6, A0AIQ9ERZ8, A0A1Q9D1P7, A0AIQ9ES05, A0AIQ9CUS2, A0A1Q9DZF4, A0A1Q9CAP7, A0A1Q9DAV4, A0A1Q9DVU6, A0A1Q9BZ14, A0A1Q9DA38, A0A1Q9EUE8, A0A1Q9DT95, A0A1Q9EUA8, A0A1Q9DLH6, A0A1Q9D262, A0A1Q9D697, A0A1Q9DPD5, A0A1Q9EBY1, A0A1Q9D3F0, A0A1Q9CMG7, A0A1Q9EUA6, A0A1Q9F599, A0AIZ9KW32, A0A1Z9L6H2, A0A2M7BBY6, A0A1V4Y3U4, A0A0N5AQ74, A0A0N5AVN4, A0A0N5AWJ9, A0A0N5AKP3, A0A0N5ASE0, A0A0N5AQE8, A0A0N5AV92, A0A0N5ABP9, A0A0N5AMT9, A0A158R5G1, A0A0N5AYU3, A0A0N5AT40, A0A0N5A8Y8, A0A0N5AUH4, A0A158R6C4, A0A158R5U4, A0A0N5AX21, A0A0N5AAC3, A0A0N5AES2, A0A0N5AR84, A0A0N5AWX5, A0A411Z4S3, A0A3S8U2P2, A0A2N4YG23, A0A0R3VTQ5, A0A3P6NAE9, A0A158R765, H0ZG36, H0YWJ9, H0ZY21, H0ZML2, H0Z9B7, H0Z415, A0A3B5KNQ7, A0A0S3P5S3, A0A3B5JVN8, H2U253, A0A3B5K7U1, H2UIX2, A0A3B5K478, H2U252, A0A3B5K8C0, H2UB48, A0A3B5KSI5, H2UB51, A0A364L110, A0AIQ5QBL9, A0A0U1LQB1, B6QAW5, B6Q7P1, B6QVR0, B8M4W0, B8MRL4, R4X8Q8, A0A163ZG59, A0A370QWB2, A0AIH6PBW2, A0A239NTK9, A0A2UOTH97, A0A420ATD9, A0A3Q0DMW9, A0A1U7SYT3, A0A1U7U5V8, A0A1U7U7M3, A0A1U7TY18, A0A3Q0DKG9, A0A1U7SMG8, A0A1U7UC10, A0A3Q0DG51, A0A3Q0DM20, A0A1P8MZ84, A0A0B4C503, A0A093CH68, A0A093CND7, A0A093CVC3, A0A093CQR8, A0A2G9TXP9, A0A2G9U3X5, A0A2G9UGS1, A0A2G9UVF5, A0A2G9UD74, A0A2G9TB90, A0A2G9UAM5, A0A2G9T9Y6, A0A2G9U041, A0A2G9UK14, A0A2G9U3J8, A0A2G9U5G6, A0A2G9U6U5, A0A2G9UCE1, A0A2G9V4H0, A0A2G9UF90, A0A2G9UOT0, A0A2G9UAE0, A0A2G9U9D4, A0A2G9UTY8, A0A2G9UH07, A0A3C1S3E6, A0A3C1S4B8, A0A081BAR4, C5BI67, A0A3G4ZLM1, A0A2J7ZPE6, A0A2J7ZJB7, A0A2J8AH70, A0A2J8AIZ8, A0A2J8A820, A0A2J8A4H3, A0A2J8AFD0, A0A2J7ZQC3, A0A2J8AFF5, A0A2J7ZX95, A0A2J8A7Q4, A0A2J7ZFF2, A0A383W609, A0A383W2V6, A0A383VA02, A0A383VTL8, A0A383VEC8, A0A383W0C6, A0A383VRV3, A0A383VVE2, A0A383VV90, A0A383WH61, A0A383VIT8, A0A383VX83, A0A383V476, A0A383WKH6, A0A383VAP4, A0A383VSI6, Q236L9, Q212L4, A0A3G5ANYO, T1L2L9, T1JTQ3, T1JZ41, T1JZ38, T1KP84, T1L6E3, T1KXB8, T1KLX8, TiKlX0, T1KJV3, T1JQKO, T1K882, T1KJA7, T1L2F0, T1JY66, T1KEM9, T1L0H5, T1KY75, T1KVC5, T1KP75, T1L6B7, T1L4K2, TiL0U8, T1KOC2, T1JY60, T1KXD7, T1KOA5, T1KP31, T1KNH0, T1L2M1, T1KVC7, TiKOC1, A0A158P574, T1L0H6, TiKOC0, T1K6H1, T1KOA4, TiL1K0, T1JXS1, T1K414, A0A158P575, T1KVB7, T1K276, TiKOA1, T1KW32, T1L1J9, T1KP56, T1L4K1, T1KP60, T1KOA3, TIK411, T1L2E5, T1KVC8, T1KOA9, T1KF99, T1KP59, TlJXR7, T1JXS5, T1KOA7, T1KOE3, T1JV21, T1KP64, T1K7E7, Q4RF08, H3CM48, Q4RX91, H3CWI4, H3DBA6, H3D0B7, Q4RF09, Q4SIS6, Q4SBT4, H3C6P0, H3DPE4, H3CV10, H3CWH8, A0A061RM82, A0A061QU17, A0A061SFA1, A0A061SOK3, A0A061R340, A0A061S6B9, A0A061RKH4, A0A061SLP2, A0A061QSN4, A0A061S913, A0A061R7K6, A0A061QSD4, A0A061R2B1, A0A061R0X8, A0A061QSK8, A0A061SA98, A0A2P0VNG3, A0A2P0VNJ5, KOR003, K0T569, KOR8M5, KOR4V6, KOTI40, KOS3W9, K0R053, KORJ23, KOR2N3, A0A0P1FYC3, A0A0P1FXN8, A0A369TVA4, A0A369TQK7, A0A0D8D6E0, J4C7F0, A0A2T7I917, A0A2T7IN49, A0A0N5D4A3, A0A0N5DAH6, A0A0N5D0V9, A0A0N5CNH4, A0A0N5D8L1, A0A0N5CN06, A0A0N5D5Z9, A0A158RB75, A0A158RB18, A0A158RCJ6, A0A0N5CL57, A0A0N5CMM8, A0A0N5D941, A0A0N5CU49, A0A0N5CLL0, A0A0N5D6J3, A0A0N5CU48, A0A0N5D3M6, A0A0N5D0H3, A0A0N5CU50, A0A0C2N594, A0A061FES3, A0A061F6M2, A0A061F6Q9, A0A061F858, A0A061F7U2, A0A061E4J3, A0A061F7S0, A0A061DSY4, A0A061F7R6, A0A061F5X2, A0A061F5W4, A0A061DK79, A0A061GFZ4, A0A061FE28, A0A061FDY8, A0A061GMY9, A0A061DT57, A0A061FE35, A0A061F6S4, A0A061ESM6, A0A061F7P5, A0A061FE32, A0A061GN00, A0A061DJB7, A0A061F829, A0A061FAS2, A0A061GJY1, A0A061GJ76, A0A061E2S8, A0A061FXH6, A0A061FQL2, A0A061GGE3, A0A061GSKO, A0A061F5V9, A0A061GGC0, A0A061F6T1, A0A061FEW2, A0A061GMZ5, A0A061FA86, A0A061E2J3, A0A061GF26, A0A061F5X6, A0A061F6R7, A0A061FII3, A0A061GA93, A0A061GGC6, A0A061FBL4, A0A061ETG0, A0A061F7N9, A0A061EAF9, A0A061GG11, A0A061F862, A0A061GF19, A0A061F9V0, A0A061F5Y4, A0A061GS10, A0A061F798, A0A061DLF5, A0A061GH37, A0A061FEJ0, A0A061F7L9, A0A061ERV1, A0A061GGD8, A0A061GFZ9, A0A061F6A9, A0A061F7T9, A0A061F463, SiRWJ4, A0A061F7A3, A0A061E4H9, A0A061F772, A0A061F7R1, A0A061F7T8, A0A061E2S4, A0A061F771, A0A061FDW5, A0A061FE49, A0A061E2U3, A0A061F6S1, A0A061F7S7, A0A061E4H6, A0A061E3P7, A0A061F784, A0A061F5W9, A0A061F6J2, A0A061FFC7, A0A061E9M0, A0A061GG05, A0A061F6R3, A0A061GF31, A0A061EQQ6, A0A061E3Q5, A0A061FDY1, A0A061FGI5, A0A061GH52, A0A061G8B1, A0A061FE26, A0A061EZN1, A0A061ERB8, A0A061EAF4, A0A061E3Q2, A0A061GN44, A0A061ETH9, A0A061GGD2, A0A061FC33, A0A061GBN6, A0A177E7S8, A0A0S3QUQ3, A0A179D652, G2QZQ3, A0A3S4ATD0, F9UF48, A0A1H2T5K1, A0A074JZA8, I3YA25, L0H1B6, G2DZW1, A0A1H6F9F4, A0A0A7CLS4, A0A0A7CM27, A0A0A7CMA6, A0AIV9ZCI3, A0A0A7CLYO, A0A1V9Y7R8, A0A1V9ZQ59, A0A1V9ZX23, A0A0A7CM73, A0A1V9ZWR3, A0A0A7CLP3, A0AIV9ZWD5, A0AIW0A8G8, A0A1V9Z6L2, A0A0A7CMR6, A0AIW0A9L7, A0A1V9Y3Z2, A0A0A7CMJ9, A0A0A7CLZ4, A0A0A7CLK9, A0A1V9Y7V8, A0A0A7CLD0, A0A0A7CM37, A0AIV9YW96, A0A0A7CMD3, A0AIV9ZV18, A0AIV9ZQU5, A0AIW0A6N9, A0A0A7CM15, A0AIV9ZX55, A0A1V9YS19, A0A1W0A3D9, A0A1W0A1E0, A0A0A7CLV9, A0A1V9YHU4, A0A1V9Y7T4, A0A1V9YS21, A0A1W0A6T9, A0A1W0A2R4, A0A1V9ZRW9, A0A0A7CMA3, A0A0A7CM84, A0A0A7CM21, A0A0A7CM83, A0A177U9L0, A0A177VNW5, A0A066V6E8, A0A099ZKM4, A0A099YWG8, A0A099ZHV8, A0A099ZGB5, R8BMT1, R8BT17, A0A2S4KXQ7, A0A2S4KU31, A0A0A1T7V7, A0A183V6M5, A0A183UMUO, A0A183V0Y5, A0A183VH40, A0A183URQ0, A0A183UCS5, A0A183UGS8, A0A183VGT9, A0A183UGB4, A0A183UJW2, A0A183VFR8, A0A183V6I3, A0A183V8F0, A0A183UKJ7, A0A183UZI3, A0A183UCS4, A0A183TZ03, A0A183UXD4, A0A183UPE8, A0A183VHD0, A0A183V3X8, A0A183UN76, A0A183V0R7, A0A183UZ80, A0A0B2V222, A0A0B2W1F4, A0A0B2VYQ4, A0A0B2VKQ9, A0A0B2VKX7, A0A0B2VCC1, A0A0B2VBP0, A0A0B2VDF6, A0A0B2VTH8, A0A0B2VE70, A0A183V0Y4, A0A183TW29, A0A3P7IEE1, A0A0B2V8X8, A0A0B2V3M1, A0A183U4G9, A0A3P7EW77, A0A0B2VCY3, A0A0B2VUZ8, A0A0B2VRS5, A0A0B2VS27, A0A0B2V756, A0A0B2V7G5, A0A0B2VLY9, A0A0B2V1Y2, A0A0B2UVF7, A0A0B2UUB7, A0A3P7F9F7, A0A0B2VJJ4, A0A183UFM8, A0A0B2UVS9, A0A0B2W5N5, A0A125YNB1, S8EWI3, A0A125YP11, S8EXK9, S8FBN1, S8GJR8, S8GTW7, S8GU33, A0A125YN33, S8GE96, S8EWJ0, S8G8W7, S8EUX1, S8F2N4, S8EVK9, S8GIC9, S8FBK4, S8F0R5, S8EZX1, S7UGM9, S7UVS3, S7W921, S7VS18, S7ULA3, S7W1D2, A0A125YUY4, S7VZR7, S7V4Y3, S7VNS6, S7VUK4, S7UNU6, S7URJ3, S7WIY4, S7UL27, S7UID9, S7UMV2, S7UFC4, A0A125YP10, S7V4X6, A0A125YMJ2, B6KPA5, B9QJ65, V5BN10, B9PXJ3, A0A0F7UY76, A0A0F7V6F0, A0A0F7UZC2, V4ZN28, V5B2C7, B9QHA2, A0A125YYV2, V4ZE44, V4ZEU8, V4Z8E3, V4Z683, A0A0F7UY36, A0A0F7VB90, A0A0F7V4E0, V4YRM6, B9QHI1, A0A0F7USH6, A0A0F7UP20, A0A0F7UTL5, A0A0F7V2Y2, A0A125YH93, V4ZAX9, V4ZEU3, V4ZQP4, A0A0F7V7R9, V4Z2B5, A0A0F7V2N5, V4ZSH6, A0A139Y8B9, A0A139XR75, A0A139XT94, A0A139XR22, A0A139XUT5, A0A139XV46, A0A139Y523, A0A139XII1, A0A139XUW2, A0A139XRH6, A0A139XI69, A0A139XIG5, A0A139XQZ9, A0A139XIW7, A0A139XYI0, A0A139XIH6, A0A139YAJ0, A0A139XI83, A0A139XUB1, A0A139XIA3, A0A425HTR7, A0A425HLG8, A0A3R8FZK1, A0A3R7YPK8, A0A3R8BM20, A0A3R8AJMO, A0A425HTJ6, A0A3R7Z551, A0A3R7ZDF1, A0A3R7ZER8, A0A3R8GAD6, A0A425HLE6, A0A425HRD9, A0A425HTP5, A0A425HNT2, A0A425I1A5, A0A425HTB8, A0A425HNI2, A0A3R8AFY2, A0A2G8XSQ7, A0A2G8XUD9, A0A2G8Y8M6, A0A2G8XUK9, A0A2G8XV07, A0A2G8XU33, A0A2G8XVA9, A0A2G8XLM8, A0A2G8XM15, A0A2G8Y7C4, A0A2G8XYW5, A0A2G8XU35, A0A2G8YB46, A0A2G8XVB5, A0A2G8XLP9, A0A2G8XM02, A0A2G8XWV2, A0A086KZL5, A0A086L8S1, A0A086KXX3, A0A086JKK2, A0A086LDQ0, A0A086JHJ8, A0A086K039, A0A086K045, A0A086JSN5, A0A086LF74, A0A086KUA3, A0A086LGR4, A0A086KUA2, A0A086LC13, A0A086KT20, A0A086KZ32, A0A086KBL4, A0A086KJB6, A0A086KPR8, A0A086JRW4, A0A086JAN6, A0A086JTG7, A0A086JAS2, A0A086JRJ6, A0A086KBL3, A0A086KAV4, A0A086J9Y0, A0A086K6N1, A0A086JBU3, A0A086KWD4, A0A086JSD4, A0A086KNW2, A0A086JH56, A0A086QPX5, A0A086QPX6, A0A086QS28, A0A086QR16, A0A086QSB4, A0A086QZQ0, A0A086QXJ7, A0A086PMI4, A0A086PJV3, A0A086QPJ5, A0A086Q7X9, A0A086QSKO, A0A086QDJ1, A0A086Q5C2, A0A086PJS5, A0A086PJV4, A0A086PP13, A0A086JYT0, A0A086JYV7, A0A086KQT7, A0A086JUX0, A0A086JEKO, A0A086K9B0, A0A086J6M2, A0A086L3H4, A0A086JYV2, A0A086J6A5, A0A086JAT8, A0A086JW34, A0A086KKD3, A0A086J6E7, A0A086KW68, A0A086JUW9, A0A086JYW7, A0A086LN03, A0A086LVF2, A0A086LIM6, A0A086LJ27, A0A086LIY4, A0A086LWU1, A0A086MAA6, A0A086LX43, A0A086LX44, A0A086LTJ4, A0A086MOU2, A0A086M4H2, A0A086LIS3, A0A086M306, A0A086LUX9, A0A086LV63, A0A086M612, A0A086MOU7, A0A086LJF7, A0A086LJF6, A0A2T6IZJ0, A0A2T6II53, A0A2T6IQ36, A0A2T6IQ28, A0A2T6IUI9, A0A2T6IIA3, A0A2T6II49, A0A2T6ID59, A0A2T6II52, A0A2T6IRS2, A0A2T6J4Y3, A0A2T6IDB2, A0A2T6ISI1, A0A2T6J2S6, A0A2T6IS88, A0A2T6IQD1, A0A151GZ57, A0A151HHT1, A0A151HQ98, A0A151GZ62, A0A151HIC2, A0A151HH74, A0A151HFL2, A0A151H2M3, A0A151H4P4, A0A151HDF6, A0A151GYR0, A0A151HIUO, A0A151HH38, A0A151HIP1, A0A151HGR3, A0A151HL78, A0A151HGN6, A0A086PTH3, A0A086PPL1, A0A086PIW8, A0A086PGB6, A0A086QHG9, A0A086PYL8, A0A086PIX2, A0A086PGB7, A0A086PG37, A0A086PFY3, A0A086PSX1, A0A086PFS3, A0A086Q6Y4, A0A086PYK3, A0A086PZC2, A0A086PIX4, A0A086Q258, A0A086PSX2, A0A086PYV6, A0A086QF78, Q1JSE8, D8L561, D8L553, D8L555, D8L551, D8L554, D8L556, D8L552, D8L559, Q1JSK6, D8L558, D8L550, Q9XZH7, D8L557, W8VX31, A0A195E6P4, A0A195DN17, A0A195DN14, A0A151JQX2, A0A151J1A2, A0A195DID1, A0A151J003, A0A151J024, A0A195F9C1, A0A195FXK7, A0A195FSC2, A0A195F1L6, A0A195EZ81, A0A195FP32, A0A151WZ08, A0A151X5L2, A0A151XCA5, A0A151WZC1, A0A151WMX4, A0A151WFX6, A0A438AHQ8, A0A2P5EEL5, A0A2P5E9P3, A0A2P5E9P1, A0A2P5D0W9, A0A2P5D0Z2, A0A2P5D0Y8, A0A2P5F2U6, A0A2P5F1R2, A0A2P5EHK8, A0A2P5D0X0, A0A2P5F9V6, A0A2P5FFA8, A0A2P5BR45, A0A2P5E9N6, A0A2P5BDV9, A0A2P5E9S9, A0A2P5E300, A0A2P5BB99, A0A2P5D0W8, A0A2P5AU86, A0A2P5ELI9, A0A2P5BDW2, A0A2P5BXZ5, A0A2P5CM83, A0A2P5BDX9, A0A2P5EJU3, A0A2P5E5Q8, A0A2P5EJYO, A0A2P5E9N1, A0A2P5E7C6, A0A2P5DNMO, A0A2P5D111, A0A2P5CM53, A0A2P5EJU9, A0A2P5EGL2, A0A2P5D0X3, A0A2P5DNJ7, A0A2P5E9Q9, A0A2P5BR70, A0A2P5E7F0, A0A2P5EM89, A0A2P5CM78, A0A2P5E7F7, A0A2P5EM81, A0A2P5EM56, A0A2P5E7E7, A0A2P5E7E4, A0A2P5BDZ8, A0A2P5D0Y7, A0A2P5E7F8, A0A2P5D0W7, A0A2P5AU55, A0A2P5E7E0, A0A2P5E9N7, A0A2P5EGQ1, A0A2P5E7C3, A0A2P5AVW6, A0A2P5E7E1, A0A2P5D0X7, A0A2P5E7E9, A0A2P5CM65, A0A2P5D0Y2, A0A2P5E9K7, A0A2P5EK01, A0A2P5E796, A0A2P5CM46, A0A2P5AU80, A0A2P5DKI1, A0A2P5EEU6, A0A2P5AW26, A0A2P5E7I1, A0A2P5AU99, A0A2P5E7K1, A0A2P5E9P7, A0A2P5EMF7, A0A2P5CM64, A0A2P5E7F2, A0A2P5DUH7, A0A2P5EGL5, A0A2P5EJX1, A0A2P5E329, A0A2P5E7G0, A0A2P5EMG7, A0A2P5E305, A0A2P5ARA6, A0A2P5C4S2, A0A2P5EJT3, A0A2P5D0X8, A0A2P5E322, A0A2P5EM74, A0A2P5BDW0, A0A2P5B3V3, A0A2P5BCJ5, A0A2P5EJU7, A0A2P5BCJ8, A0A2P5B9F2, A0A2P5E7C7, A0A2P5E9R8, A0A2P5EGP2, A0A2P5B3Y1, A0A2P5F9B9, A0A2P5EJUO, A0A2P5E9R2, A0A2P5EGN4, A0A161M4M9, A0A023EZN7, A0A023F552, D6X332, D6WSE9, D2A5T8, D6WCQ0, D6WEC2, A0A139WM22, A0A139WEU2, A0A139WF13, A0A2Y9D6R9, A0A2Y9RN69, A0A2Y9D6V5, A0A2Y9DUI6, A0A2Y9REY6, A0A2Y9DUP3, A0A2Y9RJN7, A0A2Y9QP02, A0A2Y9D6R6, A0A2Y9R8V0, A0A0VICBM7, A0A0V1CL20, A0A0V1CF23, A0A0V1D321, A0A0V1DE70, A0A0VICNZ7, A0A0VIDDL1, A0A0VICYA0, A0A0V1D3X8, A0A0V1D312, A0A0V1D2Z4, A0A0V1DGI6, A0A0V1DIR9, A0A0V1DGQ0, A0A0V1D9K9, A0A0V1DCG4, A0A0V1D9Q7, A0A0VICAII, A0A0V1CF35, A0A0VIDGL4, A0A0VICBT7, A0A0V1CQV4, A0A0V1CL47, A0A0V1D4Y2, A0A0V1CL07, A0A0V1CJ57, A0A0VICQS1, A0A0VICPE6, A0A0V1D2G1, A0A0VIDCQ6, A0A0VICGZ2, A0A0VICKH2, A0A0V1C8F4, A0A0V1D5M4, A0A0V1D5G8, A0A0V1CY65, A0A0V1CIY6, A0A0V1CSO9, A0A0V1DDG4, A0A0V1D628, A0A0V1CL51, A0A0V1D3L4, A0A0V1D9P6, A0A0V1CF47, A0A0V1CEP1, A0A0V1CR67, A0A0V1D2S7, A0A0V1CCF9, A0A0V1CI86, A0A0V1D4Z1, A0A0V1D8K4, A0A0V1CY85, A0A0V1CIZ3, A0A0V1CLE7, A0A0V1D8Y2, A0A0V1CIU3, A0A0V1CLV0, A0A0V1D4H1, A0A0V1D4T5, A0A0V1CLT4, A0A0V1CC34, A0A0VICQM7, A0A0VICIG0, A0A0V1C615, A0A0VICYG1, A0A0V1CL59, A0A0V1D176, A0A0V1CLC2, A0A0V1DDF7, A0A0V1CJ14, A0A0V0U668, A0A0V0TU85, A0A0V0U6A2, A0A0V0UGN9, A0A0V0T888, A0A0V0T7J2, A0A0V0TGR2, A0A0V0TEI1, A0A0V0TLQ5, A0A0V0TLA0, A0A0V0UAM2, A0A0V0UI40, A0A0V0TLP7, A0A0V0TBB8, A0A0V0U7B1, A0A0V0TSMO, A0A0V0TU80, A0A0V0UAP6, A0A0V0TWW5, A0A0V0UI96, A0A0V0T000, A0A0V0TLE6, A0A0V0U6B0, A0A0V0UIZ5, A0A0V0TKS4, A0A0V0U6A5, A0A0V0TUK2, A0A0V0U664, A0A0V0TGD8, A0A0V0U6L1, A0A0V0TW68, A0A0V0TZG6, A0A0V0TZB3, A0A0V0U3H5, A0A0V0TR48, A0A0V0UAV4, A0A0V0TSU0, A0A0V0U6J8, A0A0V0TQX3, A0A0V0TCV2, A0A0V0T869, A0A0V0T5X4, A0A0V0T304, A0A0V0UOF0, A0A0V0T3U0, A0A0V0TSV2, A0A0V0T8Y2, A0A0V0TLJ2, A0A0V0TTX4, A0A0V0THH3, A0A0V0TCQ5, A0A0V0UGL3, A0A0V0T7W1, A0A0V0TGF1, A0A0V0TT12, A0A0V0TV07, A0A0V0UJZ1, A0A0V0T591, A0A0V0TFQ9, A0A0V0TW29, A0A0V0U516, A0A0V0TPM2, A0A0VIKNKO, A0A0VIKLF7, A0A0VIKYR3, A0A0VIKYC8, A0A0V1LNJ6, A0A0V1LAI7, A0A0V1KPV0, A0A0V1KPB9, A0A0V1KKU9, A0A0V1LiF4, A0A1Y3EXK1, A0A0V1LJJ4, A0A0V1LAS4, A0A0V1KMH3, A0A1Y3E8Z7, A0A1Y3E6K9, A0A1Y3EHJ6, A0A0V1LIU2, A0A1Y3E6I2, A0A1Y3EHG2, A0A1Y3EE55, A0A0V1KPJ6, A0A0V1L0L9, A0A0V1LEN4, A0A0VILMK8, A0AIY3EMP1, A0A0VIKMD9, A0AIY3E8I5, A0A0V1L1M0, A0AIY3E9F8, A0A0VILIH7, A0A0V1L1N0, A0AIY3EK45, A0A0V1L5K7, A0A0VILAY9, A0A0V1L1G4, A0AIY3E3J3, A0A0V1KM43, A0A0VILBC3, A0A1Y3E9Q0, A0A0V1KYR0, A0A0V1LiE8, A0A0V1KP52, A0A0V1L0I9, A0A0VILEV0, A0A0VILAP5, A0A0V1L1M7, A0A0V1LE33, A0AIY3EH27, A0A1Y3E603, A0A0V1LJH2, A0A0V1LMI4, A0A0V1KYB0, A0A1Y3ET15, A0A0V1LiE6, A0A0V1KR57, A0A0V1L136, A0A0V1LT84, A0A0V1KLJ0, A0AIY3EP57, A0AIY3EUE7, A0A0VIKXX6, A0AIY3EF82, A0AIY3EZI1, A0A0V1KXY2, A0A1Y3E7T0, A0A0V1KPL5, A0A1Y3E8Z4, A0A0V1KX10, A0A0V1LFN7, A0A1Y3ENN2, A0A1Y3EJ87, A0A1Y3F0N2, A0A1Y3EDK3, A0A1Y3E9V6, A0A0V1KPY5, A0A1Y3EJ46, A0A0V1LE29, A0A0V1LED3, A0A0V1LLN6, A0A1Y3EUS4, A0A0V1L2N9, A0A0V1L3H8, A0A0V1KR45, A0A0V1LAL0, A0A0V1KS99, A0A1Y3EGT9, A0A0V1LCN5, A0A1Y3ETI7, A0A1Y3EAN3, A0A1Y3EH07, A0A0V1LU40, A0A1Y3E7K6, A0A0V1KSI2, A0A0VILES1, A0AIY3E7U1, A0AIY3E9F9, A0A0V1LB56, A0A0VILNE5, A0A0V1L7E6, A0A0V1L6Z9, A0A0V0RYN5, A0A0V0SCW7, A0A0V0RIUI, A0A0V0RN79, A0A0V0SH81, A0A0V0SCZ0, A0A0V0RV95, A0A0V0S538, A0A0V0RM32, A0A0V0SCMO, A0A0V0RFG5, A0A0V0RRH9, A0A0V0RRB5, A0A0V0RG56, A0A0V0S806, A0A0V0RZJ1, A0A0V0RDH2, A0A0V0RGM5, A0A0V0SNF7, A0A0V0S2V9, A0A0V0RYH3, A0A0V0RGW1, A0A0V0SCX3, A0A0V0REU3, A0A0V0RZP8, A0A0V0RT14, A0A0V0S6I1, A0A0V0RJS2, A0A0V0SEE5, A0A0V0RZB8, A0A0V0RK34, A0A0V0S5T4, A0A0V0RZM6, A0A0V0SMX2, A0A0V0RX40, A0A0V0S9B4, A0A0V0RFF5, A0A0V0RYQ4, A0A0V0S5G1, A0A0V0REL5, A0A0V0RSM5, A0A0V0RYQ1, A0A0V0SCP1, A0A0V0SCK5, A0A0V0RF35, A0A0V0RFT3, A0A0V0RPN2, A0A0V0RQB5, A0A0V0RHM2, A0A0V0RZD1, A0A0V0REM5, A0A0V0S4Z2, A0A0V0RZT2, A0A0V0RS35, A0A0V0S652, A0A0V0RG20, A0A0V0RM92, A0A0V0S584, A0A0V0S4X7, A0A0V0RDD2, A0A0V0S541, A0A0V0REJ1, A0A0V0SOD1, A0A0V0RK03, A0A0V0S3G7, A0A0V0RX96, A0A0V0RHC1, A0A0V0RSZ9, A0A0V0RIE7, A0A0V0S9G5, A0A0V0RTR4, A0A0V0RIE5, A0A0V0RYP8, A0A0V0RSK8, A0A0V0RFI6, A0A0V0S2H6, A0A0V0S5G8, A0A0V0SOF9, A0A0V0S5R1, A0A0VIMVW8, A0A0VIMAI7, A0A0VIMYP5, A0A0V1N2V7, A0A0VIMUG7, A0A0VIMUG4, A0A0VIMYQ2, A0A0VIMGC0, A0A0V1N6W8, A0A0V1N6W7, A0A0V1MF20, A0A0V1MNC5, A0A0V1MJU5, A0A0V1MYR0, A0A0V1MZ66, A0A0V1MAV6, A0A0V1MYQ0, A0A0V1N2Z9, A0A0V1MH28, A0A0V1MVV2, A0A0V1N167, A0A0V1MYJ7, A0A0V1MT15, A0A0V1MH70, A0A0V1M2G6, A0A0V1MUW3, A0A0V1M9J0, A0A0V1MHZ6, A0A0V1MTF5, A0A0V1MGN0, A0A0V1MZ68, A0A0V1N6Q2, A0A0V1MGD7, A0A0V1MIW0, A0A0V1N5S4, A0A0V1MYM3, A0A0V1MI03, A0A0V1MYL8, A0A0V1MSA4, A0A0V1MZA7, A0A0V1MZ29, A0A0V1MUK2, A0A0V1MZ89, A0A0V1MUM5, A0A0VIMIL5, A0A0V1MZ88, A0A0VIMTC8, A0A0V1M8W6, A0A0VIMON9, A0A0V1N9C6, A0A0V1MN65, A0A0VIMNB1, A0A0VIMWG2, A0A0V1N8S1, A0A0V1MYJ6, A0A0V1N750, A0A0V1MH06, A0A0V1MYI7, A0A0V1MYQ1, A0A0V0ZT98, A0A0V1AAJ2, A0A0V0ZDL2, A0A0V1AFC5, A0A0V0ZMA2, A0A0V0ZI13, A0A0V1ADL5, A0A0V0ZA63, A0A0V0ZJI5, A0A0V1AGR2, A0A0V0ZEN6, A0A0V0ZJY6, A0A0V0ZI71, A0A0V0ZJV3, A0A0VIA9E4, A0A0V0ZKA1, A0A0V1A5G3, A0A0V1AH40, A0A0V0Z9W9, A0A0V0ZMW0, A0A0V0ZPP6, A0A0V0ZJ05, A0A0V1A350, A0A0V0Z9K2, A0A0V0ZQB1, A0A0V1A5H0, A0A0V0ZQQ6, A0A0V1A5B9, A0A0V0ZCY3, A0A0VIACU1, A0A0V0ZID7, A0A0V0ZJE6, A0A0V0ZA86, A0A0VIAEG0, A0A0V0ZHM8, A0A0V0ZU82, A0A0V0ZXEI, A0A0V1A735, A0A0V0ZBC9, A0A0V1A2Y6, A0A0VIADC4, A0A0V1A315, A0A0V0ZG93, A0A0V0ZG08, A0A0V0ZBZ0, A0A0V0ZC36, A0A0V0ZBF0, A0A0V1A3P0, A0A0V0ZBK7, A0A0V1ACP2, A0A0V0ZUN7, A0A0VIACR5, A0A0V1A6I5, A0A0V0ZRK6, A0A0V0ZDM2, A0A0V0ZC11, A0A0V0ZSY6, A0A0V0ZAI4, A0A0VIAGP3, A0A0V0ZK36, A0A0VIABN4, A0A0V0ZK58, A0A0V0Z425, A0A0V1K9D8, A0A0V1JL75, A0A0V1KA64, A0A0V1IA74, A0A0V1ECJ0, A0A0V1ERM8, A0A0V1EJT7, A0A0V1E276, A0A0V1K9L3, A0A0V1IPI2, A0A0V1IKD9, A0A0V1IKA3, A0A0VIIPX2, A0A0V1KD92, A0A0VIJZQ7, A0A0V0YKR7, A0A0VIJCJ6, A0A0V1JE87, A0A0V1J8T7, A0A0V1JS72, A0A0V1II16, A0A0V0YL79, A0A0V1K592, A0A0V1IE69, A0A0V1K9L2, A0A0V0XEJ1, A0A0V1JC56, A0A0V1I359, A0A0V1K7S5, A0A0V1IQ50, A0A0V1J492, A0A0V0YE74, A0A0V0Y8V0, A0A0VIEVN0, A0A0V1K9K9, A0A0V1KD78, A0A0V1G4T5, A0A0V1IAL1, A0A0V1FH81, A0A0V1IDD8, A0A0V1JC58, A0A0V1J0K8, A0A0V0Y705, A0A0VIEVL8, A0A0V1K9B4, A0A0V0XNP3, A0A0VIFRY9, A0A0V1IAQ7, A0A0V0XX86, A0A0V1IQJ7, A0A0V1JE40, A0A0V1FHE6, A0A0V1K4X2, A0A0V1H366, A0A0V1FQ76, A0A0VIEVA7, A0A0V0XW16, A0A0V1K5I2, A0A0VIEUZ6, A0A0V0YGT7, A0A0VIIAS5, A0A0V1E2U6, A0A0V1E1V7, A0A0V1G2W2, A0A0V1JI52, A0A0V1G298, A0A0V1EV41, A0A0VIEMN5, A0A0VIJHF0, A0A0VIKDC2, A0A0V1K5A6, A0A0VIJJD4, A0A0V1K9V2, A0A0VIFLB8, A0A0V0XY64, A0A0VIFYN9, A0A0VIJ0M9, A0A0V1J4L5, A0A0V0YMV1, A0A0V1FQM2, A0A0V1GQ57, A0A0V1KEH4, A0A0V0YGT1, A0A0V0XY09, A0A0VIEUS6, A0A0V1K589, A0A0V0XTS1, A0A0V1ETL0, A0A0V1FFSO, A0A0V1JL92, A0A0V1ED64, A0A0V1ETL9, A0A0V1EV55, A0A0VIJ0J8, A0A0V1G235, A0A0VIKDB0, A0A0V1K3Q7, A0A0VIEEX1, A0A0V0XTR8, A0A0VIFEX1, A0A0V0Y6W9, A0A0V0XKW1, A0A0V0XKX5, A0A0V1K9J3, A0A0V0XKYO, A0A0VIEMK3, A0A0VIFRL6, A0A0V1F4T6, A0A0V1KDD7, A0A0V1FBJ2, A0A0V1K421, A0A0V1FBW8, A0A0VIEVA1, A0A0V0XU04, A0A0VIIRZ9, A0A0V1EV45, A0A0V0XNW4, A0A0VIEVN2, A0A0V0YMZ8, A0A0V0XJ62, A0A0V0YFK6, A0A0VIJPJ3, A0A0V1J0J3, A0A0V0Y715, A0A0V1FMV7, A0A0V1JCP9, A0A0V1E9M7, A0A0V1KDC1, A0A0V1FEU7, A0A0V1KOT0, A0A0V1DST8, A0A0V1IHH3, A0A0V1J0I8, A0A0V1G0P4, A0A0V0XL17, A0A0V1ESY5, A0A0V1K9H0, A0A0V0XUM5, A0A0V1EVN4, A0A0V1EU35, A0A0V1DXH2, A0A0V1JCS1, A0A0V0XNL7, A0A0V1E5G0, A0A0V1G1X6, A0A0VIEWX6, A0A0V1K6H8, A0A0VIFAF9, A0A0V0YNX4, A0A0V1K3Q0, A0A0V1JE80, A0A0V1HB36, A0A0V1IYZ4, A0A0V1KEG0, A0A0V0XUB1, A0A0V0YN83, A0A0V1IIS1, A0A0V1K9N4, A0A0V1EV51, A0A0V1K5S7, A0A0V0XS62, A0A0VIIVQ1, A0A0V1FQU3, A0A0V1K9E0, A0A0V1JPK7, A0A0V1ETF5, A0A0V1FAR0, A0A0VIENV6, A0A0VIFMQ4, A0A0VIJPN2, A0A0V1J380, A0A0V0XJI2, A0A0VIKDE3, A0A0VIDXF5, A0A0V0YE73, A0A0V1F7L0, A0A0VIJEB3, A0A0V1F5X2, A0A0V0YMM2, A0A0V1FBB6, A0A0V1ESS6, A0A0V1G0K7, A0A0VIEVU6, A0A0VIJBG7, A0A0V1G4S3, A0A0V0YMZ0, A0A0VIKDD9, A0A0V1IVW1, A0A0V0YGE0, A0A0V1JZL9, A0A0V0XXD7, A0A0V0XEA7, A0A0V1JE69, A0A0V1J0L5, A0A0V0XFS5, A0A0V1K9R0, A0A0V1G5W1, A0A0V1JCL5, A0A0V0XUK2, A0A0V1DX07, A0A0V0XZB1, A0A0V1IZ01, A0A0V1ET77, A0A0V1F060, A0A0V1JEA8, A0A0V1FY33, A0A0V1FQA4, A0A0V1JHS7, A0A0V1ED73, A0A0V1K5M6, A0A0V0YIZ1, A0A0V1JCM7, A0A0V1IHF8, A0A0V1J0Q3, A0A0V1IVX3, A0A0VIETT3, A0A0V1EU49, A0A0VIE0G2, A0A0V1KD83, A0A0VIFAL4, A0A0V1ET37, A0A0V0XNN7, A0A0V1K5K3, A0A0V0XRQI, A0A0VIEKP8, A0A0V1G1T4, A0A0V1JJ61, A0A0VIIHH9, A0A0VIEVH0, A0A0VIETP5, A0A0V0XUH1, A0A0V0XLI9, A0A0VIJ0K0, A0A0VIIYY2, A0A0VIJPL8, A0A0V0XNN5, A0A0V0Y6W4, A0A0VIG0P5, A0A0V0WI90, A0A0V0WS36, A0A0V0X5U7, A0A0V0WZP8, A0A0V0WF21, A0A0V0WMF7, A0A0V0WJN0, A0A0V0X647, A0A0V0WMC0, A0A0V0X8U0, A0A0V0WGF2, A0A0V0WGK4, A0A0V0WEM4, A0A0V0X8Q3, A0A0V0WH09, A0A0V0WML0, A0A0V0X6B4, A0A0V0WZT2, A0A0V0WS86, A0A0V0X7G3, A0A0V0WYZ7, A0A0V0W8L3, A0A0V0W0I6, A0A0V0WGI7, A0A0V0WYQ7, A0A0V0WX01, A0A0V0WF38, A0A0V0W1G0, A0A0V0W776, A0A0V0WXK0, A0A0V0WA24, A0A0V0X335, A0A0V0WS82, A0A0V0W1K5, A0A0V0W1Z3, A0A0V0W1S4, A0A0V0WAI4, A0A0V0WHC9, A0A0V0V0P6, A0A0V0WHA1, A0A0V0WM77, A0A0V0WJ02, A0A0V0X0M5, A0A0V0X5Y4, A0A0V0WF91, A0A0V0X004, A0A0V0X092, A0A0V0WZR3, A0A0V0X0N1, A0A0V0X081, A0A0V0WDP4, A0A0V0WD03, A0A0V0WHG0, A0A0V0W0M1, A0A0V0ULH6, A0A0V0WX65, A0A0V0WJM1, A0A0V0VIL2, A0A0V0WKK3, A0A0V0WJS6, A0A0V0W1F0, A0A0V0WET2, A0A0V0WIR2, A0A0V0WK58, A0A0V0W771, A0A0V0WAQ3, A0A0V0WJZ4, A0A0V0V151, A0A0V0WWY8, A0A0V0VUH1, A0A0V0X4G5, A0A0V0WXW5, A0A0V0WHJ7, A0A0V0WJM5, A0A0V0WF23, A0A0V0UYA2, A0A0V0W7A7, A0A0V0VH07, A0A0V0W798, A0A0V0WXH6, A0A0V0W6U1, A0A0V0X369, A0A0V0W1Y2, A0A0V0W1I5, A0A0V0WJZ8, A0A0V0W0F4, A0A0V0W1K6, A0A0V0VYK2, A0A0V0WAX9, A0A0V0X4A4, A0A0V0WY03, A0A0V0WHZ9, A0A0V0W4Y7, A0A0V0WM81, A0A0V1P5U0, A0A0V1NES3, A0A0V1P2I1, A0A0V1PI68, A0A0V1P853, A0A0V1NF35, A0A0VIPCG3, A0A0VINXA6, A0A0V1NL85, A0A0VINGD5, A0A0V1PF26, A0A0V1PK08, A0A0V1NHJ3, A0A0V1NLL0, A0A0V1PLE3, A0A0V1P795, A0A0V1PJS4, A0A0V1NY22, A0A0V1P7K0, A0A0V1P5F5, A0A0V1NLV9, A0A0V1NG13, A0A0V1P9L4, A0A0V1P7L8, A0A0V1NEN6, A0A0V1P771, A0A0V1NM54, A0A0V1P5T0, A0A0V1P8Q0, A0A0V1NE86, A0A0V1P280, A0A0V1NS27, A0A0V1P770, A0A0V1PI77, A0A0V1P7C0, A0A0V1PFJ0, A0A0V1P721, A0A0V1NYC7, A0A0V1P6T8, A0A0V1NLN2, A0A0V1NLK1, A0A0V1PAZ0, A0A0V1P2H3, A0A0V1NXE2, A0A0V1P6D6, A0A0V1NVS2, A0A0V1NVB0, A0A0V1P6Q0, A0A0V1PF39, A0A0V1PEY2, A0A0V1P7H3, A0A0V1P9E1, A0A0V1NX80, A0A0V1NTT6, A0A0V1NYS7, A0A0V1P6M8, A0A0V1NGF7, A0A0V1P4Z0, A0A0V0VJK1, A0A0V0VR93, A0A0V0UTV5, A0A0V0UQR6, A0A0V0UMOO, A0A0V0V174, A0A0V0V426, A0A0V0UTZ7, A0A0V0VREI, A0A0V0VRII, A0A0V0UNQ3, A0A0V0VRC8, A0A0V0VE39, A0A0V0VFJ5, A0A0V0UQ81, A0A0V0VFQ4, A0A0V0VFH2, A0A0V0VPC7, A0A0V0UUY9, A0A0V0VRE0, A0A0V0VP79, A0A0V0V552, A0A0V0VFW1, A0A0V0VS73, A0A0V0VWE9, A0A0V0V155, A0A0V0VCB7, A0A0V0VMC3, A0A0V0V7D6, A0A0V0UPR0, A0A0V0UUV8, A0A0V0V210, A0A0V0VE27, A0A0V0VRQ3, A0A0V0VEC8, A0A0V0UQH4, A0A0V0VFX1, A0A0V0VSL6, A0A0V0VRE9, A0A0V0V6V2, A0A0V0VP70, A0A0V0VSK7, A0A0V0UUX7, A0A0V0VED2, A0A0V0VFN7, A0A0V0V499, A0A0V0V3J5, A0A0V0V587, A0A0V0VW86, A0A0V0VQ95, A0A0V0VCJ2, A0A0V0VSS5, A0A0V0UJV4, A0A0V0UPU4, A0A0V0VGC0, A0A0V0U3P2, A0A0V0VE47, A0A0V0UTV0, A0A0V0UMK8, A0A0V0T3U1, A0A0V0VPC1, A0A0V0V159, A0A0V0VRW7, A0A0V0VTEI, A0A0V0VJ88, A0A0V0UVL3, A0A0V0UUP5, A0A0V0VE17, A0A0V0V119, A0A0V0UNR0, A0A0V0VFK5, A0A0V0VLU4, A0A0V0VDL9, A0A0V0VW85, A0A0V0VRG0, A0A0V0UQQ3, A0A0V0VEJ2, A0A0V0VDY8, A0A0V1BV44, A0A0VIASI9, A0A0VIAWR7, A0A0V1C327, A0A0V1ARX7, A0A0V1ASM2, A0A0V1BM31, A0A0V1ASMO, A0A0V1BY88, A0A0VIBSS1, A0A0V1BU64, A0A0VIBYB4, A0A0V1B1M0, A0A0VIBFH0, A0A0VIBTA4, A0A0V1BU83, A0A0VIBYZ5, A0A0VIBYW3, A0A0VIASI6, A0A0V1B1C8, A0A0VIBUM6, A0A0V1BY31, A0A0VIAWB4, A0A0VIAUR8, A0A0V1BTZ8, A0A0V1AZ75, A0A0V1BF39, A0A0V0Z9Z1, A0A0V1B1K9, A0A0V1BF98, A0A0V1AUN2, A0A0V1BG87, A0A0V1AQK3, A0A0V1BXK5, A0A0V1BGUO, A0A0V1BV17, A0A0V1BY17, AlDYF2, A0A0V1C087, A0A0V1AW82, A0A0VIARD6, A0A0VIBYW4, A0A0V1AQ90, A0A0VIARB8, A0A0V1BHN0, A0A0V1AQK5, A0A0V1BSR9, A0A0V1AV98, E5S9D6, A0A0V1ASN4, A0A0V1BZS3, A0A0V1BYF9, A0A0V1BY68, A0A0V1AWE1, A0A0V1C324, A0A0V1BV78, A0A0V1BIN6, A0A0V1BV40, A0A0V1BZG3, A0A0V1AT94, A0A0V1AZ10, A0A0V1AUX5, A0A0V1AU60, A0A0V1AQI4, A0A0V1BG93, A0A0V1BGM2, A0A0V1BT93, A0A0V1BYD9, A0A0V1C2F3, A0A0VIALY2, A0A0V1BY24, A0A0V1BY86, A0A0VIBUE7, E5SVP5, A0A0V1ARU4, A0A0V1BMR3, A0A0V1ARE7, A0A0V1HCU6, A0A0V1HT58, A0A0V1HB44, A0A0V1HUT8, A0A0V1I1I8, A0A0V1HNV1, A0A0V1GP67, A0A0V1H9N6, A0A0V1HMQ6, A0A0V1H7J5, A0A0V I1W3, A0A0V1HAB7, A0A0VIHXL0, A0A0V1HW85, A0A0V1H4C6, A0A0V1I5B5, A0A0V1HR65, A0A0V1HBV3, A0A0V1I259, A0A0V1I3Y0, A0A0V1HLA0, A0A0V1HW02, A0A0V1HLD1, A0A0V1HVP2, A0A0V1HLM5, A0A0V1I5C9, A0A0V1I289, A0A0VIHBL5, A0A0V1H4G7, A0A0V1H7I9, A0A0VIHYK5, A0A0V1I3Y7, A0A0V1GSV2, A0A0V1HE40, A0A0V1H7R5, A0A0V1I5B7, A0A0V1HAK7, A0A0V1HBJ8, A0A0V1HX30, A0A0V1HDT3, A0A0V1I4J6, A0A0V1H559, A0A0V1HQU5, A0A0V1HAU6, A0A0V1HAX6, A0A0V1HCF7, A0A0V1HEP2, A0A0V1HCA8, A0A0V1HEI2, A0A0V1HTQ9, A0A0V1H6F1, A0A183VXQ2, A0A183VLN0, A0A3P8HFW6, A0A395NMG6, A0A395NGU7, A0A395NHQ3, A0A2T3ZPF6, A0A2T3ZAJ6, A0A2T3YU56, A0A2T3YQC3, A0A2T3YQB8, A0A2T3ZA54, A0A2T3YQC0, A0A2T4B6T4, A0A2T4B5A6, A0A2KOTAN5, A0AIT3CP57, A0AIT3CIF4, A0A2T3ZRT3, A0A2T3ZXU3, A0A2T4ANA8, A0A2T3ZX63, A0A2KOTYA4, A0A2N1LZN2, A0A0F9ZNQ8, A0A2N1L2X0, A0A2N1LKG2, A0A2K0U895, A0A2N1LM61, A0A0F9X8N3, A0A2T4CJY3, A0A2T4CIH3, A0A2H2Z6Q9, A0A2H2Z2T5, A0A232F3J2, A0A232FIF8, A0A232F941, A0A232F4N8, A0A232F7T9, F2SMU2, A0A022W2V9, A0A178F2Y8, A0A022XTQ1, A0A178FP64, A0A369RWP4, A0A0N5E1P0, A0A0N5DL77, A0A0N5DPD5, A0A0N5DH91, A0A0N5DXR0, A0A0N5DPD6, A0A0N5DRN3, A0A0N5DYA9, A0A0N5DYK6, A0A0N5DUR4, A0A0N5DEG2, A0A0N5DVP1, A0A0N5DR95, A0A0N5DR76, A0A0N5E6W2, A0A0N5DHF1, A0A0N5DRT2, A0A0N5DD95, A0A0N5DM82, A0A085NT68, A0A085N873, A0A085MF87, A0A085MJD4, A0A0B1PQG5, A0A085M562, A0A085LN72, A0A085N482, A0A0B1PME0, A0A0B1PKA9, A0A085MJ81, A0A085LWZ9, A0A085NTB9, A0A0B1PI26, A0A0B1PIN9, A0A085MPH9, A0A085LXR5, A0A085NL76, A0A085NGB0, A0A085M826, A0A0B1PP49, A0A085MVC4, A0A085M330, A0A085LIA0, A0A085N5V9, A0A085MK20, A0A0BIPRQ3, A0A085NN81, A0A085LIK5, A0A085M8H3, A0A085MX09, A0A085N8R6, A0A085M2H7, A0A085NKE8, A0A085LQT6, A0A077YW89, A0A077YZL9, A0A077Z1E9, A0A077ZBA3, A0A077Z7J7, A0A077Z0B4, A0A077ZF09, A0A077Z0L9, A0A077Z8C3, A0A077Z0L5, A0A077YVM8, A0A077ZAR2, A0A077ZB27, A0A077Z4C4, A0A077ZFL2, A0A077YY03, A0A077ZGT3, A0A077Z244, A0A077ZE86, A0A392PT57, A0A392M1H5, A0A392TWG0, A0A392PBX8, A0A392N1T7, A0A392Q1K1, A0A392NG93, A0A392NDB2, A0A392RWA2, A0A392NAQ6, A0A392NVW7, A0A392PE82, A0A392PGQ4, A0A392N2X5, A0A392PG95, A0A392N4E7, A0A392PA83, A0A392NWT8, A0A392NQL4, A0A392MBY4, A0A392Q8A5, A0A392PMK5, A0A392MI51, A0A392NQF2, A0A392Q3T9, A0A392NX73, A0A392N067, A0A392P4S9, A0A392NR91, A0A392MH64, A0A392NFX7, A0A2K3N545, A0A2K3NIL3, A0A2K3NQL2, A0A2K3PPK5, A0A2K3PAC8, A0A2K3N5G0, A0A2K3PGR8, A0A2K3PAJ7, A0A2K3PNI2, A0A2K3L2K4, A0A2K3NKF3, A0A2K3MWB2, A0A2K3MNA4, A0A2K3PFA1, A0A2K3LYQ1, A0A2K3NPR5, A0A2K3PKJ0, A0A2K3KY21, A0A2K3P821, A0A2K3N0U2, A0A2K3NIX8, A0A2K3PCX5, A0A2K3MTX0, A0A2K3P0N9, A0A2K3K2X8, A0A2K3K1G0, A0A2K3P4N6, A0A2K3MMYO, A0A2K3N390, A0A2K3PRUO, A0A2K3NHE9, A0A2K3MP42, A0A2K3L7A3, A0A2K3KDT6, A0A2K3L793, A0A2K3NJM8, A0A2K3NHS7, A0A2K3M7W6, A0A2K3P4W6, A0A2K3NNI7, A0A2K3L7X0, A0A2K3LKJ0, A0A2K3MQU1, A0A2K3JN77, A0A2K3PRC7, A0A2K3LSP2, A0A2K3LQR6, A0A2K3NAV2, A0A2K3N5F0, A0A2K3P2Q7, A0A2K3NIV7, A0A2K3MTU5, A0A2K3MZSO, A0A2K3JM43, A0A2K3LPU5, A0A2K3N4M8, A0A2K3PJSO, A0A2K3NB85, A0A2K3M7I7, A0A2K3NDE7, A0A2K3KSU5, A0A2K3PLE0, A0A2K3M3U1, A0A2K3L147, A0A2K3P033, A0A2K3LVH3, A0A2K3N391, A0A2K3NHE1, A0A2K3NPS4, A0A2K3L0P8, A0A2K3MRR9, A0A2K3P8M4, A0A2K3M9G4, A0A2K3JVE5, A0A2K3LWR5, A0A2K3KZ94, A0A2K3KSU6, A0A2K3LB77, A0A2K3NP95, A0A2K3N177, A0A2K3P0P9, A0A2K3LRR4, A0A2K3PL10, A0A2K3PJV4, A0A2K3NSR0, A0A2K3KLD8, A0A2K3LCR5, A0A2K3NTH2, A0A2K3PKC0, A0A2K3MMJ1, A0A2Z6LMC9, A0A2Z6MW96, A0A2Z6M7P9, A0A2Z6NDV6, A0A2Z6MC05, A0A2Z6MAQ7, A0A2Z6N0I3, A0A2Z6LQ21, A0A2Z6MXN7, A0A2Z6M5F5, A0A2Z6MXE3, A0A2Z6P679, A0A2Z6LR55, A0A2Z6MQ73, A0A2Z6MDJ9, A0A2Z6NUW8, A0A2Z6MKZ5, A0A2Z6NWUO, A0A2Z6ME77, A0A2Z6MPN7, A0A2Z6NJ42, A0A2Z6LL70, A0A2Z6LM12, A0A2Z6P1H7, A0A2Z6LMM9, A0A2Z6MPK7, A0A2Z6N5W6, A0A2Z6MAP8, A0A2Z6N1K5, A0A2Z6NTN7, A0A2Z6LQG0, A0A2Z6LQW7, A0A2Z6LPI7, A0A2Z6MQ16, A0A2Z6MDD8, A0A2Z6P268, A0A2Z6LPS2, A0A2Z6P3R5, A0A2Z6MYS4, A0A2Z6LPQ7, A0A2Z6NJK2, A0A2Z6P441, A0A2Z6NAG1, A0A2Z6MMM3, A0A2Z6M4K0, A0A2Z6MVN2, A0A2Z6LPR4, A0A2Z6LTH3, A0A2Z6PUS7, A0A2Z6MQE3, A0A2Z6NF42, A0A2Z6MF49, A0A2Z6MQ83, A0A2Z6MRQ0, A0A2Z6MFP6, A0A2Z6NXT5, A0A2Z6N7M7, A0A2Z6P962, A0A2Z6LPY4, A0A2Z6NL64, A0A2Z6M8M8, A0A2Z6P518, A0A2Z6P2F4, A0A2Z6MYB5, A0A2Z6LTQ0, A0A2Z6LV78, A0A2Z6MVE1, A0A2Z6LKE4, A0A2Z6PDH2, A0A2Z6M528, A0A2Z6MVF6, A0A3B6NPP9, A0A3B6IPA0, A0A3B6GXA5, A0A3B6CFN8, A0A3B6GVI4, A0A3B6AVA7, A0A3B6ITK6, A0A3B6DM60, A0A3B6AVU8, A0A3B6EJV5, A0A3B6DAX4, A0A3B6I2Q0, A0A3B6FV60, A0A077RVZ3, A0A3B6GWT1, A0A3B6FV65, A0A3B5Y188, A0A3B6FQR7, A0A3B5Y1P0, A0A3B6CIE0, A0A3B5YYY7, A0A3B6GVI9, A0A3B6DAD8, A0A3B6C3X4, A0A3B6MW88, A0A3B6IKZ5, A0A3B6B802, A0A3B6C476, A0A3B5XTG1, A0A3B6CDR2, A0A3B6CFN2, A0A3B6DQD8, A0A3B6ML17, A0A3B6D535, A0A3B6TPW9, A0A3B6DN59, A0A3B6HWP0, A0A3B5Z3I2, A0A3B6KT61, A0A3B6FWV1, A0A3B5ZXT0, A0A3B6SIG2, A0A3B6CFN4, A0A3B6DQC5, A0A2X0SW72, A0A3B6FER1, A0A3B6CFL5, A0A3B6CDW1, A0A3B6QD29, A0A3B6U554, A0A3B6MWW1, A0A3B6B6Z0, A0A3B6IXZ8, A0A3B6C118, A0A3B6MWI7, A0A3B6RA84, A0A3B6DM95, A0A3B6KNX5, A0A3B5XTI8, A0A3B6RP60, A0A0R8CAF3, A0A3B6B844, A0A3B5YZH3, A0A3B5ZZY7, A0A3B5ZYM3, A0A3B6DM69, A0A3B6D841, A0A3B6IWH6, A0A3B6NXU2, A0A3B6D593, A0A3B6C633, A0A3B6UAV8, A0A3B6CDW2, A0A3B6RDT9, A0A3B6CDV2, A0A3B6LUF7, A0A3B6DQ92, A0A3B6SL80, A0A3B6N0N1, A0A3B6DLD6, A0A2X0SND0, A0A3B5ZZF6, A0A3B6B2F3, A0A3B6MYX9, A0A3B5Z1X9, A0A3B6DMT7, A0A3B6KN82, A0A3B6NWS4, A0A3B5XT21, A0A3B6C8K8, A0A3B6DMB1, A0A3B6JIP6, A0A3B6G0F8, A0A3B6CFH5, A0A3B6C728, A0A3B6QP32, A0A3B6RMYO, A0A3B6DML0, A0A3B6RPD1, A0A3B6SA65, A0A3B6DQD5, A0A3B6MP47, A0A3B6DL71, A0A3B6IV36, A0A3B6DJM6, A0A3B6MWH8, A0A3B6B8U6, A0A3B6TNK8, A0A3B6MWJ6, A0A3B6CFR7, A0A3B6GPP5, A0A2X0TGT9, A0A2X0SOA4, A0A3B6B6Z5, A0A3B6AR12, A0A3B6DDD0, A0A3B6TFJ0, A0A3B6CI24, A0A3B6TPC6, A0A3B6B568, A0A3B6IWJ1, A0A3B6SEP2, A0A3B6KLI6, A0A3B6EKY9, A0A3B6CG16, A0A3B6B8T6, A0A3B6RPA4, A0A3B6TT98, A0A3B6H304, A0A3B6EH46, A0A3B6FUN9, A0A3B6CFSO, A0A3B6KRY5, A0A3B6B571, A0A3B6TQ03, A0A3B6GVH4, A0A3B6CDR7, A0A3B6I3N0, A0A3B6B7D6, A0A3B6CFM9, A0A3B6LRY8, A0A3B6DJM2, A0A3B6MV81, A0A3B5ZNL3, A0A3B6A183, A0A3B6QP21, A0A3B6BZH0, A0A3B6J0A9, A0A3B6B640, A0A3B6DL82, A0A3B6QNH4, A0A3B6U4W3, A0A3B5Z2P0, A0A3B6CDX1, A0A3B6JP90, A0A3B6S8I2, A0A3B6MV89, A0A3B6LXS1, A0A3B6QP12, A0A3B6B6T4, A0A3B5XT90, A0A3B6DQE1, A0A3B6CH00, A0A3B6KN77, A0A077RVZ1, A0A3B5Z285, A0A3B6AZK9, A0A3B6B840, A0A3B6CFZ6, A0A3B6SBZ7, A0A3B6PVE3, A0A3B6LDI3, A0A3B6N2M7, A0A3B6C6J6, A0A3B6DN39, A0A3B6DLB0, A0A3B6B710, A0A3B6DN68, A0A3B6LQP4, A0A3B5XVZ4, A0A3B6TQM2, A0A3B6I168, A0A3B6CFM3, A0A3B6LXQ4, A0A3B6CDS2, A0A3B5Z158, A0A3B6TXD5, A0A3B6UAM6, A0A2X0SCN5, A0A3B6UC57, A0A3B6SRM5, A0A3B6TW06, A0A2X0SNF0, A0A3B6MII3, A0A3B6CEN4, A0A3B6DJN6, A0A3B6B7S8, A0A3B6KNC2, A0A3B6CDU3, A0A3B6C714, A0A3B6MXU5, A0A3B6QKM8, A0A3B6FN69, A0A3B5ZME3, A0A3B6N370, A0A3B6B8R0, A0A3B6LRZ5, A0A3B6LY19, A0A3B6B6X0, A0A3B6GTI5, A0A3B6HX59, A0A3B6LR65, A0A3B6KLI0, A0A3B6B7J7, A0A3B5Y5C6, A0A3B6SDH9, A0A3B6SPZ5, A0A3B6D4X4, A0A3B6DQM1, A0A3B6DDV3, A0A3B6TAX9, A0A3B6KKP1, A0A3B6AVA1, A0A3B6DLC1, A0A3B6EKT7, A0A3B6SQQ6, A0A3B6C001, A0A3B6H0E6, A0A3B6LEV2, A0A3B6LS79, A0A3B6B644, A0A3B6DE47, A0A3B6DL94, A0A2X0SNW6, A0A3B6AX84, A0A3B6BXA3, A0A3B6TT93, A0A3B5XUG7, A0A3B6PV04, A0A3B6CDW7, A0A3B6ARF6, A0A3B6B7Q6, A0A3B6A030, A0A3B6JCZ0, A0A3B6KKN0, A0A3B5Y464, A0A3B6B7H2, A0A3B6CDT7, A0A3B5Z410, A0A3B6BZG5, A0A3B6CFP5, A0A3B6SKQ8, A0A3B6G1T3, A0A3B6LP90, A0A3B6MPMO, A0A3B5ZQN2, A0A3B6MWW7, A0A3B6FI27, A0A3B6GSI9, A0A3B5Z165, A0A3B6CEJ7, A0A3B6H694, A0A3B5Z1E9, A0A3B6CFP7, A0A3B6HT43, A0A3B6RSP0, A0A3B5YT59, A0A3B6CFN5, A0A3B6KNX0, A0A3B6TK76, A0A3B6SIF3, A0A3B6PST4, A0A3B6CC02, A0A3B6TUP1, A0A3B6B0K5, A0A3B6CHY5, A0A3B6B7R1, A0A3B6B826, A0AID5UGG3, A0A3B5ZZV1, A0A3B6B022, A0A3B6KD22, A0A3B6KRL2, A0A3B6U115, A0A3B6GNZ2, A0A3B6JDU7, A0A3B5Y527, A0A2X0SBL5, A0A3B6CDT0, A0A3B6C3Z6, A0A3B5Y3Q9, A0A3B6QL00, A0A3B6SJY6, A0A3B6ESF4, A0A3B6DJP0, A0A3B6ER67, A0A3B6RJV3, A0A3B6CFJ4, A0A3B5Y5D0, A0A3B6CDT5, A0A3B6JF83, A0A3B6SDH4, A0A3B6DL99, A0A3B6AQB5, A0A3B6H388, A0A3B6PSS9, A0A3B6FRH5, A0A3B6NSZ0, A0A3B6CFN7, A0A3B6C3W9, A0A3B6ENW4, A0A3B6B7N8, A0A3B6DGA7, A0A3B6D4L4, A0A3B6MXW0, A0A2X0SNE0, A0A3B6DNS5, A0A3B6B820, A0A3B6DL80, A0A3B6LS69, A0A3B6CI19, A0A3B6TNR8, A0A3B5ZVB3, A0A3B6R9Q9, A0A3B6CG09, A0A3B5ZME6, A0A3B6IMW8, A0A3B6SLK7, A0A3B6H2G6, A0A3B6KNMO, A0A3B6DAE3, A0A3B6SLZ3, A0A3B6IR46, A0A3B6CDX8, A0A3B6RMR3, A0A3B6JK14, A0A3B6LP53, A0A3B6SQQ3, A0A3B6LSC8, R9UC29, A0A2X0SUV4, A0A3B6B733, A0A3B6GZL0, A0A2X0SCV8, A0A3B6BXQ7, A0A3B6RPT7, Q56DH9, A0A3B6TE76, A0A3B6SNL4, A0A3B6B7Z9, A0A3B6IZM9, A0A3B6CG90, A0A3B6TPR0, A0A3B6AWA3, A0A3B6B7V1, I0JTUI, A0A3B6GWS6, A0A3B5Z166, A0A3B6EEA5, D8L9S5, A0A3B6AYR6, A0A3B6CFM6, A0A3B6SHS1, A0A3B6KSI0, A0A3B6CFN3, A0A3B6NLJ3, A0A3B6CFN9, A0A3B5Z2J0, A0A2X0SBH9, A0A2X0UOZ3, A0A3B6MXW5, A0A3B6LQP0, A0A3B6JI87, A0A3B6REN4, A0A3B6RMR6, A0A3B6BZR8, A0A3B6B5R5, A0A3B6CI12, A0A3B6DHA3, A0A3B6KAL1, A0A3B6B715, A0A2X0SPL7, A0A3B5ZMU9, A0A3B6H6M7, A0A3B6AWQ0, A0A3B6TF32, A0A3B6HPJ6, A0A3B6RTD5, A0A3B6DKA3, A0A3B6LJM9, A0A3B6ESW5, A0A3B6DM74, A0A3B6TW12, A0A3B6B6D0, A0A3B6LRZ0, A0A3B6MXV5, A0A3B6RB18, A0A3B6NWL7, A0A3B6AVX3, A0A3B6C9J4, A0A3B6SQF7, A0A2X0U1E9, A0A3B6B7V7, A0A3B6RPG4, A0A3B6CF85, A0A3B6D812, A0A3B6LM48, A0A3B6TLB6, A0A3B6AVW3, A0A3B6EHS5, A0A3B6IK35, A0A3B6B8F8, A0A2X0SCT9, A0A3B6U7R0, A0A3B5ZP26, A0A3B5XTI4, A0A3B6U6Q9, A0A3B5ZRQ8, A0A3B6DPF6, A0A3B6RR09, R9UFG9, A0A3B6QL55, A0A3B6EM35, A0A3B6DAS8, A0A3B6EM42, A0A3B6EJL4, A0A3B6B738, A0A3B6FRT0, A0A3B6GQZ4, A0A3B6NVE3, A0A3B5Y4W4, A0A3B6HT50, A0A3B6RQ82, A0A3B6JK06, A0A2X0SCX3, A0A3B6DAS6, A0A3B6B0L1, A0A3B6U6W6, A0A3B6PSW1, A0A3B6MI22, A0A3B6EM39, A0A3B6FUKO, A0A3B6EJW4, A0A3B6C439, A0A3B5ZYD7, A0A3B6FUJ5, A0A3B6C3V9, A0A3B6DAW9, A0A3B6C3Y1, A0A3B6H012, A0A3B6NIA4, A0A2X0RZC4, A0A3B6DE52, A0A3B5ZYE5, A0A2X0U219, A0A3B6EH34, A0A3B6AVB1, A0A3B6GXA9, A0A3B6EJV9, A0A3B6DN34, A0A3B6KN67, A0A3B6C6J2, A0A3B6AX65, A0A3B6AX70, A0A3B6A137, A0A3B6CFK5, A0A3B6FRS5, A0A3B6NX53, A0A3B6DKK6, A0A3B6QCT9, A0A3B6LXE2, A0A3B6GXB4, A0A3B6C471, A0A3B6AWQ3, A0A3B6DNI2, A0A3B6A1F9, A0A3B6AVW8, A0A3B6EJK8, A0A3B6FUK5, A0A3B6JE30, A0A3B6GWT6, A0A3B6CDY1, A0A3B6DKS5, A0A3B6AWA8, A0A3B6C434, A0A3B6GVH9, A0A3B6B5S4, A0A3B6DLD1, A0A3B5XW78, A0A3B6H0F8, A0A3B6N1N1, A0A3B6AZ76, A0A3B6MWV6, A0A3B5ZZF3, A0A3B6CG64, A0A3B6H5X7, A0A2X0SDG7, A0A3B6TPX5, A0A3B6BXA2, A0A3B5XTC5, A0A3B6CFT5, A0A3B6DM84, A0A3B6BWH3, A0A3B6HTC9, A0A3B6PUX9, A0A3B5YQ73, A0A3B6DBG8, A0A3B6SK28, A0A3B6MWX7, A0A3B6CDU5, A0A3B6DMH8, A0A3B6LUSO, A0A3B6DM89, A0A3B6LRZ3, A0A3B6DJM9, A0A3B5Z2V7, A0A3B6B883, A0A3B6B6C5, A0A3B5Z2G7, A0A3B6B8V7, A0A3B6DMH3, A0A3B6EF95, A0A3B5YQF7, A0A3B6CHZ0, A0A3B5XWA5, A0A3B5XTJ9, A0A3B6LRZ4, A0A3B6HYQ9, A0A3B6RP18, A0A3B6GZZ9, A0A3B6AQC9, A0A3B6B980, A0A3B6LRX9, A0A3B6DLC6, A0A3B6NXN1, A0A3B6SLK3, A0A3B6B7T6, A0A2X0SCN7, A0A3B6CDX6, A0A3B6DMA2, A0A3B6B036, A0A3B6B576, A0A3B6C3Z0, A0A3B6ASJ6, A0A3B6HW93, A0A2X0RZB1, A0A3B6CFS5, A0A3B6DBG3, A0A3B6KSQ6, A0A3B6C6A4, A0A2X0TS56, A0A3B6PLI1, A0A3B6B719, A0A3B6CFR6, A0A3B6H004, A0A3B6C787, A0A2X0TYQ5, A0A3B5ZXN5, A0A3B6CG60, A0A3B6DLA4, A0A3B6DQ26, A0A3B6SF92, A0A2X0SBJ0, A0A3B6DLN1, A0A3B6E8J0, A0A3B6GLR2, A0A3B6KUH0, A0A3B5YQG9, A0A3B6CFK3, A0A3B6B8P2, A0A3B6DMG8, A0A2X0SBA4, A0A3B6TUB4, A0A3B6EDF4, A0A3B6RPB1, A0A3B6DMF8, A0A3B6UCL0, A0A3B6PUV2, A0A3B6H0E9, A0A3B5Y7C0, A0A3B6GN82, A0A3B6AX75, A0A3B6LQN7, A0A3B6DLB6, A0A3B6DKS9, A0A3B6LRY4, A0A3B6DKI6, A0A3B6GX38, A0A3B5ZMG1, A0A3B5Z1I1, A0A2X0SCS8, A0A3B6TI56, A0A3B6CFI4, A0A3B6AQU8, A0A3B6C782, A0A3B5ZZT3, A0A3B6KL84, A0A3B6RL94, A0A3B6TQ21, A0A3B6LRY9, A0A3B6QN56, A0A3B5YRB3, A0A3B6UAH4, A0A3B6AR71, A0A3B6KED9, A0A3B6TWJ5, A0A3B6ERL9, A0A3B6B6E3, A0A3B6H1E1, A0A3B6DN47, A0A3B6FXSO, A0A3B6MX31, A0A3B6FMU4, A0A3B6APH5, A0A2X0RZY7, A0A3B6DNR6, A0A3B6RL87, A0A3B6TMT8, A0A3B6RQ86, A0A3B6EJJ5, A0A3B6B232, A0A3B6RQ77, A0A2X0TMP3, A0AID6RPH0, A0A2X0SCP5, A0AID5UXF1, A0A077S3W5, W5D450, A0AID5UN73, W5C0L4, A0AID5UXD7, A0A077RQ16, A0AID5UVZ2, A0A077RZC2, A0A077RQQ4, A0A077RFI1, A0A341ZF31, A0AID5UKP6, A0A077S2M2, A0AID5UK73, A0AID5UY85, A0A077SOP5, A0A446MVQ3, A0A446LHZ4, A0A446REL1, A0A446MUR9, A0A446UJ83, A0A446YSZ1, A0A446TC95, A0A446SLK3, A0A446LI13, A0A446MV 11, A0A446UJ84, A0A446NKA8, A0A446Q0W6, A0A446VU93, A0A446Y561, A0A446NRX1, A0A446SJZ5, A0A446L3Y0, A0A446MRJ4, A0A446KV21, A0A446LHU9, A0A446MRR5, A0A446PDR2, A0A446Q0X4, A0A446TCN2, A0A446LI10, A0A446MVF3, A0A446LHZ0, A0A446PK86, A0A446MWF9, A0A446K644, A0A446VWF4, A0A446MWD8, A0A446M560, A0A446W8K1, A0A446YTG2, A0A446LLE9, A0A446LLA9, A0A446MV52, A0A446MRN3, A0A446LHW9, A0A446NRW8, A0A446RQK6, A0A446RQM9, A0A446M588, A0A446UJ35, A0A446UJ06, A0A446VSN4, A0A446UJB3, A0A446LMD3, A0A446S8Q2, A0A446LII1, A0A446LHV2, A0A446REN0, A0A446YTE0, A0A446MRL7, A0A446LHV8, A0A446MRL2, A0A446UJ76, A0A446TC94, A0A446IP34, A0A446NRW5, A0A446XSM0, A0A446TCA5, A0A446KA03, A0A446YTD1, A0A446MW57, A0A446M577, A0A446UJ96, A0A446K654, A0A446LLJ7, A0A446K022, A0A446WQH4, A0A446KP30, A0A446MUV2, A0A446X5A7, A0A446NRU9, A0A446MVY3, A0A446MUV4, A0A446X576, A0A446NRV0, A0A446L422, A0A446LLB4, A0A446L9C3, A0A446YVN7, A0A446NRW9, A0A446XZM9, A0A446MV59, A0A446Y2M9, A0A446LL76, A0A446MV73, A0A446UJ87, A0A446L975, A0A446Y3N5, A0A446KV41, A0A446K617, A0A446QGE1, A0A446MV32, A0A446PK69, A0A446SK23, A0A446YZ67, A0A446MI06, A0A446LI16, A0A446XY56, A0A446YP66, A0A446LL77, A0A446REX2, A0A446NK72, A0A446UJA1, A0A446YLI9, A0A446K601, A0A446UJ43, A0A446NK74, A0A446LL83, A0A446XPQ6, A0A446VUA3, A0A446NRU8, A0A446KUV2, A0A446Q9Q4, A0A446K5J4, A0A446WSK7, A0A446NRZ1, A0A446M568, A0A446JML1, A0A446SLJ0, A0A446R9Q0, A0A446UK57, A0A446LI26, A0A446M587, A0A446XU41, A0A446UJ71, A0A446T6X8, A0A446KV32, A0A446LM41, A0A446L404, A0A446MV88, A0A446NRW1, A0A446J771, A0A446M551, A0A446KUW7, A0A446KUU3, A0A446MV70, A0A446LHW7, A0A446MV74, A0A446M5B2, A0A446LL46, A0A446MV68, A0A446PK92, A0A446MVS2, A0A446XVJ2, A0A446LHU2, A0A446UKD3, A0A446M590, A0A446K035, A0A446SLG8, A0A446LKQ3, A0A446QEYO, A0A446MUR6, A0A446IP50, A0A446P3S7, A0A446QNL4, A0A446R7C5, A0A446JML0, A0A446NRW7, A0A446MRN0, A0A446MRN9, A0A446MUS6, A0A446R1Y3, A0A446LJB5, A0A446KHJ9, A0A446YPI4, A0A446XPR5, A0A446LHT9, A0A446KUZ6, A0A446MVB0, A0A446K6J7, A0A446LHY6, A0A446Q9Y2, A0A446TBU5, A0A446L175, A0A446MRK5, A0A446Q9T2, A0A446UJ22, A0A446M562, A0A446MRI6, A0A446P3S9, A0A446SK01, A0A446YS27, A0A446MI08, A0A446MCY9, A0A446MRR0, A0A446MRK8, A0A446KV16, A0A446M570, A0A446L164, A0A446M582, A0A446REF2, A0A446JA06, A0A446MRP1, A0A446M580, A0A446M565, A0A446SLJ2, A0A446J9N6, A0A446MDG6, A0A446UJC2, A0A446UJ81, A0A446SLP8, A0A446LL86, A0A446Y563, A0A446MZY1, A0A446K620, A0A446MVV2, A0A446NB74, A0A446LLA2, A0A446MUS4, A0A446TT86, A0A446LRA6, A0A446L9G2, A0A446L414, A0A446K5H4, A0A446SXH2, A0A446MV89, A0A446MRJ7, A0A446QNN2, A0A446MRS6, A0A446L9B9, A0A446NRU1, A0A446MWC8, A0A446MVB4, A0A446UJ63, A0A446NRY5, A0A446K9J8, A0A446MW84, A0A446USM4, A0A446K5G2, A0A446MRT0, A0A446UQJ9, A0A446JML2, A0A446UJ99, A0A446MSW5, A0A446MUV5, A0A446L406, A0A446RS14, A0A446SAY9, A0A446YNY1, A0A446NS01, A0A446KV78, A0A446LL87, A0A446K036, A0A446L9A2, A0A446NRY3, A0A446MUH3, A0A446NRV5, A0A446L3X8, A0A446LL80, A0A446QLZ4, A0A446UJ94, A0A446MUY4, A0A446NAL7, A0A446VV46, A0A446MVA2, A0A446LM62, A0A446UJ59, A0A446MRJ9, A0A446MV20, A0A446LHR2, A0A446LL97, A0A446MRG0, A0A446LHX1, A0A446U9T5, A0A446XVM3, A0A446M558, A0A446R9M6, A0A446JGG2, A0A446UJ85, A0A446TQK5, A0A446KVB1, A0A446YTB0, A0A446MY43, A0A446K667, A0A446MVJ1, A0A446LL78, A0A446TR27, A0A446TQK4, A0A446REI4, A0A446JA28, A0A446Q0X1, A0A446K647, A0A446RLD6, A0A446RM11, A0A446LSA0, A0A446MUX5, A0A446MVA1, A0A446K6G4, A0A446UJ65, A0A446KGH3, A0A446KV82, A0A446KV63, A0A446MV50, A0A446NK01, A0A446IHX8, A0A446MRT7, A0A446K639, A0A446MRH0, A0A446MV40, A0A446R1L1, A0A446RQM3, A0A446K5K0, A0A446MD27, A0A446MW47, A0A446LHX2, A0A446TQK1, A0A446L9F6, A0A446RDR5, A0A446XSM3, A0A446KV34, A0A446MV21, A0A446M526, A0A446R9J1, A0A446QA33, A0A446NS73, A0A446Q9P9, A0A446TT87, A0A446SMN3, A0A446P4B8, A0A446K5Z2, A0A446MV84, A0A446LHW3, A0A446UIZ6, A0A446XTI5, A0A446RZS7, A0A446LJA8, A0A446KUZ3, A0A446MRI0, A0A446QA44, A0A446MUT9, A0A446LLE5, A0A446VUA1, A0A446M5A4, A0A446LHT8, A0A446KGV8, A0A446J7A1, A0A446M579, A0A446RQK2, A0A446LR36, A0A446NRY7, A0A446NK41, A0A446NRV3, A0A446XPD9, A0A446K031, A0A446N0X5, A0A446P3S3, A0A446Q9R0, A0A446UJ70, A0A446TBW3, A0A446UJC6, A0A446S8Q6, A0A446X3F4, A0A446MVC2, A0A446WSC8, A0A446RZ79, A0A446Y556, A0A446SJZ8, A0A446Q9P3, A0A446Y367, A0A446II08, A0A446WMX9, A0A446Q9Q0, A0A446L3Z8, A0A446LJJ7, A0A446J765, A0A446UJ32, A0A446K938, A0A446NRY8, A0A446UJ23, A0A446N128, A0A446UJ69, A0A446LL91, A0A446JP82, A0A446MRL0, A0A446Q9P6, A0A446SLQ8, A0A446LHT2, A0A446LR56, A0A446MVD0, A0A446NRX7, A0A446K9M4, A0A446MVX3, A0A446K1Q8, A0A446NAD8, A0A446N9Y9, A0A446UJ82, A0A446NK52, A0A446YTG9, A0A446MRM3, A0A446UJ89, A0A446NS53, A0A446KFS7, A0A446Q8P2, A0A446TIF8, A0A446PVA8, A0A446WSF5, A0A446K631, A0A446NAK8, A0A446TBV6, A0A446L425, A0A446MW67, A0A446XZR0, A0A446NK46, A0A446U2C0, A0A446JGI5, A0A446P474, A0A446K605, A0A446YZ88, A0A446M589, A0A446PV83, A0A446YAM4, A0A446LMA9, A0A446MWH4, A0A446QNY6, A0A446QH58, A0A446LHY0, A0A446KV42, A0A446M569, A0A446Q0X5, A0A446KV52, A0A446MVE3, A0A446MAN9, A0A446MUR3, A0A446M561, A0A446MVD1, A0A446X556, A0A446M5B3, A0A446R1T8, A0A446KUY6, A0A446LHX4, A0A446P114, A0A446MRM4, A0A446LHS8, A0A446UJA5, A0A446XNN9, A0A446NRV9, A0A446NRY6, A0A446LHY1, A0A446X3F2, A0A446NRX3, A0A446WT80, A0A446NRV2, A0A446TBX2, A0A446MRI3, A0A446NS43, A0A446MWZ7, A0A446WSF8, A0A446JGF3, A0A446X5C0, A0A446NYC9, A0A446WSC2, A0A446X5E5, A0A446MRJ3, A0A446MDA4, A0A446MRG9, A0A446Y558, A0A446UJ53, A0A446NRYO, A0A446KUV4, A0A446MV57, A0A446KUW8, A0A446LL74, A0A446LHW1, A0A446SWQ4, A0A446YTB1, A0A446YP03, R9UD00, A0A446M557, A0A446MRP0, A0A446NA00, A0A446L155, A0A446MVK1, A0A446MV76, A0A446LHY3, A0A446XU55, A0A446WTD2, A0A446WSD5, A0A446XS86, A0A446LI36, A0A446J7A5, A0A446LI37, A0A446YPB5, A0A446TQL2, A0A446NAE6, A0A446IP93, A0A446MD40, A0A446NRW2, A0A446MUS9, A0A446MV97, A0A446P468, A0A446USI9, A0A446MVI9, A0A446UJ90, A0A446J796, A0A446R1M6, A0A446KHK3, A0A446RQL8, A0A446VUB8, A0A446RQK5, A0A4461P14, A0A446KV31, A0A446LLA5, A0A446Y304, A0A446MUN3, A0A446MV33, A0A446KV62, A0A446KVC1, A0A446LL47, A0A446NRX4, A0A446L993, A0A446M585, A0A446IP36, A0A446UQG9, A0A446M527, A0A446VAR7, A0A446MUY8, A0A446L401, A0A446U9S4, A0A446Y557, A0A446M553, A0A446MV07, A0A446UWT0, A0A446NBI9, A0A446M528, A0A446LLF8, A0A446Q0V8, A0A446LI24, A0A446MUU3, A0A446YLJ5, A0A446MV00, A0A446MRJ2, A0A446TBW0, A0A446KUQ8, A0A446R9L4, A0A446MUL5, A0A446TBX1, A0A4461HX1, A0A446KV07, A0A446N0X4, A0A446K677, A0A446RZ10, A0A446MI12, A0A446V4H5, A0A446MV35, A0A446VQ63, A0A446MUM6, A0A446LLB3, A0A446QEZ2, A0A446PK75, A0A446XNN5, A0A446K6I0, A0A446TC97, A0A446M5A2, A0A446KV22, A0A446TRY9, A0A446NRW4, A0A446SK08, A0A446UWU1, A0A446MVP0, A0A446MUY6, A0A446L973, A0A446MW55, A0A446K6L6, A0A446XPB9, A0A446LHU8, A0A446J9R0, A0A446MD20, A0A446LI00, A0A446UZM1, A0A446MRL6, A0A446YRY6, A0A446MRN5, A0A446LHV3, A0A446R1U8, A0A446MW53, A0A446MVW1, A0A446N457, A0A446Q0X7, A0A446KUW1, A0A446LL55, A0A446L487, A0A446JGM8, A0A446QQX8, A0A446YZ82, A0A446Y148, A0A446WQF2, A0A446MIA1, A0A446NRU7, A0A446UJ72, A0A446XU56, A0A446L9A0, A0A446PV58, A0A446WSH4, A0A446SAW2, A0A446NS67, A0A446MRL5, A0A446MRK7, A0A446YTC2, A0A446RT60, A0A446YT98, A0A446WU68, A0A446UJ54, A0A446J7B3, A0A446VLN6, A0A446VUB4, A0A446Q9P0, A0A446LHV1, A0A446UJ60, A0A446MV39, A0A446MVB2, A0A446KV26, A0A446LHV4, A0A446KUW4, A0A446KV73, A0A446Q0W0, A0A446MV61, A0A446Y562, A0A446MV98, A0A446R7D5, A0A446MV30, A0A446KV89, A0A446IHW9, A0A446PV86, A0A446WVE1, A0A446MT54, A0A446L3Y7, A0A446MRL9, A0A446LLB2, A0A446MV41, A0A446RZ83, A0A446X395, A0A446U371, A0A446UW14, A0A446MUU4, A0A446QA53, A0A446SLI6, A0A446LM93, A0A446NRZ4, A0A446KVA1, A0A446PV26, A0A446L9A8, A0A446QLB6, A0A446MRN4, A0A446L4A2, A0A446UJ74, A0A446UW15, A0A446UJ12, A0A446WUB2, A0A446MRL4, A0A446MVE2, A0A446KUU1, A0A446SLN0, A0A446Y3A1, A0A446L430, A0A446Q8F8, A0A446SK49, A0A446KUU5, A0A446RQP4, A0A446Y2R3, A0A446LHV9, A0A446UJA0, A0A446NRV7, A0A446NRW6, A0A446UEC6, A0A446MRI7, A0A446MRJ5, A0A446SXG6, A0A446MRQ6, A0A446MZY9, A0A446P423, A0A446M576, A0A446Q9N8, A0A446LZ06, A0A446IP45, A0A446M592, A0A446X394, A0A446KUU6, A0A446LNU4, A0A446KUX7, A0A446M574, A0A446UJB1, A0A446MVG4, A0A446MVM9, A0A446LSA5, A0A446WSC9, A0A446YSZ5, A0A446VU91, A0A446MVE4, A0A446KV50, A0A446MUZ5, A0A446UJA3, A0A446N116, A0A446RDR4, A0A446L474, A0A446LLE4, A0A446MT19, A0A446UJA4, A0A446MRI4, A0A446LHS2, A0A446MVL2, A0A446Q9R4, A0A446YS15, A0A446MUR2, A0A446MRQ2, A0A446M578, A0A446KUZ2, A0A446RS84, A0A446QR39, A0A446UJ79, A0A446LHUO, A0A446MVA5, A0A446NRV8, A0A446R7D1, A0A446KUY2, A0A446LLE1, A0A446M599, A0A446UZL9, A0A446MV19, A0A446II24, A0A446MV85, M7YPH8, M7ZVA3, M7ZF50, M7YWV4, M7Z5B8, M7YN61, M8AE45, M7ZNB1, M7YXB0, M8AST1, M7YR24, M7ZA47, M7YT26, M7YJ40, M8ADD0, M7ZJA7, M7YQ54, M8AUM9, M8ANE0, M7ZXQ8, M8AUZ7, M7ZHG8, M7ZXN9, M7ZBE9, M7YEQ0, M7ZCG6, M8AAR3, M8A1P2, T1NTJ1, T1N2R3, T1LF75, T1N4J2, T1MJA6, T1M819, T1LC35, T1MQA5, T1LSX1, T1MIZ5, T1LNZ0, T1N671, T1MUZ3, T1NI12, T1NKJ6, T1NR99, T1LEM3, TiMOW0, TiNAZ0, T1MFP5, T1LZ01, T1NPH7, T1NHL1, T1MDZ0, T1NSO3, T1M9E3, TiLM18, T1LAC9, T1LBZ0, T1MXQ5, T1MQD4, T1MPW5, T1LM09, T1NPT5, T1LM19, T1LUO9, T1NK32, T1LUD5, T1NF00, T1NC78, T1NN82, T1MA35, T1L9J1, T1MBB1, T1LXJ4, T1LKE2, T1LGV7, TiMASO, T1MPD3, T1MSD0, T1LRP0, T1MF28, T1L9J3, TiMlJ0, T1NWB7, T1M5P2, T1NBE0, T1LGV4, T1LSN9, T1NGW2, TiLNY8, T1MDI5, T1NVC5, T1NHJ6, T1LSX0, T1L8X2, T1MZ83, T1LD28, T1LAN4, T1LAN5, T1NM62, T1LF74, T1LGV3, T1LF77, T1MQD3, T1LI56, T1NW29, T1N4G6, T1M6J5, T1LC36, T1M1I9, T1LHM4, T1MAK7, T1ME31, T1MKF8, T1LI57, T1LM22, T1LBJ3, T1LAN6, M7YNN7, M7Z646, M8A2P4, A0A0P1GFE6, A0A0P1GTJ8, A0A238J853, A0A239KUI1, A0A1V9XN76, A0A1V9XRJ6, A0A1V9XWL9, A0A1V9XIR5, A0A1V9X1B6, A0A1V9Y2Z8, A0A1V9X236, A0A1V9X4X2, A0A144ZKR4, L9J9W1, L9J9Q4, L8YOB6, A0A2K9L9B3, A0A2K9L3H7, A0A2K9L521, A0A2K9L700, A0A2K9L519, A0A2K9L1A9, A0A2K9L2G0, A0A2K9L0F4, A0A2K9L648, A0A2K9L6M7, A0A2U4CNJ2, A0A2U3V1A2, A0A2U4CNJ3, A0A2U4A725, A0A2U4CNU7, A0A2U4CNK8, A0A2U4BS66, A0A2U4A724, A0A093FWK3, A0A093FQ32, A0AIW5CXSO, A0AIW5CXL4, K2AG36, A0A212KZK8, A0A452RK74, A0A452RW34, A0A452QNT0, A0A452RK69, A0A3Q7VTE7, A0A3Q7W7U3, A0A3Q7WRV2, A0A3Q7XCY5, A0A3Q7WHD8, A0A3Q7V5V4, A0A3Q7X1Z0, A0A3Q7WRV6, A0A3Q7XCZ2, A0A3Q7XNM7, A0A384BVT0, A0A384DPH5, A0A384BP91, A0A384D4U8, A0A384BPA7, A0A384DNT9, A0A452VCB3, A0A452U7G2, A0A452VCC2, A0A452U7G7, A0A194V1F2, A0A194UVJ5, A0A194ULX3, A0A194VCV2, A0A194W0K5, A0A194W6W9, A0A194VR95, A0A194W4H9, A0A423VPM3, A0A423VSL1, A0A423WD94, A0A423W9F7, A0A423WZH0, A0A423WV28, A0A423WIT3, A0A423WH16, A0A423VR78, A0A423VA80, A0A0S3PPN4, A0A1E7U9P5, A0A250DK61, E6V8Z5, A0A0P9AFP2, A0A2G6X860, A0AII5PZ82, A0A2N4SJ19, A0A2E9C131, A0A370IXJ3, A0A370JCW3, A0A2V5TIP4, A0A2D5MT73, A0A0D1XT13, A0A0D1YG70, C9SWV4, G2XFJ0, A0A2J8BUS4, A0A444SAT6, A0A366Q651, A0A0G4M299, A0A0G4MAI0, A0A0G4MID9, A0A0G4MA18, A0A3M9Y2Y5, A0A0S3SI41, A0A0S3S9L1, A0A0S3SVK9, A0A0S3RUP5, A0A0S3RDA5, A0A0S3S2D1, A0A0S3SFB8, A0A0S3SI12, A0A0S3S983, A0A0S3SHB1, A0A0S3S2C4, A0A0S3RAQ9, A0A0S3SI52, A0A0S3SBH8, A0A0S3SHD6, A0A0S3RUY5, A0A0S3RV54, A0A0S3SWB4, A0A0S3SI31, A0A0S3SIJ7, A0A0S3S2E3, A0A0S3T100, A0A0S3SAD2, A0A0S3SFJ3, A0A0S3S9H7, A0A0S3QZQ6, A0A0S3SKU3, A0A0S3SHG6, A0A0S3S9C6, A0A0S3RUP4, A0A0S3SYJ7, A0A0S3RUT8, A0A0S3SII5, A0A0S3RTMO, A0A0S3QZS6, A0A0S3RQC6, A0A0S3SBP8, A0A0S3SKN8, A0A0S3S9A9, A0A0S3S9H4, A0A0S3S2C1, A0A0S3S9B5, A0A0S3RV98, A0A0S3RBJ5, A0A0S3QZN0, A0A0S3SHY7, A0A0S3SA88, A0A0S3SU71, A0A0S3SVC8, A0A0S3RUR6, A0A0S3SHH7, A0A0S3RUS8, A0A0S3SB63, A0A0S3T043, A0A0S3RZL4, A0A0S3S969, A0A0S3RZ03, A0A0S3SB96, A0A0S3QZV8, A0A0S3SHB5, A0A0S3S9G1, A0A0S3QZX3, A0A0S3S2F1, A0A3Q0FG34, A0A1S3UY68, A0AIS3URP2, A0AIS3URH0, A0A3Q0ET99, A0A3Q0EYW5, A0AIS3TL02, A0A1S3W0Y9, A0A1S3U2W6, A0A1S3W0T2, A0A1S3W0T7, A0A1S3W144, A0A1S3U4J5, A0AIS3TNA8, A0A1S3T9S3, A0AIS3VJI6, A0AIS3TMS8, A0A3Q0ESJ5, A0AIS3TLG9, A0AIS3VUM7, A0AIS3TUL8, A0AIS3TSI3, A0A1S3UON3, A0A3Q0EZS4, A0A1S3VLI3, A0A1S3U6F1, A0A3Q0EVU4, A0A3Q0ET57, A0AIS3VJJ8, A0A1S3T8I0, A0AIS3W0Y5, A0A3Q0F2X9, A0AIS3TGW0, A0AIS3VEP0, A0AIS3VT28, A0A1S3U039, A0A3Q0FL44, A0A1S3W0L1, A0A1S3VU23, A0A3Q0EIT8, A0A3Q0EQK2, A0A1S3TYK8, A0A1S3U5X8, A0AIS3TUD5, A0A3Q0F6W5, A0A1S3U6A4, A0AIS3TSG7, A0A3Q0ERZ1, A0A3Q0FGU0, A0A3Q0EP47, A0A3Q0ELC1, A0A1S3U943, A0A3Q0EVT5, A0A3Q0F3I1, A0AIS3VE13, A0AIS3VKA1, A0AIS3TI08, A0A3Q0FK37, A0A1S3VLA4, A0A3Q0F5J2, A0A1S3U186, A0A3Q0FKN7, A0A3Q0FHK9, A0A1S3TXM9, A0A1S3U6A9, A0A1S3TLE5, A0A1S3W149, A0A1S3U6B7, A0AIS3VCH7, A0AIS3TU21, A0A1S3U951, A0A3Q0F0J3, A0A3Q0EUC6, A0A3Q0EX51, A0A1S3USU6, A0A1S3VJQ9, A0A1S3VSU2, A0A3Q0FH19, A0A3Q0EQ28, A0A3Q0EWF0, A0A3Q0F0F3, A0A3Q0FGP8, A0AIS3VBZ6, A5C4W8, A0A438HK40, F6GZ95, A0A438HSA8, A0A438K4K8, F6H2A3, F6H2N4, A0A438HPG9, F6GXQ1, F6GYD8, F6HRT0, A5BHL6, A5B7V9, A0A438IDB7, A0A438JV19, A0A438J4D4, A0A438J4P8, F6H2B3, F6HL29, A5BJG8, A5AH51, A5BYN3, F6H549, F6HD38, F6HD40, F6HL32, A5BFJ9, A5ALH9, F6I0H1, F6GZ96, F6H296, D7U4I0, F6I7L4, A5ACT5, F6H5E3, A5BH08, F6H5E2, A5BRV2, A5AD43, F6HW07, F6HL33, F6GYE5, A0A438G9R9, A0A438GDMO, A0A438H0P0, A5ASQ2, F6GXQ3, F6HJX6, F6GZ98, A5BY61, A5B986, D7T5C7, A5C5N7, E0CSX4, F6H7Z9, D7TJ25, A5BIS9, F6H543, F6GZX7, A5BXA4, F6H1X1, A5BQP9, F6H2A5, A5BH06, D7TRQ9, A0A438HNC4, A5BKS7, D7UAZ5, D7U6R5, A5ALKO, F6HL31, F6H2G3, A5AQY5, F6HYF4, F6H2B6, F612E8, E0CSZ7, F6H2G5, F6HL30, F611Y7, A5BRU4, A5BQQ0, F6H298, F6H2C0, A5C1U2, F6GZ97, F6HH96, F6H2A9, F6HW06, A5BXZ3, A0A438EUR3, A0A438DP60, A0A438IDB3, F6GYG6, A0A438G921, A0A438BSL5, A0A438G9P6, A0A438HN97, A0A438GDR0, A5AG42, A0A438GF75, A0A438HLK8, A0A438G9V2, A0A438G9Q8, A0A438H0K0, A0A438ID79, A0A438HSB1, A0A438HK35, A0A438G270, A0A438G9S5, A0A438HAT9, A5BHL5, A5AUE6, A0A438BPR1, F6H559, A0A438BSE3, A0A438DBA0, A0A438CR06, A0A438BPQ2, A0A438G973, A0A438JAW2, A0A438FCT8, A0A438HSB6, A0A438JS26, A0A438EA03, A0A438GV90, A0A438CSM6, A0A438HS93, A0A438BPP3, A0A438GVE3, A0A438CK55, A0A438G8Z3, A0A438BSB7, A0A4381TA4, A0A438K4I2, A0A438BSD0, A0A438FQF7, A0A438C0M0, A0A438GDR3, A0A438BSF9, A0A438G3D9, A0A438KNQ7, A0A438G911, A5BHL9, A0A438EGN8, F6H2A8, A0A438J4V0, A0A438K4L8, A0A438GDM4, A0A438DY93, A0A438HN88, A0A438HN57, A0A438GVF0, A0A438GDM2, A0A438GVA2, A0A438BS99, A0A438DBE8, A0A438JAQ1, A0A438DP71, A0A438BS80, F6H2A0, A0A438G9S9, A0A438HU56, A0A438BPN8, A0A438HS63, F6GXQ9, A0A438GVC3, A0A438FWU9, A0A438GDN5, A0A438GDN4, A0A438GDQ3, A0A438EUS9, A0A438E5D3, A0A438I408, A0A438G271, A0A438DE69, F6GYD9, A0A438HPI4, A0A438BPY9, A0A438BSI7, A0A438I405, A0A438BS96, A0A438K4H2, A0A438GDQ0, A0A438C054, A0A438H4L4, A0A438G257, A0A438BSC6, A0A438G9Q4, A0A438BPQ7, A0A438HSC6, A0A438GVB8, A0A438IDD7, A0A438HPK3, A0A438G9Q3, A0A438BSC1, A0A438G5Q0, A0A438BSM6, A0A438BSC0, A0A438DP46, A0A438DGJ9, F6GYE3, A0A438JLB1, A0A438IDA7, A0A438GVD0, A0A438GDQ6, A0A438DF00, A0A438DP50, A0A438G9P1, A0A438GBQ1, A0A438JKA7, A0A438KF31, A0A438IDB0, A0A438G907, A0A438H6B4, A0A438GV93, A0A438G943, A0A438CYC5, A0A438BSE2, A0A438BSD9, A0A438DB91, A0A438G9Q7, A0A438BSA2, A0A438GVA1, A0A438GDN6, A0A438FCSO, A5AQ78, A0A438C802, A0A438HLD8, A0A438HZ10, A0A438GVD3, A0A438G908, A0A438GVB4, A5BLUO, A0A438HPJ7, A0A438G5P6, A0A438DE77, A0A438C111, A0A438HCT6, D7SRN6, F6GXQ6, A5BKZ4, A0A438BSD1, F6H2G7, F6GZX9, F6GZD2, F6H294, F61011, A5ACT7, A5BRU5, A5BVG8, F6H542, F6HL34, F612A7, F6H291, E0CT00, A5AQI0, A5C9P3, F6HFB8, F6GXQ0, A5BGQ9, E0CSJ3, F6HRS4, A5C0J8, F6HD39, F6H801, F611Y9, F6H2B7, F6GZX6, F6H2G6, F6GYE4, A5BEU8, E0CT03, F6H295, F612E9, A0A438GVB5, A0A438DPA7, A0A438DP66, A0A438HAT4, A0A438CQW8, A0A438HLG1, A5C437, F6GZX1, A0A438DBZ7, A5BEK3, D7TK53, F6HYF3, F6H329, A5BIW6, A0A438D3S7, A5APY4, F6HL08, F6H2A2, A5BHL4, A0A438EA11, A0A438CFB9, A0A438G8X0, A0A438EAE1, A0A438GDP7, A0A438BPQ9, A0A438JP69, A0A438HLN0, A0A438G975, A0A438HPKO, A0A438BSA0, A0A438FHJ0, A0A438G906, F6GXR2, A0A438G919, A0A438F254, A0A438EGP7, A0A438BS88, A0A438HS95, A0A438GVD6, A0A438E9X1, A5AWEl, A0A438GVB1, A0A438G952, A0A438IDD5, A0A438G9P4, A0A438BSF2, A0A438G5Q1, A0A438HS43, A0A438JK94, A0A438JAS5, A0A438ID88, A0A438ITJ2, A0A438HLS7, A0A438DP63, A0A438HPH4, A0A438BPR7, A0A438G918, A0A438JAY5, A0A438G9T4, A5BFKO, A0A438BSE1, A5C187, F6H290, F6I1Y8, A5AS83, A5C1Y4, F6H292, A5AST3, F6HCT4, A0A438HS08, A0A438IT97, A0A0G4H1K6, A0A0G4FPW0, A0A0G4EVW8, A0A0G4G2P6, A0A0G4EX32, A0A0G4EK41, A0A0G4GV94, A0A0G4FK32, A0A0G4FSG0, A0A0G4GBE1, A0A0G4FEA5, A0A0G4G667, A0A0G4G5L3, A0A0G4GUX0, A0A0G4H7X7, A0A0G4FED4, A0A0G4GC70, A0A0G4EL21, A0A0G4EFA5, A0A0G4FS69, A0A0G4G6X5, A0A0G4GSW5, A0A0G4FB70, A0A0G4FJV3, A0A0G4ELW9, A0A0G4F302, A0A0G4H591, A0A0G4GFR4, A0A0G4FNT8, A0A0G4H797, A0A0G4FKH8, A0A0G4GBQ5, A0A0G4G8Z9, A0A0G4FBG5, A0A0G4FUC6, A0A0G4EESO, A0A0G4GY87, A0A0G4FN79, A0A0G4FG97, A0A0G4GUO4, A0A0G4GAX4, A0A0G4EKA7, A0A0G4E9C4, A0A0G4GA99, A0A0G4GC21, A0A0G4F4H5, A0A0G4GTL7, A0A0G4EC69, A0A0G4EDQ0, A0A0G4FIH0, A0A0G4GX24, A0A0G4GSZ4, A0A0G4GQI0, A0A0G4FAD2, A0A0G4EYY7, A0A0G4ECR3, A0A0G4FRN1, A0A0G4F3I1, A0A0G4GJ27, A0A0G4FWKO, A0A0G4FT88, A0A0G4ER04, A0A0G4FH06, A0A0G4GJ92, A0A0G4EN10, A0A0G4FC64, A0A0G4G604, A0A0G4H617, A0A0G4EE30, A0A0G4EMH2, A0A0G4EXD3, A0A0G4F467, A0A0G4ESG9, A0A0G4H5A7, A0A0G4H147, A0A0G4ELP5, A0A0G4EW80, A0A0G4EYN3, A0A0G4EVX9, A0A0G4G194, A0A0G4FYF0, A0A0G4ENT8, A0A0G4E913, A0A0G4EZ90, A0A0G4GDZ1, A0A0G4EV77, A0A0G4F2L7, A0A0G4FW07, A0A0G4END2, A0A0G4G025, A0A0G4EJ16, A0A0G4G3W1, A0A0G4ED81, A0A0G4EA18, A0A0G4GZE5, A0A0G4FZK6, A0A0G4F356, A0A0G4GY92, A0A0G4EP61, A0A0G4ERN6, A0A0G4F257, A0A0G4FG25, A0A0G4GJ87, A0A0G4GFW8, A0A0G4GN70, A0A0G4EGD8, A0A0G4GV35, A0A0G4EDK7, A0A0G4FDV3, A0A0G4EN36, A0A0G4FU39, A0A0G4G6I2, A0A0G4FAX9, Q948Y6, D8U662, D8UDJ2, D8UHS7, D8UHS1, D8U663, D8TZF2, D8TWV3, D8TZP2, D8ULZ4, D8UOL1, D8THZ2, D8U661, D8U9Q0, D8TUD4, D8TWS3, D8U5V3, D8TWS6, D8U656, D8UHS6, D8UOK8, D8TWV6, D8U3S1, A0A3Q7TWA6, A0A3Q7T0P7, A0A3Q7SNP7, A0A3Q7V334, A0A3Q7TKS7, A0A3Q7TAD2, A0A3Q7TW96, A0A3Q7UEY3, A0A0D0QEL7, A0A0D0PB00, A0AIM6EL45, A0A0C9S462, A0A0C9QPG3, A0A0C9RV88, A0A0C9RSUO, A0A0C9QPV3, A0A0C9RSN7, A0A0C9S6M8, A0AII8E9A3, J9DND2, A0A3P7DL77, A0A1I8ELN8, A0A3P7EWD2, A0A1I8EZC2, J9ETT3, A0AII8EY91, A0A1I8EL26, A0A1I8EZH7, A0A3P7GM02, J9DZA9, A0A3P7FT60, A0A3P7GCY4, J9E1R8, A0AII8EFL2, A0AII8EF35, A0A3P7EEQ9, A0A3P7FCC9, A0A3P7DVJ1, A0A3P7DV86, A0AII8ET18, A0A3P7DDB0, A0A3P7DLR8, A0AII8F0Y1, J9F728, J9F5K7, A0AII8ELS4, A0A3P7EE66, A0AII8EA12, J9ELM2, J9EVZ5, J9E6Y8, J9ESJ0, J9AHN9, A0A3P7GJ34, A0AII8ESS3, J9E0L8, J9EHN5, A0AII8ECU1, J9FCK3, A0A3P7FKH0, A0A3P7EG90, J9F005, J9FHZ7, J9F0L7, J9EDU4, A0A1I8EPI5, A0A1I8ENJ3, A0A1I8EHS2, A0A3P7E3Y0, A0A1I8ER89, J9AZF1, J9B8V2, A0A1L8G2F6, Q91691, A0A1L8GHB0, A0A1L8G2M6, A0A1L8GP52, Q7ZTN9, A0AIL8GTY1, A0AIL8GZD1, Q6GP14, Q6AZS7, P70006, F7EDW8, F6YRB8, F7AR31, Q91402, A0A3P5XFD2, A0A3B5KRY2, A0A3B5KXI9, A0A3B5KR76, A0A3B5MPU6, A0A3B5M999, A0A3B5KPF4, A0A3B5KR79, A0A3B5KQW4, A0A3B5MUP9, A0A3B5M3B3, A0A3B5MDS7, A0A3B5KJF0, A0A3B5MW25, M4ASA0, A0A3B5R264, M4AQQ5, A0A3B5R3X9, M4AAJ3, M3ZXK8, M3ZTSO, M3ZXL1, M3ZXJ9, M3ZQC7, A0A3B5PQ81, A0A439DFT7, A0A439CN67, A0A439CTF6, A0A2E1D5Y3, A0A2T6KMQ7, A0AII6G2U0, A0A3L6D9L1, A0A3L6E7F7, A0A317YAC8, A0A3L6DRV9, A0A3L6E476, A0A317YET9, A0A3L6E218, A0A317YKF8, B6TY46, A0A317Y521, P17801, A0AID6KQI4, C0P6F5, A0A1D6KH09, A0A096T7Z1, A0A1D6MOL4, A0A1D6LBB2, A0A1D6DXI3, A0A1D6F8W0, A0A1D6N9S7, A0A1D6DXH8, A0A1D6H9Q0, A0A1D6GN29, K7UZN2, A0AID6IEQ3, A0A1D6P507, A0AID6DXM7, A0A1D6J6V6, K7VD35, A0A1D6IAX9, C0P9N1, A0A1D6JF78, A0A1D6GA13, A0A1D6FGJ5, K7VRP4, A0A1D6DXM5, A0A1D6ML66, A0A1D6GA09, A0A1D6N9S3, A0A1D6HLB4, A0A1D6IER4, A0A1D6N9Q9, C0HFM8, A0A1D6KIF9, A0A1D6IEQ5, A0A1D6GA12, A0AID6KAQ9, A0AID6IEI3, A0AID6FSS8, A0AID6IEI1, A0A1D6N9R8, A0A1D6MFC2, A0A1D6M2E6, A0A1D6J6V7, A0A3L6E4L3, A0A1D6DXH6, A0A1D6J6V1, A0A1D6J6V2, A0A1D6MON6, A0A1D6EWT3, B4G1J2, A0A1D6ITQ0, A0A1D6KQI3, A0A1D6KL97, A0A1D6N9R3, K7UIF8, A0A1D6MON7, A0A1D6EB48, A0A1D6QAA8, A0A1D6P2B1, A0A1D6MON8, A0A3L6DU79, A0A3L6EU45, A0A317Y2S5, B7ZXB0, A0A3L6FU88, A0A3L6DL74, B6U2B7, A0A3L6FAJ7, A0A3L6DY96, A0A1Q1B749, A0A3L6ET80, B6SWL2, A0A3L6GBB9, A0A3L6G7X6, A0A3L6E4K1, A0A3L6D8A7, A0A3L6DCB9, A0A3L6DDA9, B4F952, A0A3L6G155, 024571, A0A3L6G2G8, B6ST43, A0A3L6FWI8, A0A3L6EU60, B4FYC6, A0A1V0E7H4, B4F8X7, A0A1D6MOMO, A0A1D6KIH3, A0A1D6N9S8, A0A1D6G7A2, A0A1D6KNU3, A0A1D6P015, K7TKD2, C0P3V1, A0A3L6G196, A0A1D6DXM6, A0A1D6HWK7, A0A1D6DXF1, A0A1D6IEQ4, A0A1D6KII4, B4FB05, A0A1D6IBG7, A0A1D6DXI2, A0A1D6N9R2, K7UC64, A0A1D6G136, A0A1D6LHG9, A0A1D6Q5J8, A0A1D6DXH9, A0A1D6FSS9, A0A1D6DXI7, A0A1D6G137, A0AID6IEI2, A0AID6JTK4, A0AID6JF77, K7U3E7, A0AID6QHL5, A0AID6HLB7, A0A1D6I5R1, A0AID6GA10, A0AID6LGF6, A0AID6DXM4, A0AID6EUL5, A0A317YAM4, A0A3L6ETW0, A0A3L6E3M3, 049974, A0A3L6FYL3, A0A3L6EW75, A0A317Y7G4, A0A317Y3N4, A0A3L6FI05, A0A3L6DN41, C0PE10, A0A3L6G6F6, A0A3L6G1E6, A0A3L6DU70, C0HEN9, A0A3L6G8Y0, A0A3L6FT98, A0A3L6EX22, A0A3L6EHP0, 082103, A0A3L6DLS3, B7ZXT8, A0A3L6F189, A0A3L6FL65, A0A3L6E7I6, B7ZWT1, A0A3L6G679, A0A3L6DII2, A0A3L6FF64, A0A3L6DKE6, A0A3L6FXI5, A0A3L6GCE3, A0A3L6FWN1, A0A3L6FGU1, A0A3L6GBY8, A0A3L6EFC7, A0A3L6FT74, A0A3L6EBY7, A0A317Y6J9, C0P3U3, A0A0AIXIZI, A0A0A1XS55, A0A0A1X650, A0A0A1XQ87, A0A0A1X1C0, A0A0AIXB00, A0A0A1X8W7, A0A0AIWII0, A0A0AIXIF3, A0A0AIWSF6, A0A2G1QQ59, A0A067RG78, A0A067RN91, A0A067R6A7, A0A067RBZ2, A0A067RU5, A0A0K9NV78, A0A0K9Q5Q1, A0A0K9PK14, A0A0K9P5Q8, A0A0K9NYD2, A0A0K9PEI3, A0A0K9PPY6, A0A0K9PXR7, A0A0K9PLY7, A0A0K9NWC4, A0A0K9PKC6, A0A0K9NQT5, A0A0K9P3S2, A0A0K9PQYO, A0A0K9PLW0, A0A0K9P3C4, A0A0K9PPJ9, A0A0K9P0C4, A0A0K9PY30, A0A0K9PNK6, A0A0K9PP88, A0A0K9P1W2, A0A0K9NS31, A0A0K9NWR2, A0A0K9NMN3, A0A0K9NJ97, A0A0K9PD87, A0A0K9NTS5, A0A0K9Q4R1, A0A0K9NIZ5, A0A0K9NQH7, A0A0K9PG70, A0A0K9NRX4, A0A0K9NV14, A0A0K9NLX9, A0A0K9P5H7, A0A0K9NPE2, A0A0K9P819, A0A0K9PRP8, A0A0K9P5Q0, A0A0K9PM56, A0A0F4GX70, A0A0F4GNN3, A0A0F4GDY3, A0A0F4G7R0, A0A0F4GJL4, A0A0F4G525, A0A0F4GKZ5, A0A0F4GK64, A0A0F4GK12, A0A0F4GVA7, A0A0F4GTQ5, A0A0F4GJJ0, A0A0F4GPQ0, A0A0F4GLI2, F9X4T1, F9XHT4, F9XH72, F9XJG6, F9XDZ3, F9X146, F9XK84, F9XCF6, F9XP87, F9X6W1, F9XFV9, F9XM39, F9XAK9, F9XNE3, A0A1Y6LP08, A0A1Y6LZ79, A0A1Y6LY30, A0A1Y6LT89, A0A1Y6L814, A0A1Y6LQH5, A0A1Y6LQU6, A0A1Y6L5Q9, A0A1Y6LXG6, A0A1Y6LN68, A0A1Y6LFZ2, A0A1Y6LBN0, A0A1Y6LDV9, A0A1Y6L8C9, A0A1Y6LRA7, A0A2H1FJ88, A0A2H1GP22, A0A2H1H488, A0A2H1FZK2, A0A2H1H533, A0A2H1FLY6, A0A2H1H4S8, A0A2H1GT42, A0A2H1GHG1, A0A2H1H5M8, A0A2H1GTE8, A0A2H1FQ02, A0A2H1G5G9, A0A2H1GXW6, A0A2H1GZKO, A0A2H1GNUO, A0A2H1GST3, A0A2H1H7P5, A0A1X7RYL6, A0A1X7RF14, A0A1X7RL97, A0A1X7RUL5, A0A1X7S239, A0A1X7RV33, A0A1X7RNL6, A0A1X7RY43, A0A1X7S9D0, A0A1X7S2M2, A0A1X7S7L9, A0A1X7RBY3, A0A1X7S0P9, and A0A1X7RID6.

1.-7. (canceled)

8. A method for enhancing immune response to a pathogen in a plant comprising inactivating a gene encoding a PAN domain-containing protein in the plant.

9. The method of claim 8, wherein the inactivating is achieved by introducing a non-silent mutation in the gene at a position encoding a conserved cysteine residue of the PAN domain.

10. The method of claim 8, wherein the PAN domain-containing protein is selected from plant G-type receptor-like protein kinases.

11. The method of claim 9, wherein the conserved cysteine residue is selected from a residue equivalent to a cysteine at position 1, 32, 36, 40, 42, or 57 of SEQ ID NO: 25.

12. The method of claim 9, wherein the non-silent mutation is a nonconservative mutation.

13. The method of claim 12, wherein the nonconservative mutation is a cysteine to alanine mutation.

14. The method of claim 8, wherein the inactivating of the gene encoding a PAN domain-containing protein is achieved by introducing an exogenous nucleic acid inhibitor of the gene to the plant.

15. The method of claim 14, wherein the nucleic acid inhibitor is selected from the group consisting of an antisense RNA, a small interfering RNA, an RNAi, a microRNA, an artificial microRNA, and a ribozyme.

16. The method of claim 8, wherein the inactivating of the selected gene is achieved by genome editing, which is achieved by a method selected from the group consisting of CRISPR/Cas system, Cre/Lox system, TALEN system, ZFNs system and homologous recombination.

17. The method of claim 16, wherein the CRISPR-mediated genome editing comprises introducing into the plant a first nucleic acid encoding a Cas9 nuclease, a second nucleic acid comprising a guide RNA (gRNA), wherein said gRNA is specific to the gene encoding a PAN domain containing protein.

18. The method of claim 16, wherein the CRISPR-mediated genome editing comprises introducing into the plant a first nucleic acid encoding a Cas9 nuclease, a second nucleic acid comprising a guide RNA (gRNA), wherein said gRNA is specific to the gene encoding a PAN domain containing protein gene, and a third nucleic acid comprising a template for homologous recombination.

19. The method of claim 8, wherein the pathogen is selected from a bacterium, a virus or a fungus.

20. A genetically engineered plant, wherein an endogenous gene encoding a PAN domain-containing protein has been inactivated as a result of a genetic modification.

21. The genetically engineered plant of claim 20, wherein the inactivating is achieved by introducing a non-silent mutation in the gene at a residue encoding a conserved cysteine residue of the PAN domain.

22. The genetically engineered plant of claim 20, wherein the PAN domain-containing protein is selected from G-type receptor-like protein kinases.

23. The genetically engineered plant of claim 21, wherein the conserved cysteine residue is selected from a residue equivalent to a cysteine at position 1, 32, 36, 40, 42, or 57 of SEQ ID NO: 25.

24. The genetically engineered plant of claim 21, wherein the non-silent mutation is a nonconservative mutation.

25. The genetically engineered plant of claim 24, wherein the nonconservative mutation is a cysteine to alanine mutation.

26.-59. (canceled)
